(19) United States
(12) Patent Application Publication
(10) Pub. No.: US 2018/0016314 A1
(43) Pub. Date: Jan. 18, 2018

(54) TREATMENT OF DISEASE VIA TRANSCRIPTION FACTOR MODULATION

(71) Applicant: Children's Hospital Medical Center, Cincinnati, OH (US)

(72) Inventors: John Barker Harley, Cincinnati, OH (US); Leah Kottyan, Cincinnati, OH (US); Matthew Weirauch, Cincinnati, OH (US)

(21) Appl. No.: 15/647,672

(22) Filed: Jul. 12, 2017

Related U.S. Application Data
(60) Provisional application No. 62/361,174, filed on Jul. 12, 2016.
(60) Provisional application No. 62/385,197, filed on Sep. 8, 2016.
(60) Provisional application No. 62/455,649, filed on Feb. 7, 2017.
(60) Provisional application No. 62/459,326, filed on Feb. 15, 2017.
(60) Provisional application No. 62/479,685, filed on Mar. 31, 2017.

(51) Int. Cl. C07K 14/47; C12Q 1/68; C07K 16/28; A61K 31/00

(52) U.S. Cl. CPC C07K 14/4702; C07K 16/2896; C07K 16/2812; C12Q 1/6806; C07K 16/2884; A61K 31/00; C12Q 1/6853

(57) ABSTRACT

Disclosed herein are methods of treatment of various disease states in which an individual in need thereof if administered one or more therapeutic agents capable of modulating one or more transcription factors. Also disclosed are methods by which an individual may be treated for one or more disease states, in which loci in which transcription factors bind are detected.

### CROSS REFERENCE TO RELATED APPLICATIONS

This application claims the benefit of and priority to U.S. Provisional Patent Application Ser. No. 62/361,174, filed Jul. 12, 2016, entitled “Role for Epstein-Barr Virus EBNA2 in Autoimmunity,” U.S. Provisional Patent Application Ser. No. 62/385,197, filed Sep. 8, 2016, entitled “Transcription Factors Operating Across Disease Loci:EBNA2 in Autoimmunity,” U.S. Provisional Patent Application Ser. No. 62/455,649, filed Feb. 7, 2017, entitled “Drug Discovery in Lupus with Allele Specific Reporters,” U.S. Provisional Patent Application Ser. No. 62/459,326, filed Feb. 15, 2017, entitled “Drug Discovery in Lupus with Allele Specific Reporters,” and U.S. Provisional Patent Application Ser. No. 62/479,685, filed Mar. 31, 2017, entitled “Drug Discovery for Allele Specific Gene Regulation,” the contents of which are incorporated herein in their entirety for all purposes.

### STATEMENT REGARDING FEDERALLY SPONSORED RESEARCH

This invention was made with government support under A1024717 awarded to the National Institutes of Health. The government has certain rights in the invention.

### BACKGROUND

While modern medicine had advanced treatments for many different diseases, there remains an unmet need for treatment of disease and/or conditions associated with or contributing to disease states for which additional treatment is needed or for which no treatment currently exists. The instant disclosure addresses one or more such needs in the art.

### BRIEF SUMMARY

Disclosed herein are methods of treatment of various disease states in which an individual in need thereof if administered one or more therapeutic agents capable of modulating one or more transcription factors. Also disclosed are methods by which an individual may be treated for one or more disease states, in which loci in which transcription factors bind are detected.

### BRIEF DESCRIPTION OF THE DRAWINGS

The application file contains at least one drawing executed in color.

Copies of this patent or patent application publication with color drawing(s) will be provided by the Office upon request and payment of the necessary fee.

FIGS. 1-132 depict disease states and transcription factors (TFs). The X-axis displays disease associated loci. The Y axis displays the top TFs, based on the RELI P-value (Pc<0.01), sorted by the number of loci they occupy. A gray box indicates that the given locus contains at least one variant associated with the disease of interest located within a ChIP-seq peak for the given TF. The most significant ChIP-seq dataset cell type for the given TF is indicated in parentheses. TFs that participate in “EBNA2 super-enhancers” are in grey.

FIGS. 133A-133G. Intersection between autoimmune loci and TF binding interactions with the genome. FIG. 133A. Intersect between TF ChIP-seq datasets and SLE risk loci. The X-axis displays SLE-associated loci (P<5×10⁻⁸). The Y-axis displays the top 25 TFs, based on the RELI P-value (Pc), sorted by the number of loci they occupy. A colored box indicates that the given locus contains at least one SLE-associated variant located within a ChIP-seq peak for the given TF. The most significant ChIP-seq dataset cell type for the given TF is indicated in parentheses (all are EBV-infected B cell lines). TFs that participate in “EBNA2 super-enhancers”25 are colored red. The red rectangle identifies those loci and TFs that optimally cluster together. Bottom panel, left: comparison of EBV-infected B cell lines (grey bars) to EBV negative B cells (white bars). The Y-axis shows the distribution of the RELI −log (Pcs) for each of the eight TFs with available data. Bars indicate mean. Error bars indicate standard deviation. Red dots indicate the most extreme data point. Horizontal dashed line indicates the Pc<10-6 RELI significance threshold used in this study. Bottom panel, right: The top 10 TFs (based on RELI Pc-values) with data available in at least one EBV-infected B cell line (grey bars) and at least one other cell type (white bars). FIGS. 133B-133G. Results for the other six EBNA2 disorders. Full results are available in FIG. 148A-G.

FIGS. 134A-134D. Properties of EBNA2-bound autoimmune disease loci. FIG. 134A depicts a schematic of the RELI algorithm. FIG. 134B depicts TFs intersecting loci also occupied by EBNA2 at autoimmune risk loci. The RELI algorithm was re-executed using EBNA2 disorder variants intersecting EBNA2 ChIP-seq peaks as input. The results thus identify potential EBNA2 co-factors at EBNA2 disorder risk loci. The most synergistic TFs are indicated. NFκB subunits are shown in red. Members of the basal transcriptional machinery are shown in blue. FIG. 134C. Most EBNA2-occupied loci are associated with only a single EBNA2 disorder. EBNA2-bound loci were categorized by the number of EBNA2 disorders with which the given locus is associated (X-axis). The Y-axis indicates the number of loci in each category. FIG. 134D. Functional properties of EBNA2 disorder EBNA2-occupied loci. Functional importance of EBNA2-occupied loci, assessed with four criteria—intersection with eQTLs in EBV-infected B cells (top left), intersection with RNA Pol-II ChIP-seq peaks in EBV-infected B cells (top right), intersection with “super-enhancers” in GM12878 cell lines (bottom left), and intersection with “active chromatin states44” in EBV-infected B cells (bottom right). Variants are segregated into two categories—all common variants (minor allele frequency >1%) (left bars) and common variants associated with at least one EBNA2 disorder (right bars). Each category is divided into three types of variants—the full set of variants (blue bars), variants located within open chromatin regions in EBV-infected B cells (as indicated by DNase-seq peaks) (red bars), and variants located within EBNA2 ChIP-seq peaks (black bars). The Y-axis of each plot indicates the percent of variants in each group that are, for example, eQTLs in EBV-infected B cells (top left plot). Error bars indicate results from sampling (with replacement) of 50% of the variants in each category. Horizontal bars at the top indicate sampling-derived P-values based on Welch's one-sided t-test.

FIGS. 135A-135D. Allele-dependent binding of EBNA2 to autoimmune-associated genetic variants. FIG. 135A. Theoretical models explaining allele-dependent action of EBNA2. FIG. 135B. Allelic co-binding of EBNA2 with multiple proteins. ChIP-seq datasets from EBV-infected B cell lines were examined for evidence of allele-dependent binding at heterozygotes. Datasets are sorted by the proportion of EBNA2 GM12878 allelic events (MARIO ARS value >0.40, see Supplementary Methods) that favor the same allele (X-axis). Values (N) indicate total number of variants. FIG. 135C. Allele-dependent binding of EBNA2 and human proteins at the CD44 locus. Top to bottom: chromosomal band (multi-colored bar), location of EBV-infected B cell line ChIP-seq peaks for various TFs, location of rs3794102 variant, allele-dependent binding events (green bars). The X-axis indicates the preferred allele, along with a value indicating the strength of the allelic behavior, calculated as one minus the ratio of the weak to strong reads (e.g., 0.5 indicates the strong allele has twice the reads of the weak allele). FIG. 135D. Allele and EBV-dependent expression of CD44. Allelic qPCR of CD44 expression in EBV positive and EBV negative Ramos B cells. Fold-change in expression is given relative to the C (reference) allele. Error bars represent standard deviation (n=4). P-values were calculated using a two-way ANOVA with a Tukey post-hoc test. EBV status and variant genotype were used as the two factors.

FIGS. 136A-136D. Global view of cell types and TFs at disease-associated loci. FIG. 136A. SLE variants significantly intersect H3K27ac-marked regions in EBV-infected B cells. H3K27ac ChIP-seq peaks were collected from 175 different cell lines and types. The Y-axis indicates the negative log of the RELI P-value for the intersection of SLE-associated variants with H3K27ac peaks in each dataset. The 77 different EBV-infected B-cell lines are shown as red bars; all other cell types are shown as gray bars, except for the primary B cell dataset, which is in black. FIG. 136B. SLE variants intersect active chromatin regions in EBV-infected B cells. Same as (a), but instead using “active chromatin” regions, which are based on combinations of histone marks44. FIG. 136C. Global view of RELI results—all diseases against all TFs. Columns and rows show the 94 phenotypes/diseases and 212 TFs with at least one significant (Pc<10⁻⁶) RELI result. Color indicates negative log of the RELI P-value (see key). FIG. 136D. Cluster of TFs at breast cancer loci. Intersection between disease loci with TF-bound DNA sequences, as in FIGS. 133A-133G. However, here the cluster of TFs and risk loci instead largely operate in ductal epithelial cells.

FIG. 137. Comparison between standard RELI null model and an alternative null model that matches variants based on their distance to the nearest gene transcription start site. The set of lupus-associated variants were used as input. Each point represents a single TF with at least one ChIP-seq dataset available. The X-axis indicates the best P-value achieved for the “alternative” null model for any available ChIP-seq dataset for the given TF. The Y-axis indicates the P-value obtained from RELI's “standard” null model. Strong agreement is observed between the two null models (R=0.99). The dashed lines indicate the RELI significance threshold, which effectively divide the plot into four quadrants: the upper right and lower left are shared “positive” and “negative” predictions, respectively; the upper left and lower right represent “RELI standard null model-only” and “RELI alternative null model-only” predictions, respectively. From these quadrants, we calculate the overall concordance between the two methods as the percentage of agreements (i.e., the sum of the upper right and lower left quadrants). Overall, a very strong concordance was observed between these two methods—13.1% of the plot represents “shared positives”, and 82.5% is “shared negatives”, for an overall concordance of 95.6%. A combined null model was implemented, which randomly selects variants located within EBV+B cell open chromatin (again using DNase-seq data), while matching based on allele frequency AND distance to the TSS/TES. This new null model again has high concordance with the current RELI “EBV+B cell open chromatin” null model (which does not consider distance to TSS/TES): R=0.99, Concordance=98.5%. The null model currently employed by RELI is highly consistent with this alternative null model.

FIG. 138. Comparison between standard RELI null model and the null model used by the GoShifter method, which locally repositions the genomic features (here, ChIP-seq peaks) within a locus, while keeping the variant positions fixed. The set of lupus-associated variants were used as input. Each point represents a single TF with at least one ChIP-seq dataset available. The X-axis indicates the best P-value achieved for the null model employed by GoShifter (Trynka et al. 2015). The Y-axis indicates the P-value obtained from RELI's “standard” null model. The dashed lines indicate the RELI significance threshold, which effectively divide the plot into four quadrants: the upper right and lower left are shared “positive” and “negative” predictions, respectively; the upper left and lower right represent “RELI standard null model-only” and “RELI alternative null model-only” predictions, respectively. From these quadrants, we calculate the overall concordance between the two methods as the percentage of agreements (i.e., the sum of the upper right and lower left quadrants). Overall, we observe very strong concordance between these two methods—8.1% of the plot represents “shared positives”, and 77.9% is “shared negatives”, for an overall concordance of 86.1%. We conclude that the null model currently employed by RELI is consistent with this independent, alternative null model. Note that the null model “universes” are many orders of magnitude different, in terms of their size. The standard RELI null model randomly picks from all of the variants in the genome. With GoShifter, the detection power is heavily limited by both the number of simulations used in generating the null distribution and the nature of the “local shift” performed by the algorithm, which can only select from a small subset of the genome. Thus, the P-values achieved by GoShifter cannot possibly approach the significance levels of RELI. As a consequence, the GoShifter publication uses a much lower P-value threshold of 0.05, which is employed in this figure. TFs tend to bind in ‘homotypic’ clusters, both within a single enhancer, and across enhancers at a given locus (Gotea et al. 2010, Ezer et al. 2014). Thus, the GoShifter null model, which scrambles variants within an LD block, can shuffle a given variant into another ChIP-seq peak for the same TF, which would decrease the significance even though the connection between the variant and the TF is still important biologically.

FIG. 139. Global allelic EBNA2 co-binding results using additional EBNA2 ChIP-seq datasets as input. ChIP-seq datasets from EBV-infected B cell lines were examined for evidence of allele-dependent binding at heterozygotes. Datasets are sorted by the proportion of EBNA2 allelic events (MARIO ARS value >0.40, see Methods) that favor the same allele (X-axis). Values (N) indicate total number of variants. One plot is provided for each of the three available EBNA2 ChIP-seq datasets.

FIG. 140. Western blot confirming the anticipated presence and absence of EBNA2 in Ramos cell lines. Whole cell lysate from Ramos cells with or without EBV infection were probed for EBNA2 (clone PE2-ab90543 (Abcam, Cambridge, Mass.), anticipated molecular weight of 75 kDa) using a secondary antibody that fluoresces at 800 nm. As a control, (3-actin (ab8227 (Abcam), anticipated molecular weight of 42 kDa) was probed using a secondary antibody that fluoresces at 700 nm. A merged overlap is shown with one lane cropped as indicated.

FIG. 141. The rs3794102 variant loops to the promoter region of CD44 in EBV infected B cell lines. Hi-C data performed in GM12878 EBV infected B cell lines localizing to the rs3794102 locus. Bars at the top depict genes, with exons indicated as thick bars and introns as thin bars. Arrows indicate direction of transcription. Vertical bar indicates the location of the rs3794102 variant. Magenta lines indicate chromatin looping interactions emanating from the rs3794102 locus, as indicated by Hi-C data taken from the Washington University EpiGenome browser (http://epigenomegateway.wustl.edu/browser/).

FIG. 142. Overview of the MARIO (Measurement of Allelic Ratio Informatics Operator) pipeline. The procedure begins with a cell type with available whole genome sequence or genotyping data, a reference human genome with all common variants masked to N, and a set of parameters (see Methods). For a given ChIP-seq dataset, each experiment (referenced here by NCBI SRR IDs) is downloaded from the NCBI Sequence Read Archive (SRA), and the sequencing reads are mapped to the masked reference genome. Peaks are called using MACS2, and all variants that are heterozygous within the given cell type are identified within each peak. For each such variant, the number of reads mapping to each allele are counted. This procedure is repeated for all available experimental replicates for the given dataset. Allelic Reproducibility Score (ARS) values are then calculated for each variant, and additional statistics and annotations are compiled in the final report summary. See Methods for additional details.

FIGS. 143A-143D. Identification of predictive variables of reproducible allele-dependent behavior across replicates. FIG. 143A, Schematic for the detection of allelic behavior. Definition of alleles is based on the number of aligned ChIP-seq reads. The “strong allele” corresponds to the allele with the higher number of aligned reads. The “weak allele” has the fewest aligned reads. FIG. 143B, Definitions of datasets and variables used to derive ARS values. A set of 7 ChIP-seq datasets {D}, each containing four experimental replicates {Rd} was identified. Each variant Vdr is characterized in replicate Rd with a set of four variables {Xdrv}: the ratio of weak-to-strong reads, the number of strong reads, peak width, and normalized distance to the center of the peak. FIG. 143C, Identification of the set of reproducible variants for each dataset D. The set of reproducible variants {Hd} is defined as those variants in the set Vdr with the same strong base in all four experimental replicates Rd. All other variants are denoted non-reproducible. FIG. 143D, Comparison of reproducible variants (green) and non-reproducible variants (dark brown). The four panels illustrate the ability of each of the four variables to distinguish between reproducible and non-reproducible variants. Cumulative counts are calculated for each variant type for each variable Xdrv. Plots indicate the normalized cumulative frequency of counts. The set of reproducible variants shows an enrichment in low WS reads ratio values (left-most plot), which represent preferences for one of the alleles. A value of 0.5 means a variant has twice the number of reads in the strong allele compared to the weak allele. The set of reproducible variants also has enrichment for a higher number of reads (second plot from left), evidenced by the frequency starting close to zero, and the slower saturation of the green curve. The remaining two variables did not show an appreciable ability to distinguish between reproducible and non-reproducible variants, and thus were deemed uninformative.

FIGS. 144A-144C. Calculation of MARIO Allelic Reproducibility Score (ARS) values. FIG. 144A, Prediction of the set of reproducible variants. Three possible real-world scenarios involving the number of experimental replicates (1, 2, or 3) that are available for a given dataset were simulated. For each variant, different values of the two informative variables were explored: the total number of reads (num_reads, X-axis) and the ratio between the amount of reads mapping to the weak vs. the strong allele (WS_ratio, curves). Each point in the plots indicates the fraction of variants {Hd} that belong to the set of reproducible variants (heterozygous variants sharing the same strong base across all four experimental replicates), for the given values of WS_ratio and num_reads. The values of the WS_ratio for each curve are indicated at the right. FIG. 144B, ARS values as a function of WS_ratio and num_reads. The calculation of ARS values is described in the Supplementary Methods. The solid lines represent the best fit of a saturating curve to the points. FIG. 144C, Correspondence between ARS values and WS_ratios. High ARS values correspond to low WS_ratios (i.e., higher ARS values are indicative of stronger allelic behavior).

FIGS. 145A-145G. Locus plots of EBV+/−analysis for all 7 EBNA2 disorders. The X-axis depicts the disease-associated loci. The Y-axis depicts results from each of the datasets for the eight TFs with at least one EBV-infected B cell and one EBV-negative B cell ChIP-seq dataset. A colored box indicates that the given locus contains at least one disease-associated variant located within a ChIP-seq peak for the given dataset. EBV-infected datasets are colored; EBV-negative datasets are shown in white. The total number of intersections for each dataset is indicated at the right, along with the TF and cell line. Each of the seven EBNA2 disorders is shown, one per page.

FIGS. 146A-146J. Locus plots for additional phenotypes of interest. This figure is an extension of FIGS. 133A-133G, but additional space is used here to label the TFs and disease loci. Additional diseases of interest are included at the end. See description for FIGS. 133A-133G legend for details.

FIGS. 147A-147G. This figure is an extension of FIGS. 136A and 136C. Additional datasets are provided for SLE and the other EBNA2 disorders. See FIGS. 136A-136D legend for text for details. P-values in upper right indicate the significance of the degree to which the EBV-infected B cell lines (red bars) rank towards the top, based on a Wilcoxon rank-sum test.

FIGS. 148A-148G. Locus plots broken into EBV-infected B cell and T cell datasets for the 7 EBNA2 disorders. Two plots are presented for each of the EBNA2 disorders. The top plot shows the top 25 EBV-infected B cell datasets (based on RELI P-values). The bottom plot shows all available T cell datasets with at least one intersection. A colored box indicates that the given locus contains at least one SLE-associated variant located within a ChIP-seq peak for the gen TF. Loci are classified into one of four categories, based on comparisons between EBV-infected B cell and T cell datasets: loci with substantially more EBV-infected B cell intersections (red bards), loci with substantially more T cell intersections in both (white bars). The following procedure was used for these classifications. For a given locus, we defined Fb and Ft as the fraction of ChIP-seq datasets that intersect that locus in B and T cells, respectively. We defined A=Fb-Ft. Loci with Fb<0.2 and Ft<0.2 were classified as “Neither” (white bars). For the remaining loci, those with delta >0.4 were classified as “B cell only” (red bars). Those with Δ<-0.4 were classified as “T cell only” (blue bars). The remaining loci, which have Fb>0.2 and Ft>0.2, but small A values, were classified as “Both” (yellow bars).

FIG. 149. Intersection between TF binding and genomic loci for the seven EBNA2 disorders. TF ChIP-seq datasets are presented as columns. Loci associated with the seven EBNA2 disorders are shown as rows. An entry is black if the given locus contains at least one EBNA2 disorder-associated variant that is located within a ChIP-seq peak in EBV-infected B cells for the given TF. Loci intersecting at least a quarter of the TFs are shown. Labels at the right indicate the corresponding EBNA2 disorders, the name of the gene most centrally located within the locus, and the genomic coordinates. TF columns are clustered using hierarchical clustering with Euclidean distance and complete linkage criterion.

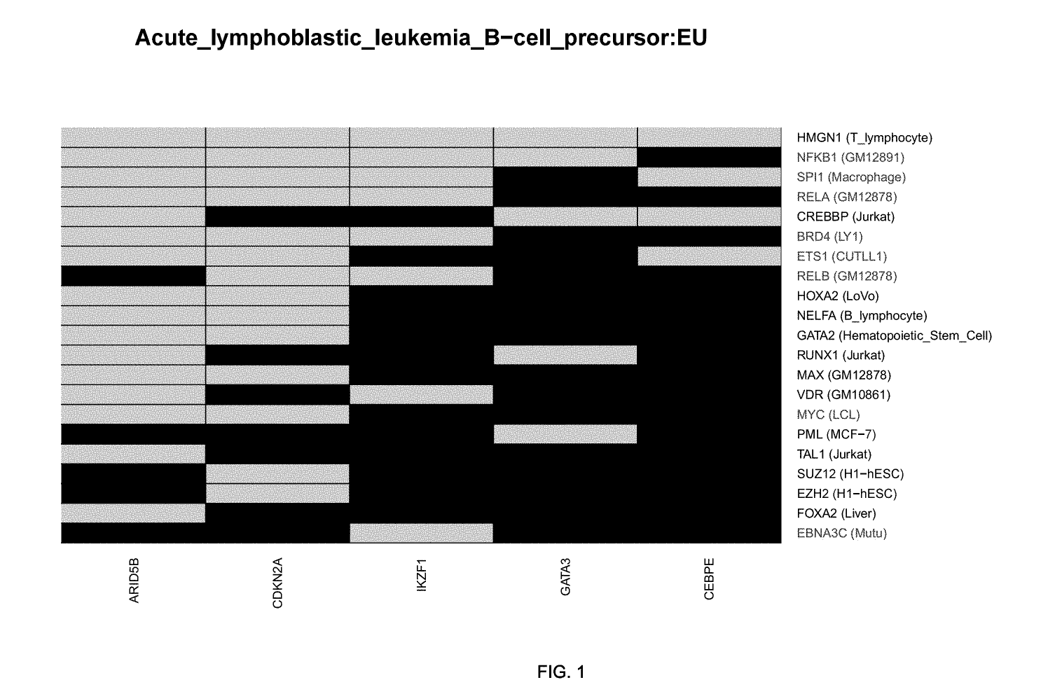

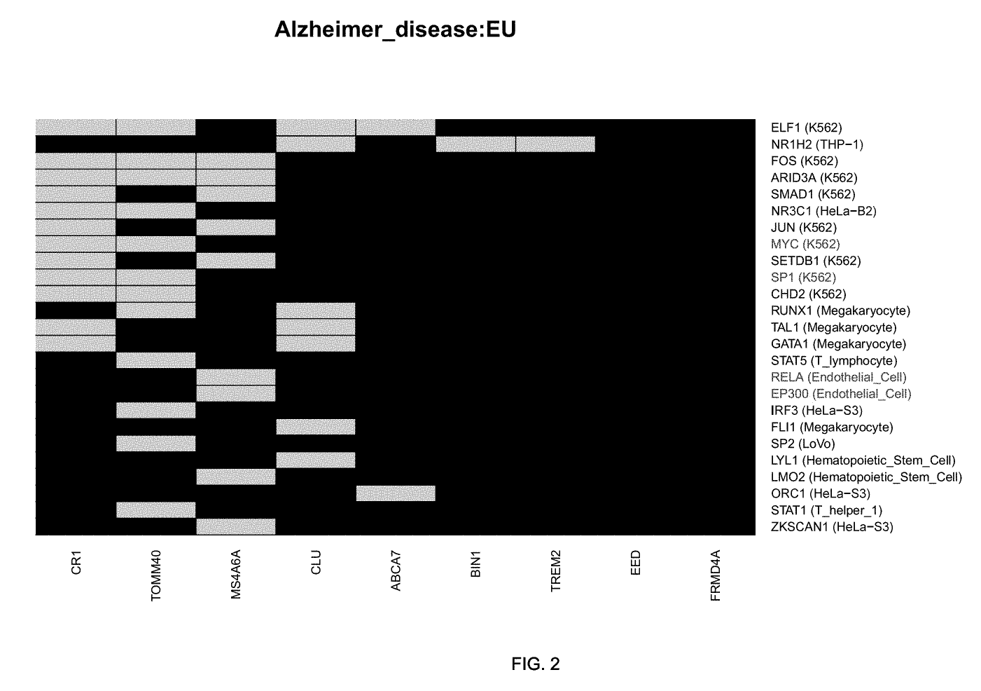

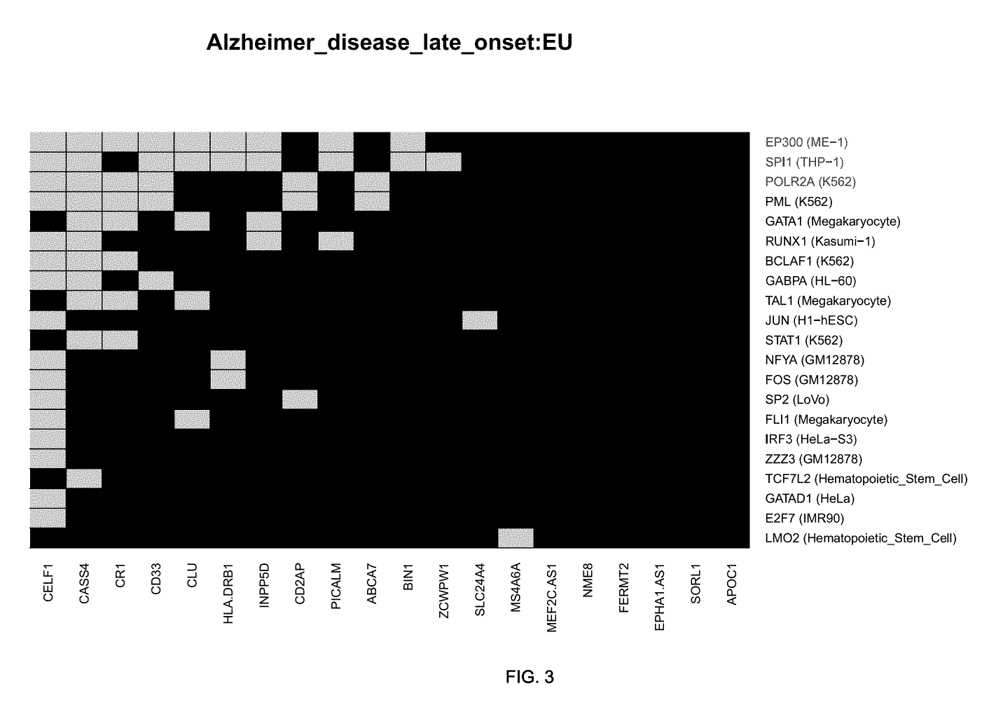

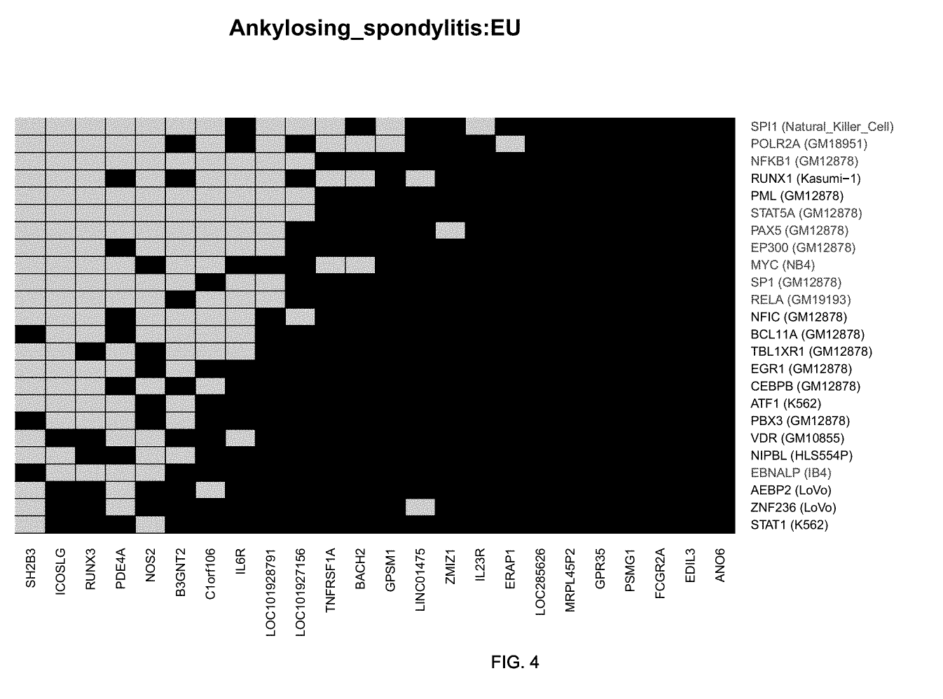

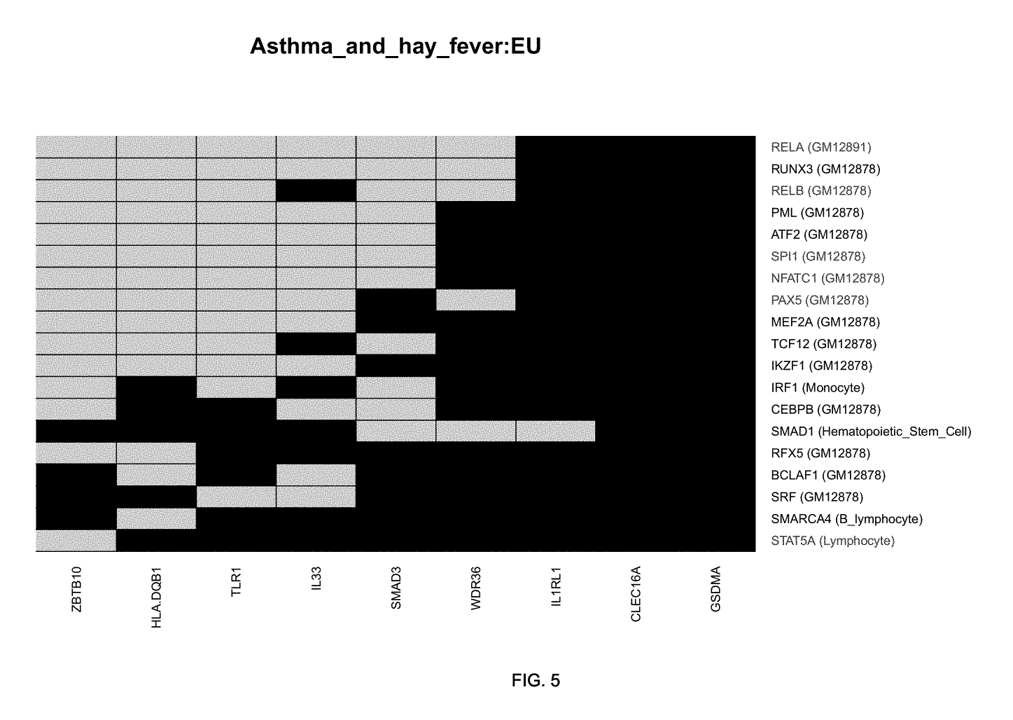

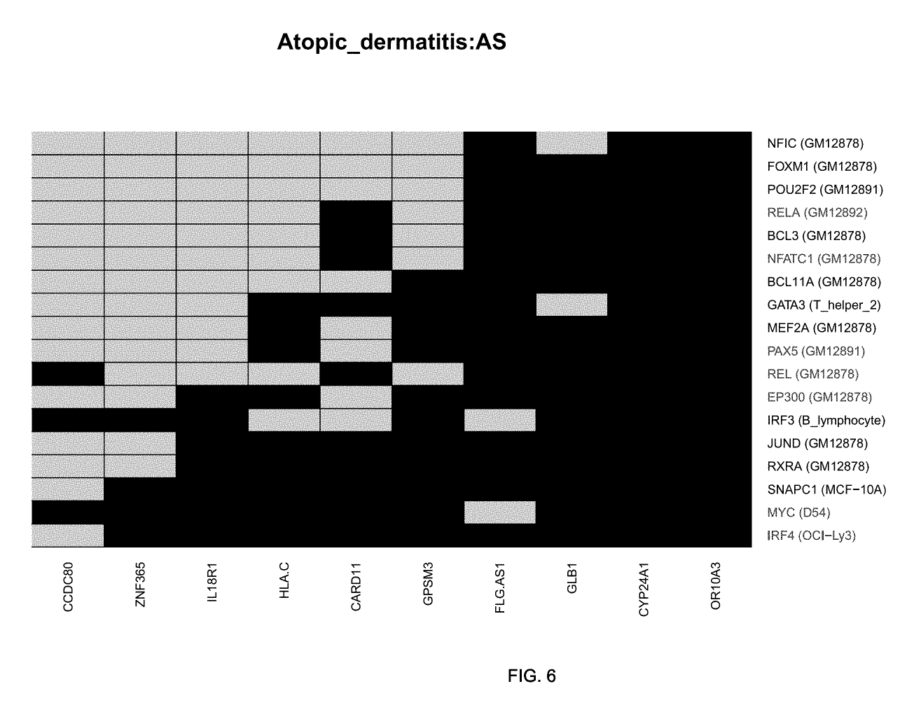

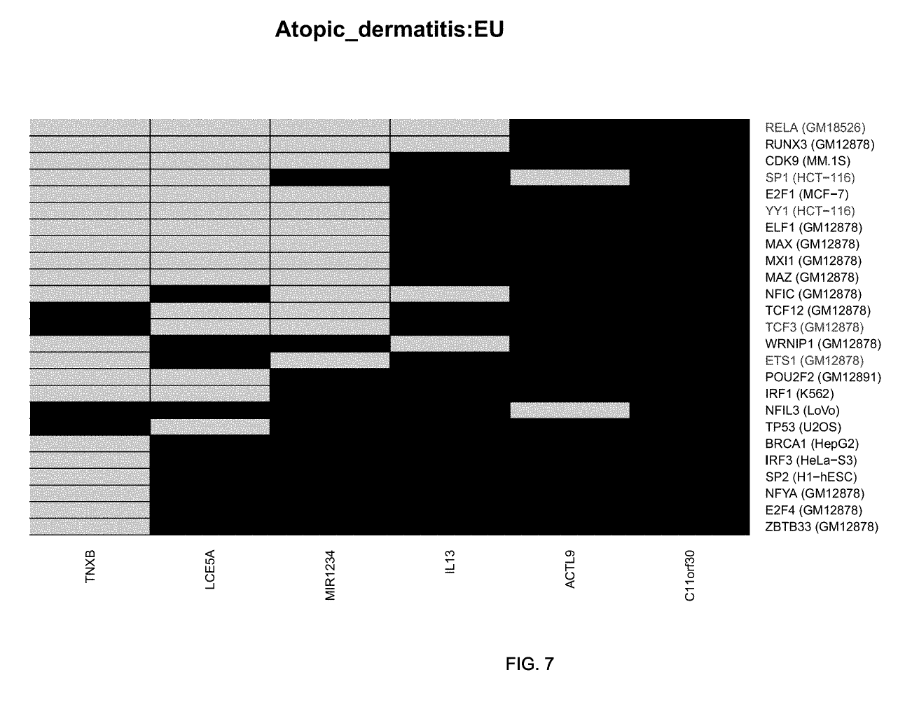

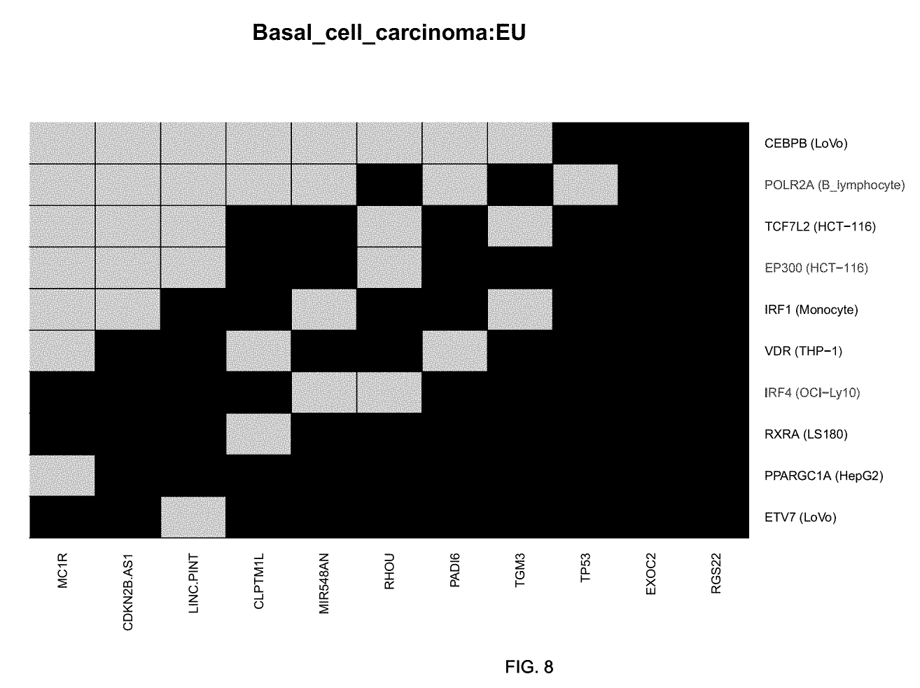

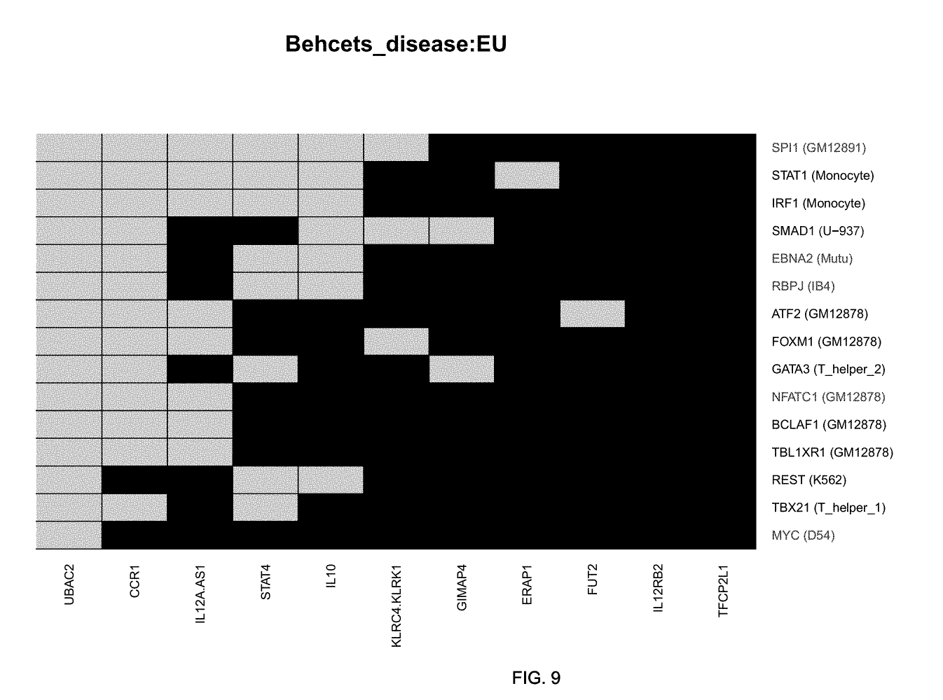

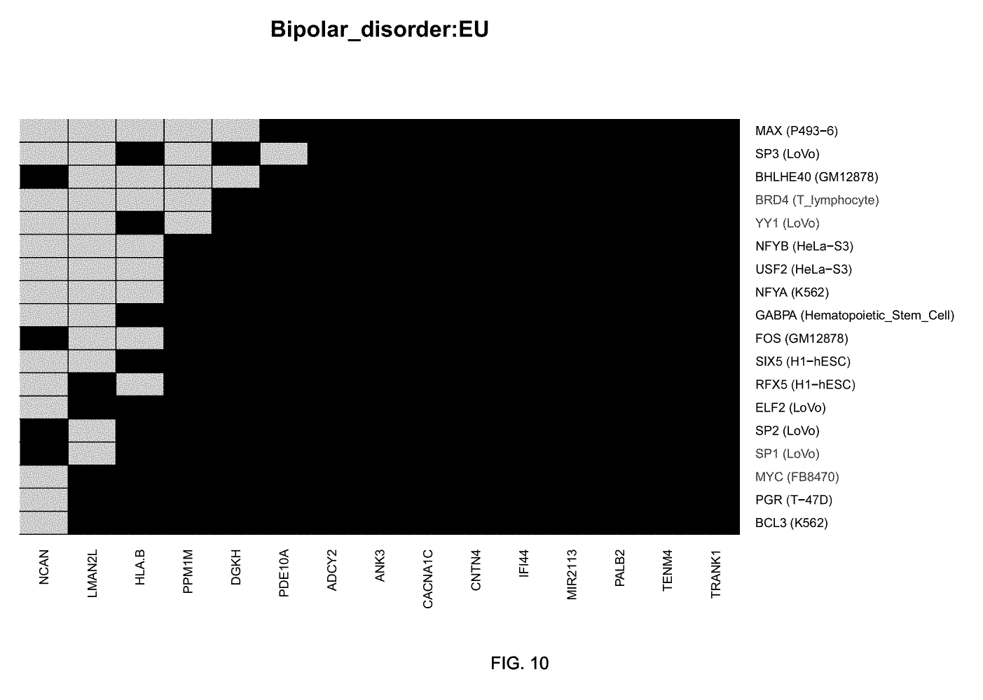

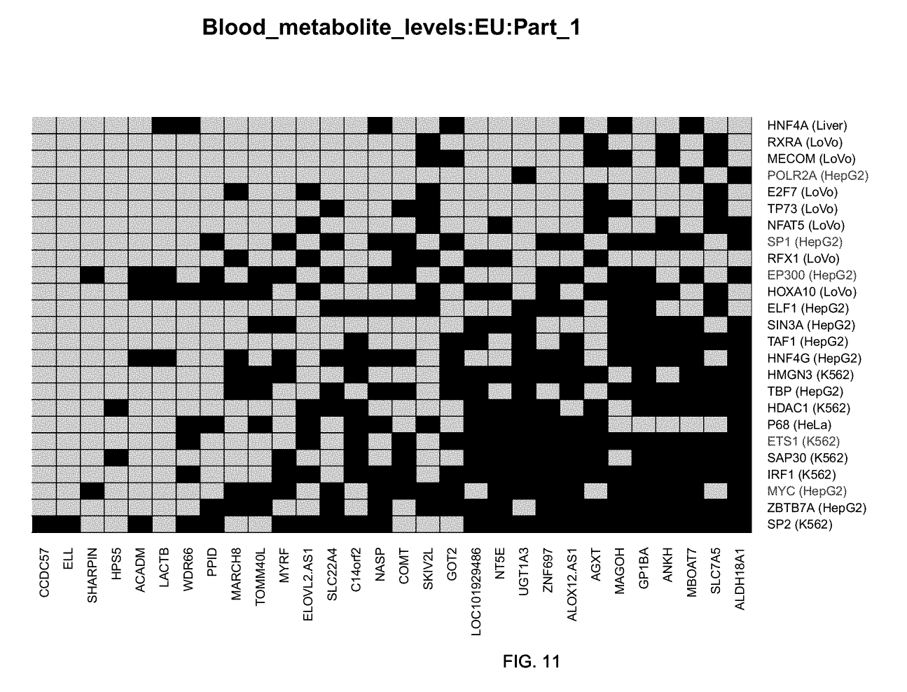

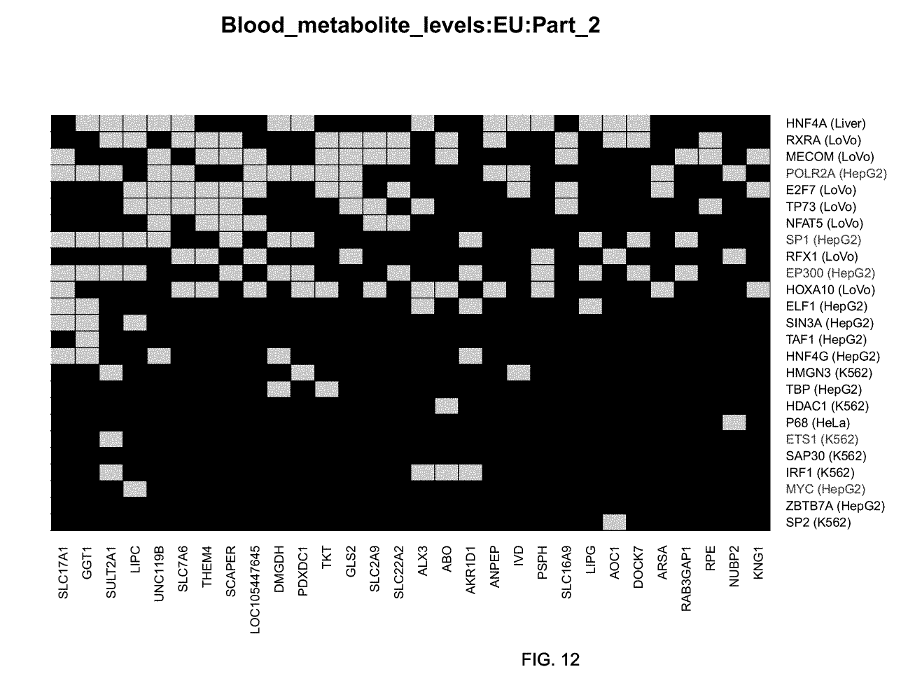

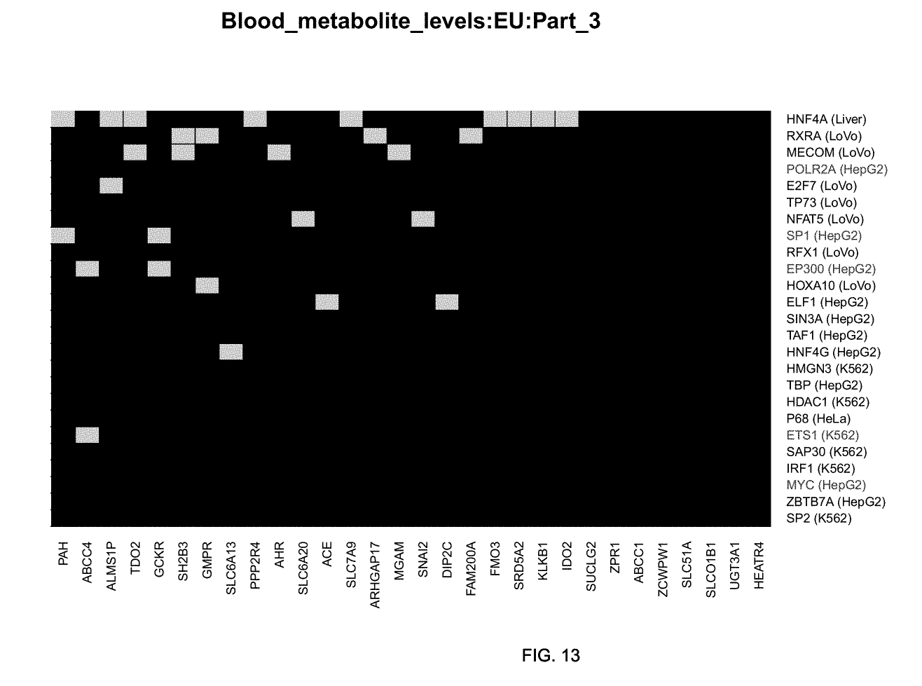

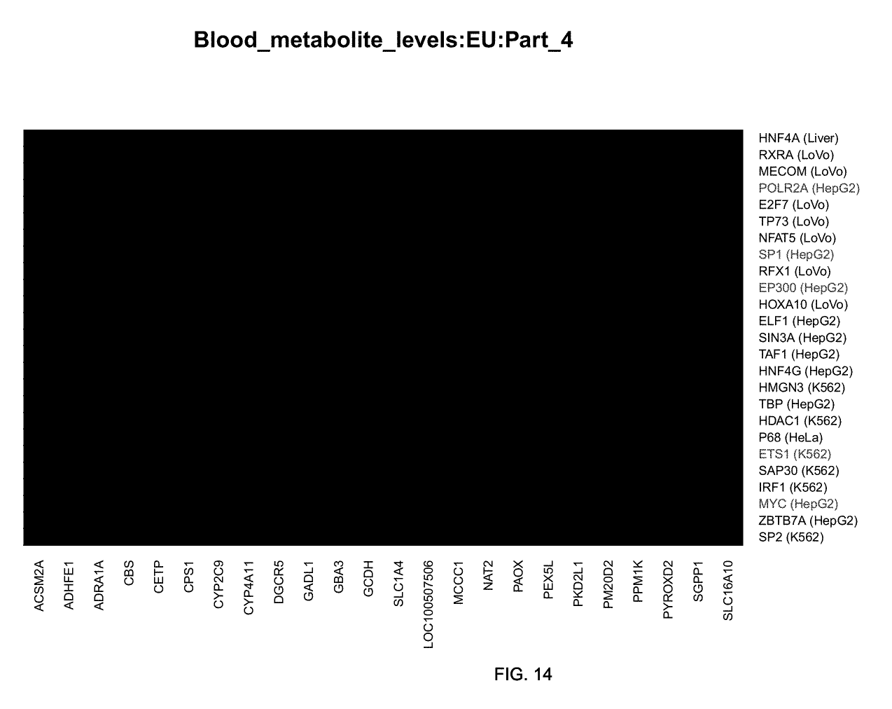

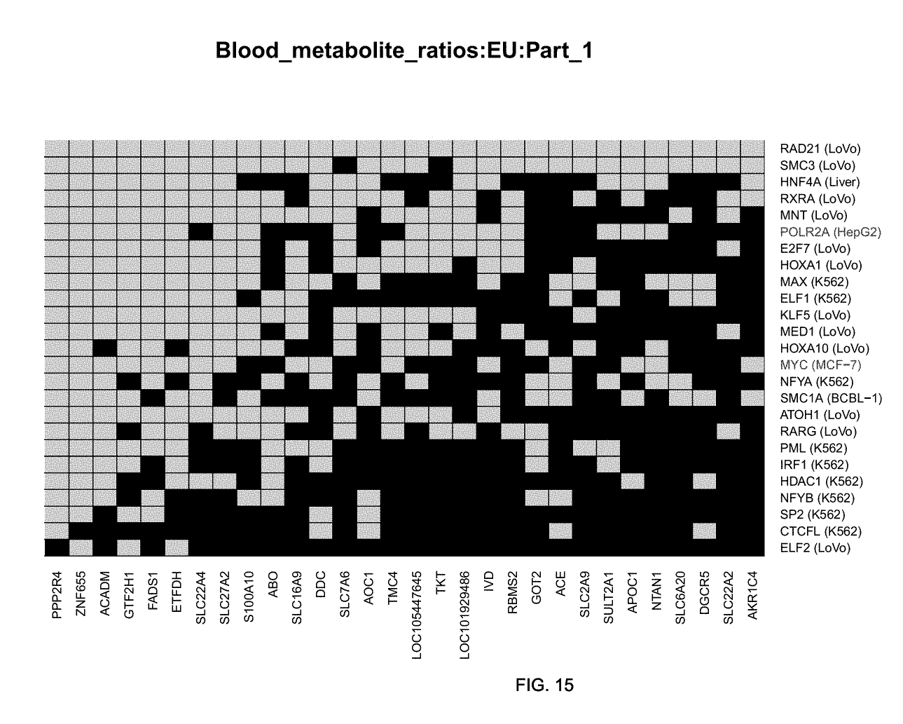

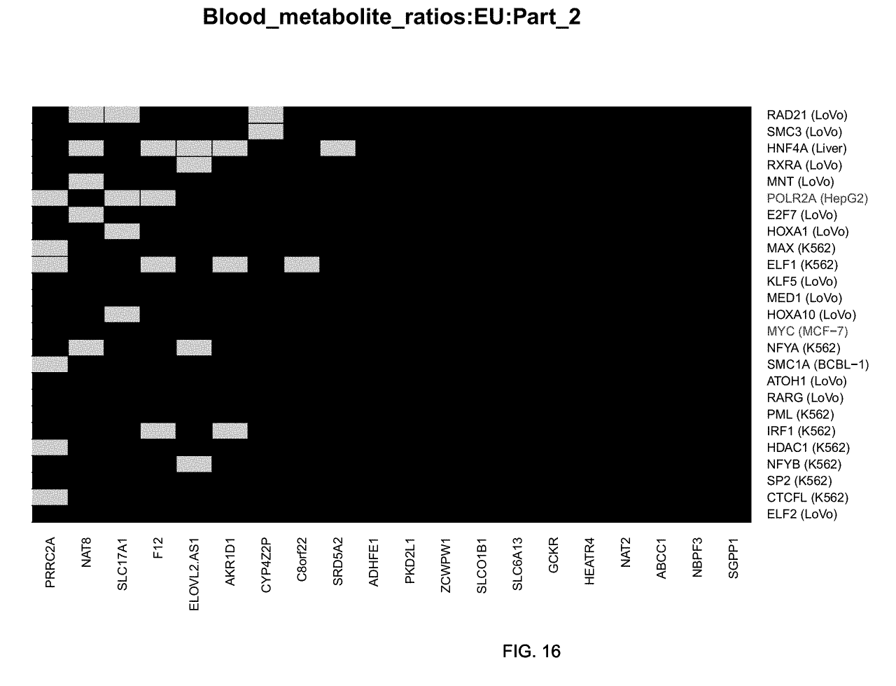

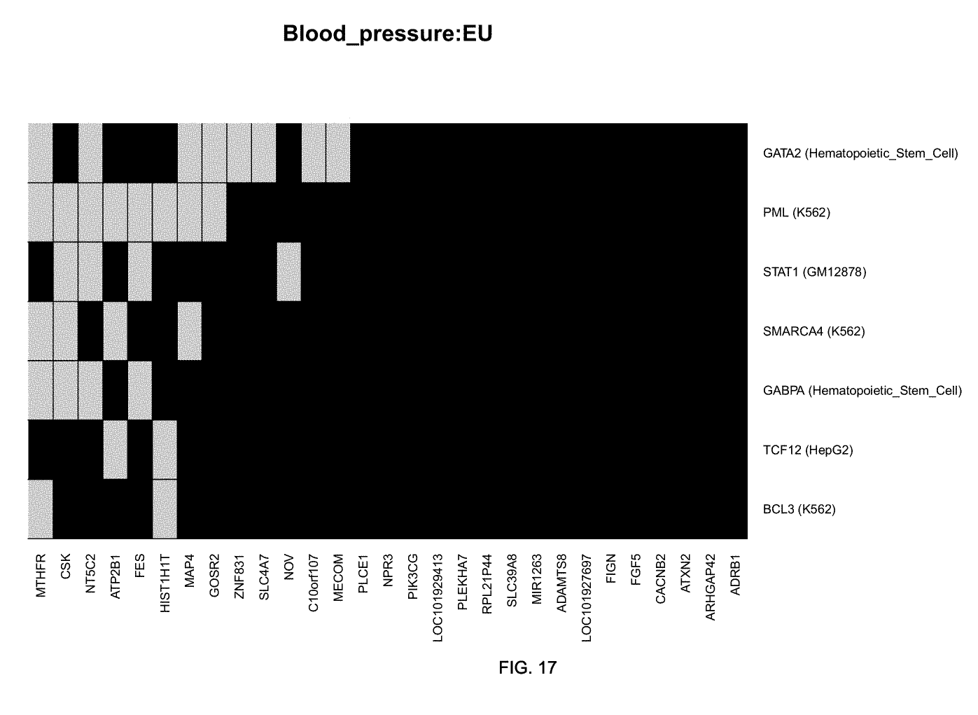

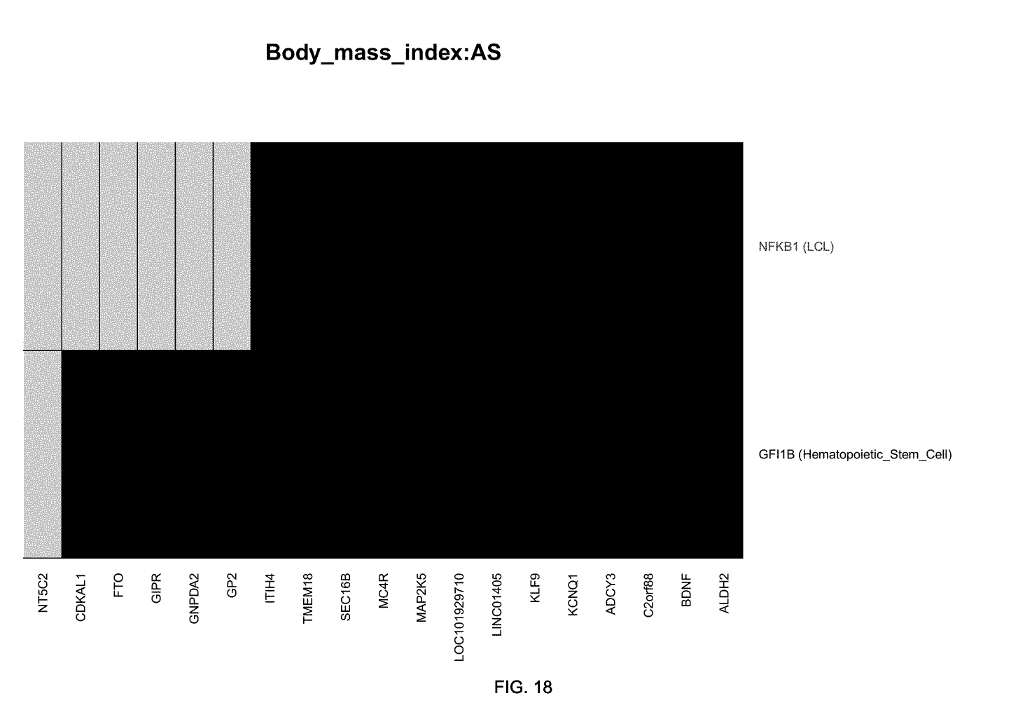

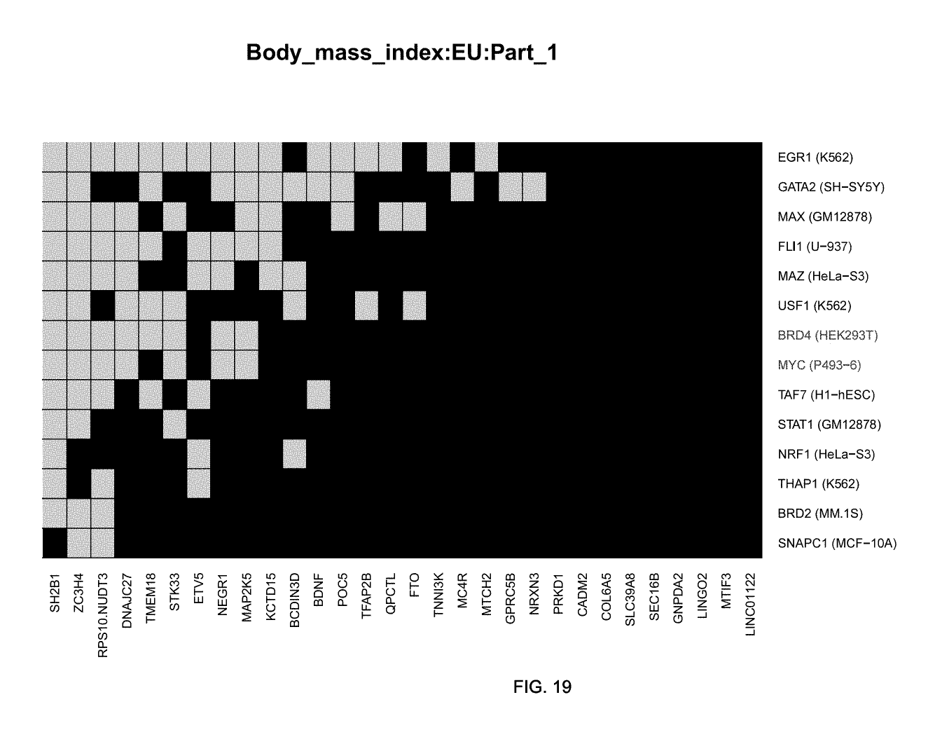

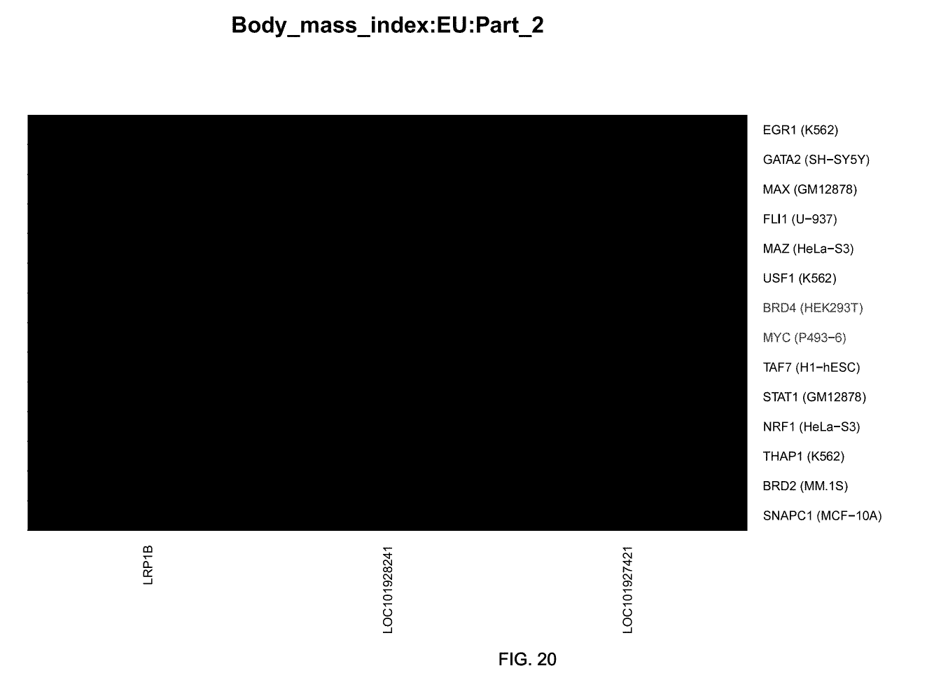

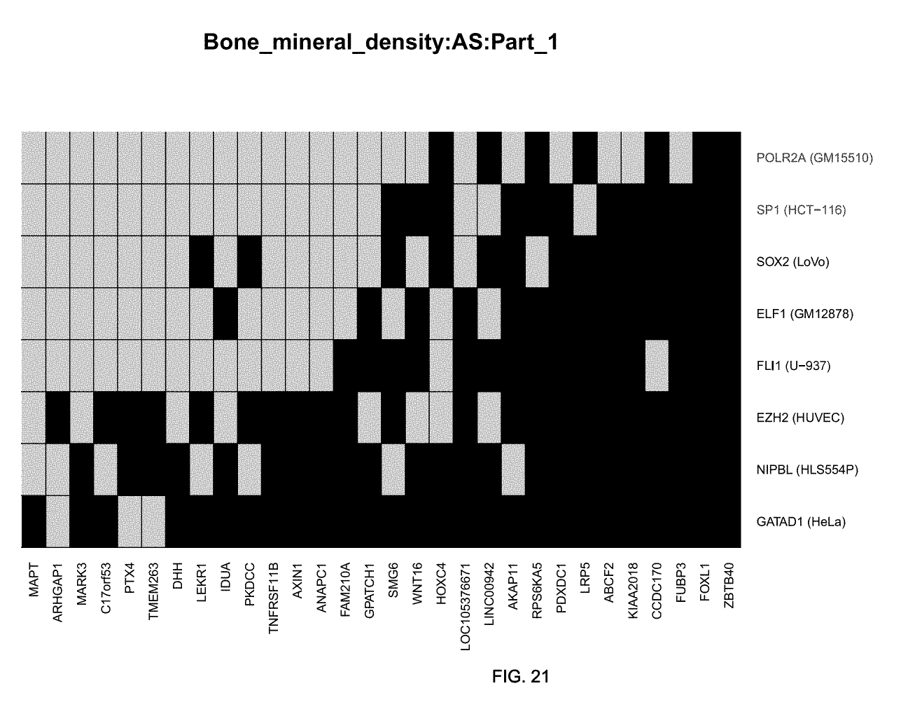

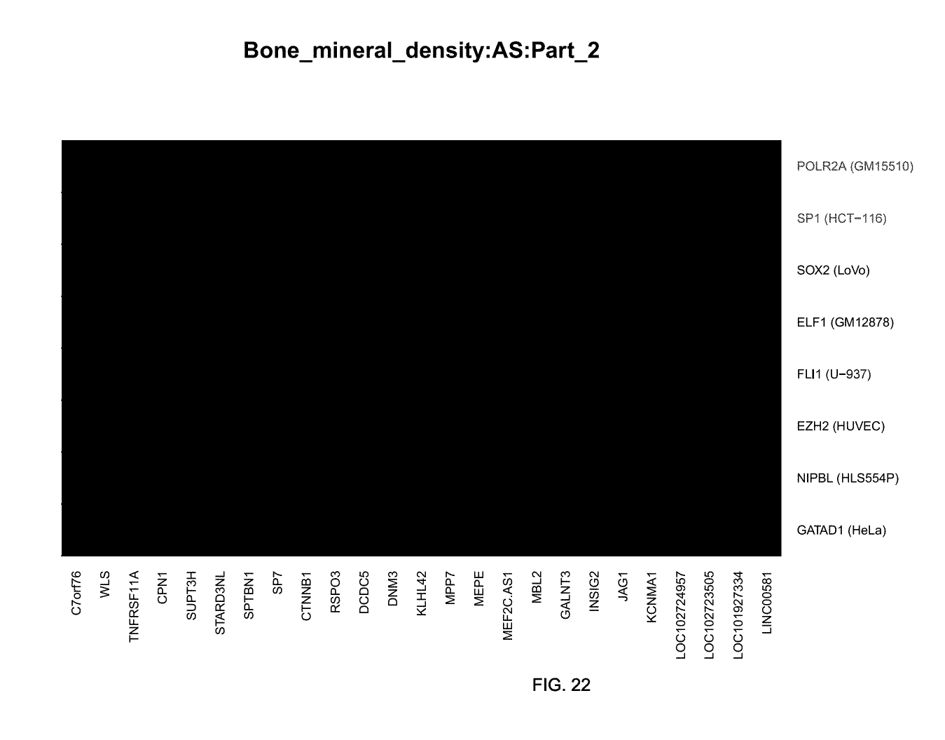

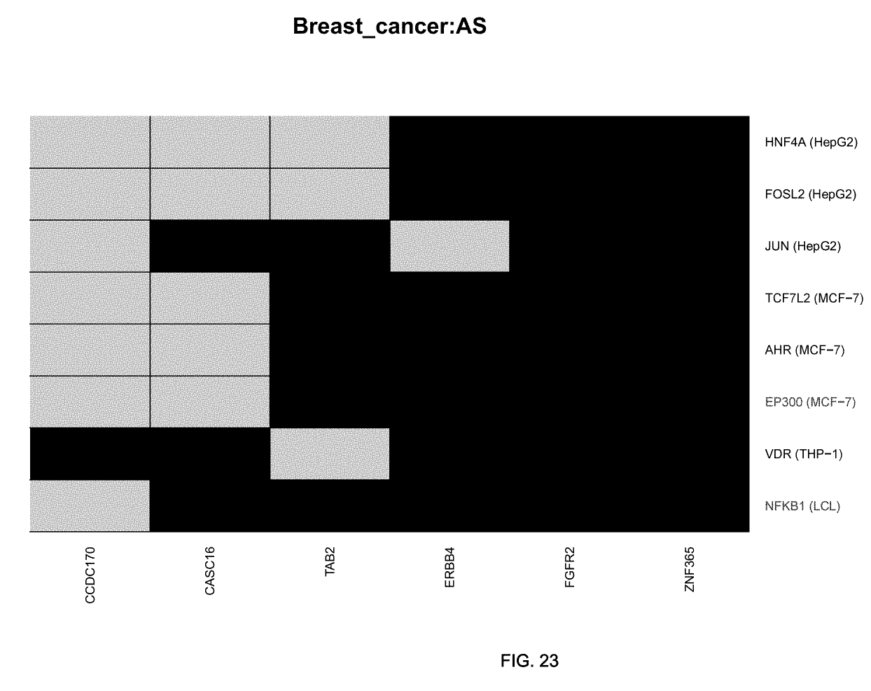

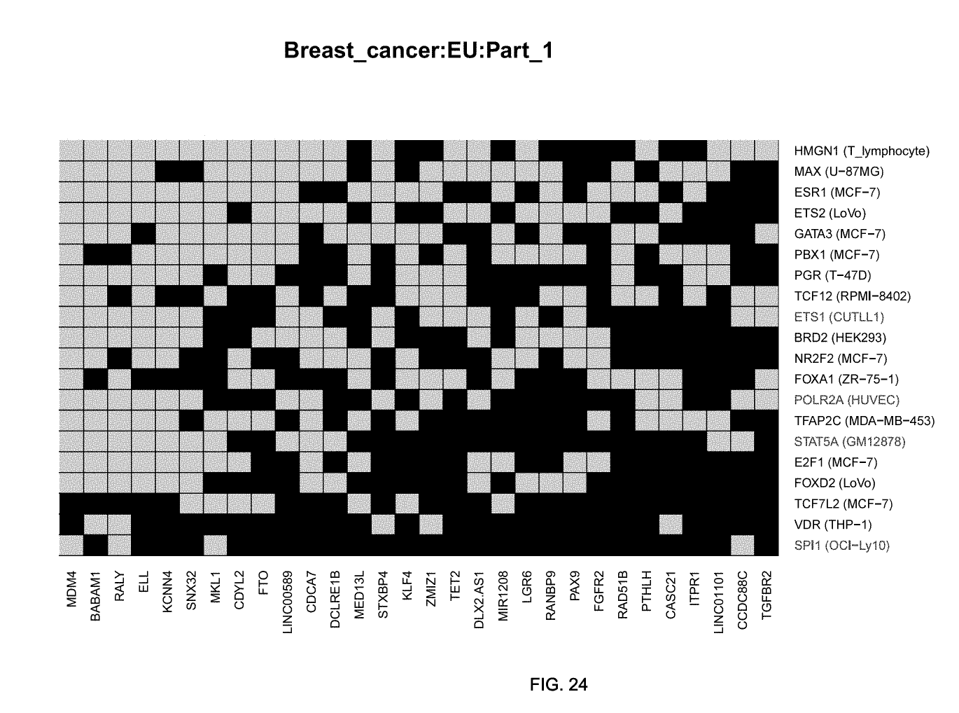

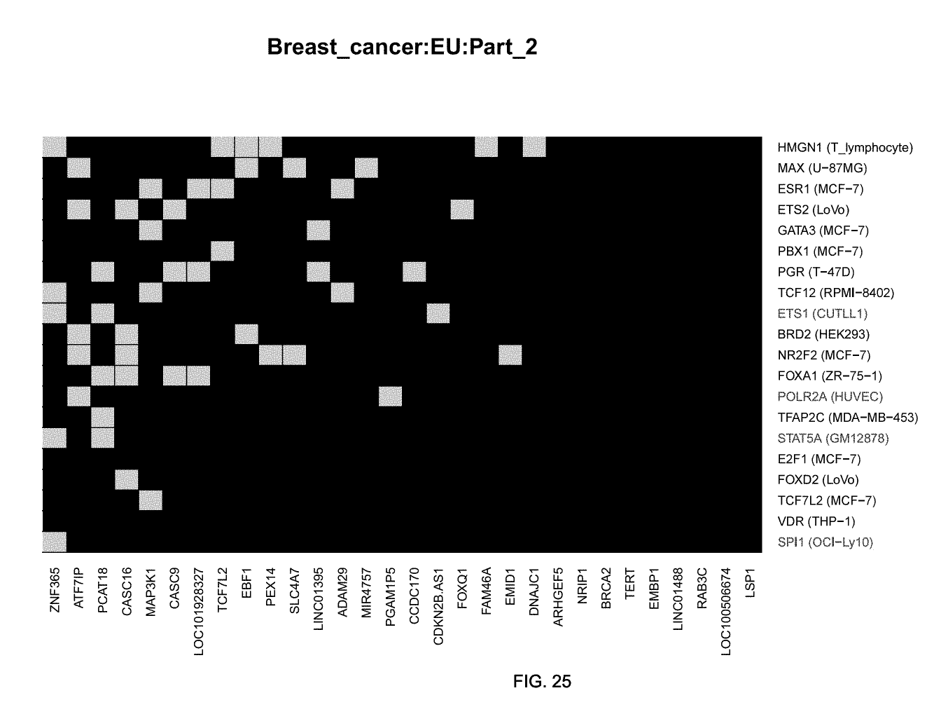

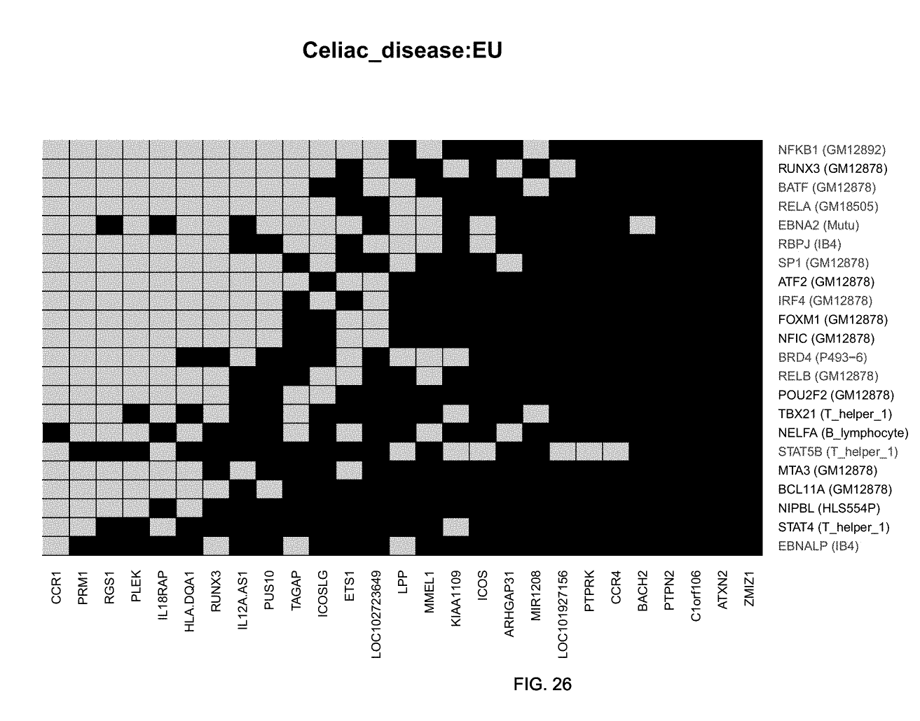

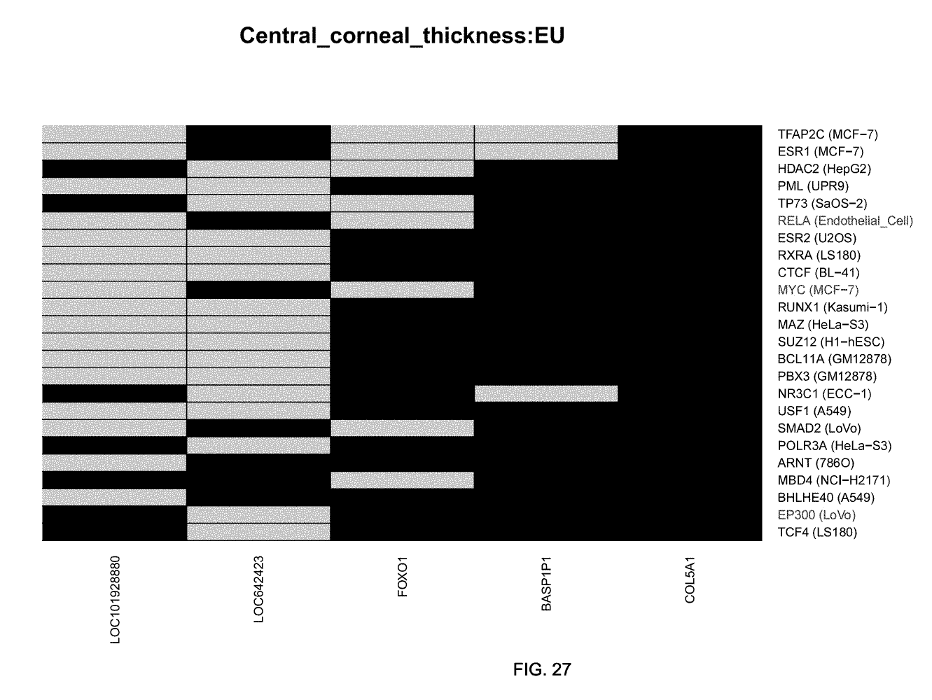

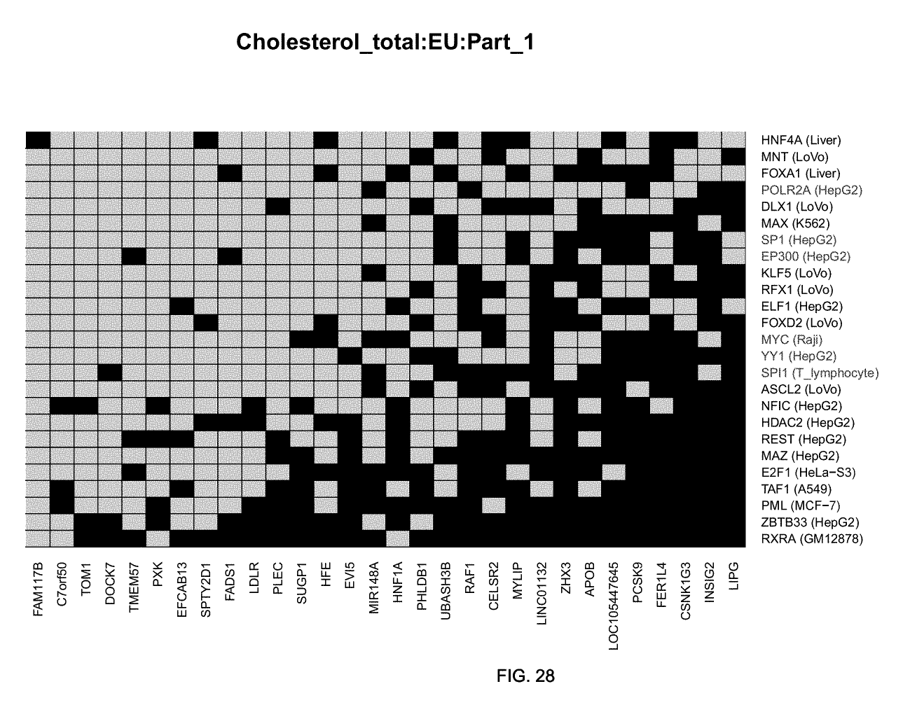

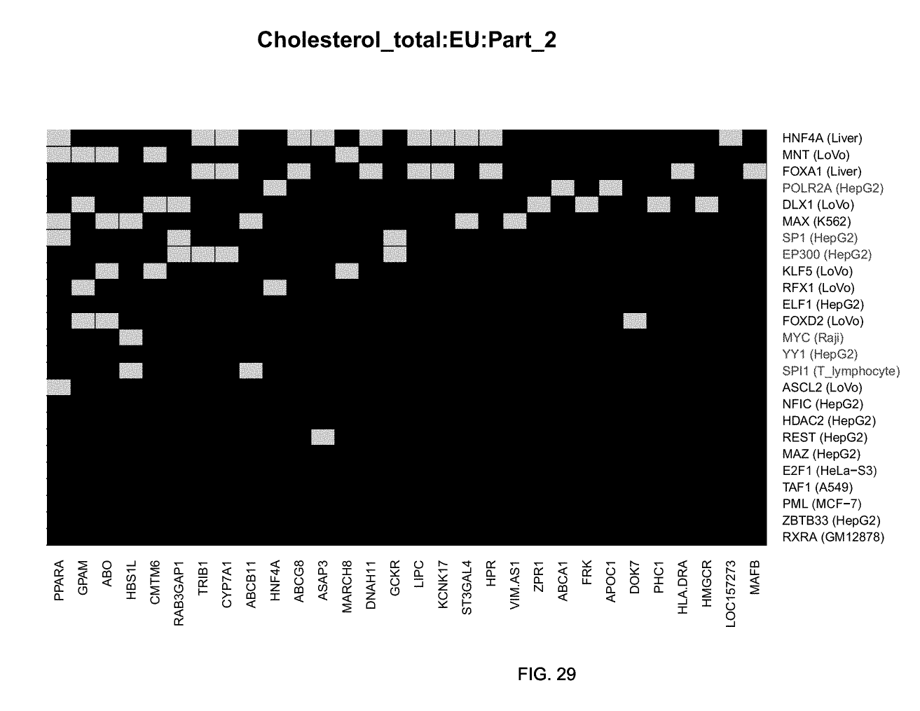

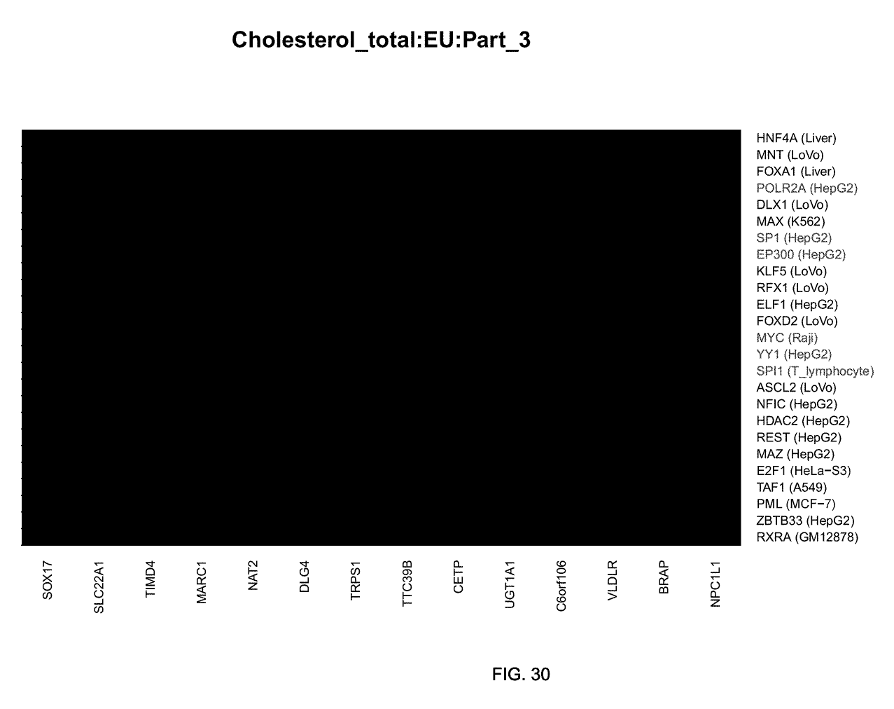

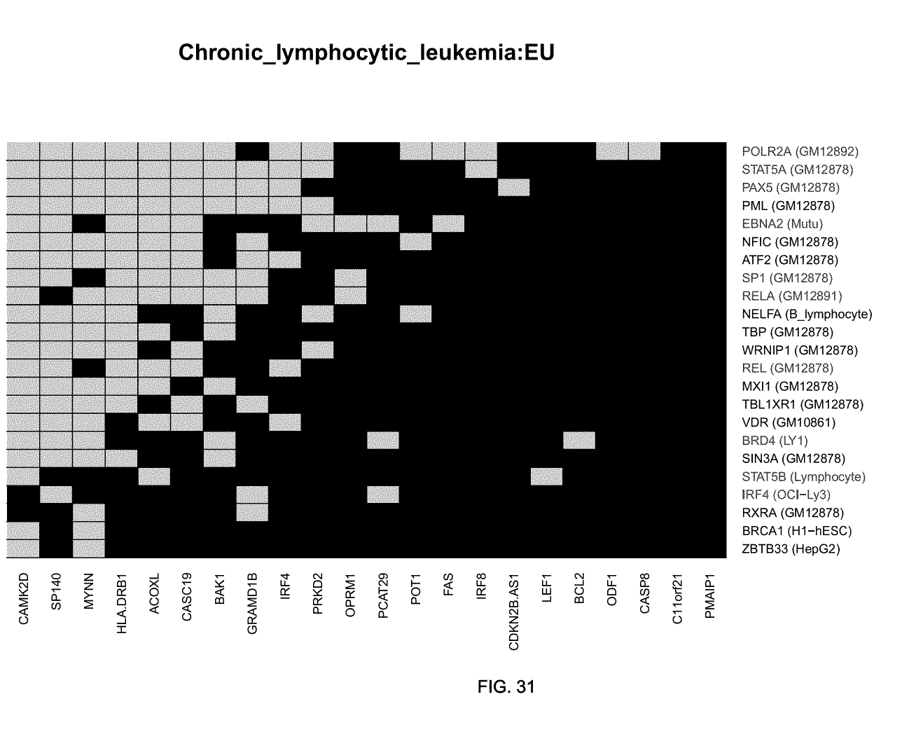

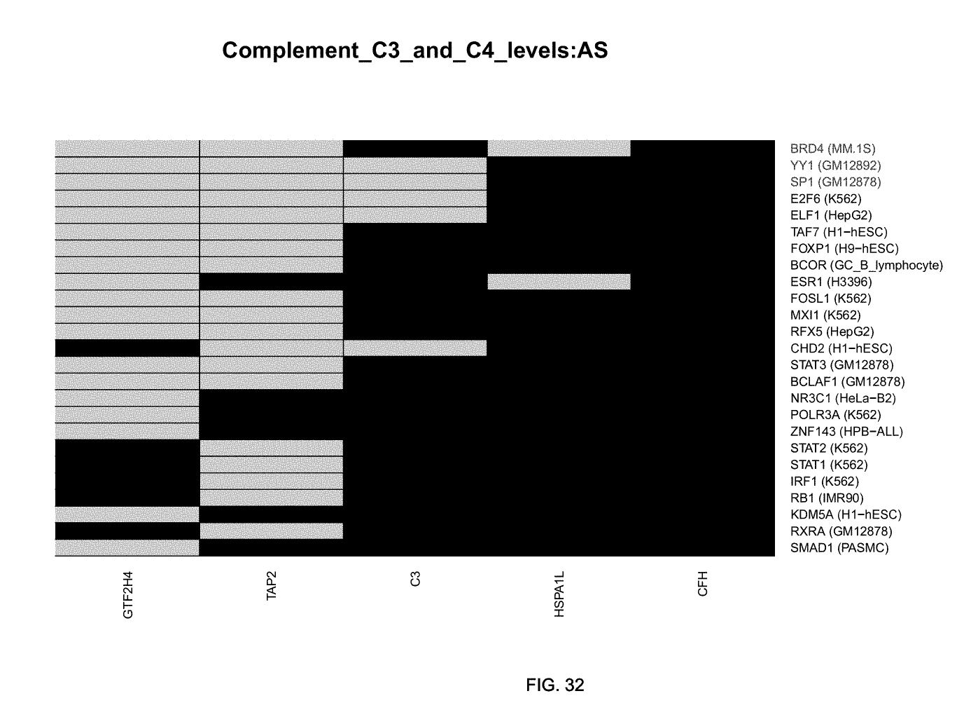

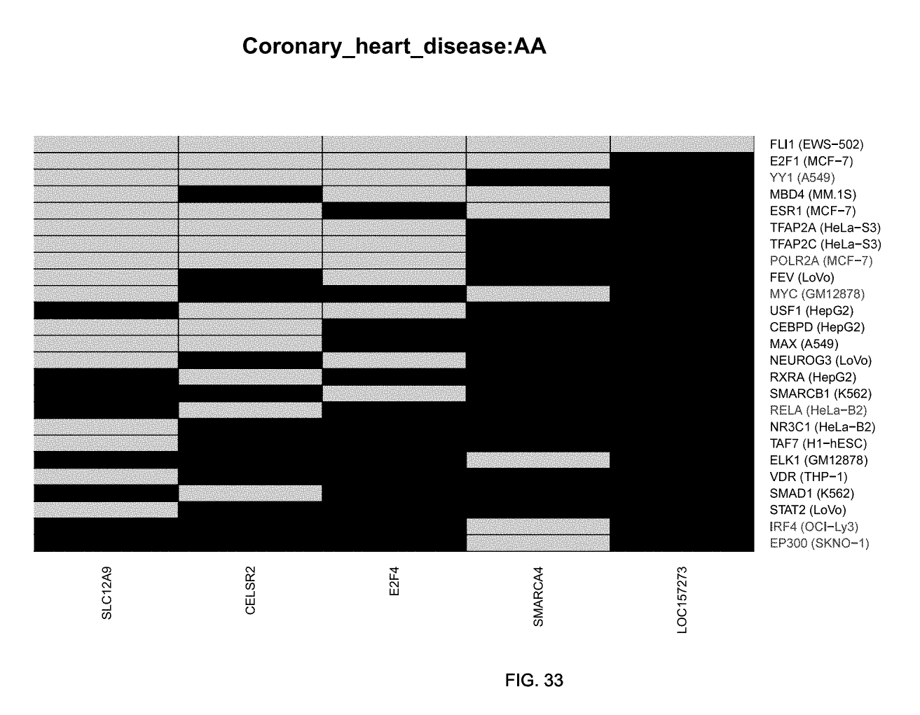

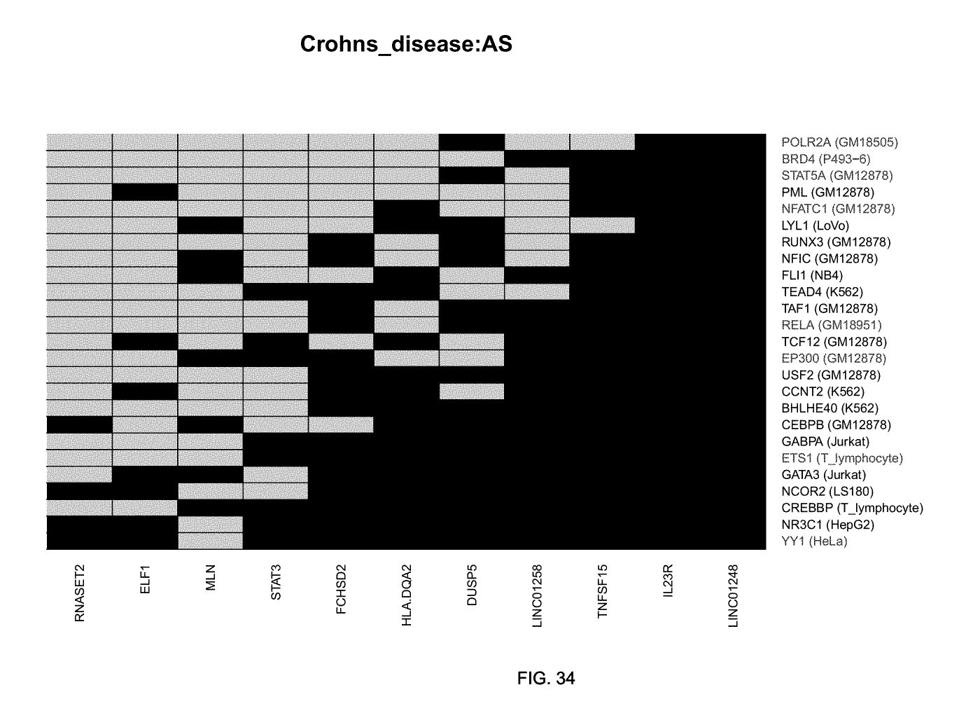

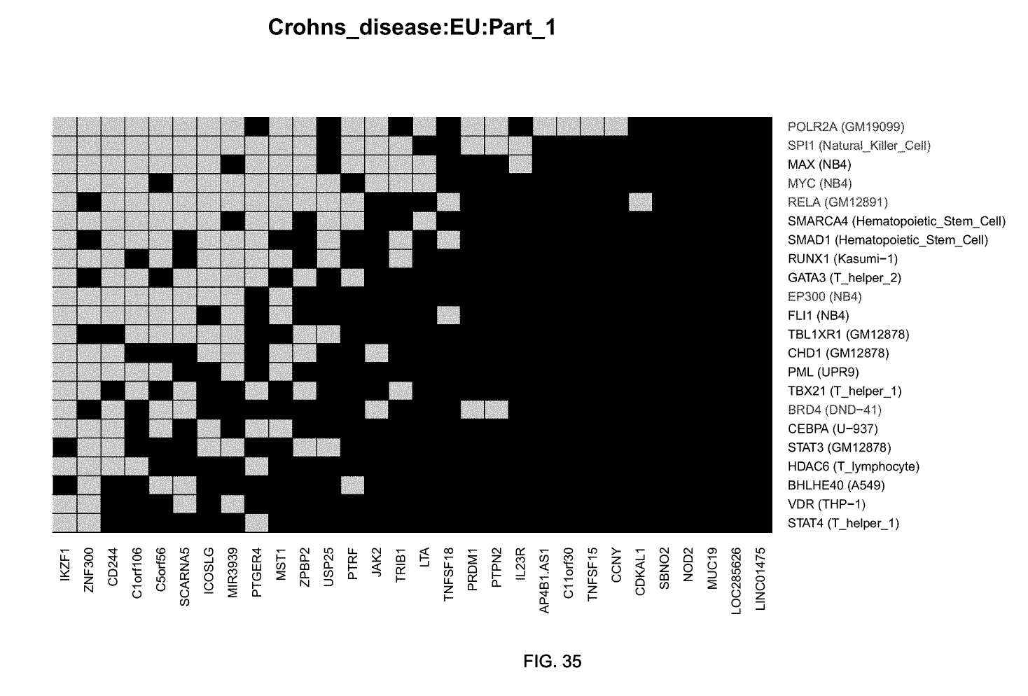

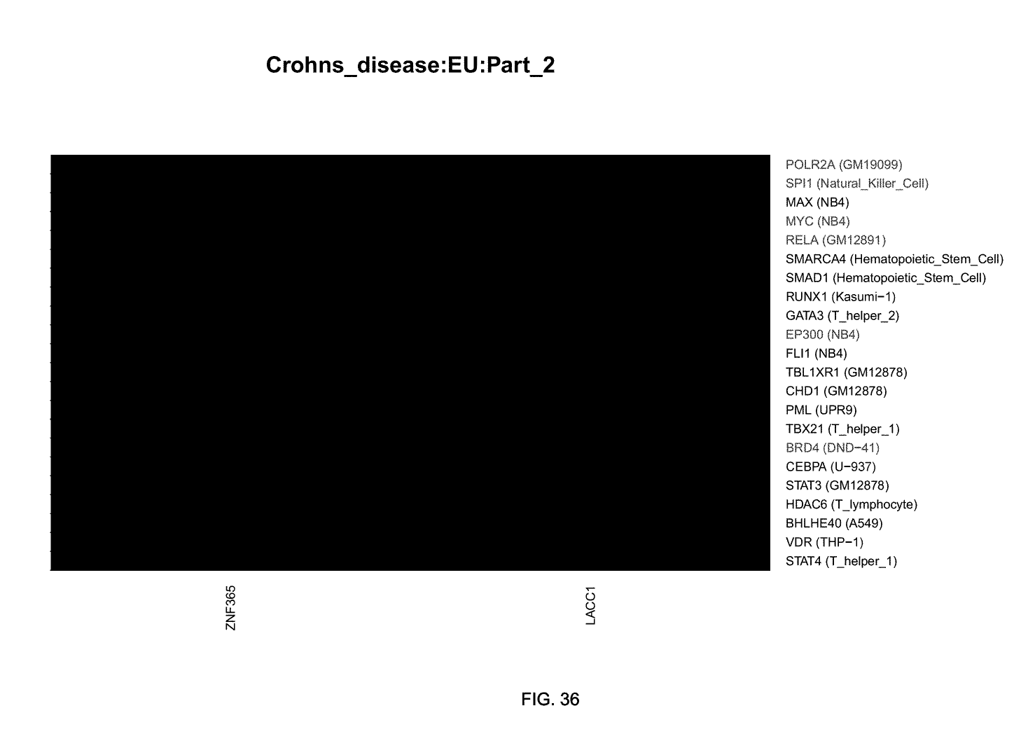

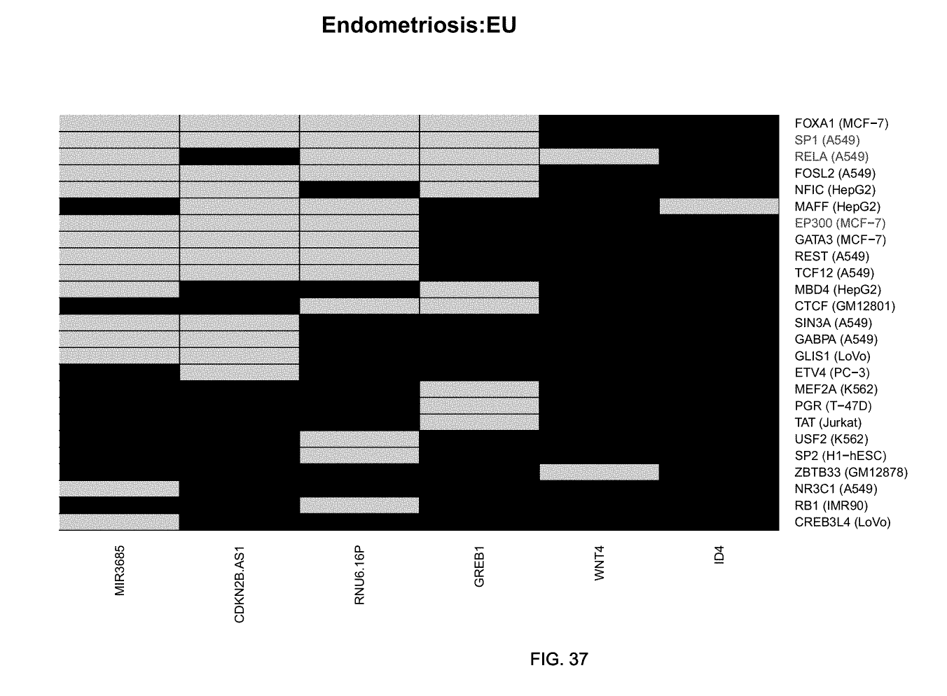

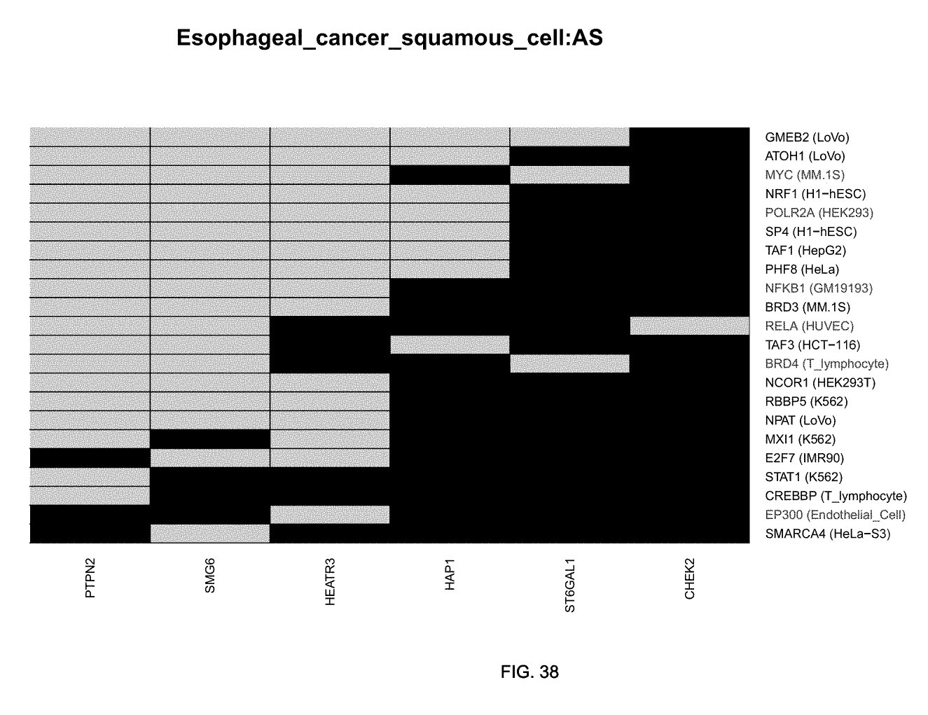

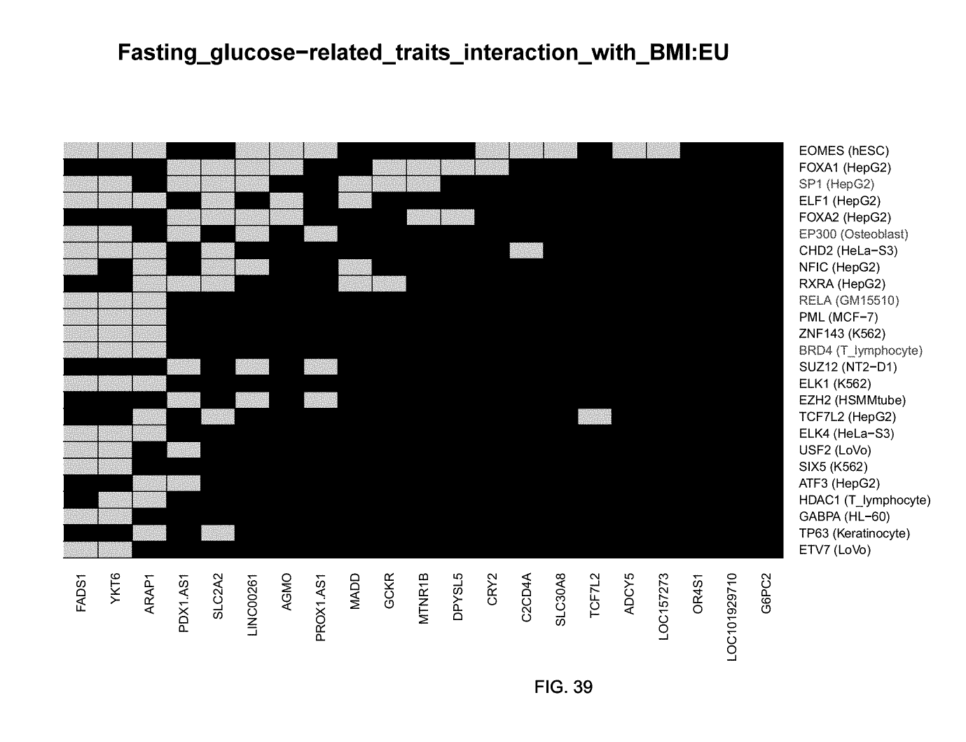

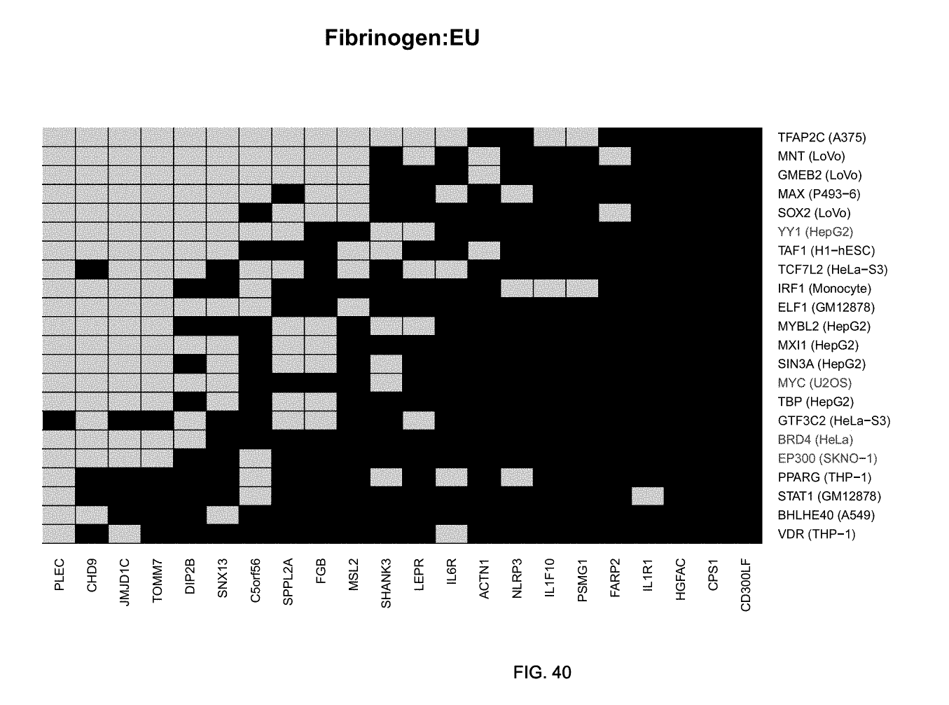

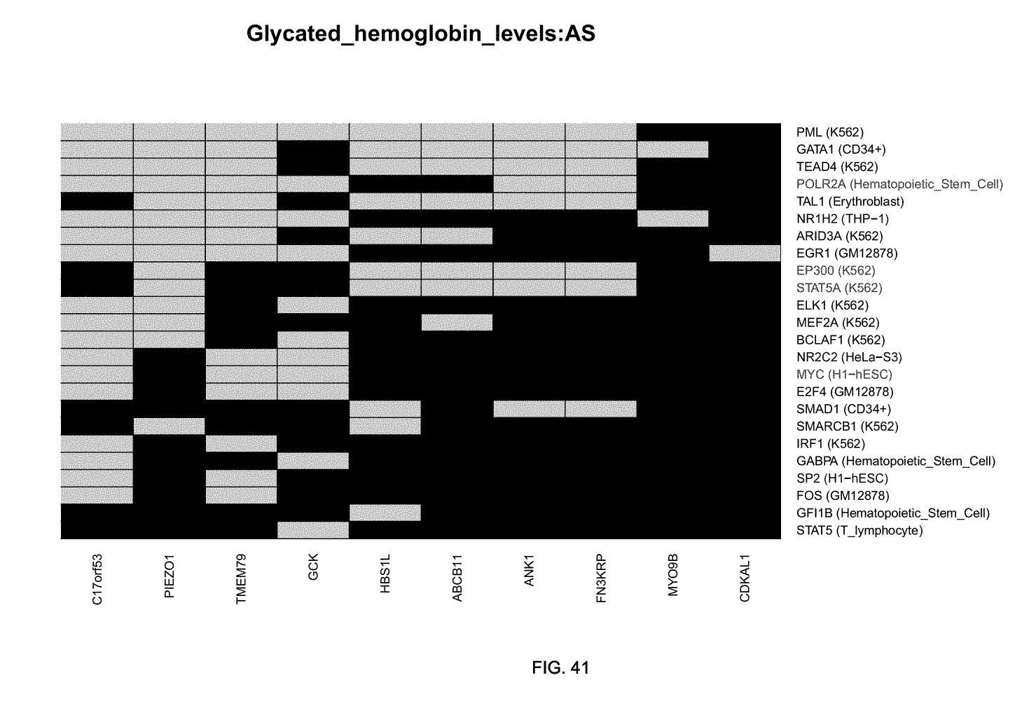

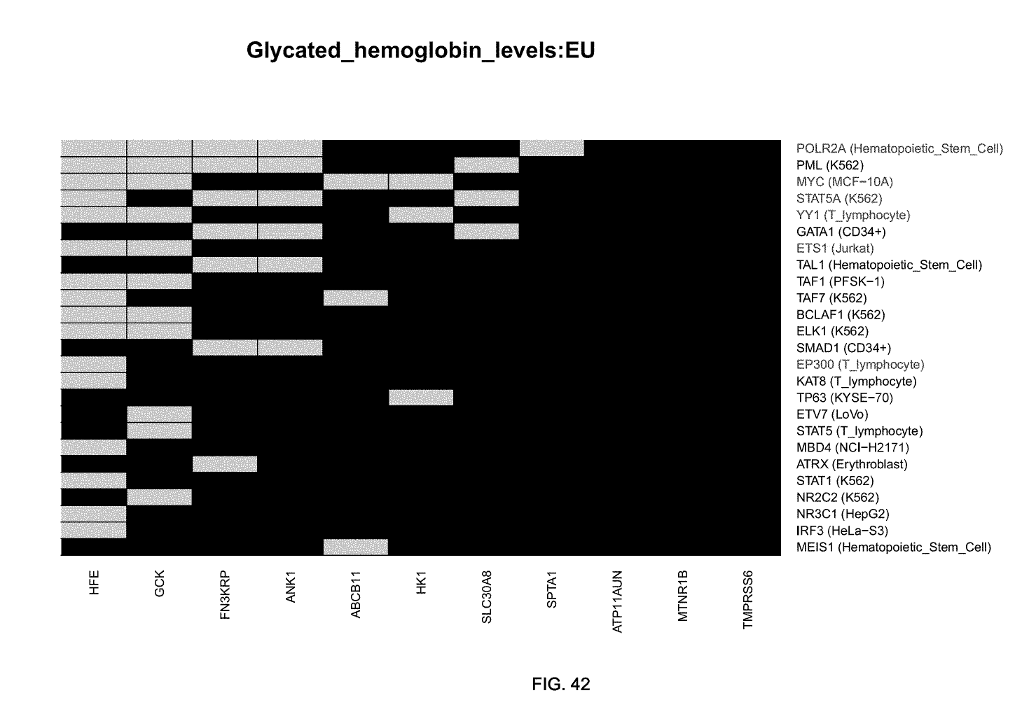

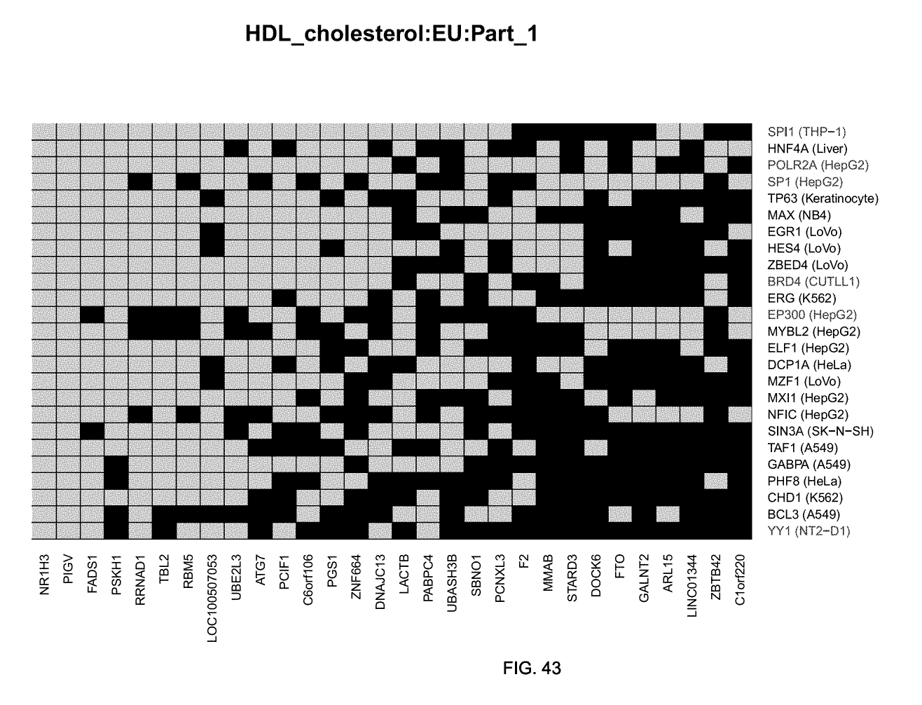

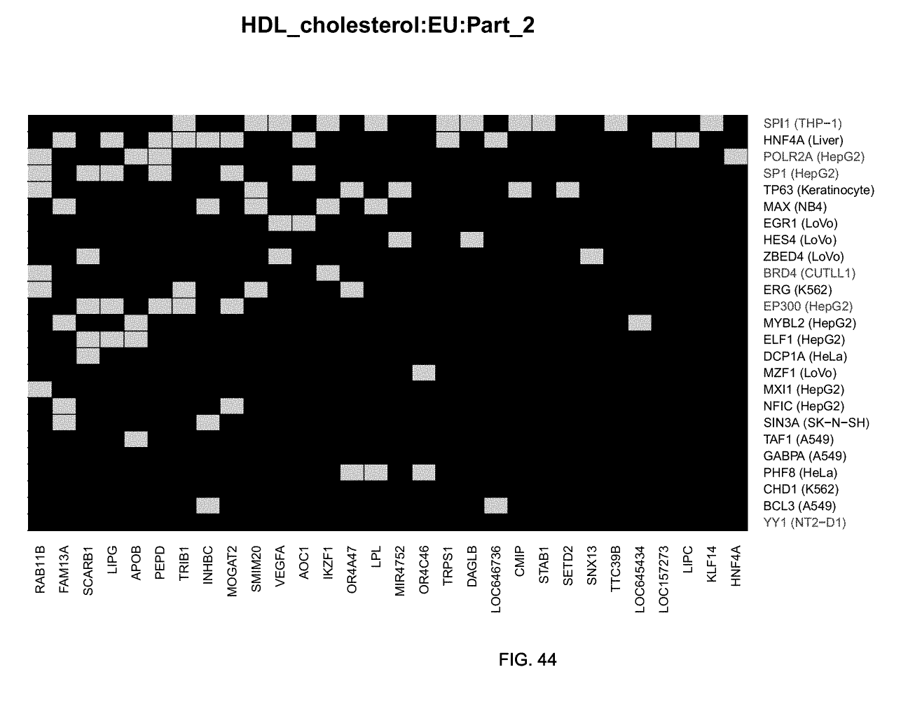

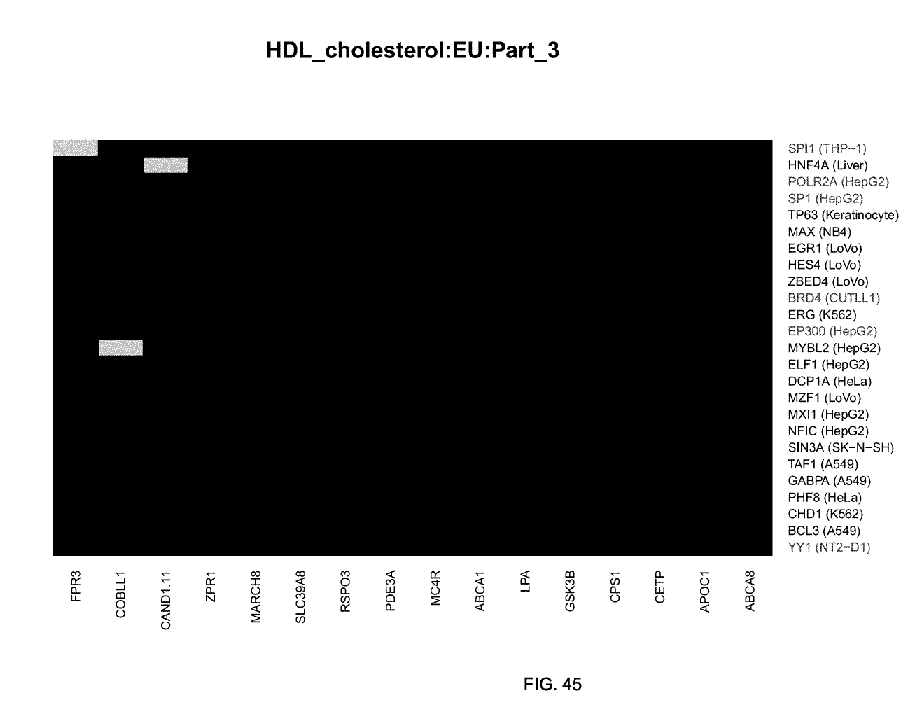

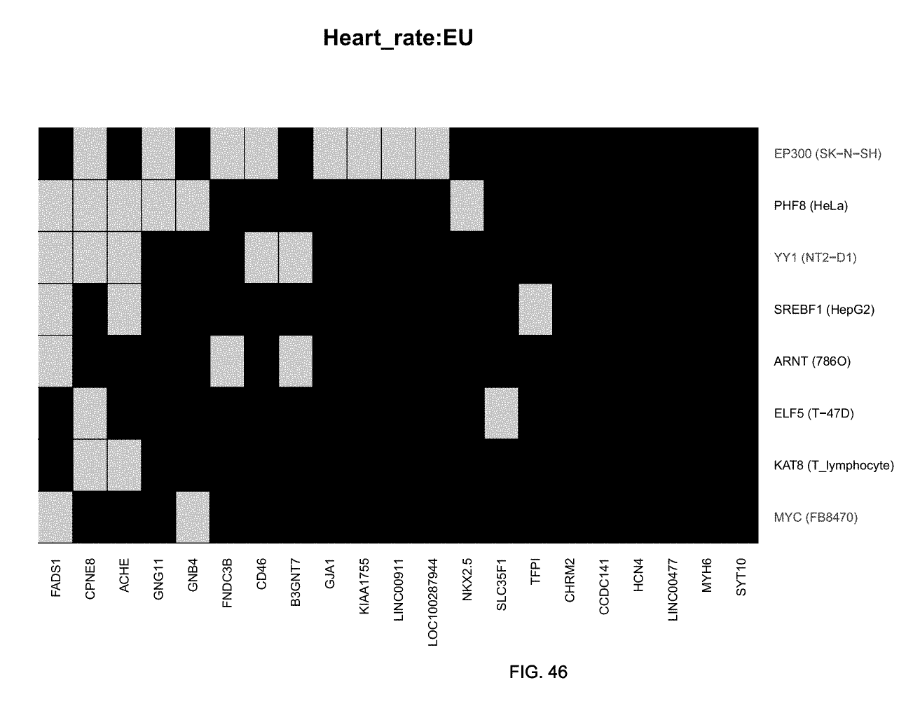

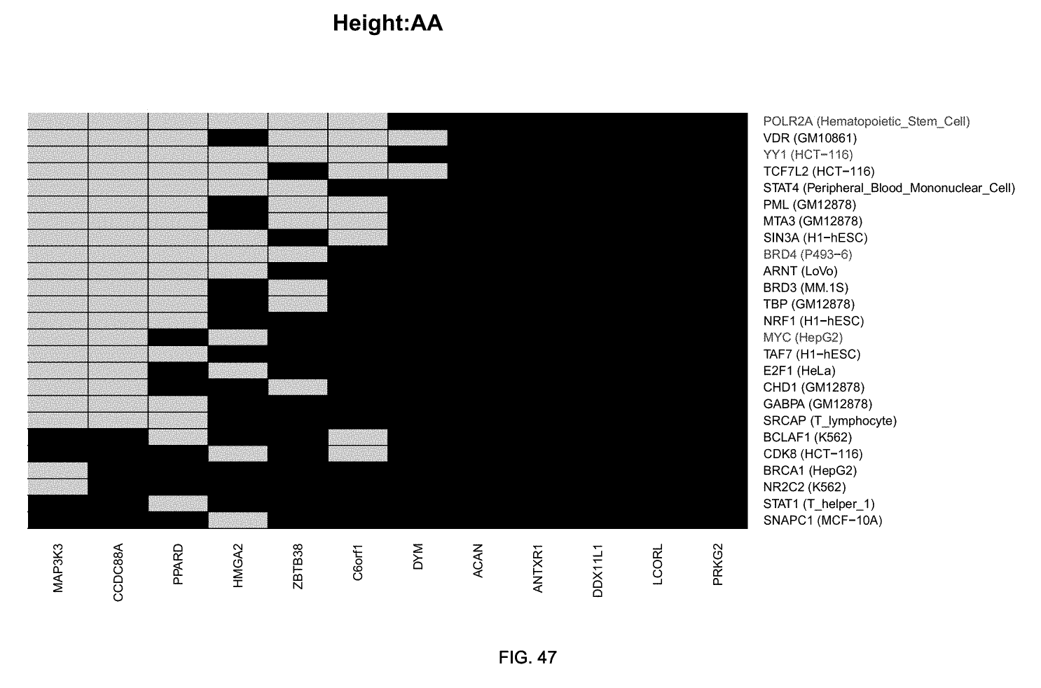

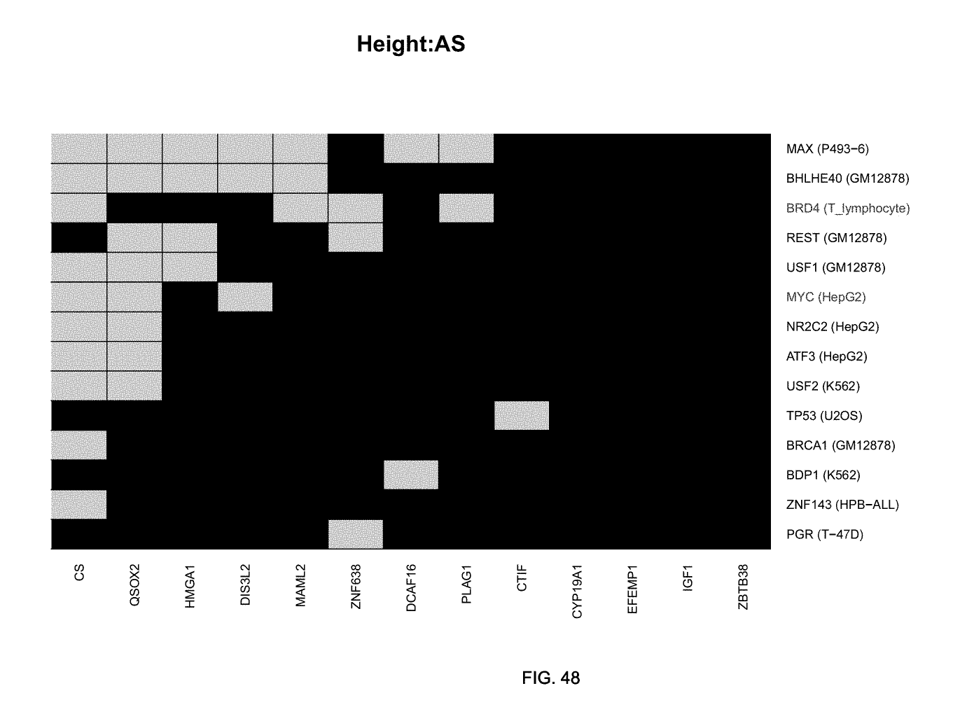

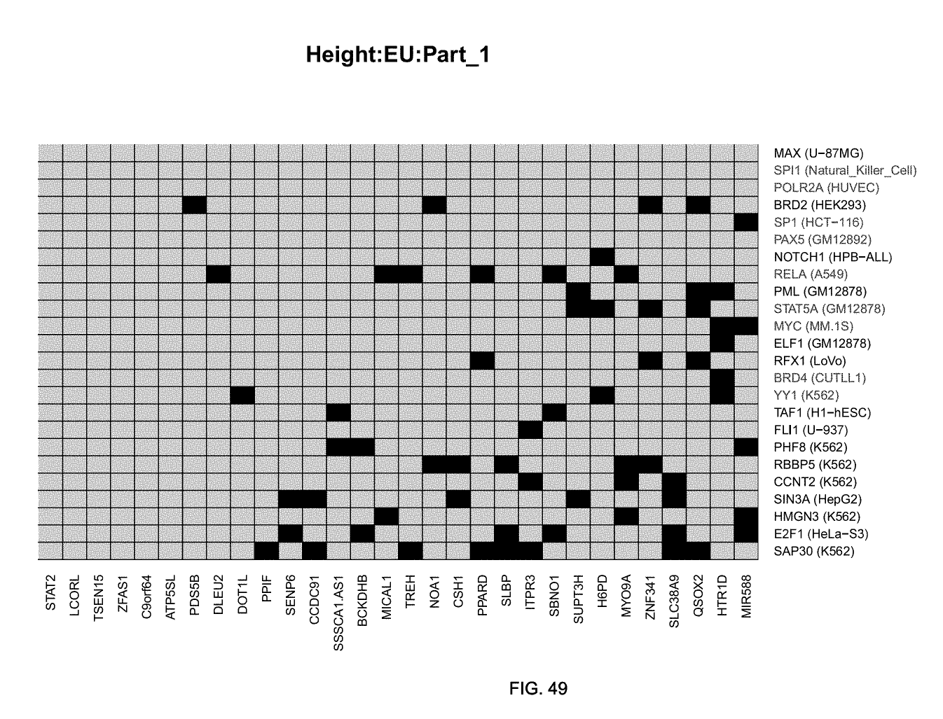

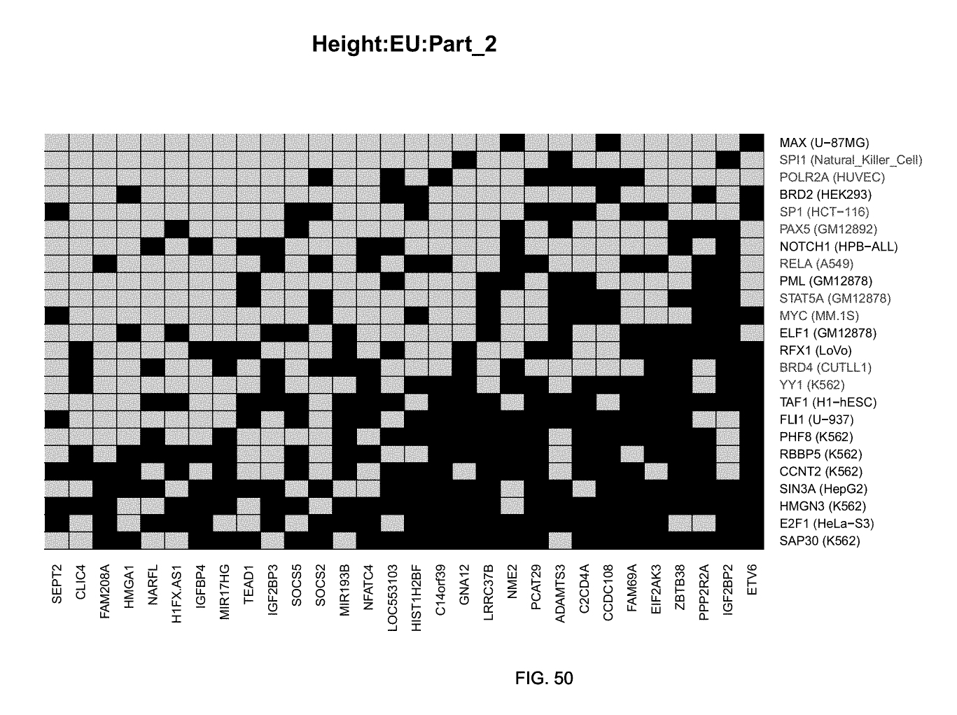

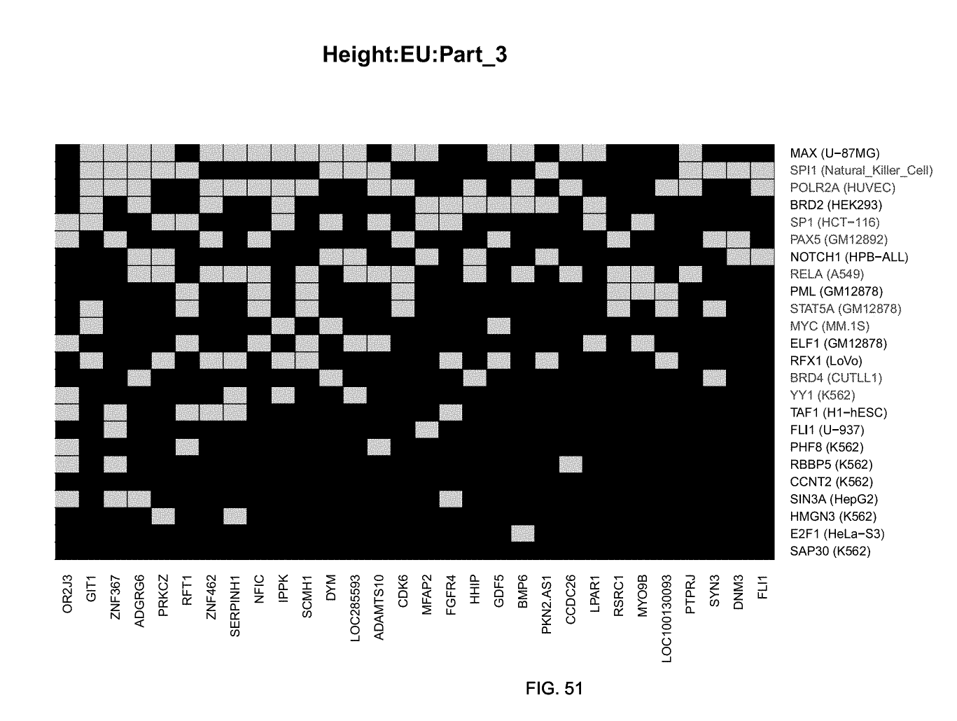

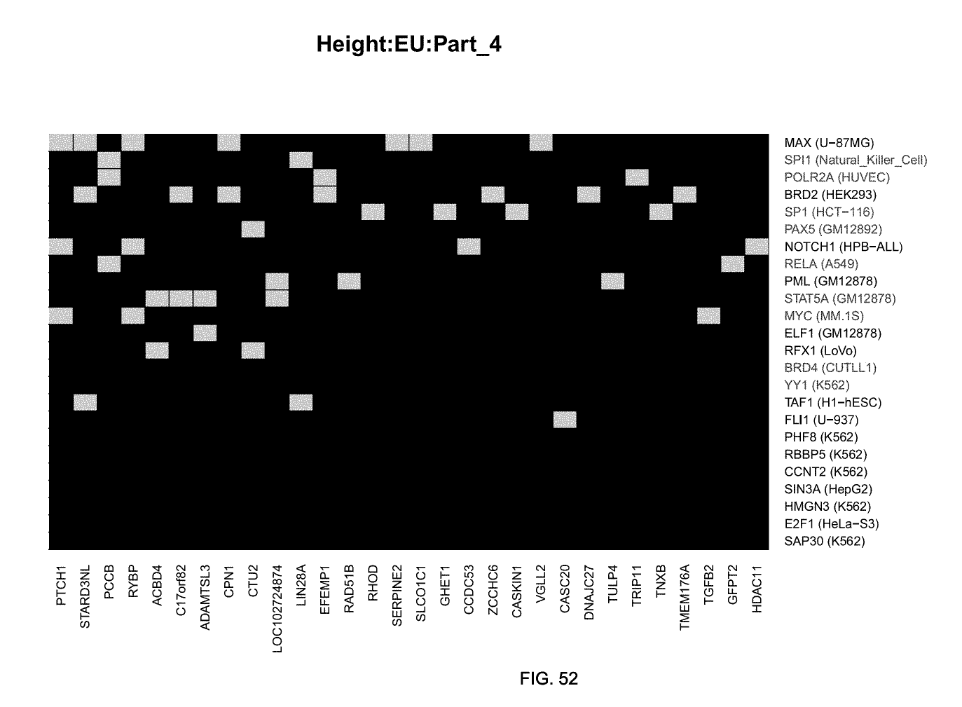

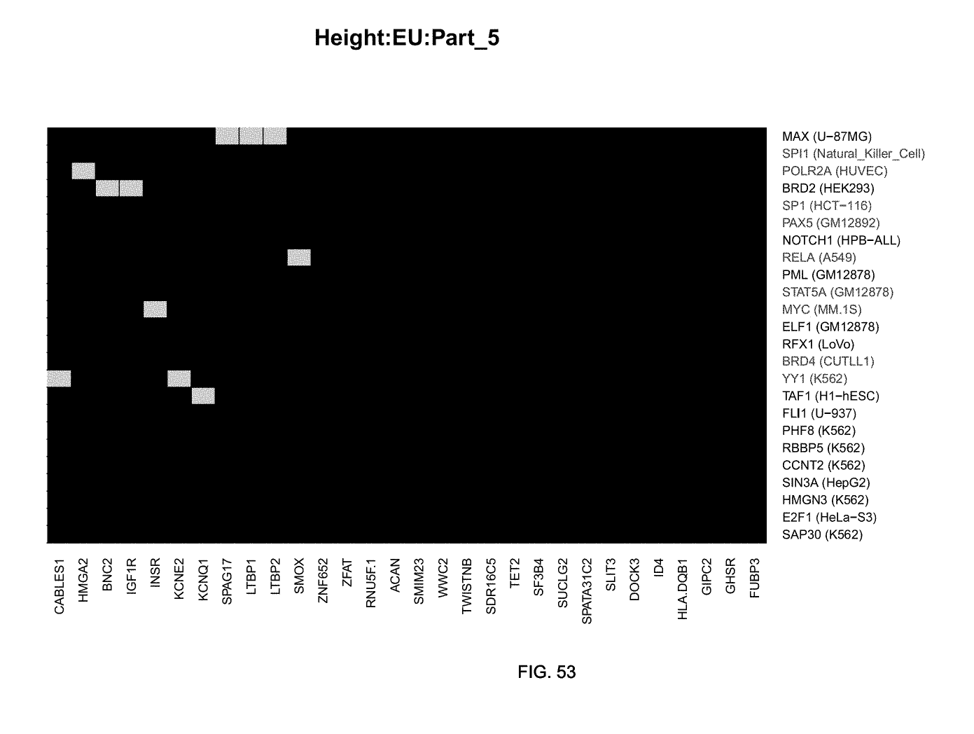

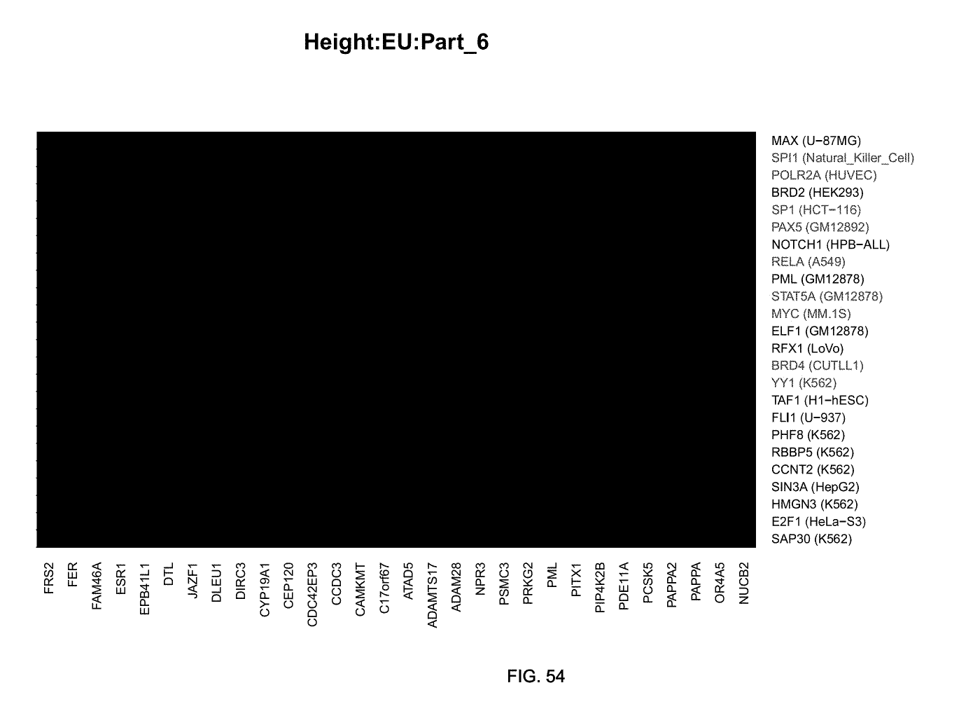

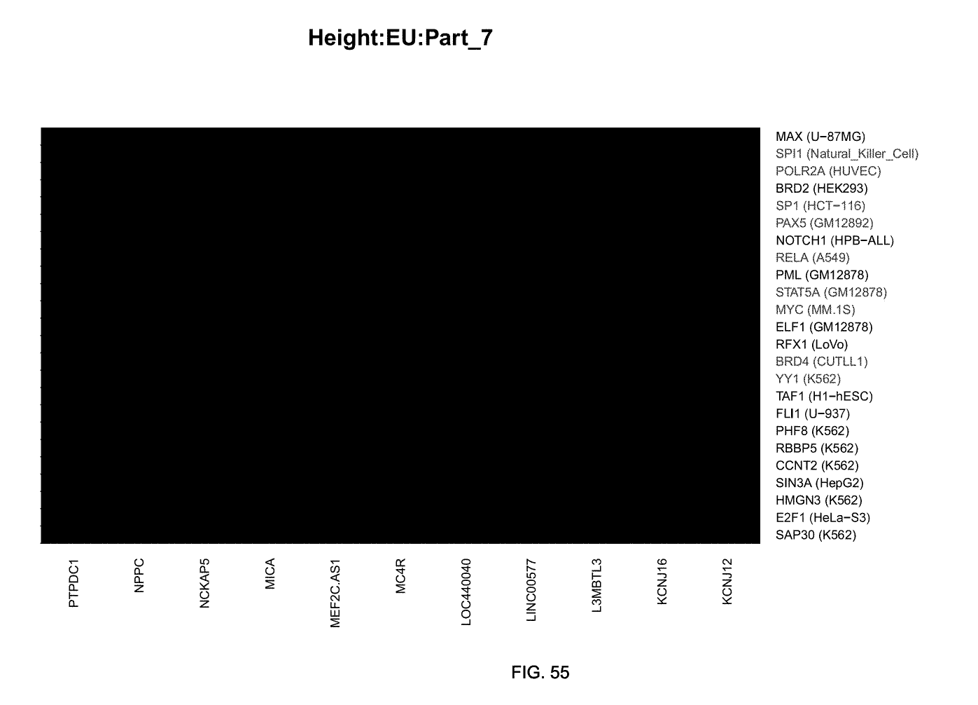

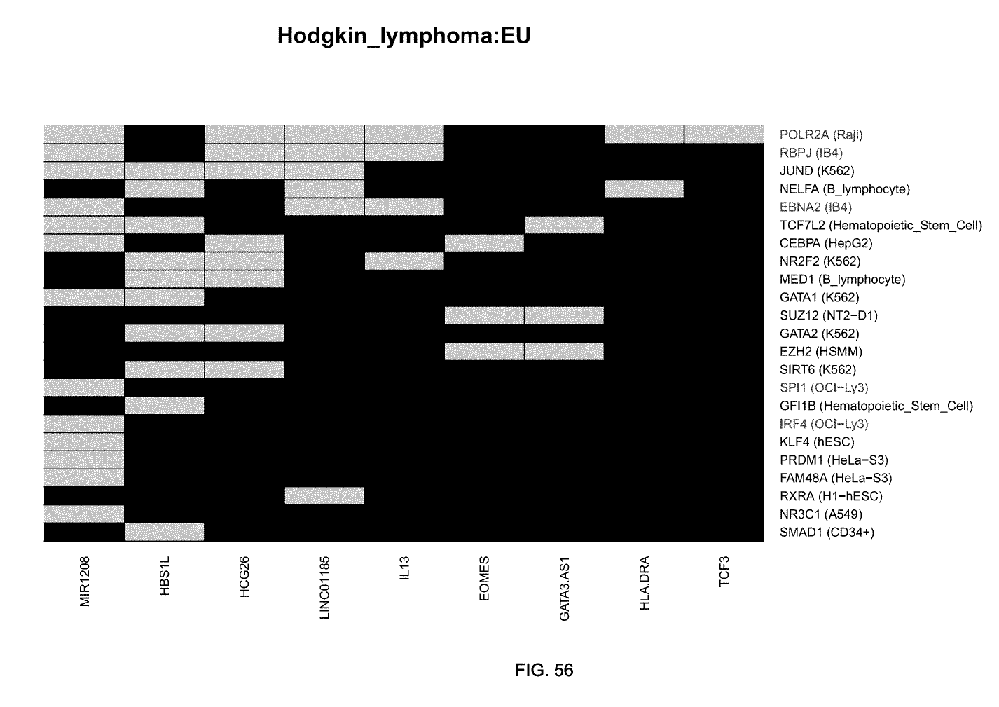

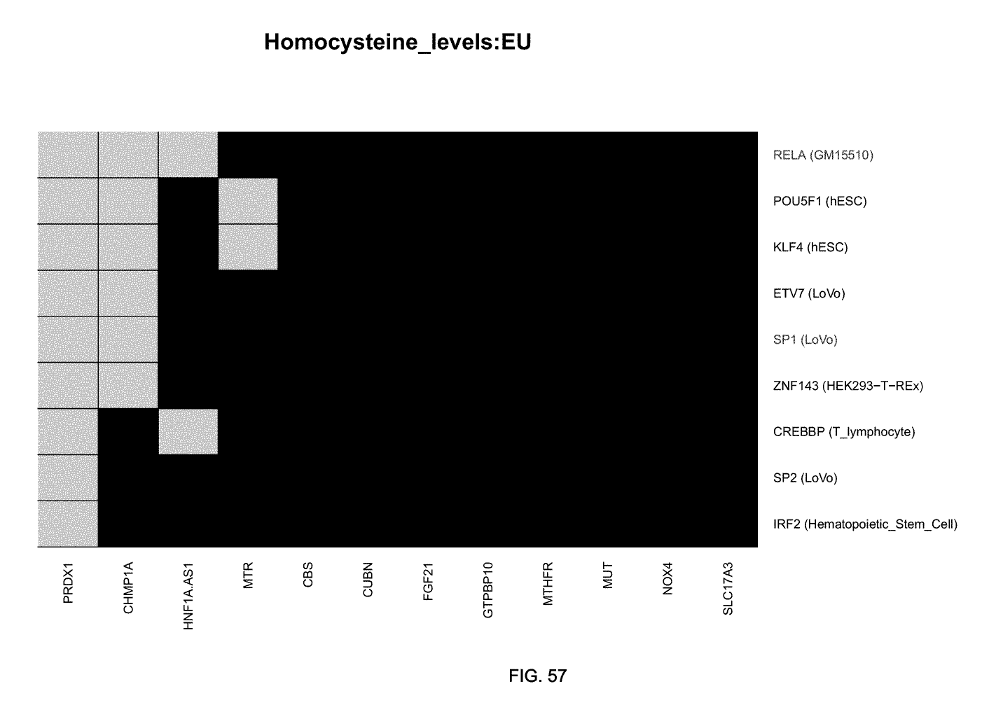

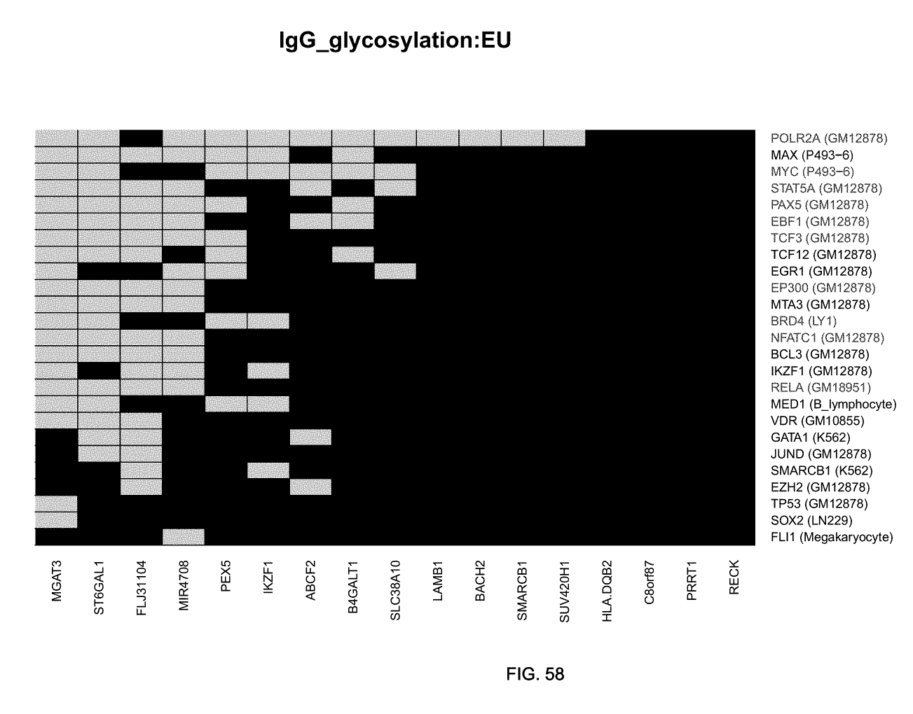

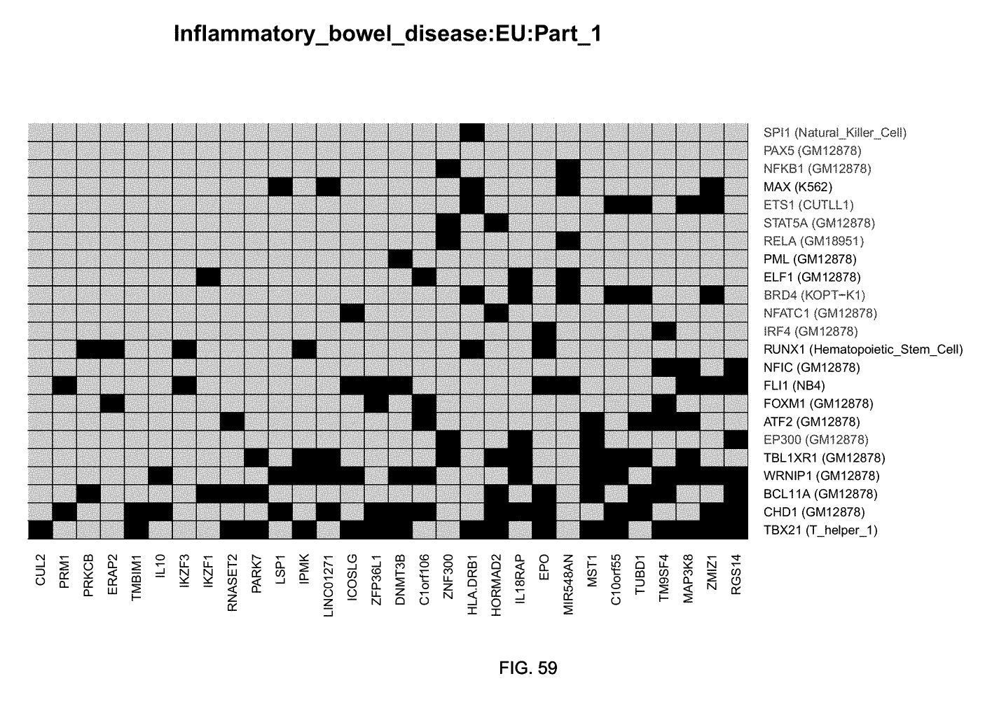

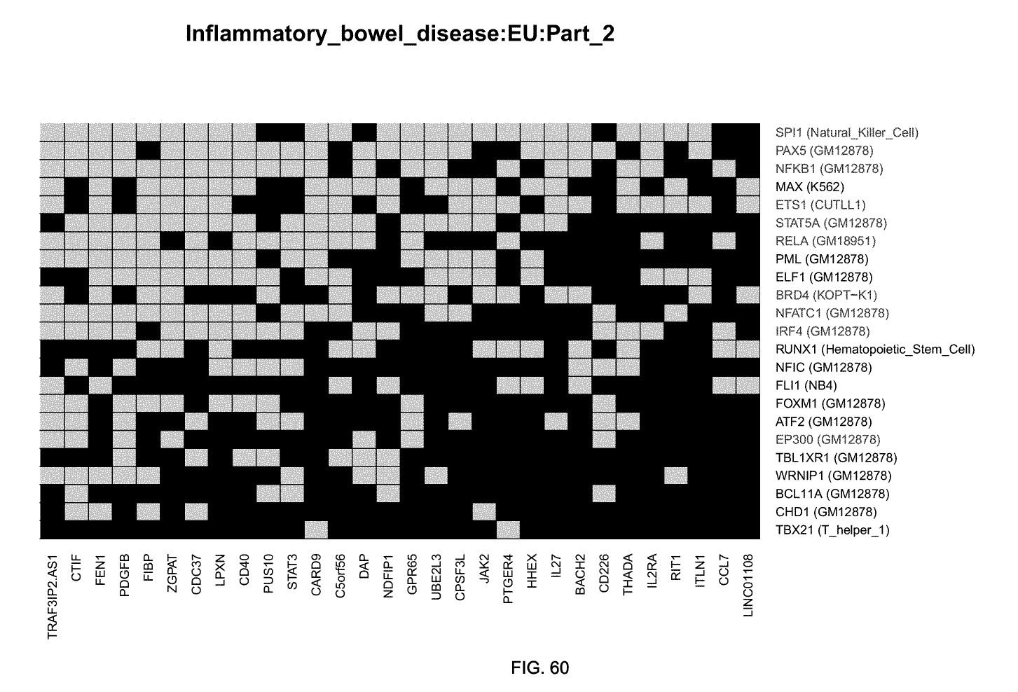

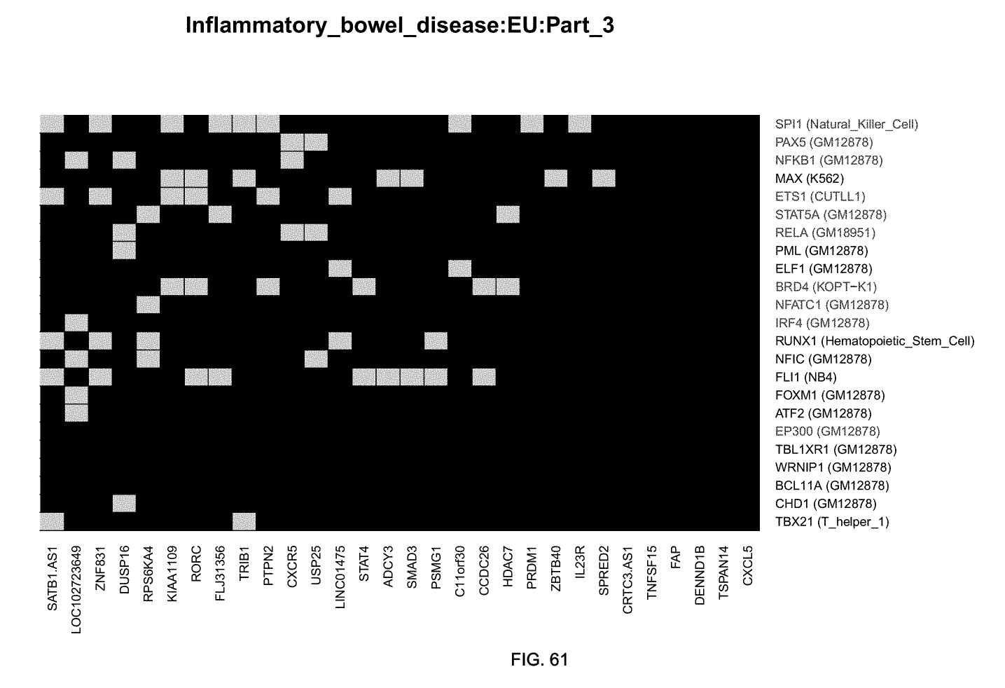

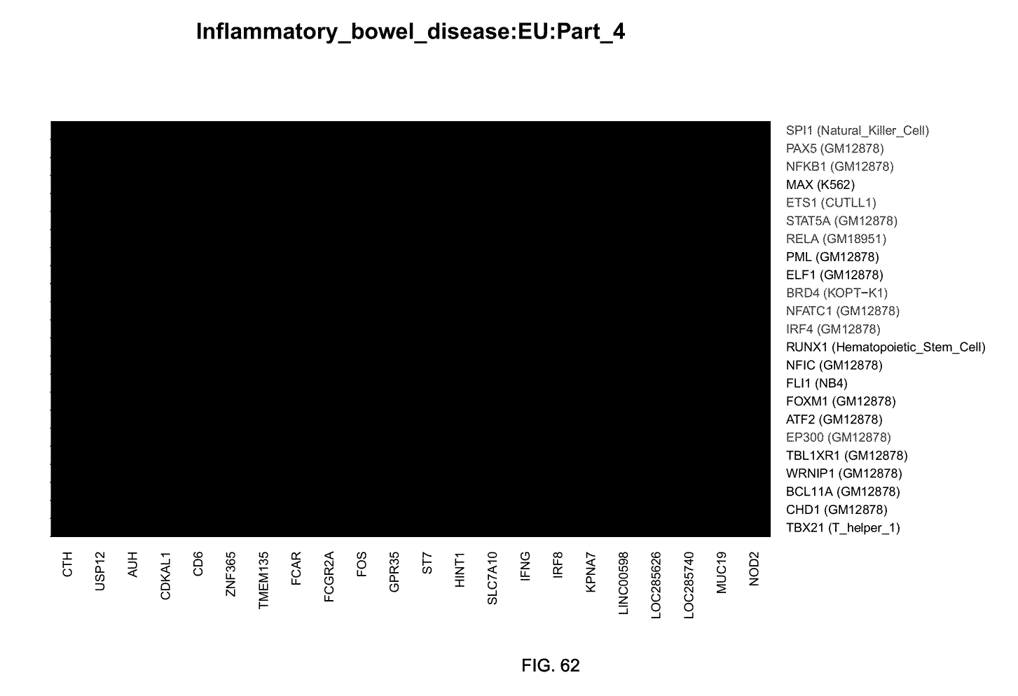

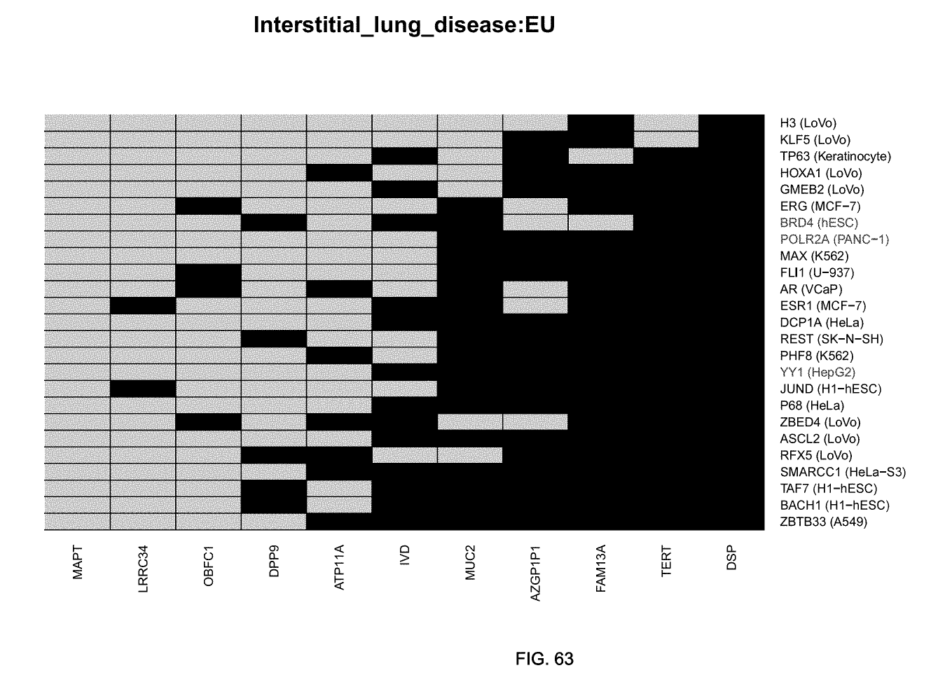

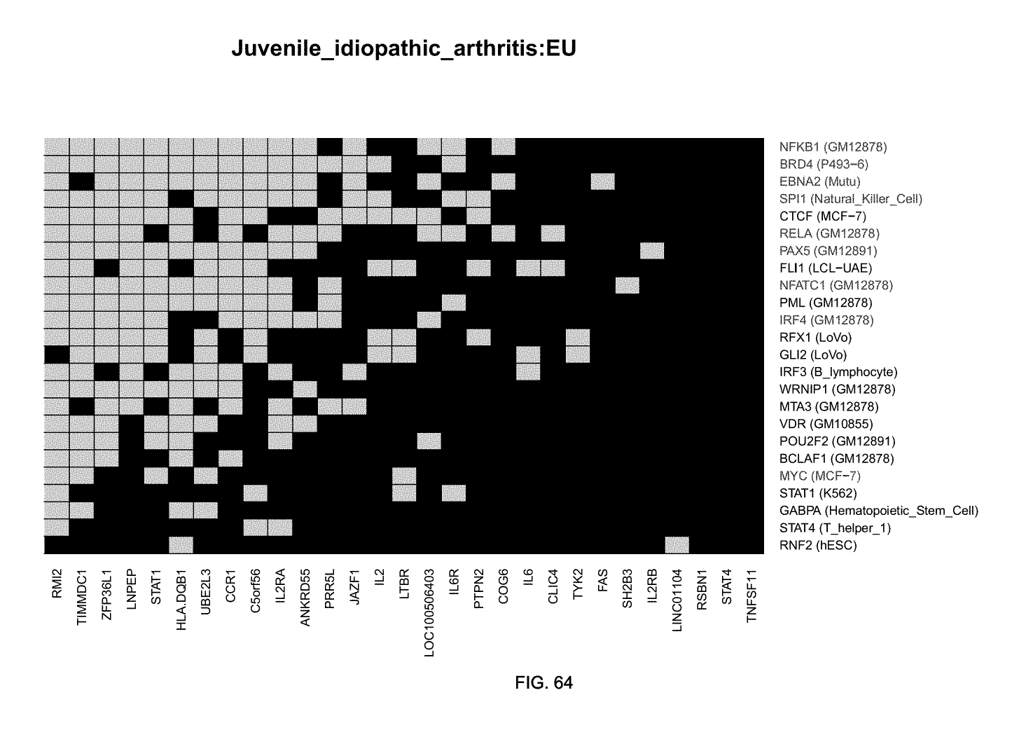

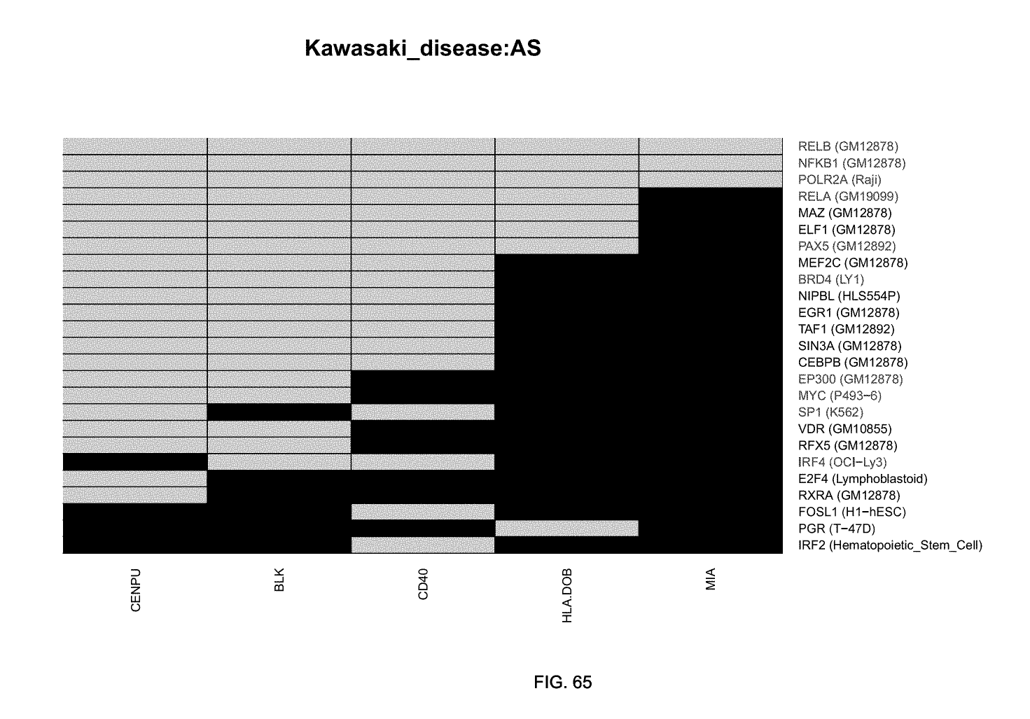

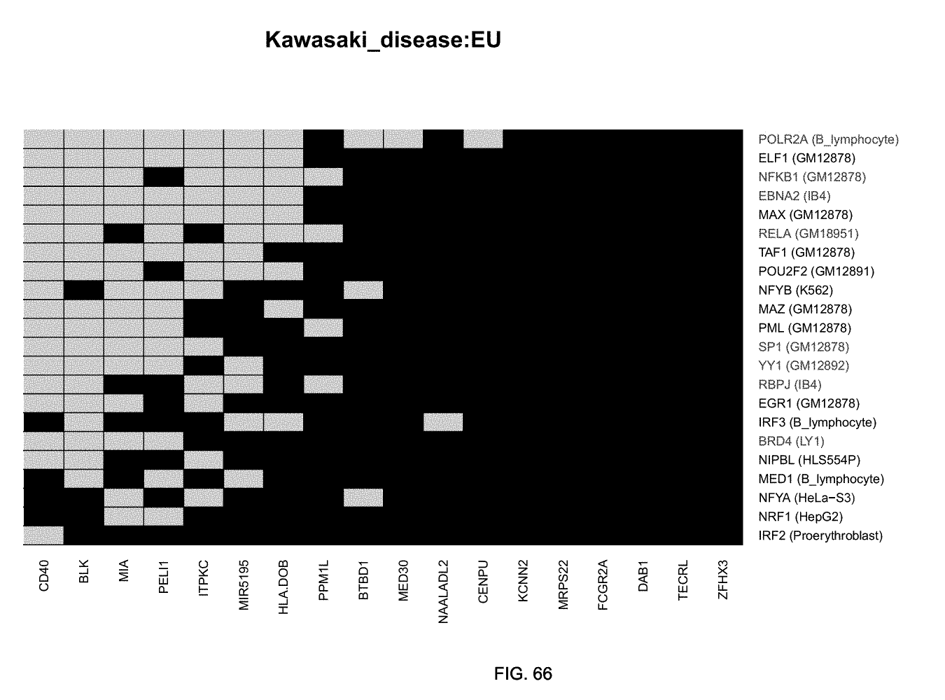

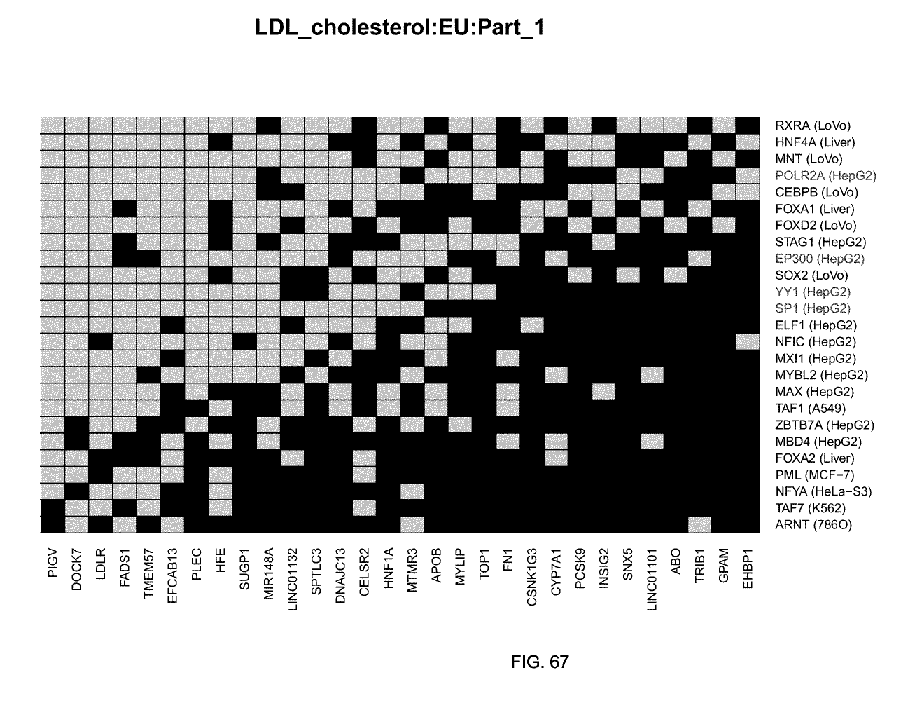

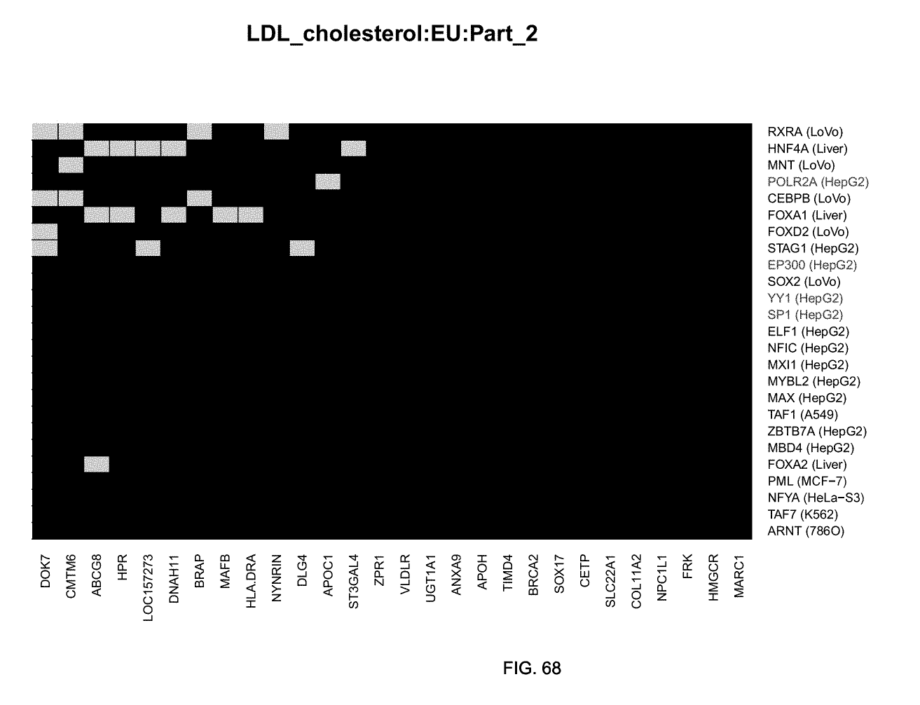

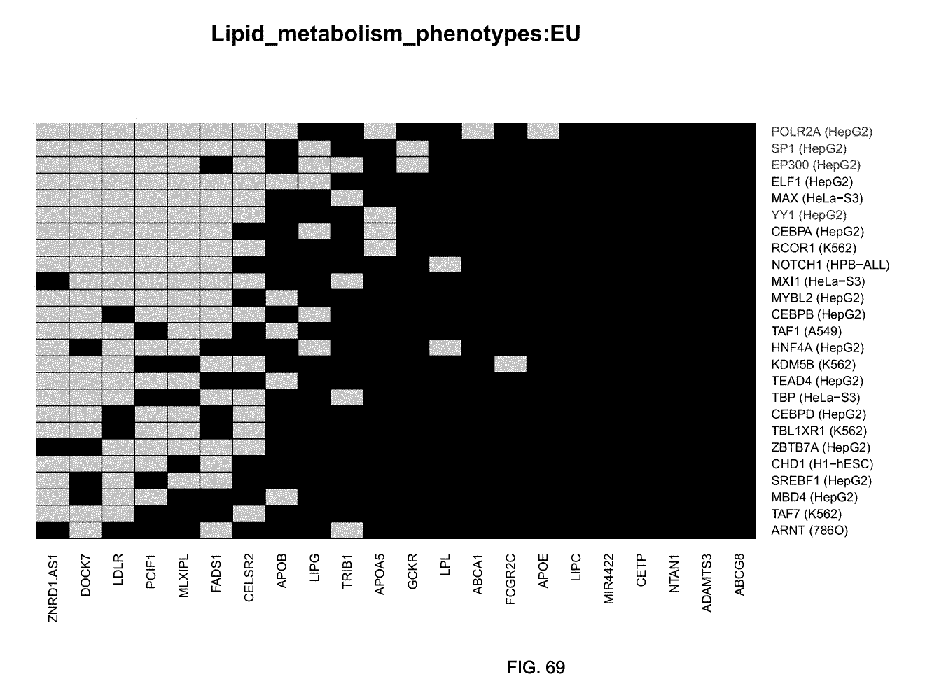

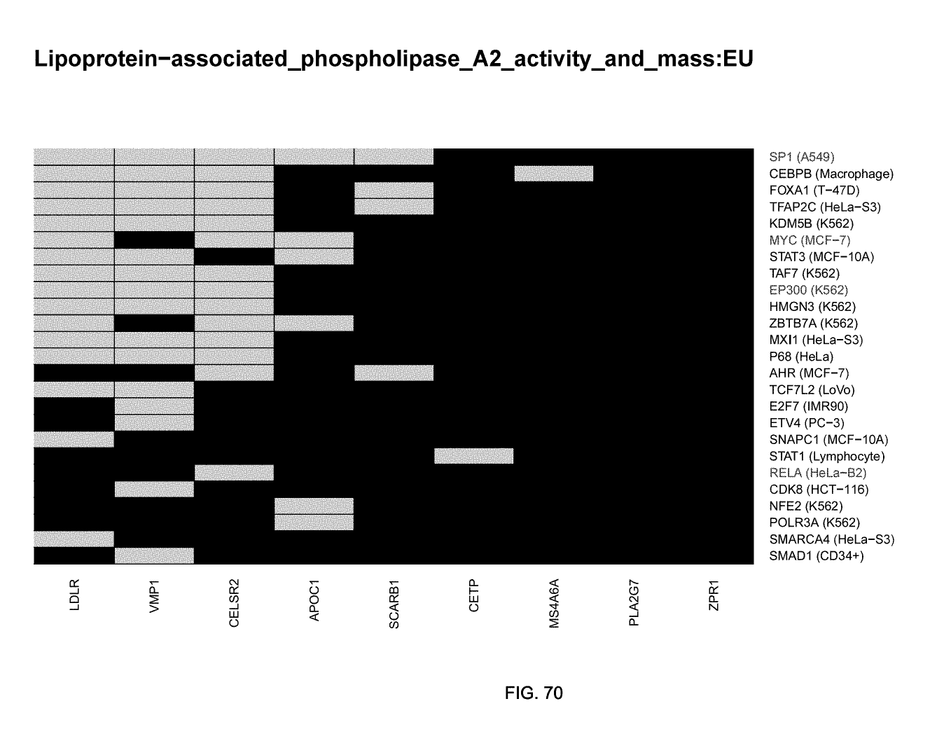

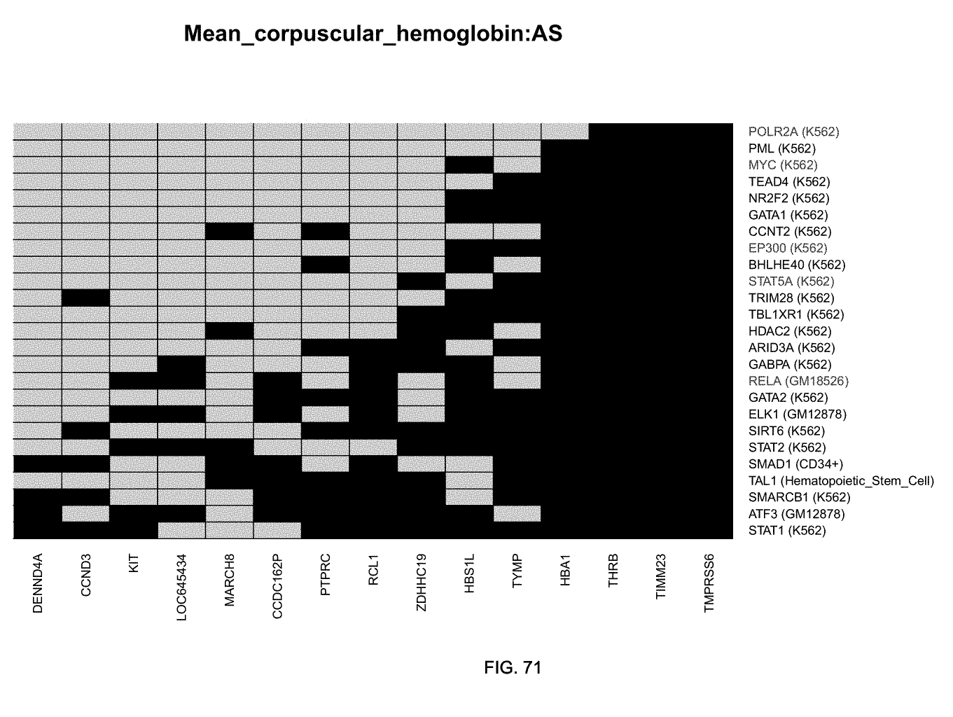

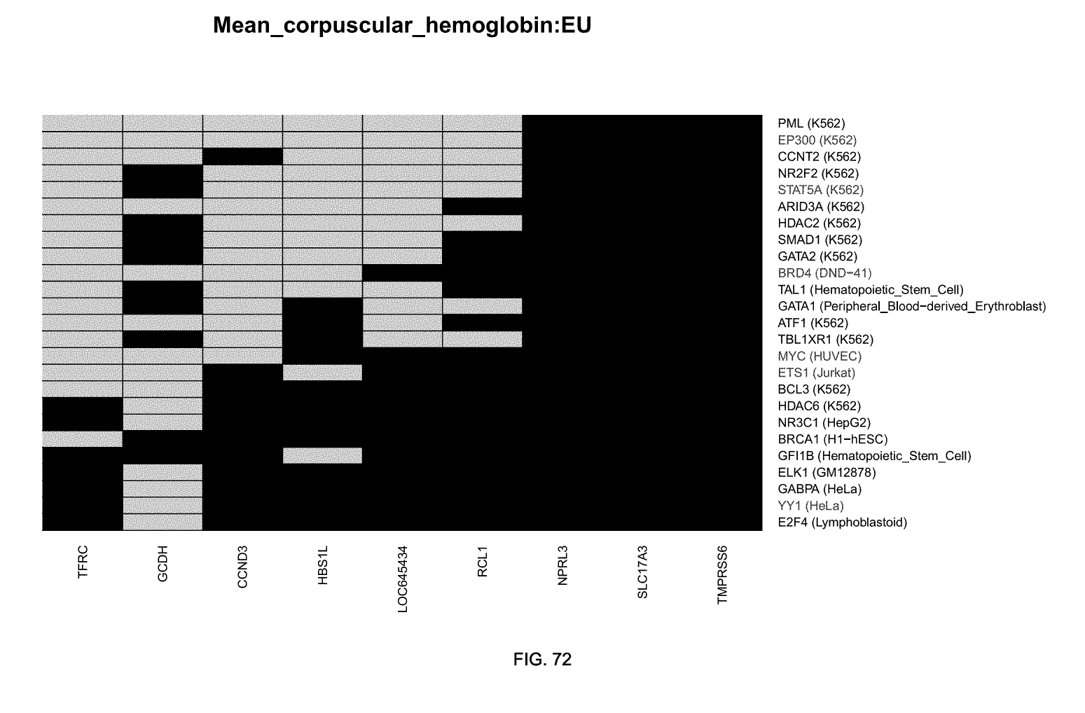

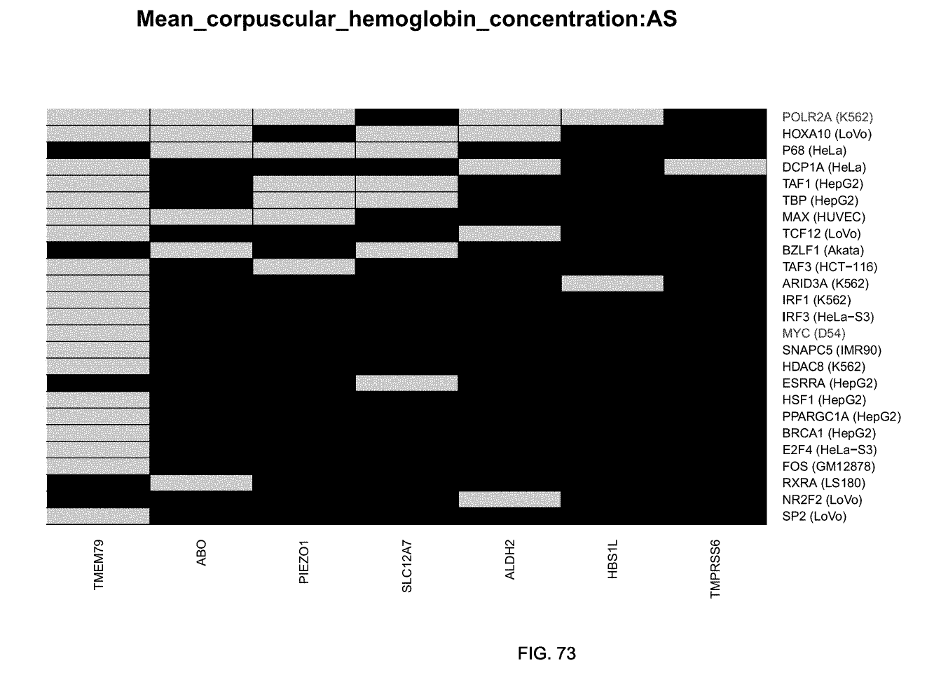

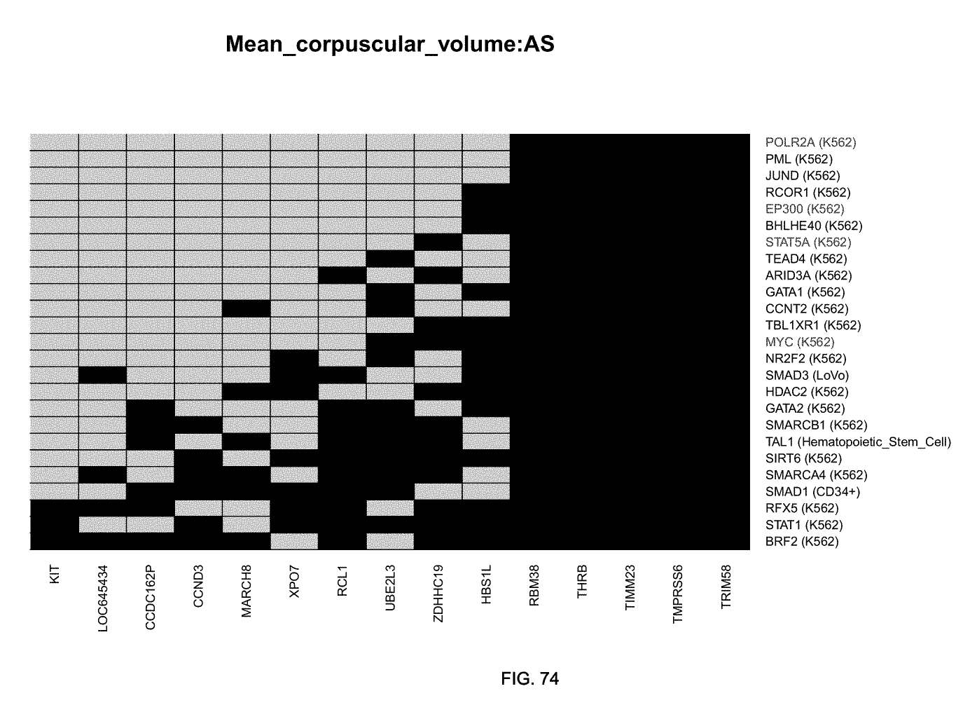

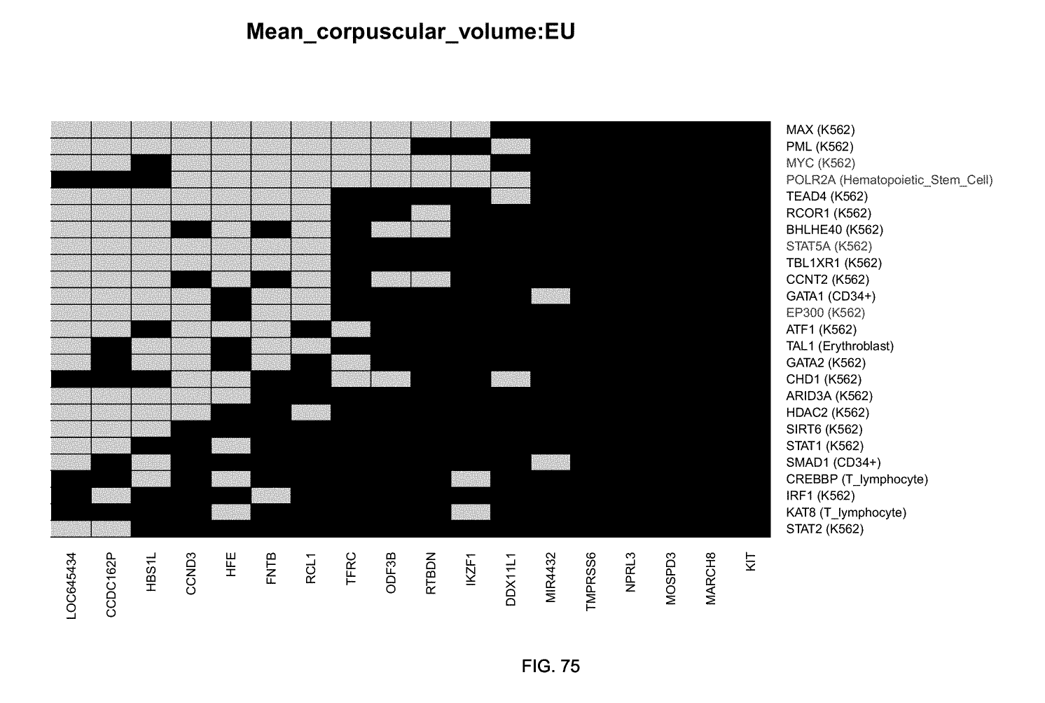

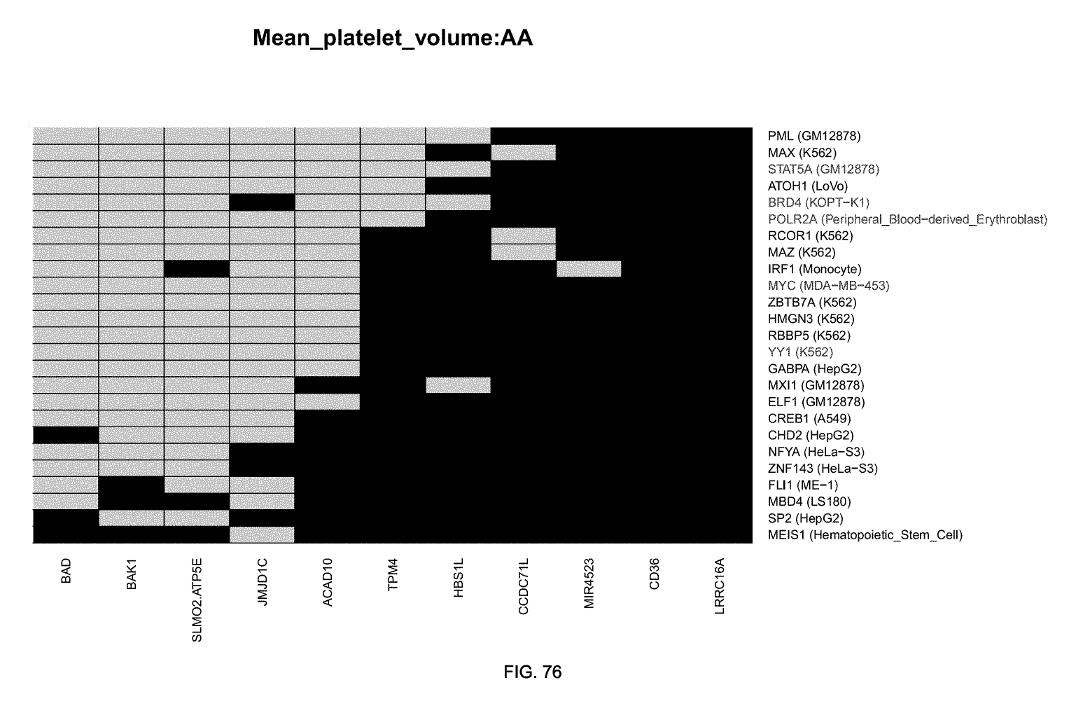

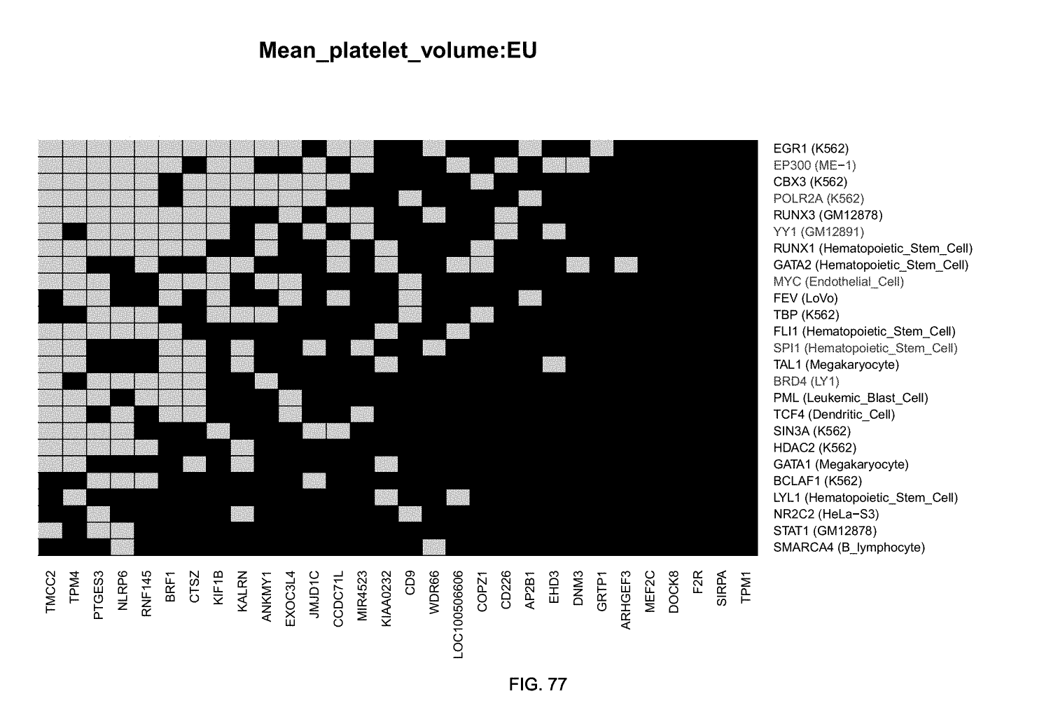

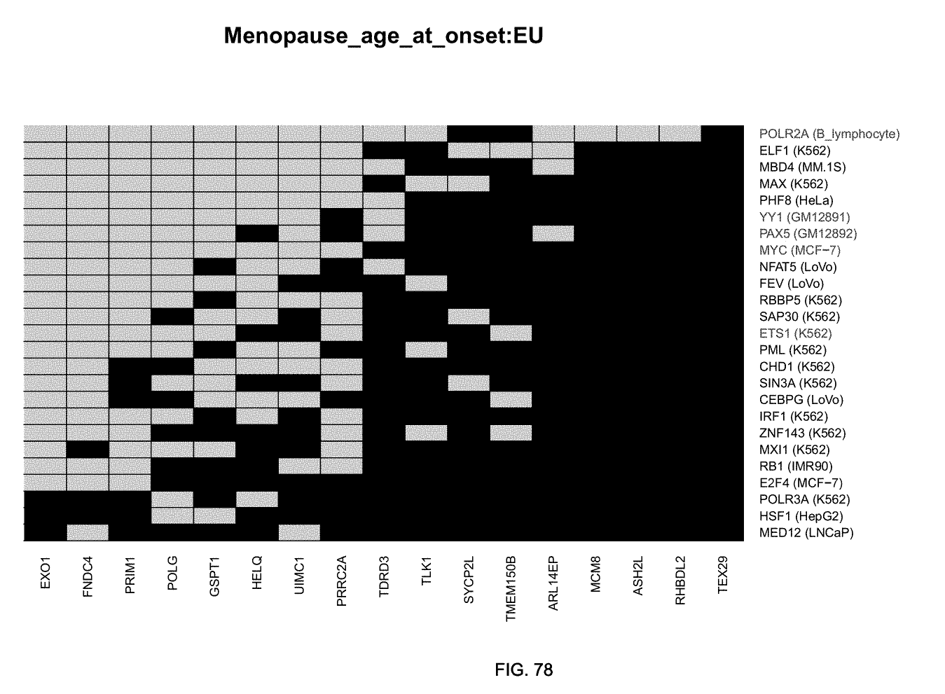

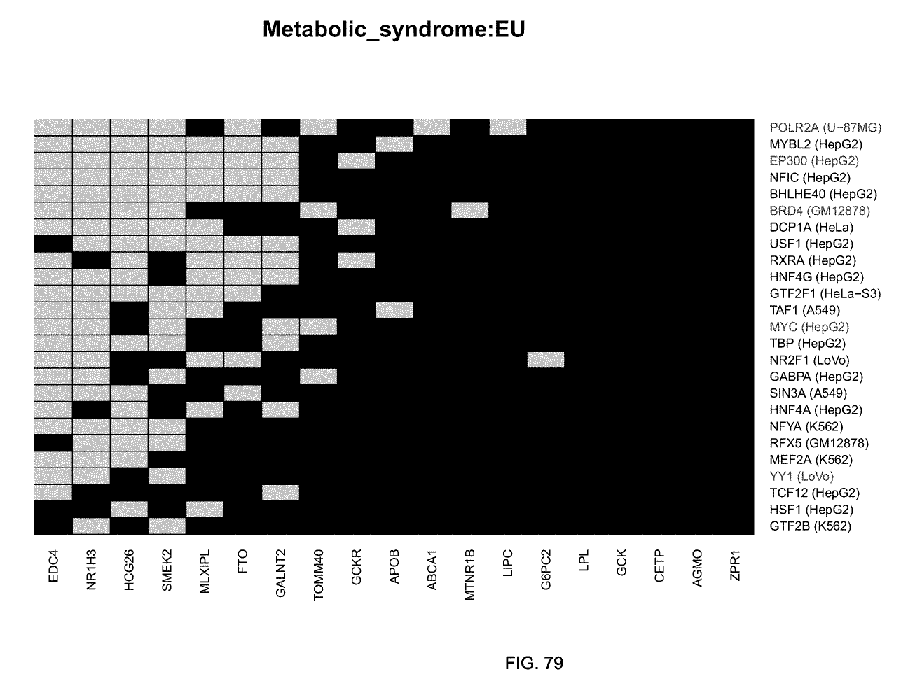

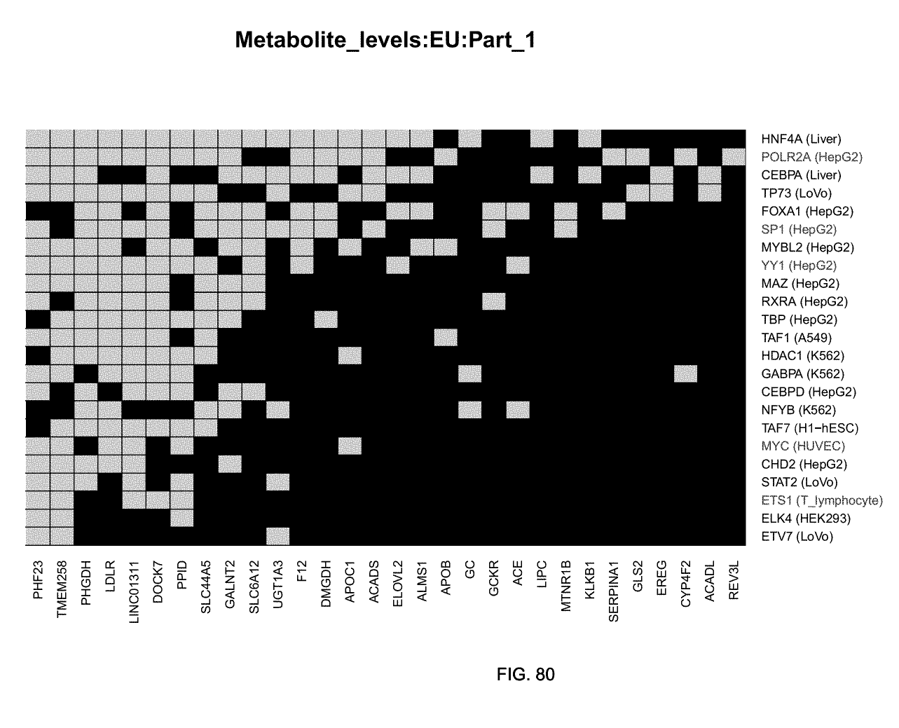

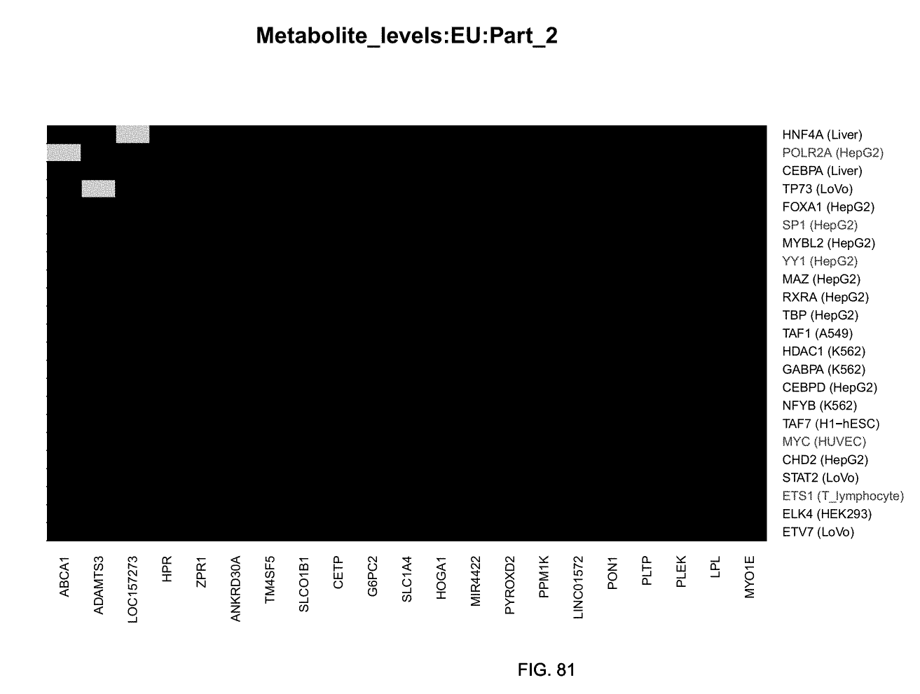

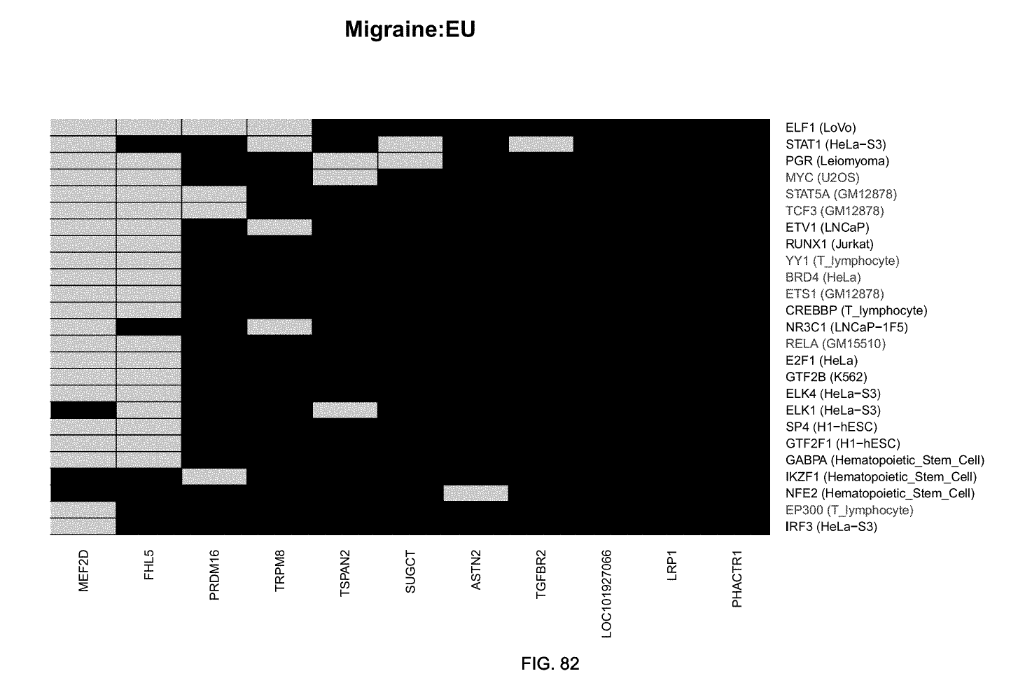

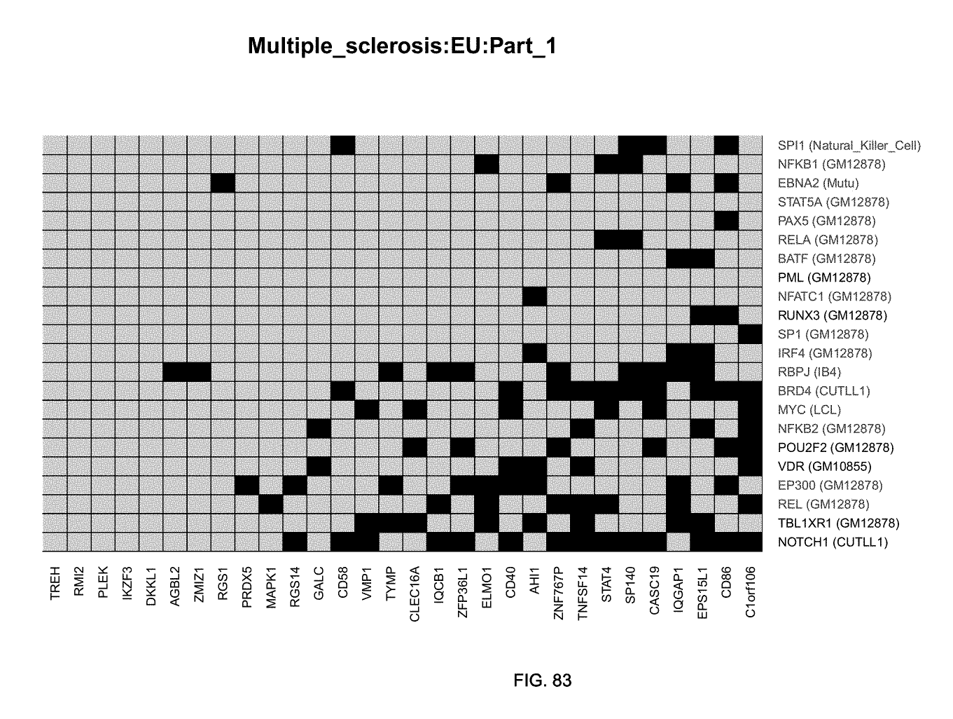

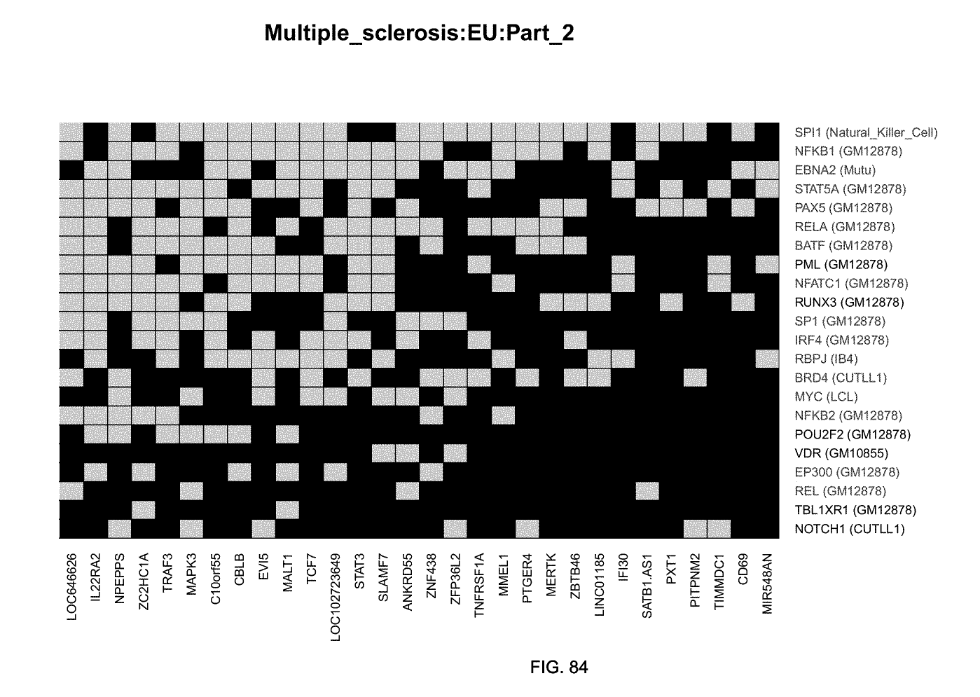

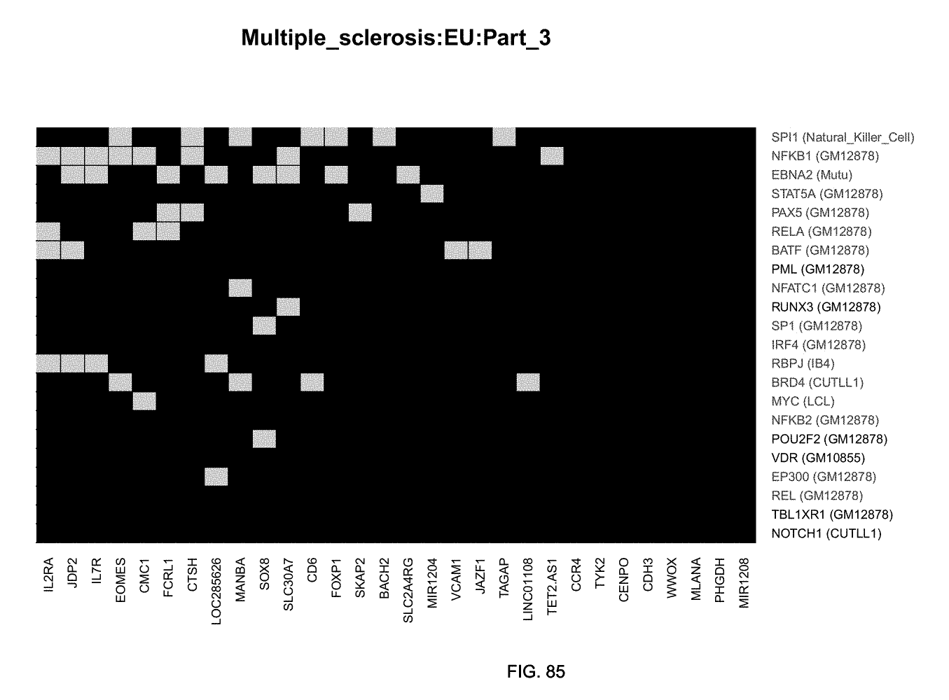

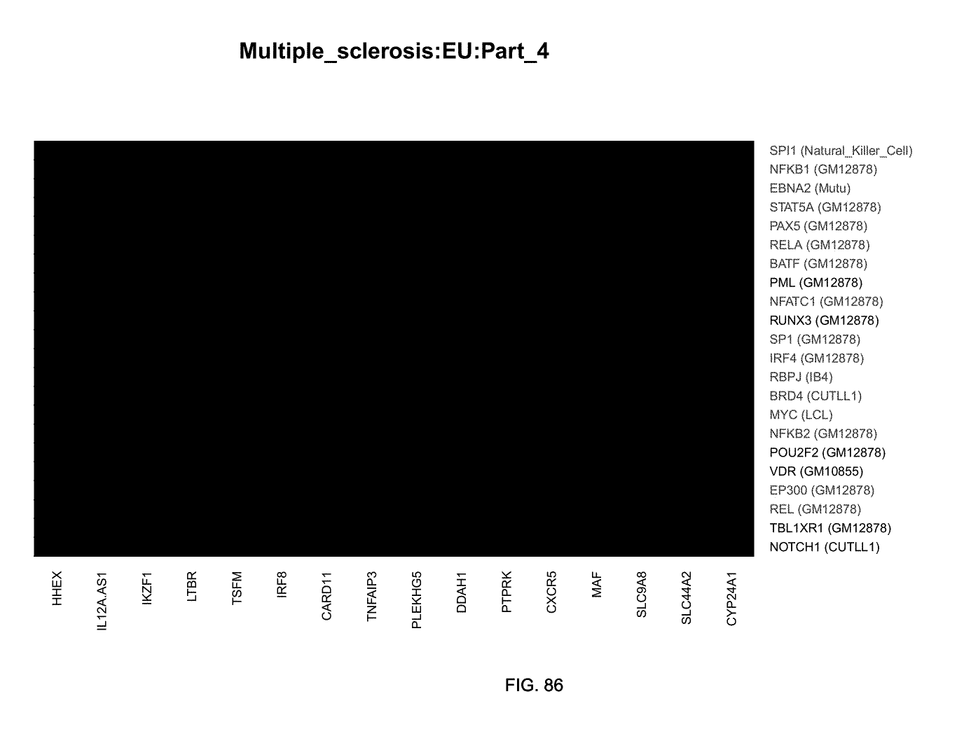

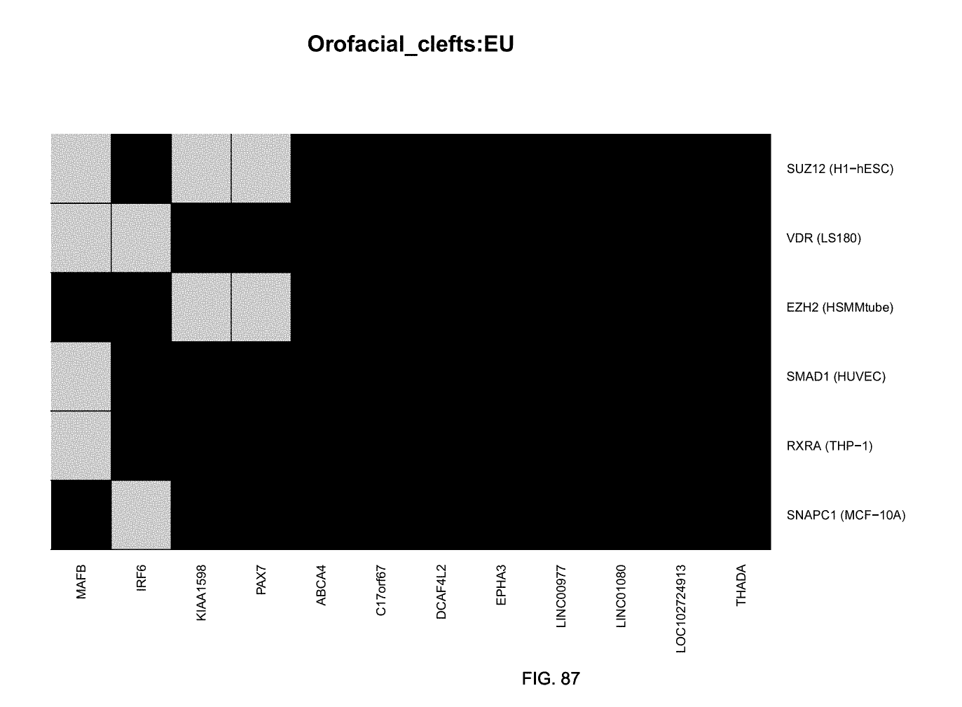

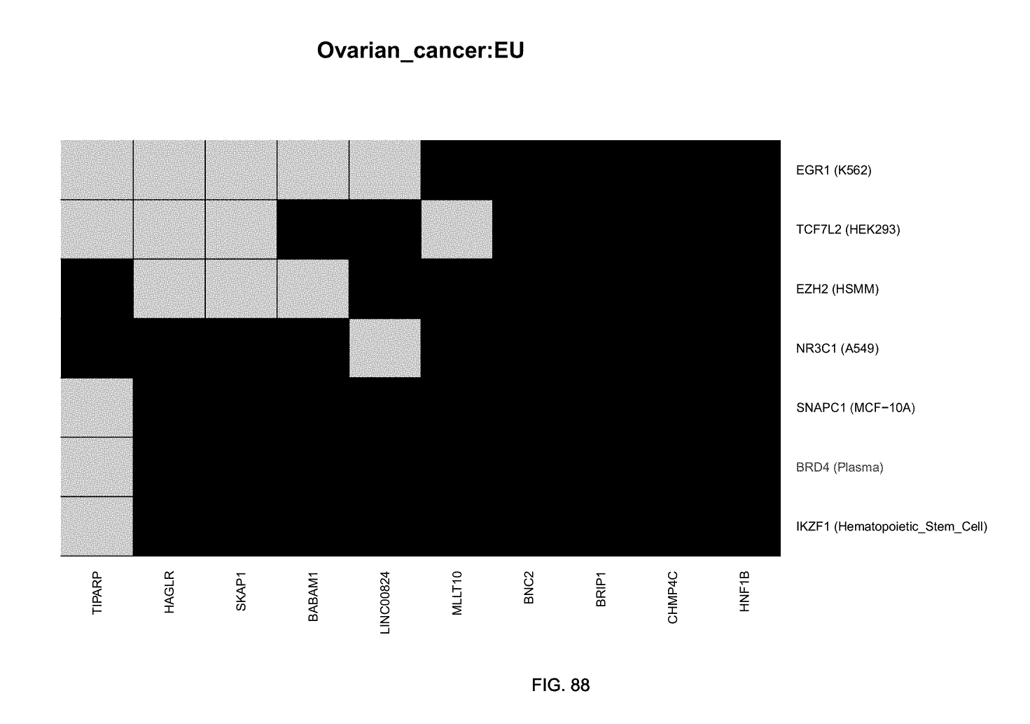

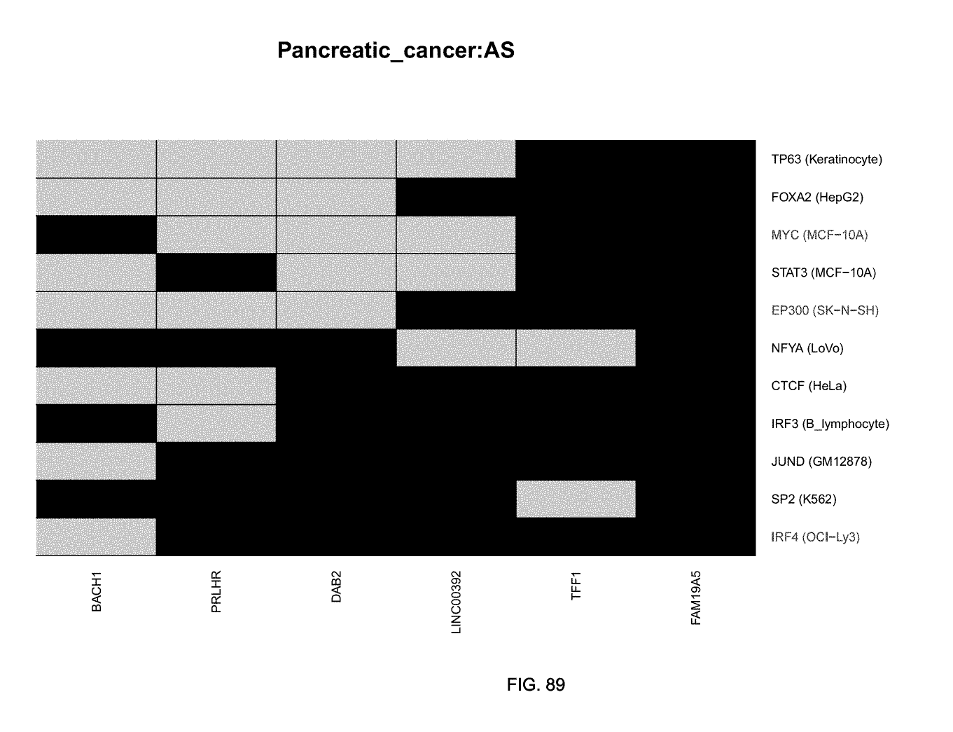

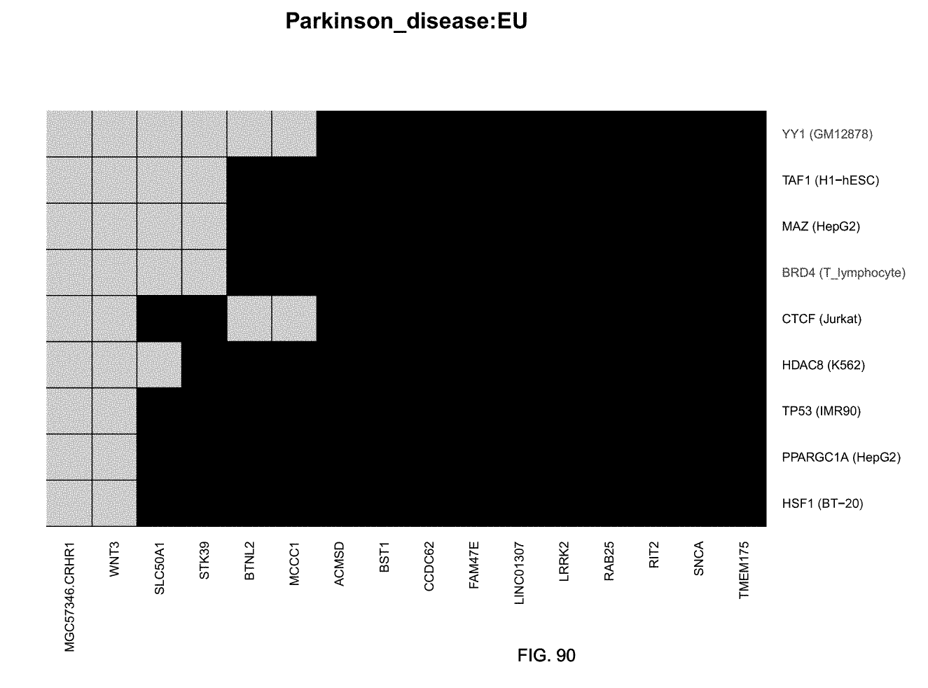

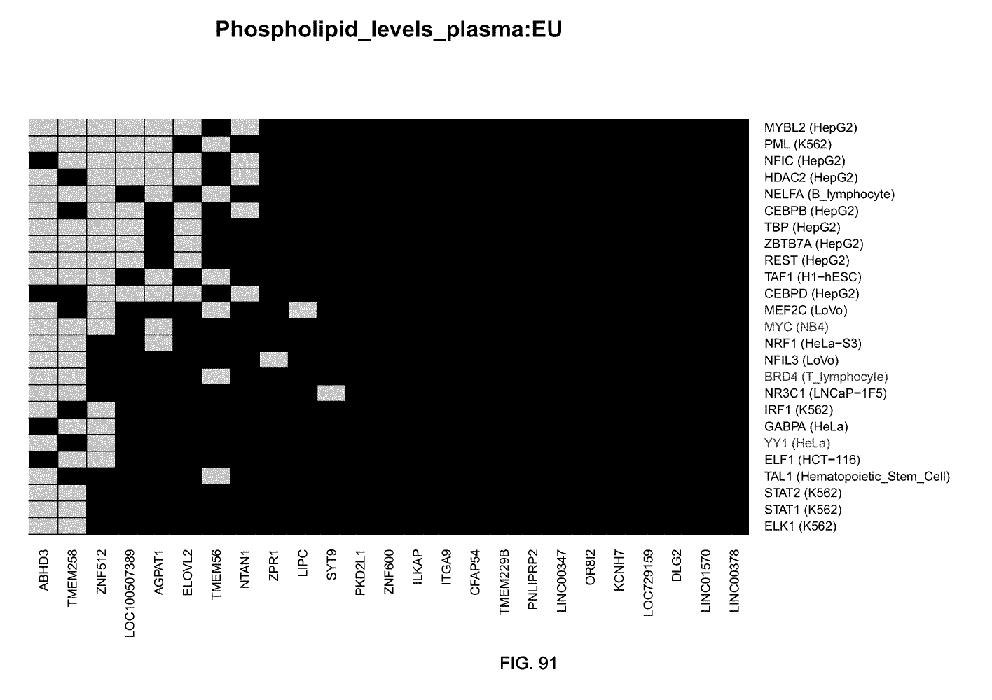

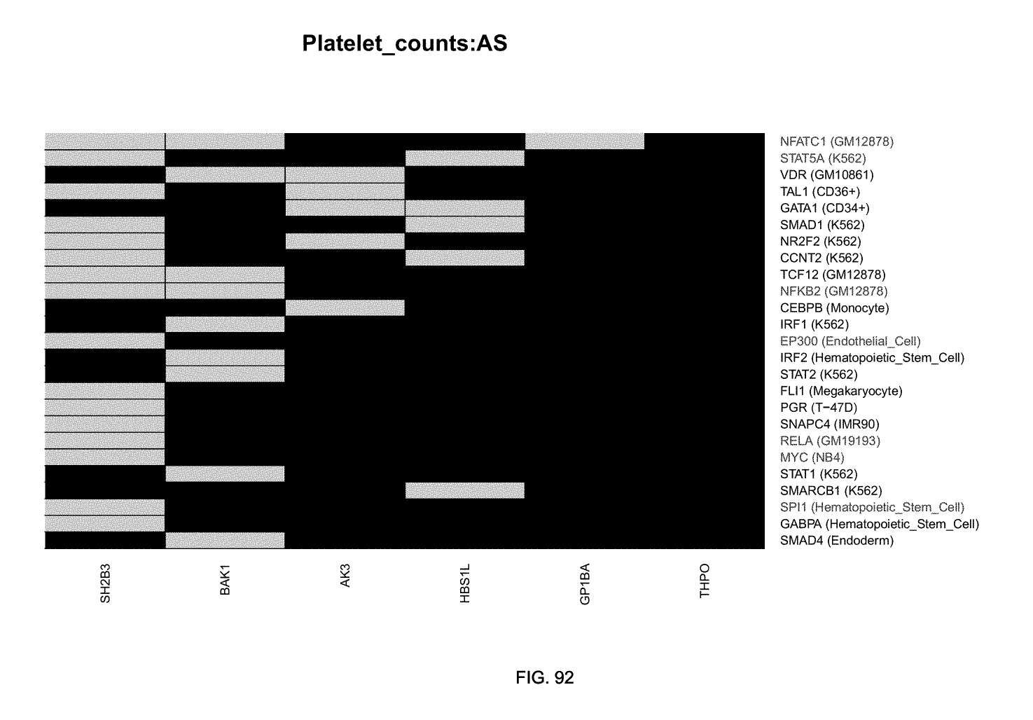

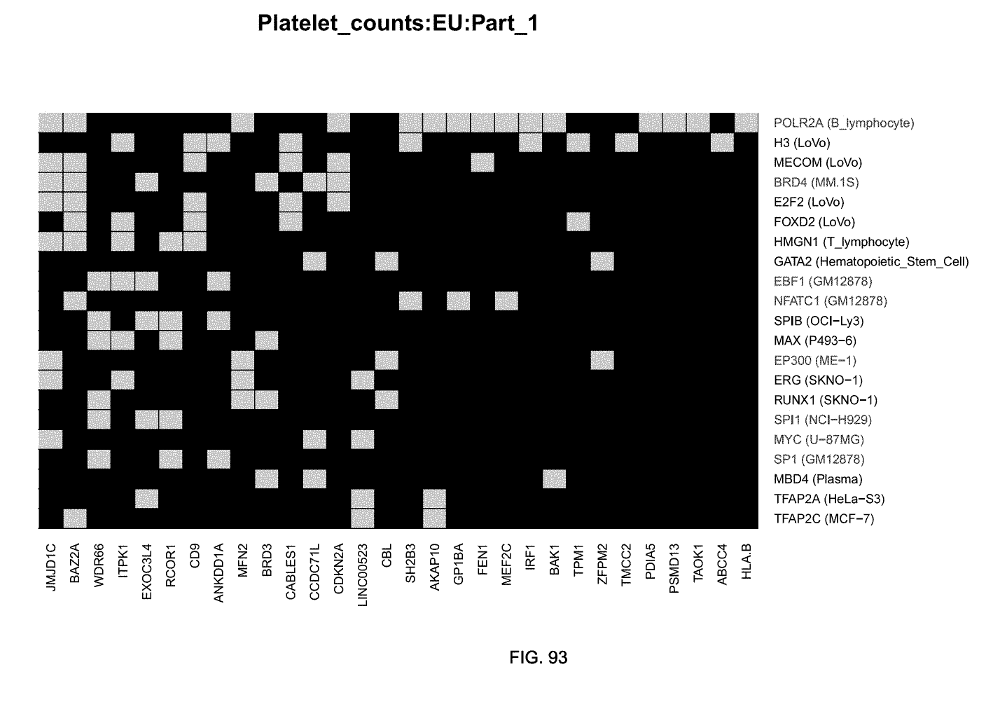

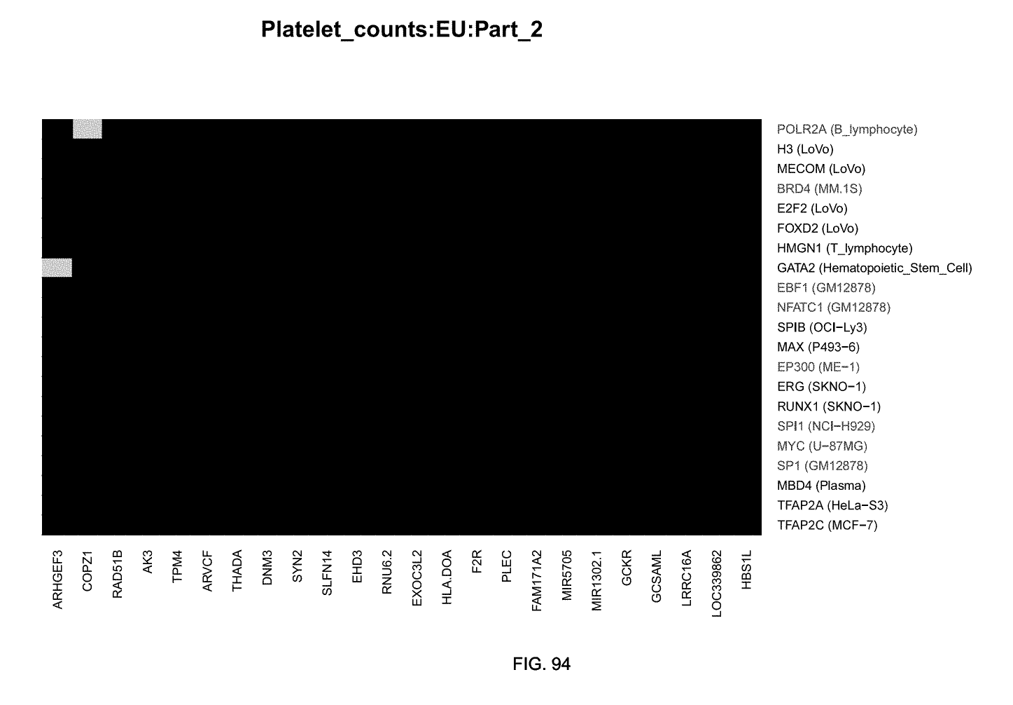

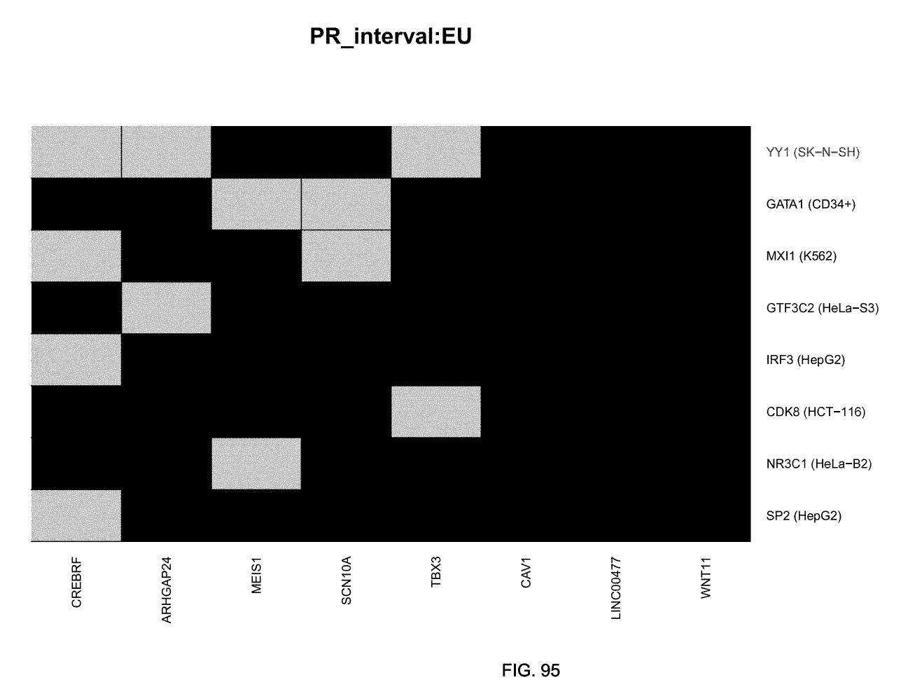

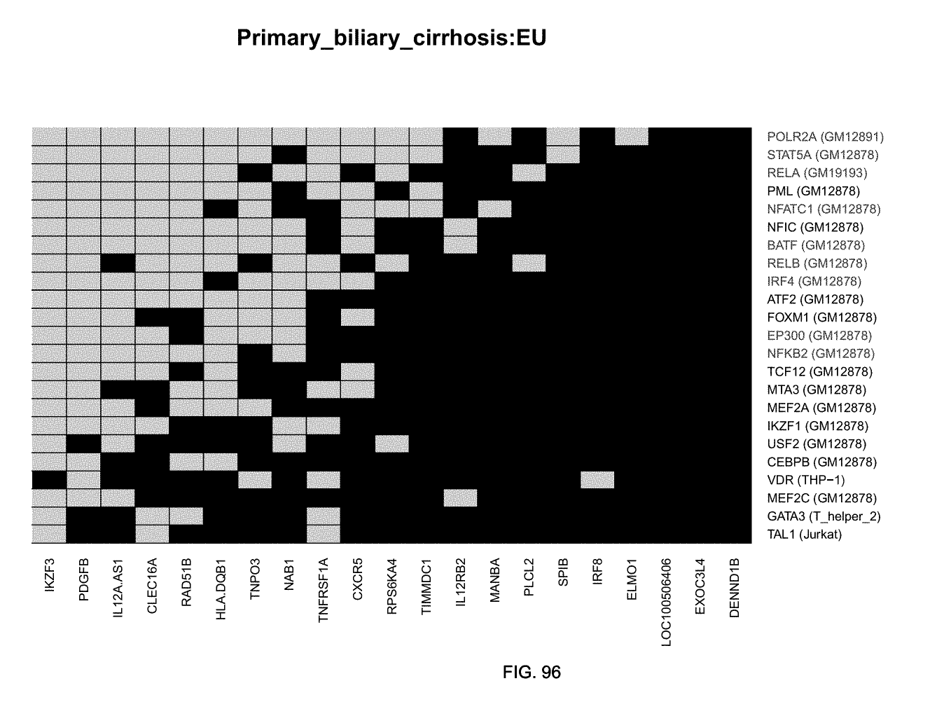

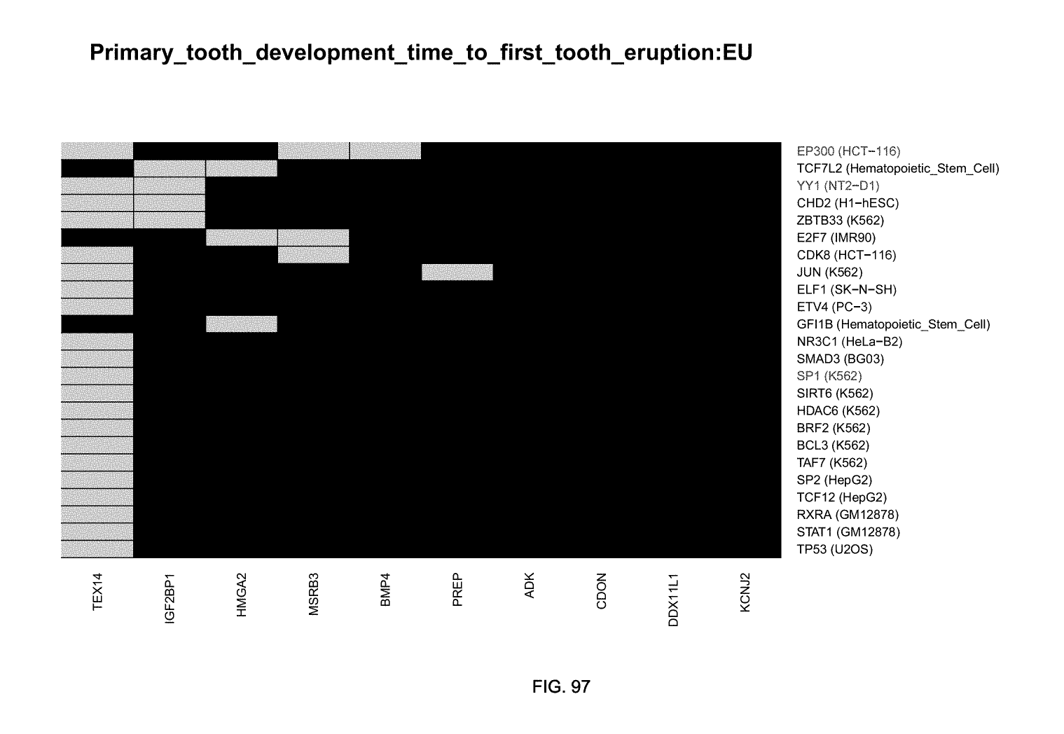

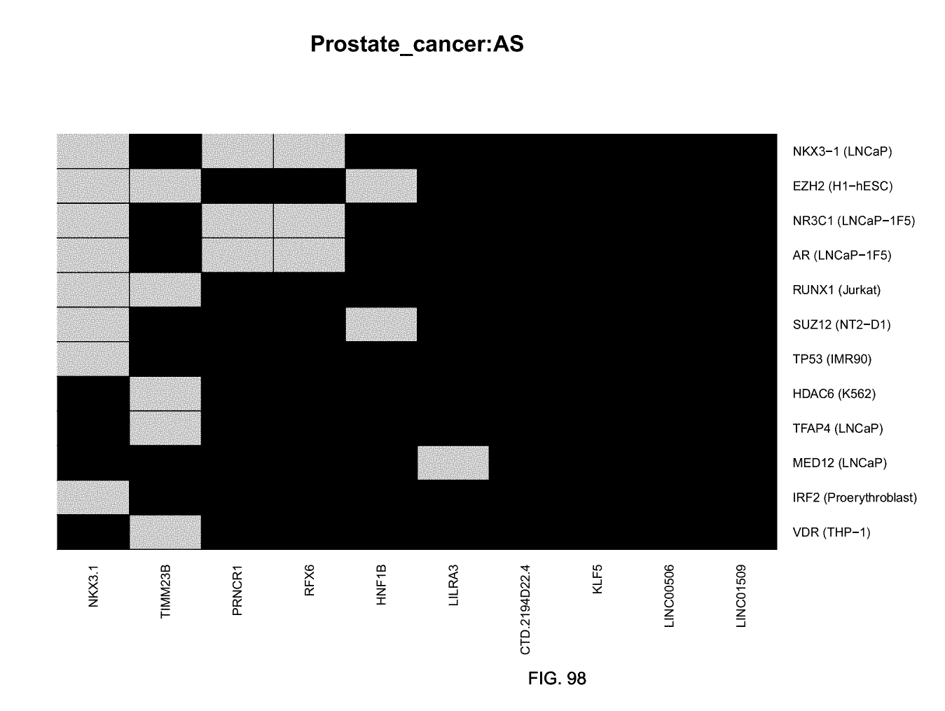

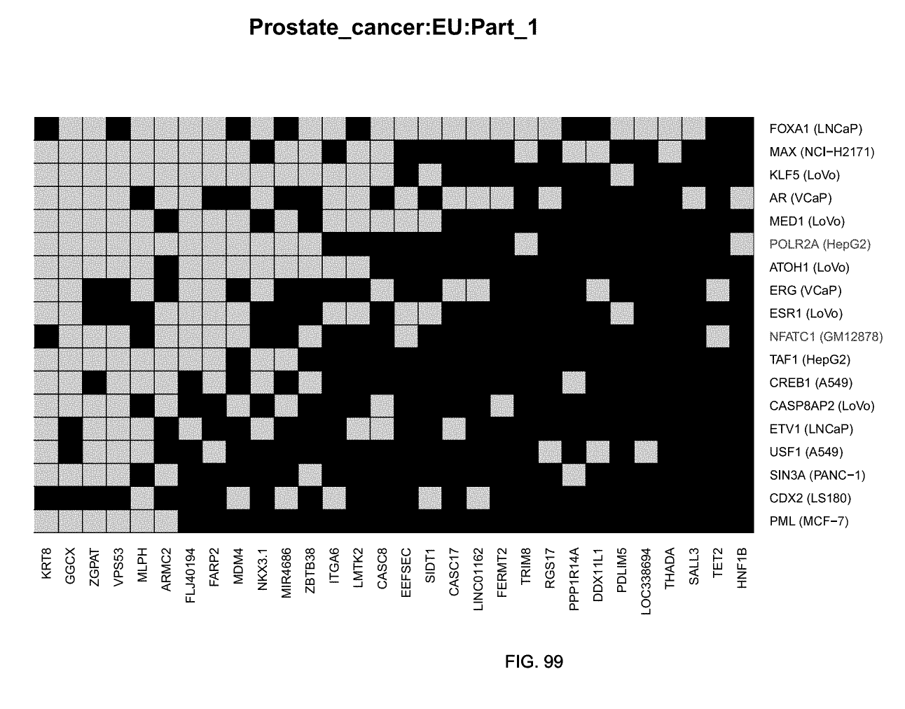

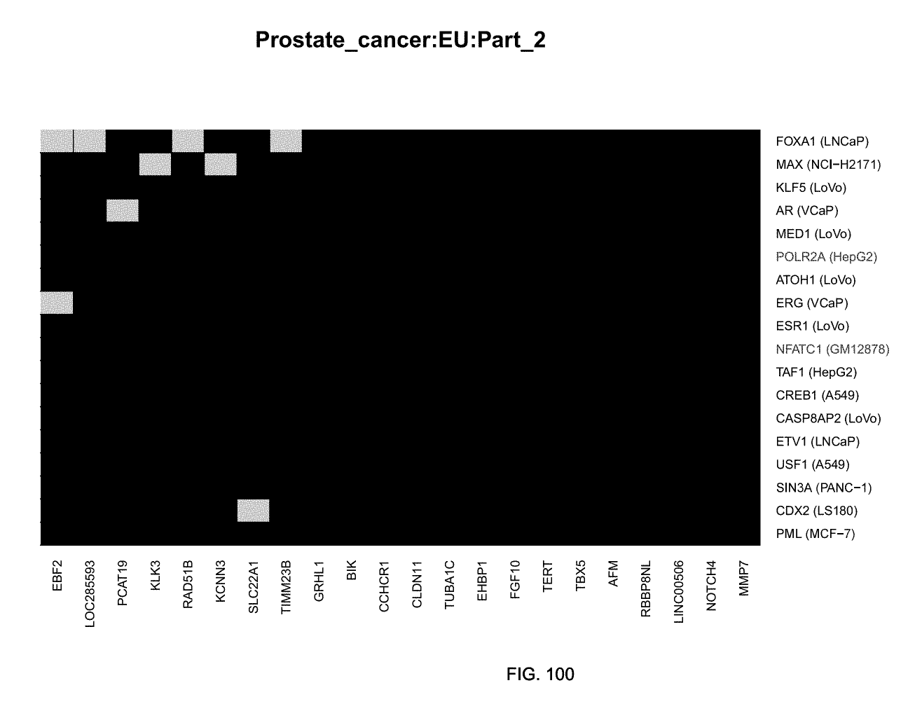

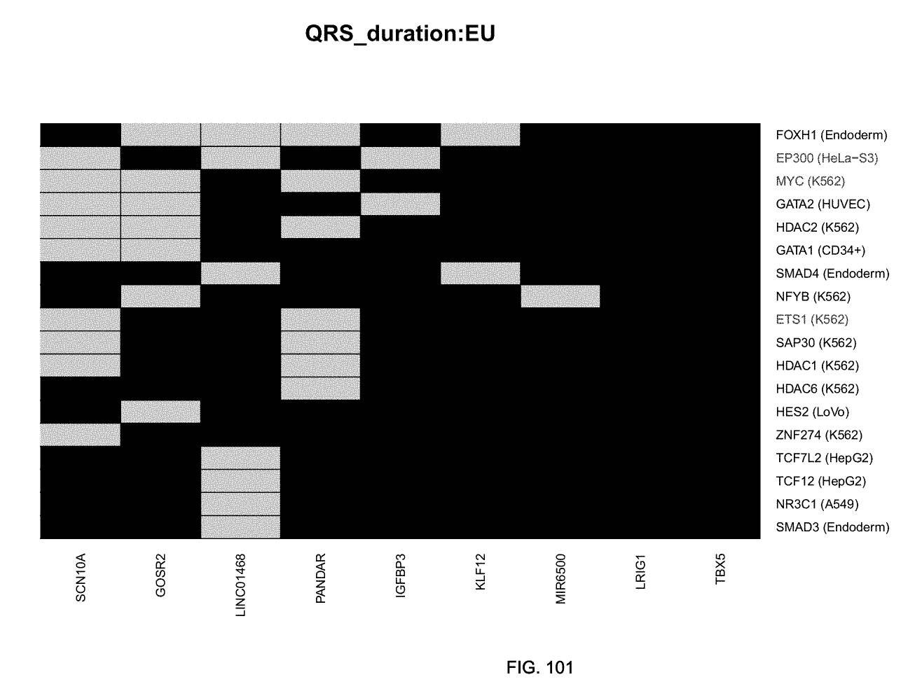

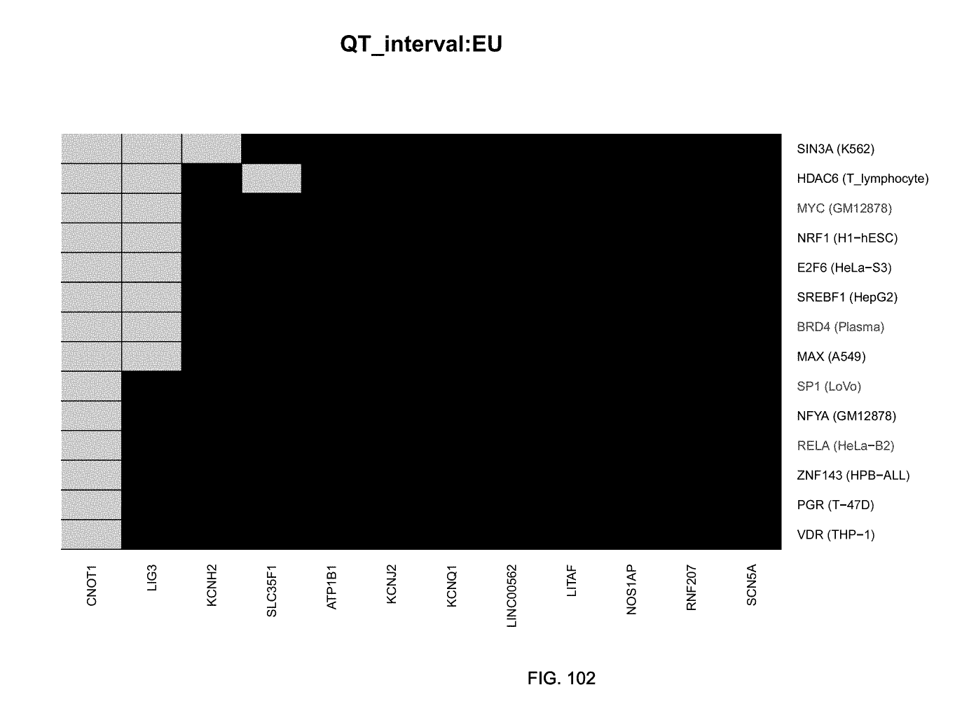

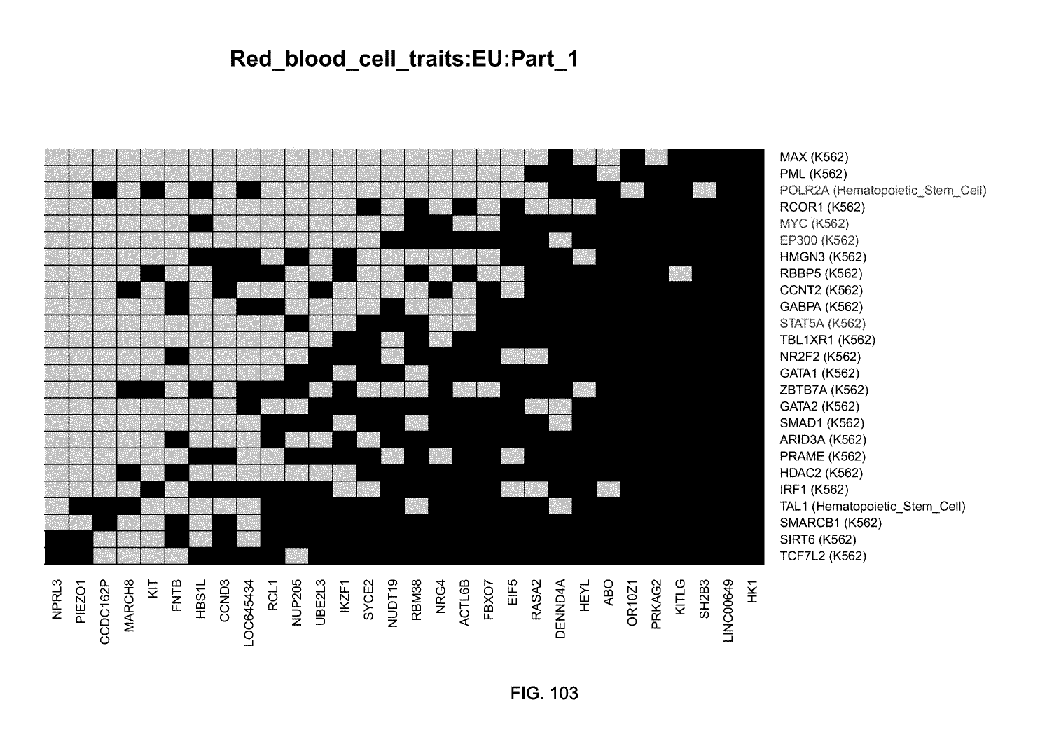

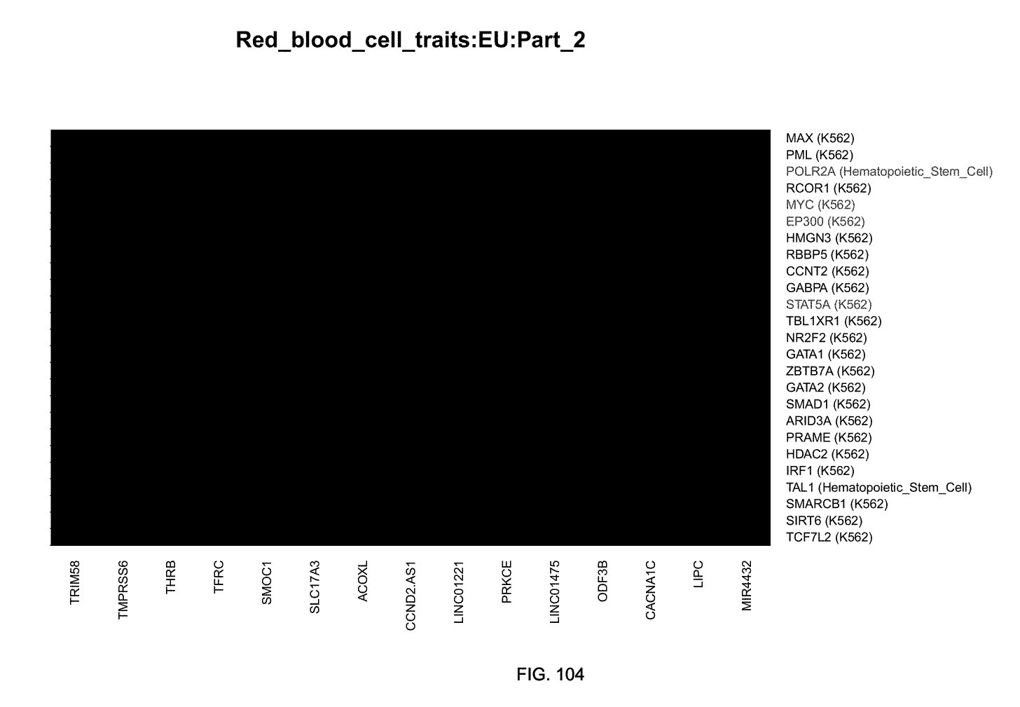

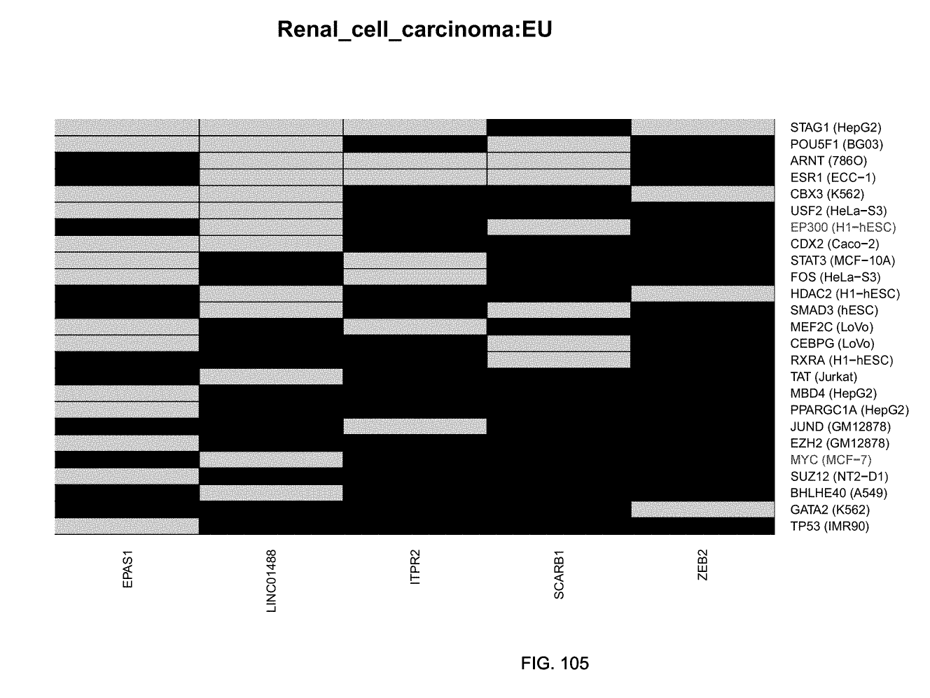

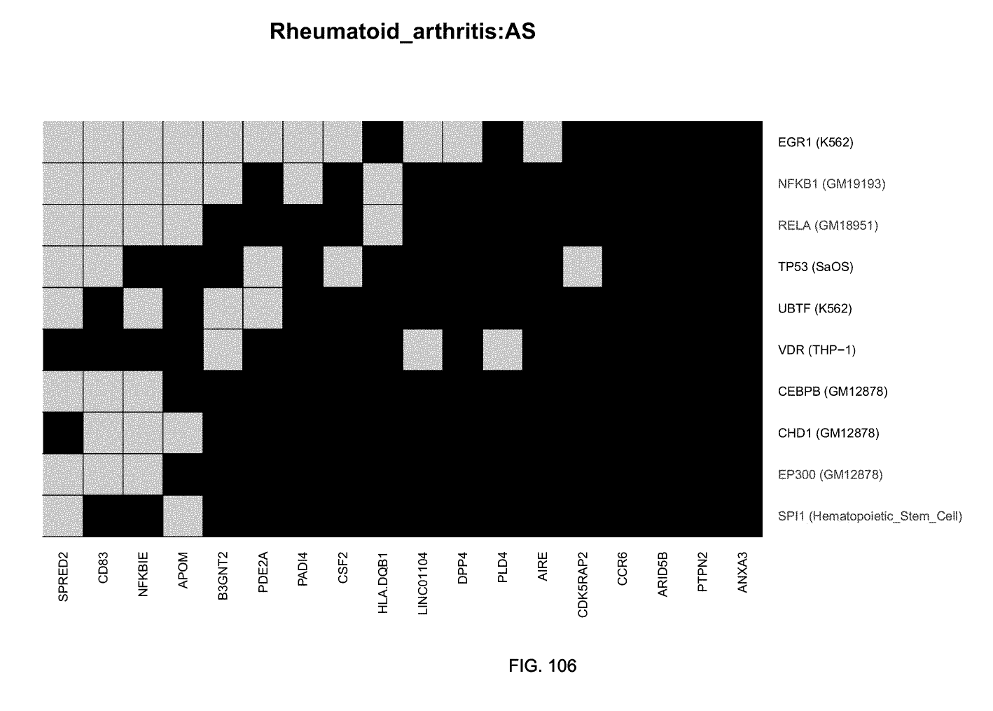

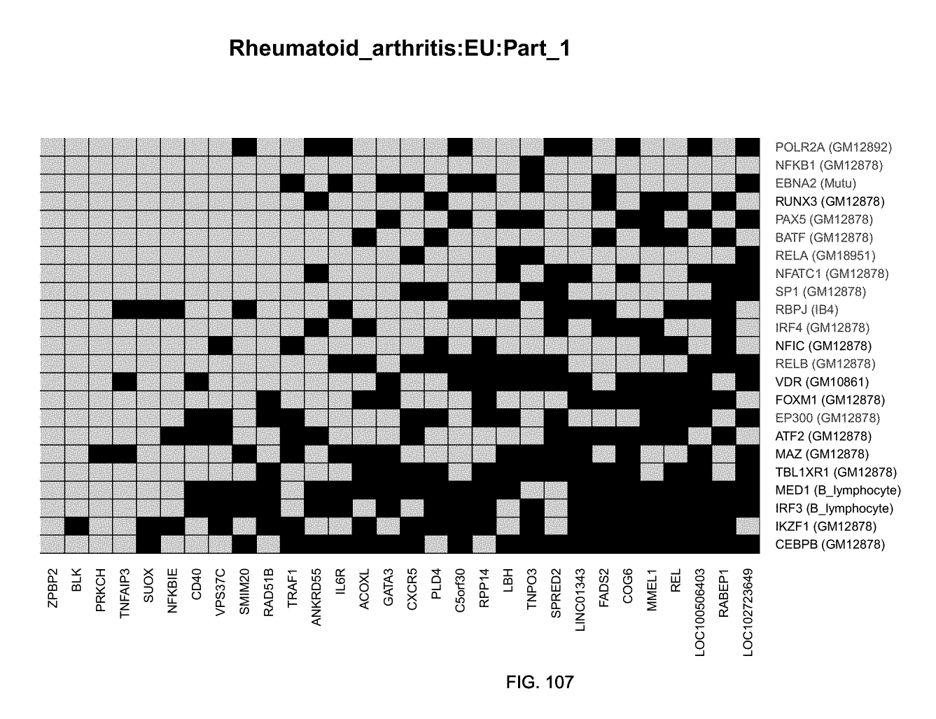

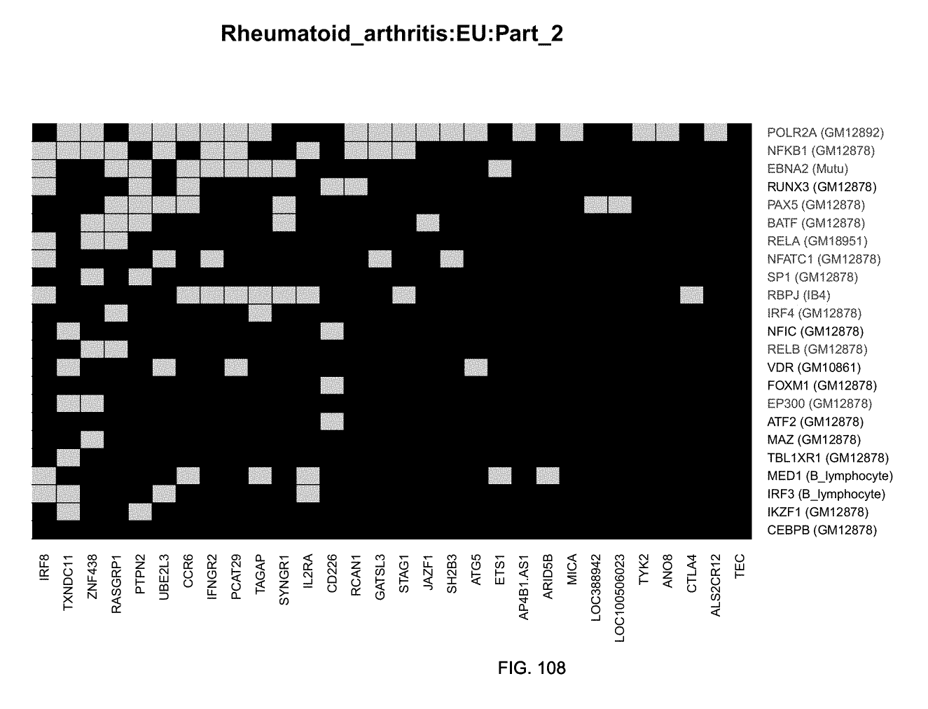

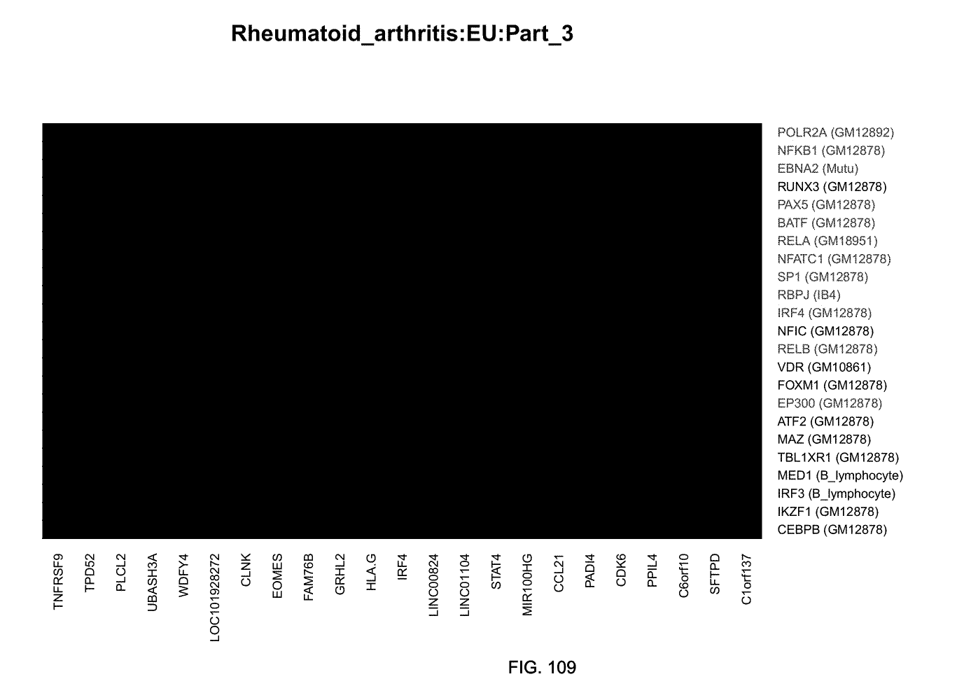

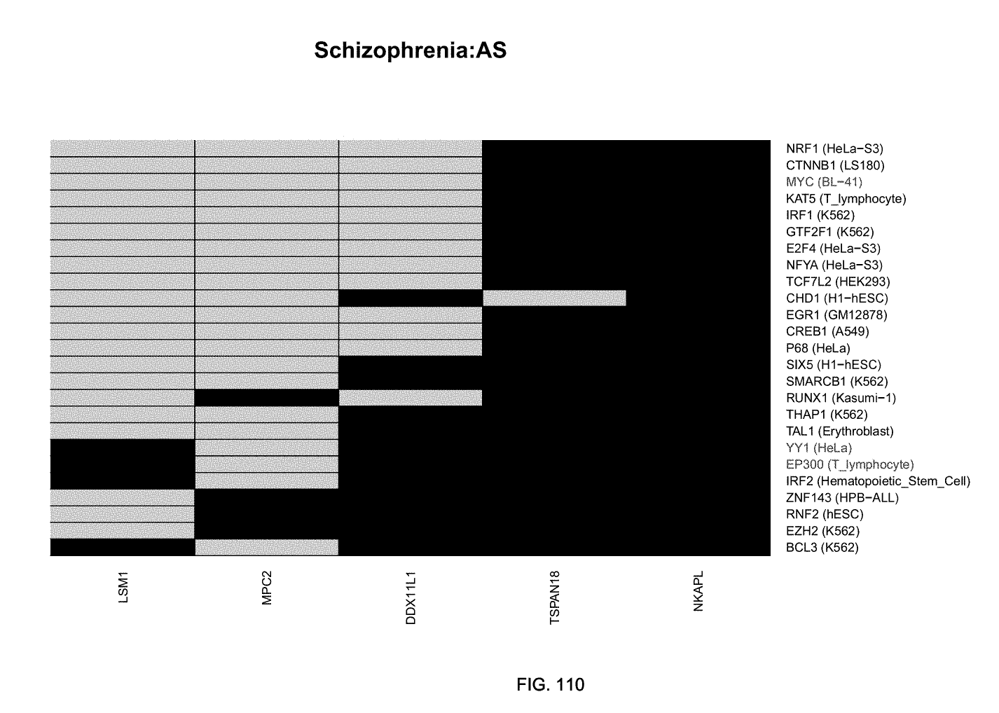

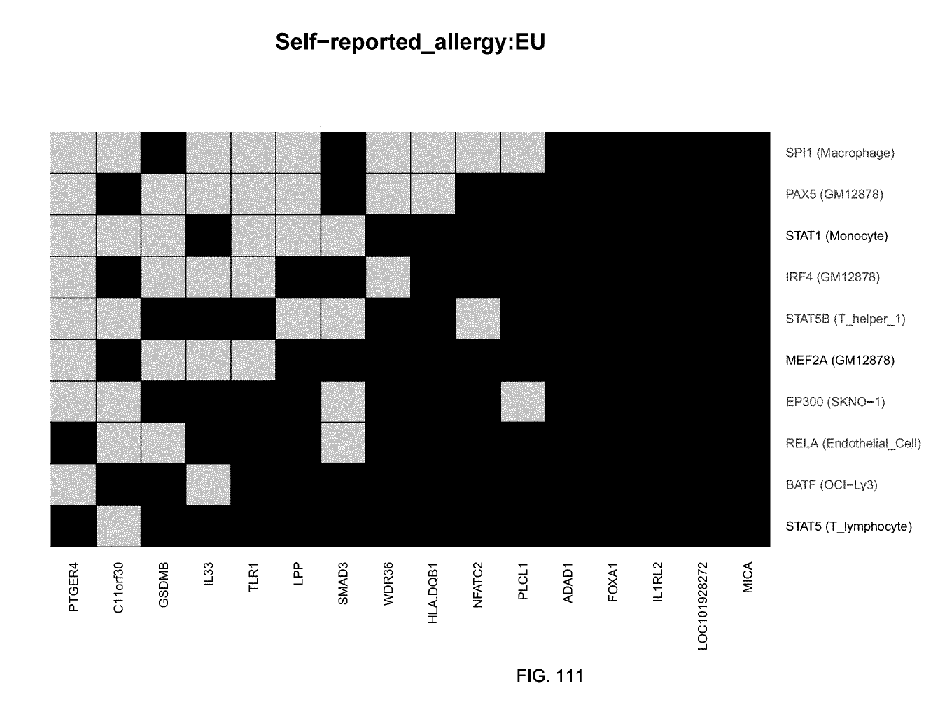

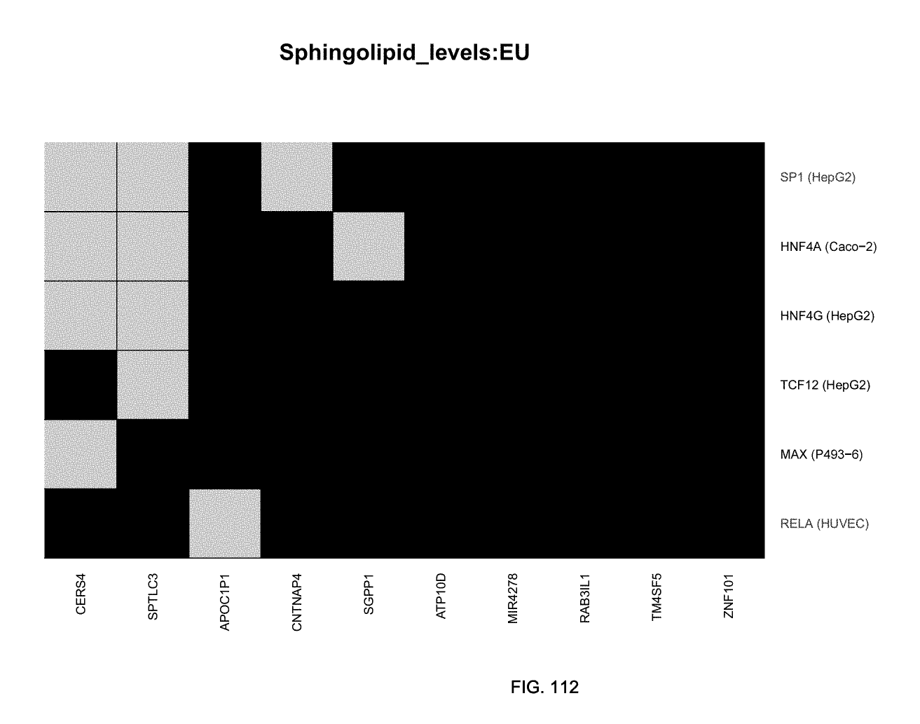

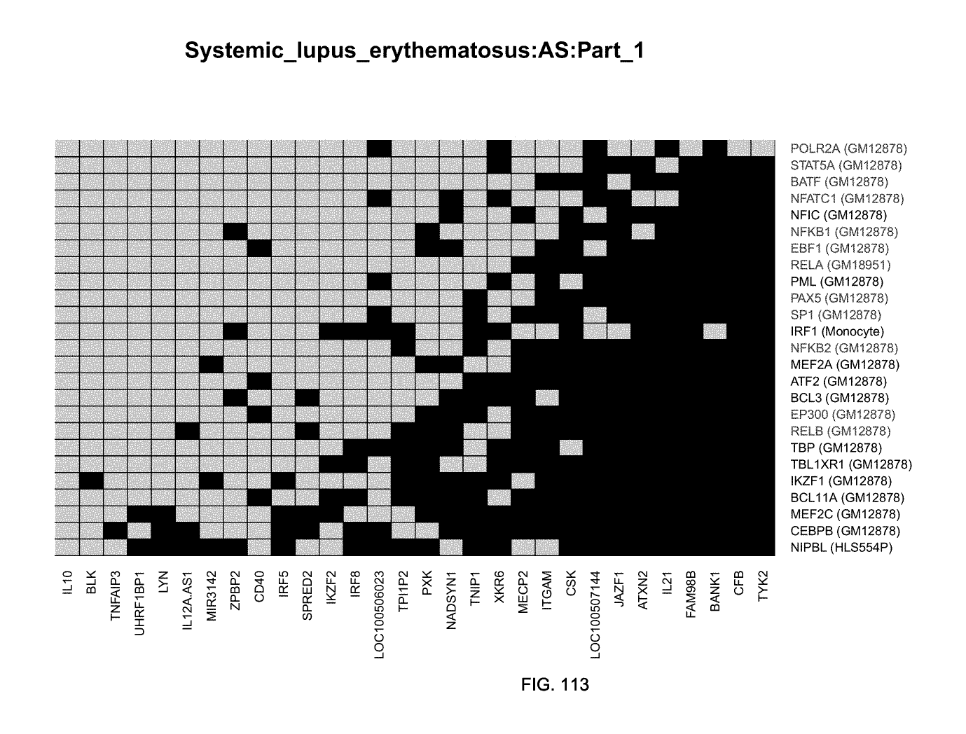

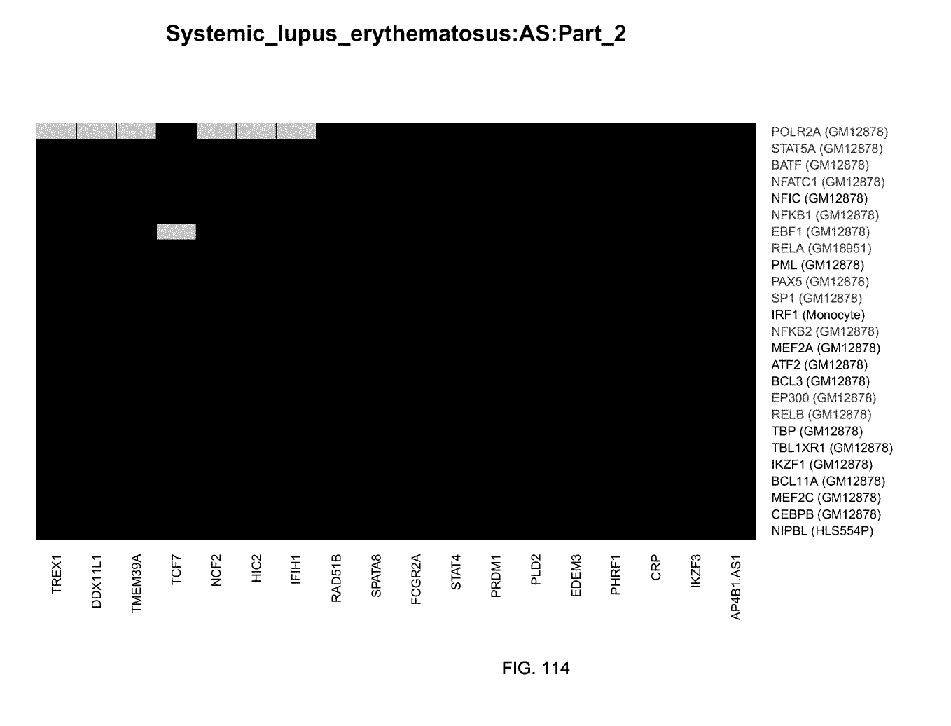

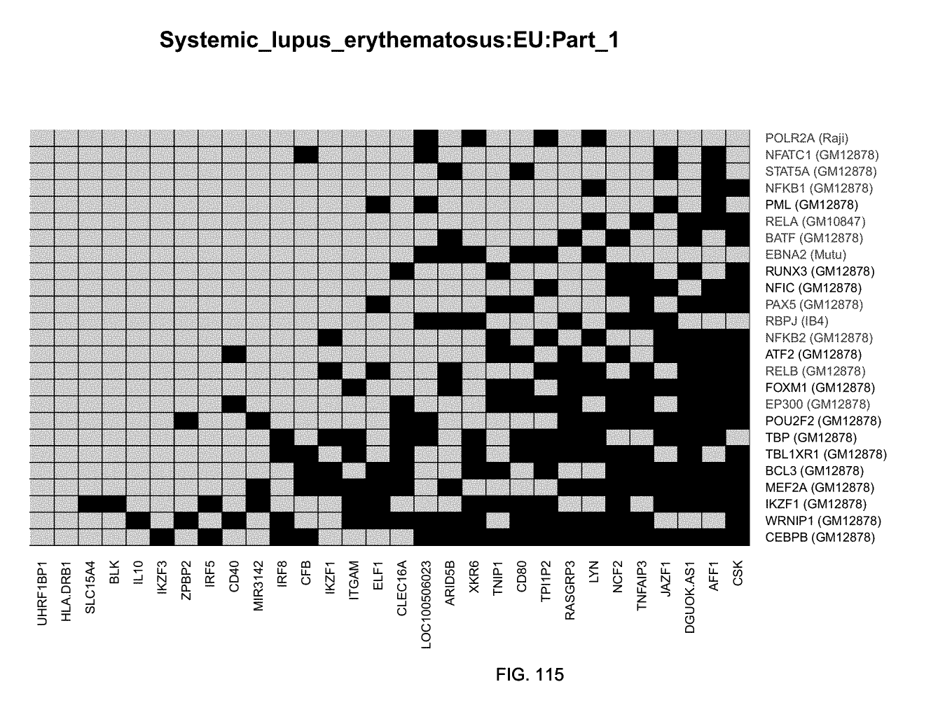

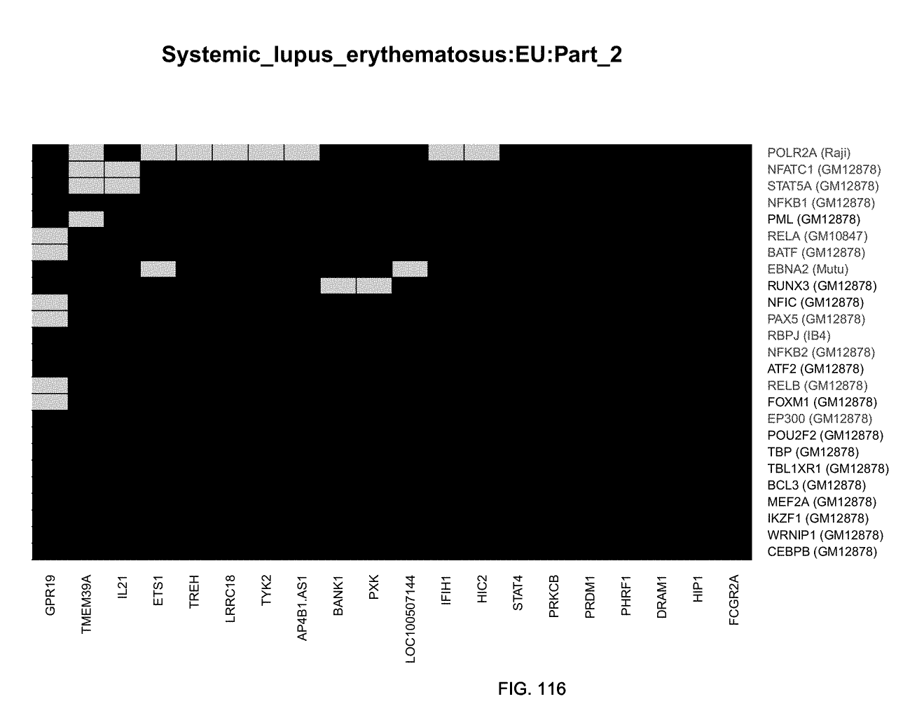

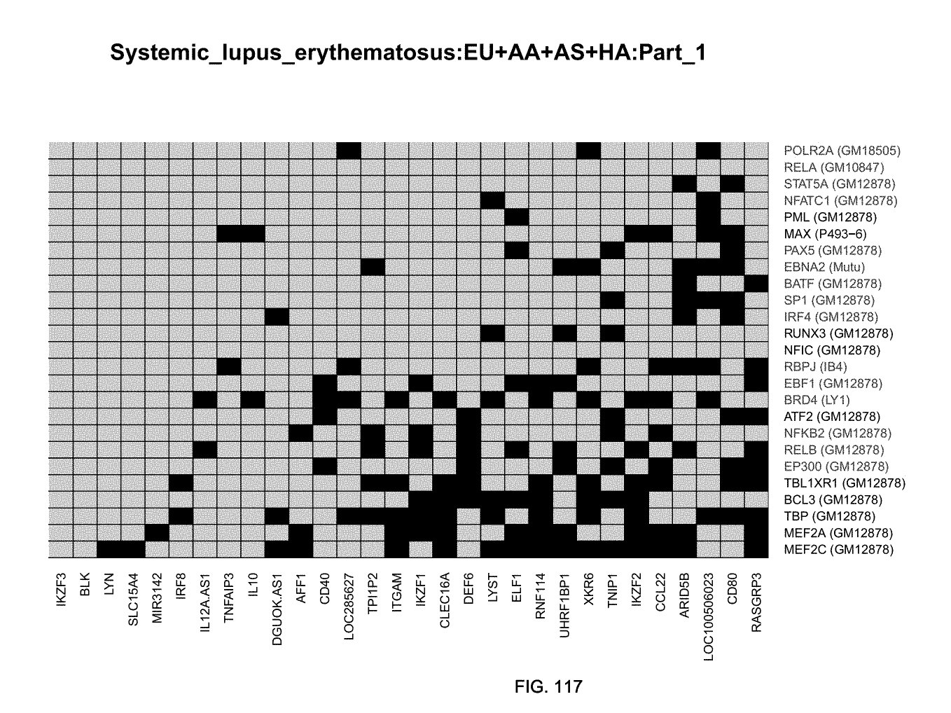

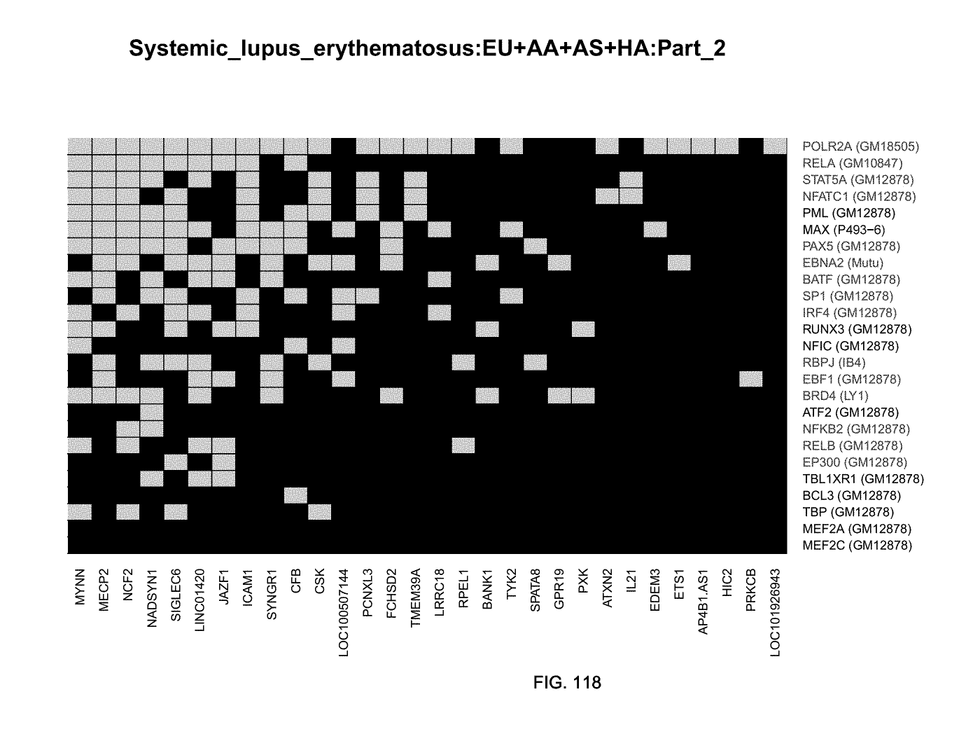

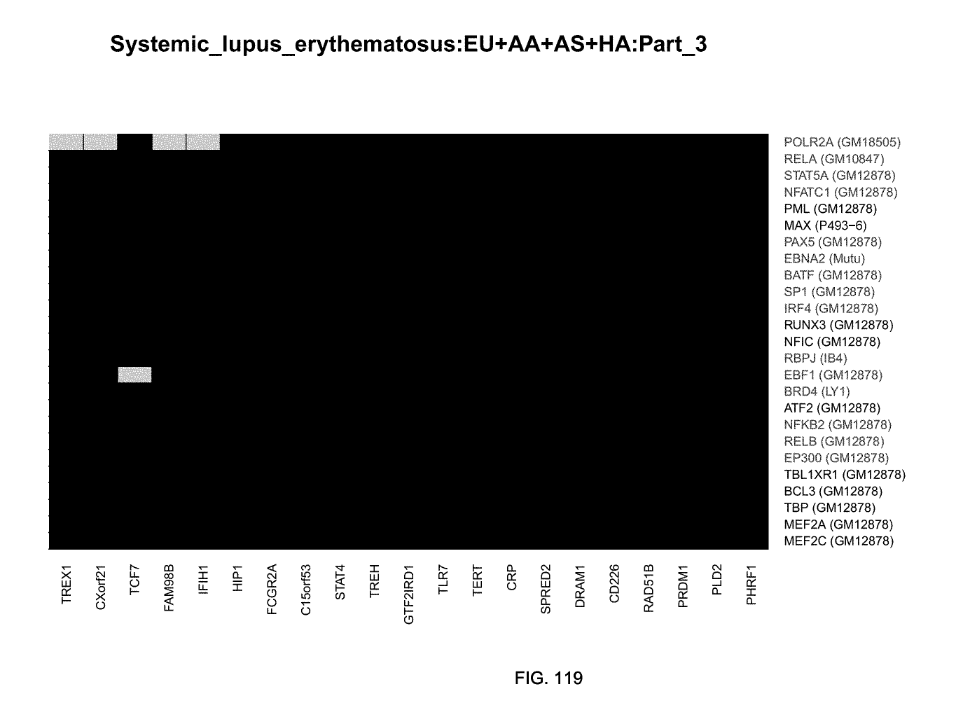

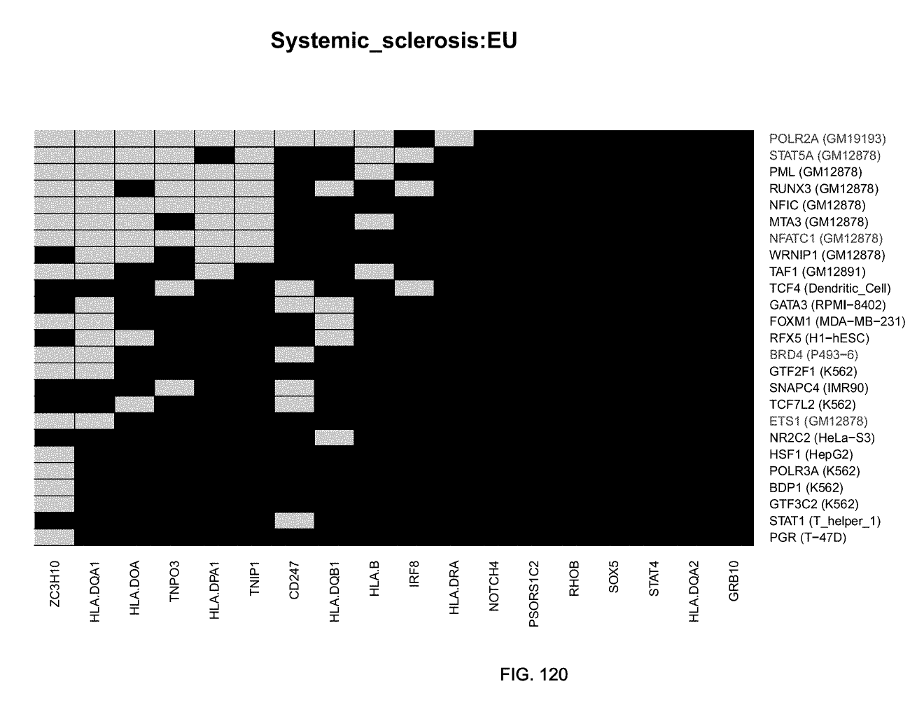

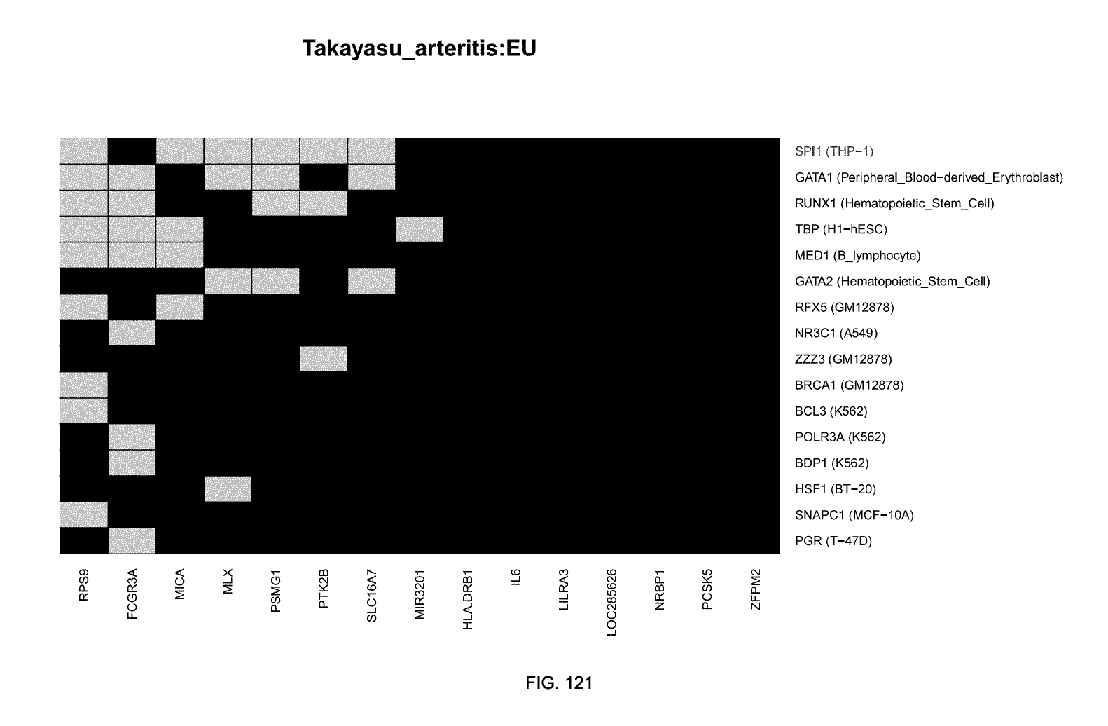

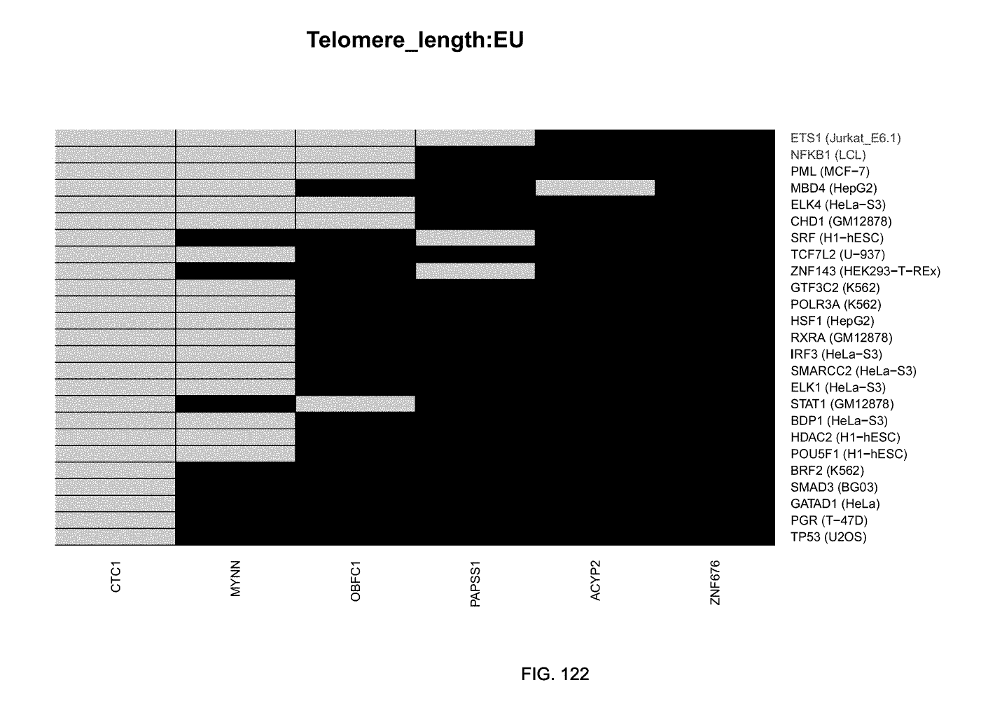

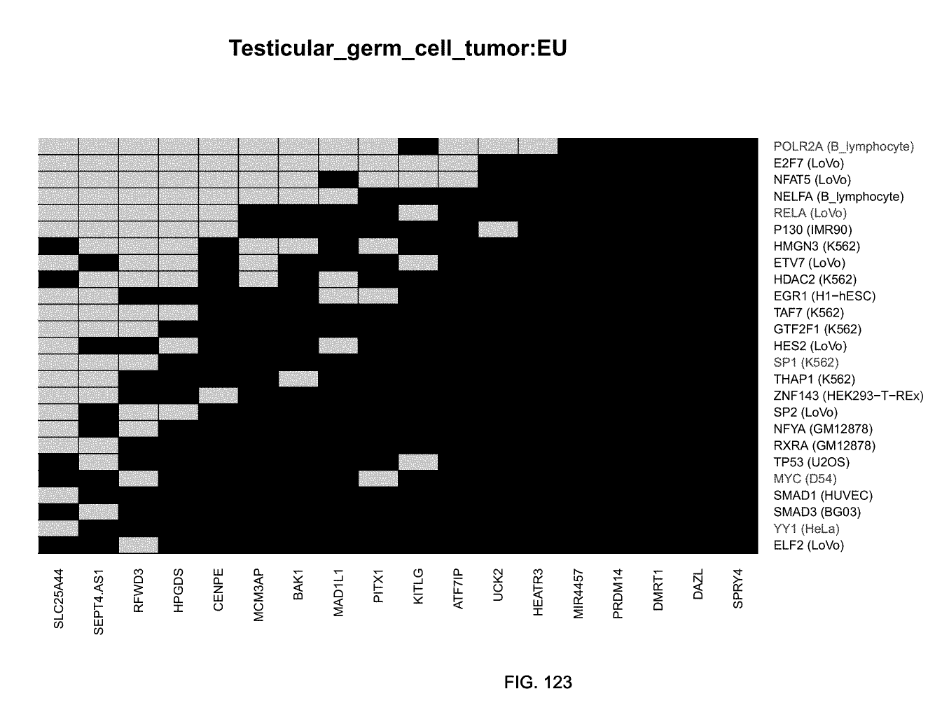

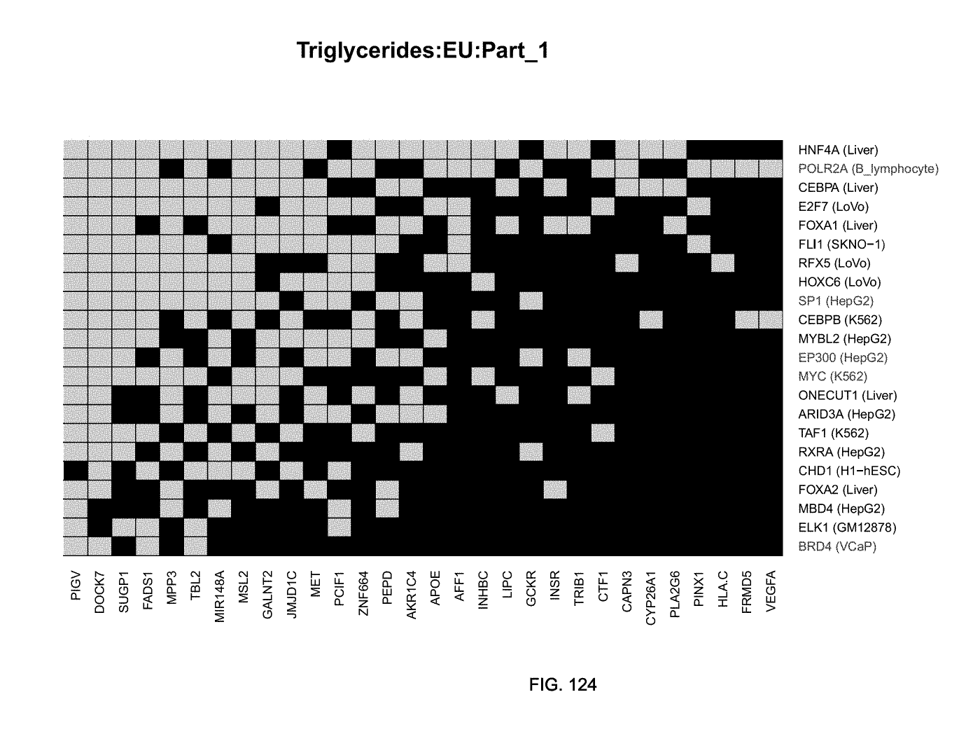

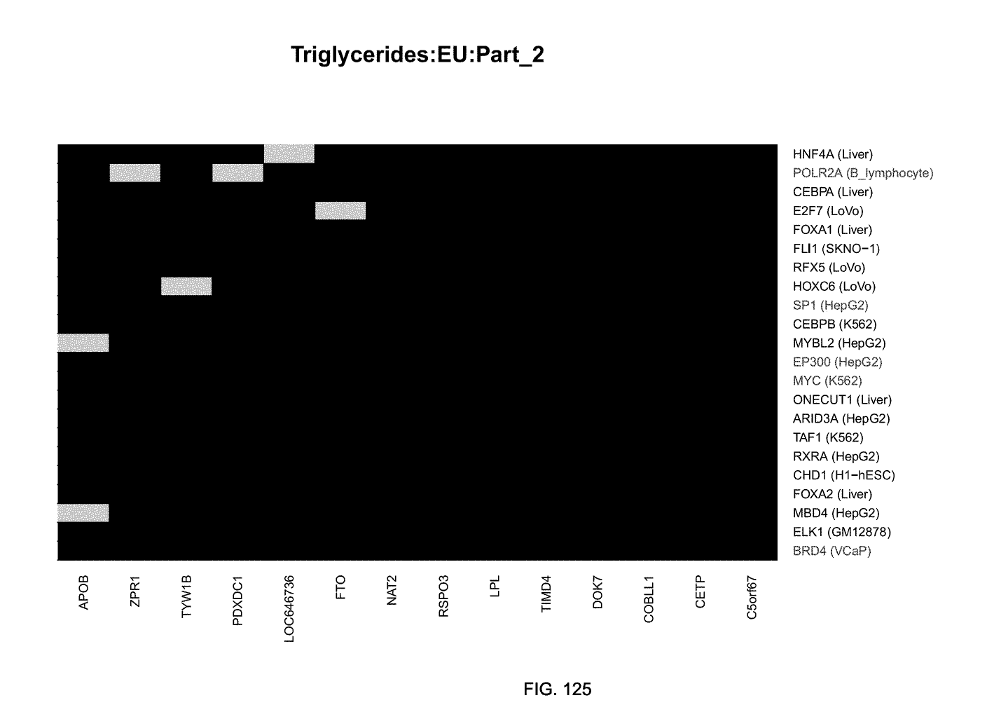

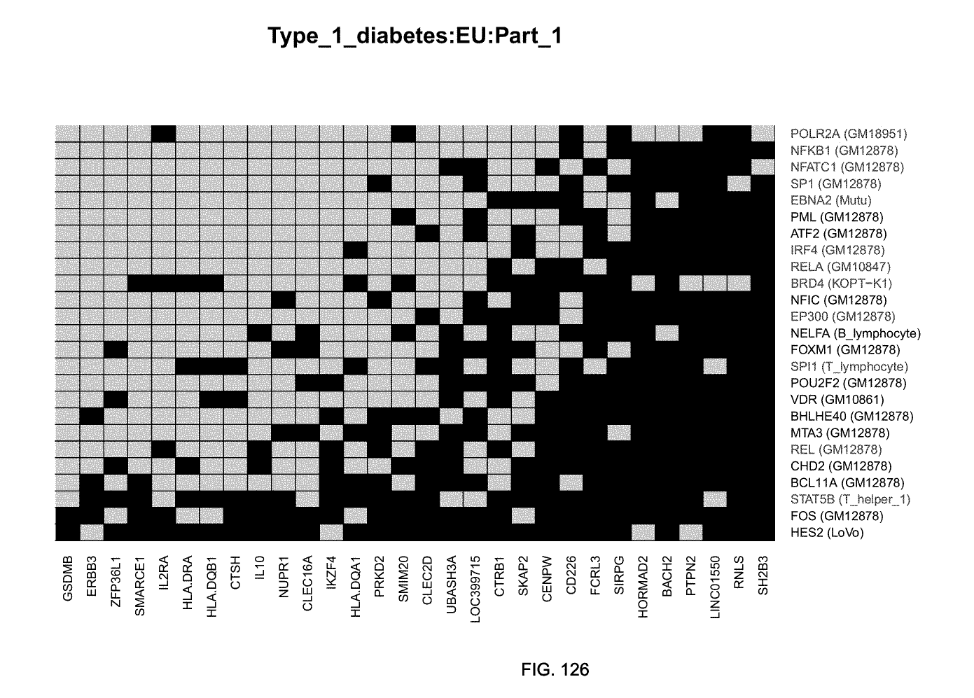

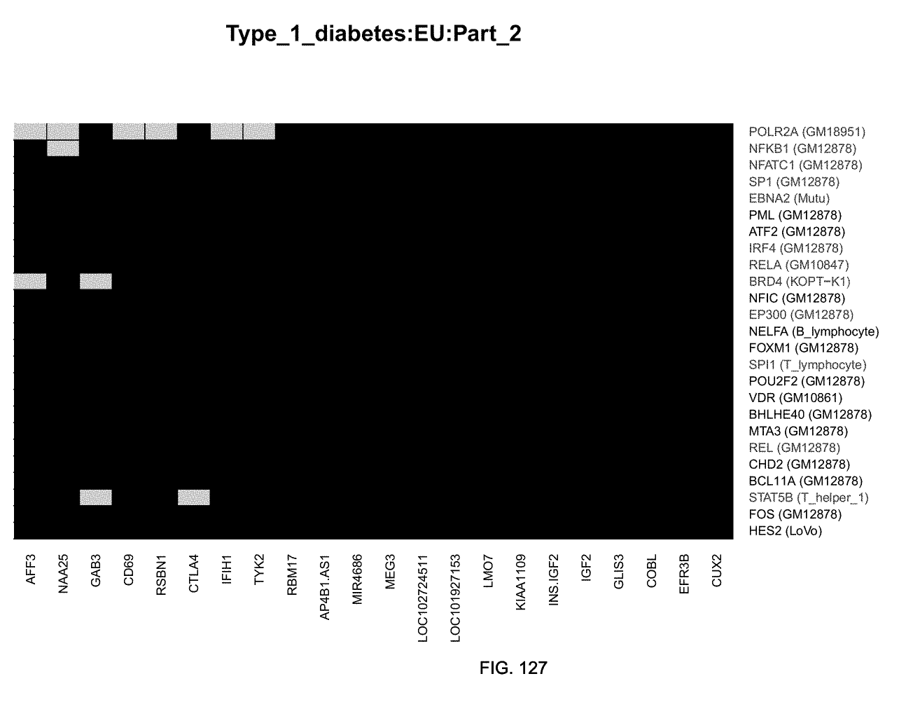

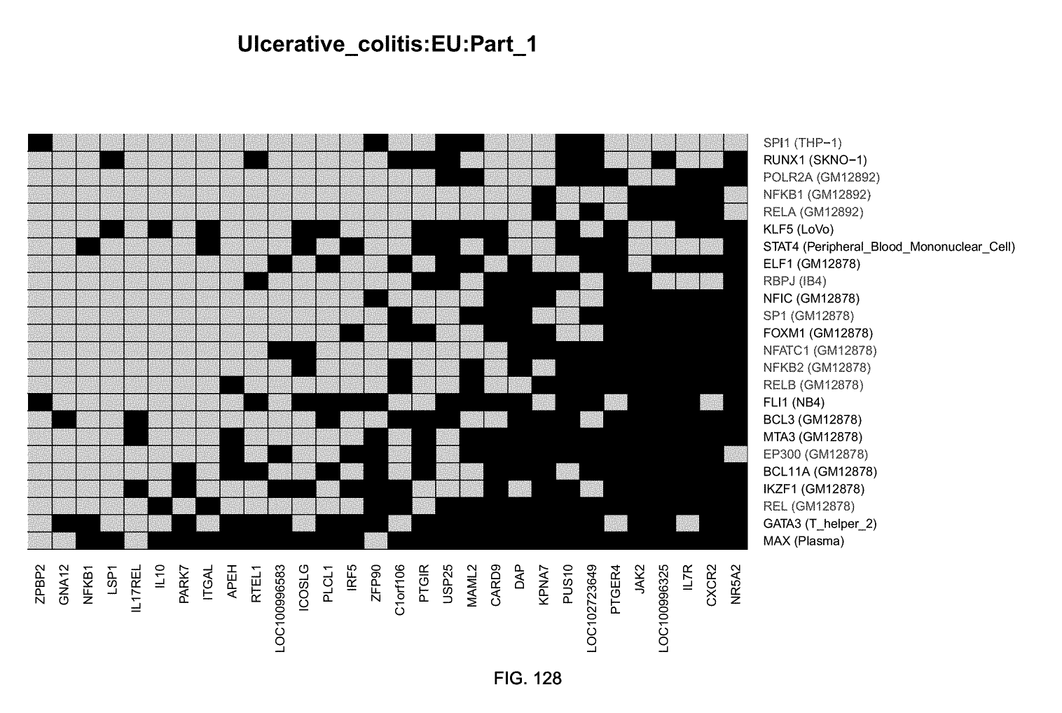

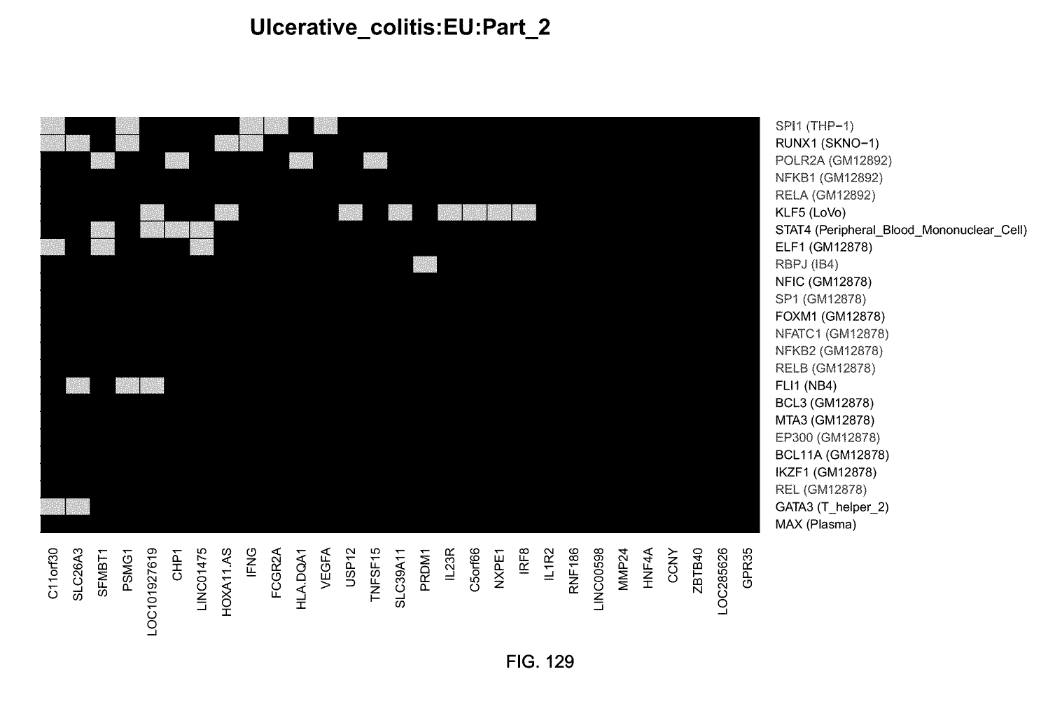

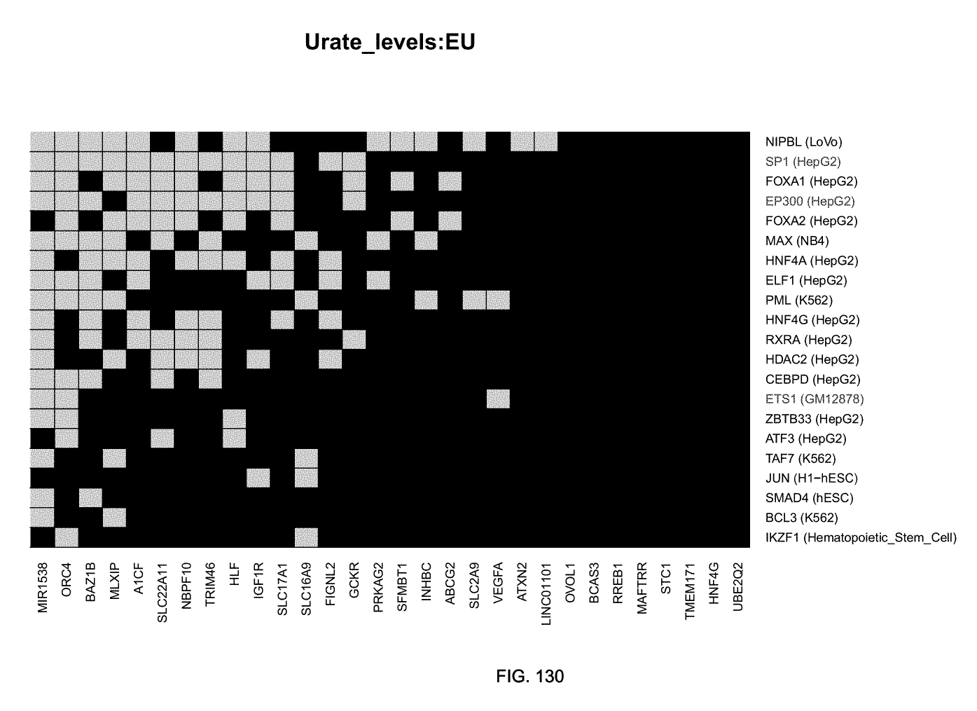

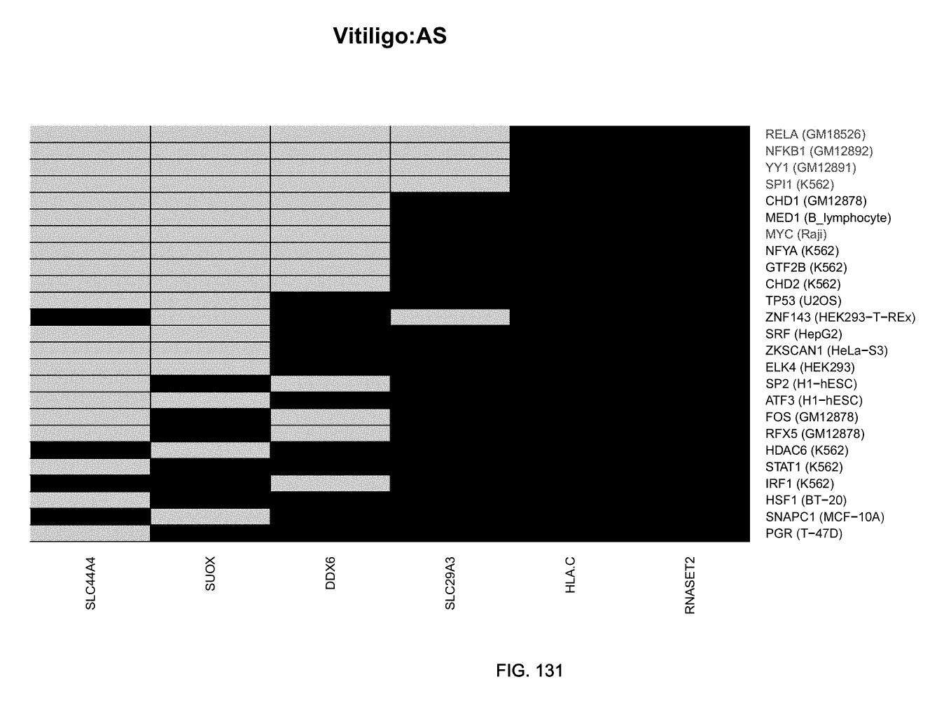

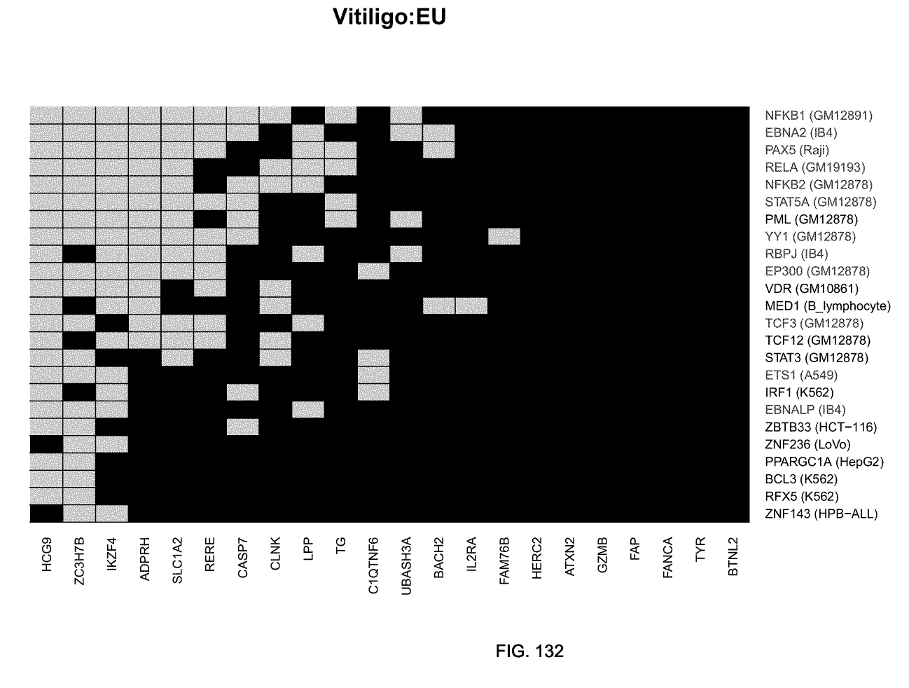

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The term “therapeutically effective amount,” as used herein, refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The terms “treat,” “treating” or “treatment,” as used herein, refers to any treatment of a disease or condition associated with a disease or physiological parameter that is dysregulated (such as blood pressure dysregulation), particularly in a human, and includes a) preventing the disease from occurring in a subject that may be predisposed to the disease and or condition but has not yet been diagnosed as having it; b) inhibiting the disease or condition, and c) relieving the disease and/or condition. “Treatment” can also encompass delivery of an agent or administration of a therapy in order to provide for a pharmacological effect, even in the absence of a disease or condition. The term “treatment” is used in some aspects to refer to administration of a compound disclosed herein to mitigate a disease or disorder in a host, for example a mammal, more specifically a human. The term “treatment” can include preventing a disorder from occurring in a host, particularly when the host is predisposed to acquiring the disease, but has not yet been diagnosed, inhibiting the disorder; and/or alleviating or reversing the disorder. Insofar as the methods describe “preventing” a disease or disorder, it is understood that the term “prevent” does not require that the disease state be completely thwarted. Rather, the term “preventing” refers to the ability of the skilled artisan to identify a population that is susceptible to disorders, such that administration of the compounds disclosed herein can occur prior to onset of a disease. The term does not mean that the disease state must be completely avoided.

The term “pharmaceutically acceptable,” as used herein, refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “pharmaceutically acceptable salt,” as used herein, refers to a formulation of a compound that does not cause significant irritation to an organism to which it is administered and does not abrogate the biological activity and properties of the compounds described herein.

The terms “composition” or “pharmaceutical composition,” as used herein, refers to a mixture of at least one compound, such as the compounds provided herein, with at least one and optionally more than one other pharmaceutically acceptable chemical components, such as carriers, stabilizers, diluents, dispersing agents, suspending agents, thickening agents, and/or excipients.

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like. Suitable pharmaceutical carriers are described in “Remington's Pharmaceutical Sciences” by E. W. Martin, 18th Edition.

The term “modulated” or “modulation” or “regulated” or “regulation” can refer to both up regulation, activation, or stimulation, for example, by agonizing or potentiating, and down regulation, inhibition or suppression, for example by antagonizing, decreasing or inhibiting, unless otherwise specified or clear from the context of a specific usage.

Explaining the genetics of many diseases is challenging because most associations localize to regulatory regions. Applicant has tested the hypothesis that transcription factors (TFs) are associated with multiple loci of individual complex genetic disorders with a novel computational method for discovering disease-driving mechanisms.

TABLE 1

| Column A | | |
|---|---|---|
| Transcription | Column B | Column C |
| Factor | Disease/Condition | Treatment Agent |
| TF: ESR1 | Breast cancer | 1-[4-(OCTAHYDRO-PYRIDO[1,2- |
| | Breast_cancer_early_onset | A]PYRAZIN-2-YL)-PHEN . . . |
| | Central_corneal_thickness | 1-[4-(OCTAHYDRO-PYRIDO[1,2- |
| | Coronary_heart_disease | A]PYRAZIN-2-YL)-PHENYL]-2-PHENYL- |
| | Inflammatory_bowel_disease | 1,2,3,4-TETRAHYDRO-ISOQUINOLIN-6- |
| | Interstitial_lung_disease | OL |
| | Juvenile_idiopathic_arthritis | 17-METHYL-17-ALPHA- |
| | Lipoprotein- | DIHYDROEQUILENIN |
| | associated_phospholipase_A2_activity_and_mass | 2-PHENYL-1-[4-(2-PIPERIDIN-1-YL- |
| | Prostate_cancer | ETHOXY)-PHENYL] . . . |
| | Renal_cell_carcinoma | 2-PHENYL-1-[4-(2-PIPERIDIN-1-YL- |
| | | ETHOXY)-PHENYL]-1,2,3,4- |
| | | TETRAHYDRO-ISOQUINOLIN-6-OL |
| | | (2R,3R,4S)-3-(4-HYDROXYPHENYL)-4- |
| | | METHYL-2-[4-(2 . . . |
| | | (2R,3R,4S)-3-(4-HYDROXYPHENYL)-4- |
| | | METHYL-2-[4-(2-PYRROLIDIN-1- |
| | | YLETHOXY)PHENYL]CHROMAN-6-OL |
| | | (3AS,4R,9BR)-2,2-DIFLUORO-4-(4- |
| | | HYDROXYPHENYL)-1 . . . |
| | | (3AS,4R,9BR)-2,2-DIFLUORO-4-(4- |
| | | HYDROXYPHENYL)-1,2,3,3A,4,9B- |
| | | HEXAHYDROCYCLOPENTA[C]CHROMEN- |
| | | 8-OL |
| | | (3AS,4R,9BR)-4-(4-HYDROXYPHENYL)- |
| | | 1,2,3,3A,4,9B- . . . |
| | | (3AS,4R,9BR)-4-(4-HYDROXYPHENYL)- |
| | | 1,2,3,3A,4,9B- |
| | | HEXAHYDROCYCLOPENTA[C]CHROMEN- |
| | | 9-OL |
| | | (3AS,4R,9BR)-4-(4-HYDROXYPHENYL)- |
| | | 6-(METHOXYMETH . . . |
| | | (3AS,4R,9BR)-4-(4-HYDROXYPHENYL)- |
| | | 6-(METHOXYMETHYL)-1,2,3,3A,4,9B- |
| | | HEXAHYDROCYCLOPENTA[C]CHROMEN- |
| | | 8-OL |
| | | 3-CHLORO-2-(4-HYDROXYPHENYL)-2H- |
| | | INDAZOL-5-OL |
| | | 3-ETHYL-2-(4-HYDROXYPHENYL)-2H- |
| | | INDAZOL-5-OL |
| | | 4-[(1S,2R,5S)-4,4,8-TRIMETHYL-3- |
| | | OXABICYCLO[3.3 . . . |
| | | 4-[(1S,2R,5S)-4,4,8-TRIMETHYL-3- |
| | | OXABICYCLO[3.3.1]NON-7-EN-2- |
| | | YL]PHENOL |
| | | 4-[(1S,2S,5S)-5-(HYDROXYMETHYL)- |
| | | 6,8,9-TRIMETHYL . . . |
| | | 4-[(1S,2S,5S)-5-(HYDROXYMETHYL)- |
| | | 6,8,9-TRIMETHYL-3- |
| | | OXABICYCLO[3.3.1]NON-7-EN-2- |
| | | YL]PHENOL |
| | | 4-[(1S,2S,5S)-5-(HYDROXYMETHYL)-8- |
| | | METHYL-3-OXAB . . . |
| | | 4-[(1S,2S,5S)-5-(HYDROXYMETHYL)-8- |
| | | METHYL-3-OXABICYCLO[3.3.1]NON-7- |
| | | EN-2-YL]PHENOL |
| | | 4-[(1S,2S,5S,9R)-5-(HYDROXYMETHYL)- |
| | | 8,9-DIMETHYL . . . |
| | | 4-[(1S,2S,5S,9R)-5-(HYDROXYMETHYL)- |
| | | 8,9-DIMETHYL-3- |
| | | OXABICYCLO[3.3.1]NON-7-EN-2- |
| | | YL]PHENOL |
| | | 4-(6-HYDROXY-1H-INDAZOL-3- |
| | | YL)BENZENE-1,3-DIOL |
| | | [5-HYDROXY-2-(4-HYDROXYPHENYL)- |
| | | 1-BENZOFURAN-7-Y . . . |
| | | [5-HYDROXY-2-(4-HYDROXYPHENYL)- |
| | | 1-BENZOFURAN-7-YL]ACETONITRILE |
| | | (9ALPHA,13BETA,17BETA)-2-[(1Z)-BUT- |
| | | 1-EN-1-YL]ES . . . |
| | | (9ALPHA,13BETA,17BETA)-2-[(1Z)-BUT- |
| | | 1-EN-1-YL]ESTRA-1,3,5(10)-TRIENE-3,17- |
| | | DIOL |
| | | (9BETA,11ALPHA,13ALPHA,14BETA,17ALPHA)- |
| | | 11-(METH . . . |
| | | (9BETA,11ALPHA,13ALPHA,14BETA,17ALPHA)- |
| | | 11-(METHOXYMETHYL)ESTRA- |
| | | 1(10),2,4-TRIENE-3,17-DIOL |
| | | AFIMOXIFENE |
| | | ALLYLESTRENOL |
| | | ANASTROZOLE |
| | | ARZOXIFENE |
| | | BAZEDOXIFENE |
| | | CHLOROTRIANISENE |
| | | CLOMIFENE |
| | | CLOMIPHENE |
| | | CLOMIPHENE CITRATE |
| | | COMPOUND 19 |
| | | COMPOUND 4-D |
| | | CONJUGATED ESTROGENS |
| | | DANAZOL |
| | | DEHYDROEPIANDROSTERONE |
| | | DESOGESTREL |
| | | DIENESTROL |
| | | DIENOGEST |
| | | DIETHYL (1R,2S,3R,4S)-5,6-BIS(4- |
| | | HYDROXYPHENYL)- . . . |
| | | DIETHYL (1R,2S,3R,4S)-5,6-BIS(4- |
| | | HYDROXYPHENYL)-7- |
| | | OXABICYCLO[2.2.1]HEPT-5-ENE-2,3- |
| | | DICARBOXYLATE |
| | | DIETHYLSTILBESTROL |
| | | DIMETHYL (1R,4S)-5,6-BIS(4- |
| | | HYDROXYPHENYL)-7-OXA . . . |
| | | DIMETHYL (1R,4S)-5,6-BIS(4- |
| | | HYDROXYPHENYL)-7- |
| | | OXABICYCLO[2.2.1]HEPTA-2,5-DIENE- |
| | | 2,3-DICARBOXYLATE |
| | | ENDOXIFEN |
| | | ESTRADIOL |
| | | ESTRADIOL CYPIONATE |
| | | ESTRADIOL VALERATE |
| | | ESTRAMUSTINE |
| | | ESTRIOL |
| | | ESTRONE |
| | | ESTROPIPATE |
| | | ETHINYL ESTRADIOL |
| | | ETHYNODIOL |
| | | ETHYNODIOL DIACETATE |
| | | ETONOGESTREL |
| | | EXEMESTANE |
| | | FISPEMIFENE |
| | | FLUOXYMESTERONE |
| | | FULVESTRANT |
| | | GENISTEIN |
| | | HEXESTROL |
| | | IODINE |
| | | LASOFOXIFENE |
| | | LEFLUNOMIDE |
| | | LETROZOLE |
| | | LEVONORGESTREL |
| | | MEGESTROL |
| | | MELATONIN |
| | | MESTRANOL |
| | | METHYL-PIPERIDINO-PYRAZOLE |
| | | MITOTANE |
| | | NALOXONE |
| | | NORELGESTROMIN |
| | | NORGESTIMATE |
| | | NORGESTREL |
| | | OSPEMIFENE |
| | | PROGESTERONE |
| | | QUINESTROL |
| | | RALOXIFEN |
| | | RALOXIFENE |
| | | RALOXIFENE CORE |
| | | TAMOXIFEN |
| | | TAMOXIFEN CITRATE |
| | | TOREMIFENE |
| | | TRILOSTANE |

TABLE 2

| Column A | | |
|---|---|---|
| Transcription | Column B | Column C |
| Factor | Disease/Condition | Treatment Agent |
| TF: ESR2 | Parkinson_disease | 1-CHLORO-6-(4-HYDROXYPHENYL)-2-NAPHTHOL |
| | Type_1_diabetes | 2-(4-HYDROXY-PHENYL)BENZOFURAN-5-OL |
| | | 2-(5-HYDROXY-NAPHTHALEN-1-YL)-1,3- |
| | | BENZOOXAZOL-6-OL |
| | | 3-(3-FLUORO-4-HYDROXYPHENYL)-7-HYDROXY-1- |
| | | NAPHTH . . . |
| | | 3-(3-FLUORO-4-HYDROXYPHENYL)-7-HYDROXY-1- |
| | | NAPHTHONITRILE |
| | | 3-(6-HYDROXY-NAPHTHALEN-2-YL)- |
| | | BENZO[D]ISOOXAZOL . . . |
| | | 3-(6-HYDROXY-NAPHTHALEN-2-YL)- |
| | | BENZO[D]ISOOXAZOL-6-OL |
| | | (3AS,4R,9BR)-2,2-DIFLUORO-4-(4- |
| | | HYDROXYPHENYL)-1 . . . |
| | | (3AS,4R,9BR)-2,2-DIFLUORO-4-(4- |
| | | HYDROXYPHENYL)-1,2,3,3A,4,9B- |
| | | HEXAHYDROCYCLOPENTA[C]CHROMEN-8-OL |
| | | (3AS,4R,9BR)-2,2-DIFLUORO-4-(4- |
| | | HYDROXYPHENYL)-6 . . . |
| | | (3AS,4R,9BR)-2,2-DIFLUORO-4-(4- |
| | | HYDROXYPHENYL)-6-(METHOXYMETHYL)- |
| | | 1,2,3,3A,4,9B- |
| | | HEXAHYDROCYCLOPENTA[C]CHROMEN-8-OL |
| | | (3AS,4R,9BR)-4-(4-HYDROXYPHENYL)-6- |
| | | (METHOXYMETH . . . |
| | | (3AS,4R,9BR)-4-(4-HYDROXYPHENYL)-6- |
| | | (METHOXYMETHYL)-1,2,3,3A,4,9B- |
| | | HEXAHYDROCYCLOPENTA[C]CHROMEN-8-OL |
| | | 3-BROMO-6-HYDROXY-2-(4-HYDROXYPHENYL)- |
| | | 1H-INDEN- . . . |
| | | 3-BROMO-6-HYDROXY-2-(4-HYDROXYPHENYL)- |
| | | 1H-INDEN-1-ONE |
| | | 4-(4-HYDROXYPHENYL)-1-NAPHTHALDEHYDE |
| | | OXIME |
| | | 571-20-0 |
| | | 5-HYDROXY-2-(4-HYDROXYPHENYL)-1- |
| | | BENZOFURAN-7-CA . . . |
| | | 5-HYDROXY-2-(4-HYDROXYPHENYL)-1- |
| | | BENZOFURAN-7-CARBONITRILE |
| | | [5-HYDROXY-2-(4-HYDROXYPHENYL)-1- |
| | | BENZOFURAN-7-Y . . . |
| | | [5-HYDROXY-2-(4-HYDROXYPHENYL)-1- |
| | | BENZOFURAN-7-YL]ACETONITRILE |
| | | AFIMOXIFENE |
| | | BAZEDOXIFENE |
| | | BISPHENOL A |
| | | CHLOROTRIANISENE |
| | | DEHYDROEPIANDROSTERONE |
| | | DIETHYLSTILBESTROL |
| | | ESTRADIOL |
| | | ESTRAMUSTINE |
| | | ESTRAMUSTINE PHOSPHATE SODIUM |
| | | ESTRIOL |
| | | ESTRONE |
| | | ESTROPIPATE |
| | | ETHINYL ESTRADIOL |
| | | EXEMESTANE |
| | | FISPEMIFENE |
| | | FULVESTRANT |
| | | GENISTEIN |
| | | HPTL |
| | | LASOFOXIFENE |
| | | N-BUTYL-11-[(7R,8R,9S,13S,14S,17S)-3,17- |
| | | DIHYDRO . . . |
| | | N-BUTYL-11-[(7R,8R,9S,13S,14S,17S)-3,17- |
| | | DIHYDROXY-13-METHYL-7,8,9,11,12,13,14,15,16,17- |
| | | DECAHYDRO-6H-CYCLOPENTA[A]PHENANTHREN- |
| | | 7-YL]-N-METHYLUNDECANAMIDE |
| | | OSPEMIFENE |
| | | PHTPP |
| | | PRINABEREL |
| | | RALOXIFEN |
| | | RALOXIFENE |
| | | RALOXIFENE HYDROCHLORIDE |
| | | TAMOXIFEN |
| | | TOREMIFENE |
| | | TRILOSTANE |

TABLE 3

| Column A | | |
|---|---|---|
| Transcription | Column B | Column C |
| Factor | Disease/Condition | Treatment Agent |
| TF: AR | Interstitial_lung_disease | (2S)-N-(4-CYANO-3-IODOPHENYL)-3-(4- |
| | Mean_platelet_volume | CYANOPHENOXY . . . |
| | Prostate_cancer | (2S)-N-(4-CYANO-3-IODOPHENYL)-3-(4- |
| | | CYANOPHENOXY)-2-HYDROXY-2- |
| | | METHYLPROPANAMIDE |
| | | 4-{[(1R,2S)-1,2-DIHYDROXY-2-METHYL-3- |
| | | (4-NITROPH . . . |
| | | 4-{[(1R,2S)-1,2-DIHYDROXY-2-METHYL-3- |
| | | (4-NITROPHENOXY)PROPYL]AMINO}-2- |
| | | (TRIFLUOROMETHYL)BENZONITRILE |
| | | 4-[(7R,7AS)-7-HYDROXY-1,3- |
| | | DIOXOTETRAHYDRO-1H-PY . . . |
| | | 4-[(7R,7AS)-7-HYDROXY-1,3- |
| | | DIOXOTETRAHYDRO-1H-PYRROLO[1,2- |
| | | C]IMIDAZOL-2(3H)-YL]-1- |
| | | NAPHTHONITRILE |
| | | (5S,8R,9S,10S,13R,14S,17S)-13-{2-[(3,5- |
| | | DIFLUORO . . . |
| | | (5S,8R,9S,10S,13R,14S,17S)-13-{2-[(3,5- |
| | | DIFLUOROBENZYL)OXY]ETHYL}-17- |
| | | HYDROXY-10- |
| | | METHYLHEXADECAHYDRO-3H- |
| | | CYCLOPENTA[A]PHENANTHREN-3-ONE |
| | | ABIRATERONE |
| | | ANDARINE |
| | | ARN-509 |
| | | ASC-J9 |
| | | BICALUTAMIDE |
| | | BISPHENOL A |
| | | CALUSTERONE |
| | | CYPROTERONE |
| | | CYPROTERONE ACETATE |
| | | DANAZOL |
| | | DROMOSTANOLONE |
| | | DROSPIRENONE |
| | | DROSTANOLONE |
| | | ENOBOSARM |
| | | ENZALUTAMIDE |
| | | EPALRESTAT |
| | | ETHYLESTRENOL |
| | | FIDARESTAT |
| | | FLUDROCORTISONE |
| | | FLUFENAMIC ACID |
| | | FLUOXYMESTERONE |
| | | FLUTAMIDE |
| | | GALETERONE |
| | | GLPG0492 |
| | | HYDROXYFLUTAMIDE |
| | | KETOCONAZOLE |
| | | LEVONORGESTREL |
| | | LGD-2941 |
| | | METHYLTESTOSTERONE |
| | | METHYLTRIENOLONE |
| | | MIBOLERONE |
| | | MIFEPRISTONE |
| | | NANDROLONE |
| | | NANDROLONE DECANOATE |
| | | NANDROLONE PHENPROPIONATE |
| | | NILUTAMIDE |
| | | OXANDROLONE |
| | | OXYMETHOLONE |
| | | PRASTERONE |
| | | SORBINIL |
| | | SPIRONOLACTONE |
| | | STANOZOLOL |
| | | TESTOSTERONE |
| | | TESTOSTERONE CYPIONATE |
| | | TESTOSTERONE ENANTHATE |
| | | TESTOSTERONE PROPIONATE |
| | | TESTOSTERONE UNDECANOATE |
| | | ZENARESTAT |

TABLE 4

| Column A | | |
|---|---|---|
| Transcription | Column B | Column C |
| Factor | Disease/Condition | Treatment Agent |
| TF: PGR | Breast_cancer | ALLYLESTRENOL |
| | Migraine | ANASTROZOLE |
| | Polycystic_ovary_syndrome | ASOPRISNIL |
| | | DANAZOL |
| | | DESOGESTREL |
| | | DIENOGEST |
| | | DROSPIRENONE |
| | | DYDROGESTERONE |
| | | ETHYNODIOL |
| | | ETHYNODIOL |
| | | DIACETATE |
| | | ETONOGESTREL |
| | | FLUTICASONE |
| | | PROPIONATE |
| | | GESTODENE |
| | | HYDROXY- |
| | | PROGESTERONE |
| | | CAPROATE |
| | | LETROZOLE |
| | | LEVONORGESTREL |
| | | MEDROXY- |
| | | PROGESTERONE |
| | | MEDROXY- |
| | | PROGESTERONE |
| | | ACETATE |
| | | MEGESTROL |
| | | MEGESTROL ACETATE |
| | | METHYLTRIENOLONE |
| | | MIFEPRISTONE |
| | | NORELGESTROMIN |
| | | NORETHINDRONE |
| | | NORETHINDRONE |
| | | ACETATE |
| | | NORETHYNODREL |
| | | NORGESTIMATE |
| | | NORGESTREL |
| | | ONAPRISTONE |
| | | PROGESTERONE |
| | | PROMEGESTONE |
| | | SPIRONOLACTONE |
| | | TAMOXIFEN |
| | | TANAPROGET |
| | | TELAPRISTONE |
| | | ULIPRISTAL |
| | | ULIPRISTAL ACETATE |
| | | ZK112993 |

TABLE 5

| Column A | | |
|---|---|---|
| Transcription | Column B | Column C |
| Factor | Disease/Condition | Treatment Agent |
| TF: HDAC2 | Blood_metabolite_levels | 4SC-202 |
| | Cholesterol_total | AMINOPHYLLINE |
| | Chronic_kidney_disease | APICIDIN |
| | Height | BELINOSTAT |
| | Mean_corpuscular_hemoglobin | BUTYRIC ACID |
| | Mean_corpuscular_volume | CHIDAMIDE |
| | Mean_platelet_volume | CHR-3996 |
| | Multiple_sclerosis | CUDC-101 |
| | Phospholipid_levels_plasma | DACINOSTAT |
| | QRS_duration | ENTINOSTAT |
| | Red_blood_cell_traits | GIVINOSTAT |
| | Testicular_germ_cell_tumor | LOVASTATIN |
| | Type_2_diabetes | MOCETINOSTAT |
| | Urate_levels | OXTRIPHYLLINE |
| | | PANOBINOSTAT |
| | | PCI-24781 |
| | | PIVANEX |
| | | PRACINOSTAT |
| | | RESMINOSTAT |
| | | ROMIDEPSIN |
| | | SCRIPTAID |
| | | THEOPHYLLINE |
| | | TRICHOSTATIN A |
| | | VALPROIC ACID |
| | | VORINOSTAT |

TABLE 6

| Column A | | |
|---|---|---|
| Transcription | Column B | Column C |
| Factor | Disease/Condition | Treatment Agent |
| TF: NR3C1 | Bladder_cancer | ALCLOMETASONE |
| | Height | ALCLOMETASONE |
| | Inflammatory_bowel_disease | DIPROPIONATE |
| | Intracranial_aneurysm | AMCINONIDE |
| | Phospholipid_levels_plasma | BECLOMETHASONE |
| | Prostate_cancer | BECLOMETHASONE |
| | Vitiligo | DIPROPIONATE |
| | | BETAMETHASONE |
| | | BETAMETHASONE |
| | | ACETATE |
| | | BETAMETHASONE |
| | | DIPROPIONATE |
| | | BETAMETHASONE |
| | | SODIUM |
| | | PHOSPHATE |
| | | BETAMETHASONE |
| | | VALERATE |
| | | BUDESONIDE |
| | | CICLESONIDE |
| | | CLOBETASOL |
| | | CLOBETASOL |
| | | PROPIONATE |
| | | CLOCORTOLONE |
| | | CLOCORTOLONE |
| | | PIVALATE |
| | | CORTISONE ACETATE |
| | | DESONIDE |
| | | DESOXIMETASONE |
| | | DEXAMETHASONE |
| | | DEXAMETHASONE |
| | | ACETATE |
| | | DEXAMETHASONE |
| | | SODIUM |
| | | PHOSPHATE |
| | | DIFLORASONE |
| | | DIFLORASONE |
| | | DIACETATE |
| | | DIFLUPREDNATE |
| | | FLUDROCORTISONE |
| | | FLUMETHASONE |
| | | PIVALATE |
| | | FLUNISOLIDE |
| | | FLUOCINOLONE |
| | | ACETONIDE |
| | | FLUOCINONIDE |
| | | FLUOROMETHOLONE |
| | | FLUOROMETHOLONE |
| | | ACETATE |
| | | FLUOXYMESTERONE |
| | | FLUPREDNISOLONE |
| | | FLURANDRENOLIDE |
| | | FLUTICASONE |
| | | FLUTICASONE |
| | | FUROATE |
| | | FLUTICASONE |
| | | PROPIONATE |
| | | HALCINONIDE |
| | | HALOBETASOL |
| | | PROPIONATE |
| | | HYDROCORTAMATE |
| | | HYDROCORTISONE |
| | | HYDROCORTISONE |
| | | ACETATE |
| | | HYDROCORTISONE |
| | | BUTYRATE |
| | | HYDROCORTISONE |
| | | CYPIONATE |
| | | HYDROCORTISONE |
| | | SODIUM |
| | | PHOSPHATE |
| | | HYDROCORTISONE |
| | | SODIUM |
| | | SUCCINATE |
| | | HYDROCORTISONE |
| | | VALERATE |
| | | LOTEPREDNOL |
| | | LOTEPREDNOL |
| | | ETABONATE |
| | | MEDRYSONE |
| | | MEGESTROL ACETATE |
| | | MEPREDNISONE |
| | | METHYL- |
| | | PREDNISOLONE |
| | | METHYL- |
| | | PREDNISOLONE |
| | | ACETATE |
| | | MIFEPRISTONE |
| | | MOMETASONE |
| | | MOMETASONE |
| | | FUROATE |
| | | ONAPRISTONE |
| | | PARAMETHASONE |
| | | PARAMETHASONE |
| | | ACETATE |
| | | PREDNICARBATE |
| | | PREDNISOLONE |
| | | PREDNISOLONE |
| | | ACETATE |
| | | PREDNISOLONE |
| | | SODIUM |
| | | PHOSPHATE |
| | | PREDNISOLONE |
| | | TEBUTATE |
| | | PREDNISONE |
| | | RIMEXOLONE |
| | | SPIRONOLACTONE |
| | | TRIAMCINOLONE |
| | | TRIAMCINOLONE |
| | | ACETONIDE |
| | | TRIAMCINOLONE |
| | | DIACETATE |
| | | TRIAMCINOLONE |
| | | HEXACETONIDE |
| | | ZK112993 |

TABLE 7

| Column A | | |
|---|---|---|
| Transcription | Column B | Column C |
| Factor | Disease/Condition | Treatment Agent |
| TF: VDR | Ankylosing_spondylitis | 1,25-DIHYDROXYVITAMIN D3 |
| | Basal_cell_carcinoma | 1,3-CYCLOHEXANEDIOL, 4- |
| | Breast_cancer | METHYLENE-5-[(2E)-[(1S,3 . . . |
| | Celiac_disease | 1,3-CYCLOHEXANEDIOL, 4- |
| | Cholesterol_total | METHYLENE-5-[(2E)- |
| | Chronic_lymphocytic_leukemia | [(1S,3AS,7AS)-OCTAHYDRO-1-(5- |
| | Crohns_disease | HYDROXY-5-METHYL-1,3- |
| | Fibrinogen | HEXADIYNYL)-7A-METHYL-4H- |
| | Height | INDEN-4- |
| | IgG_glycosylation | YLIDENE]ETHYLIDENE]-, |
| | Inflammatory_bowel_disease | (1R,3S,5Z) |
| | Juvenile_idiopathic_arthritis | 19356-17-3 |
| | Mean_corpuscular_hemoglobin | 5-{2-[1-(1-METHYL-PROPYL)-7A- |
| | Mean_platelet_volume | METHYL-OCTAHYDRO-I . . . |
| | Multiple_sclerosis | 5-{2-[1-(1-METHYL-PROPYL)-7A- |
| | Primary_biliary_cirrhosis | METHYL-OCTAHYDRO-INDEN-4- |
| | Rheumatoid_arthritis | YLIDENE]-ETHYLIDENE}-2- |
| | Systemic_lupus_erythematosus | METHYLENE-CYCLOHEXANE- |
| | Type_1_diabetes | 1,3-DIOL |
| | Ulcerative_colitis | ALFACALCIDOL |
| | Vitiligo | BONEFOS |
| | | CALCIFEDIOL |
| | | CALCIPOTRIENE |
| | | CALCIPOTRIOL |
| | | CALCITRIOL |
| | | CALCIUM |
| | | CHOLECALCIFEROL |
| | | DIHYDROTACHYSTEROL |
| | | DOXERCALCIFEROL |
| | | ELOCALCITOL |
| | | ERGOCALCIFEROL |
| | | INECALCITOL |
| | | LEXACALCITOL |
| | | LITHOCHOLIC ACID |
| | | PARICALCITOL |
| | | SEOCALCITOL |
| | | TACALCITOL |

TABLE 8

| Column A | | |
|---|---|---|
| Transcription | Column B | Column C |
| Factor | Disease/Condition | Treatment Agent |
| TF: RXRA | Blood_metabolite_levels | ACITRETIN |
| | Blood_metabolite_ratios | ADAPALENE |
| | Cholesterol_total | ALITRETINOIN |
| | Chronic_kidney_disease | BEXAROTENE |
| | Fasting_glucose- | ETODOLAC |
| | related_traits_interaction_with_BMI | ETRETINATE |
| | Height | METHOPRENE |
| | Inflammatory_bowel_disease | ACID |
| | LDL_cholesterol | R-ETODOLAC |
| | Mean_platelet_volume | |
| | Metabolic_syndrome | |
| | Metabolite_levels | |
| | Triglycerides | |
| | Urate_levels | |

TABLE 9

| Column A | | |
|---|---|---|
| Transcription | Column B | Column C |
| Factor | Disease/Condition | Treatment Agent |
| TF: RARG | Blood_metabolite_levels | ACITRETIN |
| | Blood_metabolite_ratios | ADAPALENE |
| | Height | AHPN |
| | Inflammatory_bowel_disease | ALITRETINOIN |
| | Juvenile_idiopathic_arthritis | CD564 |
| | Multiple_sclerosis | DODECYL-ALPHA-D- |
| | Red_blood_cell_traits | MALTOSIDE |
| | Serum_albumin_level | ETRETINATE |
| | | FENRETINIDE |
| | | MM 11253 |
| | | TAZAROTENE |
| | | TTNPB |

TABLE 10

| Column A | | |
|---|---|---|
| Transcription | Column B | Column C |
| Factor | Disease/Condition | Treatment Agent |
| TF: NFKB1 | Acute_lymphoblastic_leukemia_B- | BARDOXOLONE |
| | cell_precursor | BORTEZOMIB |
| | Ankylosing_spondylitis | THALIDOMIDE |
| | Atopic_dermatitis | TRIFLUSAL |
| | Body_mass_index | |
| | Celiac_disease | |
| | Chronic_lymphocytic_leukemia | |
| | Height | |
| | IgG_glycosylation | |
| | Inflammatory_bowel_disease | |
| | Juvenile_idiopathic_arthritis | |
| | Kawasaki_disease | |
| | Mean_corpuscular_hemoglobin | |
| | Mean_corpuscular_volume | |
| | Multiple_sclerosis | |
| | Primary_biliary_cirrhosis | |
| | Rheumatoid_arthritis | |
| | Systemic_lupus_erythematosus | |
| | Type_1_diabetes | |
| | Ulcerative_colitis | |
| | Vitiligo | |

TABLE 11

| Column A | | |
|---|---|---|
| Transcription | Column B | Column C |
| Factor | Disease/Condition | Treatment Agent |
| TF: CHD1 | Crohns_disease | EPIRUBICIN |
| | HDL_cholesterol | |
| | Height | |
| | Inflammatory_bowel_disease | |
| | Lipid_metabolism_phenotypes | |
| | Mean_corpuscular_volume | |
| | Menopause_age_at_onset | |
| | Multiple_sclerosis | |
| | Red_blood_cell_traits | |
| | Rheumatoid_arthritis | |
| | Schizophrenia | |
| | Systemic_lupus_erythematosus | |
| | Systemic_sclerosis | |
| | Telomere_length | |
| | Triglycerides | |
| | Type_1_diabetes | |
| | Ulcerative_colitis | |
| | Vitiligo | |

TABLE 12

| Column A | | |
|---|---|---|
| Transcription | Column B | Column C |
| Factor | Disease/Condition | Treatment Agent |
| TF: | Ankylosing_spondylitis | RO4929097 |
| NOTCH1 | Cholesterol_total | |
| | Crohns_disease | |
| | Graves_disease | |
| | Height | |
| | Inflammatory_bowel_disease | |
| | Lipid_metabolism_phenotypes | |
| | Mean_corpuscular_hemoglobin | |
| | Multiple_sclerosis | |
| | Red_blood_cell_traits | |
| | Rheumatoid_arthritis | |
| | Schizophrenia | |
| | Systemic_lupus_erythematosus | |

TABLE 13

| Column A | | |
|---|---|---|
| Transcription | Column B | Column C |
| Factor | Disease/Condition | Treatment Agent |
| TF: STAT5B | Celiac_disease | DASATINIB |
| | Chronic_lymphocytic_leukemia | |
| | Graves_disease | |
| | Inflammatory_bowel_disease | |
| | LDL_cholesterol | |
| | Multiple_sclerosis | |
| | Rheumatoid_arthritis | |
| | Self-reported_allergy | |
| | Systemic_lupus_erythematosus | |
| | Type_1_diabetes | |
| | Ulcerative_colitis | |

TABLE 14

| Column A | | |
|---|---|---|
| Transcription | Column B | Column C |
| Factor | Disease/Condition | Treatment Agent |
| TF: HDAC1 | Blood_metabolite_levels | 4SC-202 |
| | Blood_metabolite_ratios | APICIDIN |
| | Cholesterol_total | BELINOSTAT |
| | Glycated_hemoglobin_levels | BUTYRIC ACID |
| | Height | CBHA |
| | Inflammatory_bowel_disease | CHEMBL152543 |
| | Mean_corpuscular_hemoglobin | CHEMBL191091 |
| | Mean_corpuscular_volume | CHEMBL491491 |
| | Metabolite_levels | CHIDAMIDE |
| | Red_blood_cell_traits | CHLAMYDOCIN |
| | Systemic_lupus_erythematosus | CHR-3996 |
| | | CUDC-101 |
| | | DACINOSTAT |
| | | DEPUDECIN |
| | | ENTINOSTAT |
| | | GIVINOSTAT |
| | | MOCETINOSTAT |
| | | NEXTURASTAT A |
| | | OXAMFLATIN |
| | | PANOBINOSTAT |
| | | PCI-24781 |
| | | PIVANEX |
| | | PRACINOSTAT |
| | | PYROXAMIDE |
| | | RESMINOSTAT |
| | | RG2833 |
| | | ROMIDEPSIN |
| | | SB-639 |
| | | SCRIPTAID |
| | | SK-7041 |
| | | TRICHOSTATIN A |
| | | VALPROIC ACID |
| | | VORINOSTAT |

TABLE 15

| Column A | | |
|---|---|---|
| Transcription | Column B | Column C |
| Factor | Disease/Condition | Treatment Agent |
| TF: CDK9 | Atopic_dermatitis | DINACICLIB |
| | Complement_C3_and_C4_levels | FLAVOPIRIDOL |
| | Height | P276-00 |
| | Mean_platelet_volume | RGB-286638 |
| | Menopause_age_at_onset | |
| | Systemic_lupus_erythematosus | |

TABLE 16

| Column A | | |
|---|---|---|
| Transcription | Column B | Column C |
| Factor | Disease/Condition | Treatment Agent |
| TF: HDAC6 | Ankylosing_spondylitis | ACY-1215 |
| | Chronic_lymphocytic_leukemia | BELINOSTAT |
| | Crohns_disease | BUFEXAMAC |
| | Mean_corpuscular_volume | CUDC-101 |
| | Multiple_sclerosis | DACINOSTAT |
| | QT_interval | GIVINOSTAT |
| | | NEXTURASTAT A |
| | | PANOBINOSTAT |
| | | PCI-24781 |
| | | PRACINOSTAT |
| | | RESMINOSTAT |
| | | ROMIDEPSIN |
| | | SCRIPTAID |
| | | TRICHOSTATIN A |
| | | TUBACIN |
| | | VORINOSTAT |

TABLE 17

| Column A | | |
|---|---|---|
| Transcription | Column B | Column C |
| Factor | Disease/Condition | Treatment Agent |
| TF: JUN | Blood_metabolite_levels | IRBESARTAN |
| | Mean_corpuscular_volume | T-5224 |
| | Red_blood_cell_traits | VINBLASTINE |
| | Ulcerative_colitis | |

TABLE 18

| Column A | | |
|---|---|---|
| Transcription | Column B | Column C |
| Factor | Disease State | Treatment Agent |
| TF: HDAC8 | Parkinson_disease | 4-DIMETHYLAMINO-N-(6- |
| | Red_blood_cell_traits | HYDROXYCARBAMOYETHYL)BENZA . . . |
| | Systemic_lupus_erythematosus | 4-DIMETHYLAMINO-N-(6- |
| | | HYDROXYCARBAMOYETHYL)BENZAMIDE- |
| | | N-HYDROXY-7-(4- |
| | | DIMETHYLAMINOBENZOYL)AMINOHEPTANAMIDE |
| | | 4SC-202 |
| | | 5-(4-METHYL-BENZOYLAMINO)- |
| | | BIPHENYL-3,4′-DICARBO . . . |
| | | 5-(4-METHYL-BENZOYLAMINO)- |
| | | BIPHENYL-3,4′-DICARBOXYLIC |
| | | ACID 3-DIMETHYLAMIDE-4′- |
| | | HYDROXYAMIDE |
| | | APICIDIN |
| | | BELINOSTAT |
| | | BUTYRIC ACID |
| | | CHR-3996 |
| | | CUDC-101 |
| | | DACINOSTAT |
| | | ENTINOSTAT |
| | | GIVINOSTAT |
| | | N-HYDROXY-4-(METHYL{[5-(2- |
| | | PYRIDINYL)-2-THIENYL] . . . |
| | | N-HYDROXY-4-(METHYL{[5-(2- |
| | | PYRIDINYL)-2- |
| | | THIENYL]SULFONYL}AMINO)BENZAMIDE |
| | | PANOBINOSTAT |
| | | PCI-24781 |
| | | PIVANEX |
| | | PRACINOSTAT |
| | | RESMINOSTAT |
| | | ROMIDEPSIN |
| | | SCRIPTAID |
| | | TRICHOSTATIN A |
| | | VALPROIC ACID |
| | | VORINOSTAT |

TABLE 19

| Column A | | |
|---|---|---|
| Transcription | Column B | Column C |
| Factor | Disease/Condition | Treatment Agent |
| TF: EP300 | Alzheimer_disease | ANACARDIC ACID |
| | Alzheimer_disease_late_onset | CURCUMIN |
| | Ankylosing_spondylitis | GARCINOL |
| | Atopic_dermatitis | LYS-COA |
| | Basal_cell_carcinoma | PLUMBAGIN |
| | Blood_metabolite_levels | |
| | Celiac_disease | |
| | Central_corneal_thickness | |
| | Cholesterol_total | |
| | Chronic_lymphocytic_leukemia | |
| | Crohns_disease | |
| | Endometriosis | |
| | Fasting_glucose- | |
| | related_traits_interaction_with_BMI | |
| | Fibrinogen | |
| | Glycemic_traits | |
| | HDL_cholesterol | |
| | Heart_rate | |
| | Height | |
| | IgG_glycosylation | |
| | Inflammatory_bowel_disease | |
| | LDL_cholesterol | |
| | Lipid_metabolism_phenotypes | |
| | Lipoprotein- | |
| | associated_phospholipase_A2_activity_and_mass | |
| | Mean_corpuscular_hemoglobin | |
| | Mean_corpuscular_volume | |
| | Mean_platelet_volume | |
| | Metabolic_syndrome | |
| | Migraine | |
| | Multiple_sclerosis | |
| | Pancreatic_cancer | |
| | Platelet_counts | |
| | Primary_biliary_cirrhosis | |
| | Primary_tooth_development_time_to_first_tooth_eruption | |
| | Pulmonary_function | |
| | Pulmonary_function_interaction | |
| | QRS_duration | |
| | Red_blood_cell_traits | |
| | Renal_cell_carcinoma | |
| | Renal_function-related_traits_BUN | |
| | Rheumatoid_arthritis | |
| | Self-reported_allergy | |
| | Systemic_lupus_erythematosus | |
| | Triglycerides | |
| | Type_1_diabetes | |
| | Ulcerative_colitis | |
| | Urate_levels | |
| | Vitiligo | |

TABLE 20

| Column A | | |
|---|---|---|
| Transcription | Column B | Column C |
| Factor | Disease/Condition | Treatment Agent |
| TF: MYC | Ankylosing_spondylitis | ALISERTIB |
| | Bladder_cancer | DINACICLIB |
| | Blood_metabolite_levels | |
| | Blood_metabolite_ratios | |
| | Body_mass_index | |
| | Cholesterol_total | |
| | Chronic_lymphocytic_leukemia | |
| | Crohns_disease | |
| | Esophageal_cancer_squamous_cell | |
| | Fibrinogen | |
| | Glycated_hemoglobin_levels | |
| | Glycemic_traits_pregnancy | |
| | HDL_cholesterol | |
| | Heart_rate | |
| | Height | |
| | IgG_glycosylation | |
| | Inflammatory_bowel_disease | |
| | Interstitial_lung_disease | |
| | Juvenile_idiopathic_arthritis | |
| | Lipid_metabolism_phenotypes | |
| | Lipoprotein- | |
| | associated_phospholipase_A2_activity_and_mass | |
| | Lung_cancer | |
| | Mean_corpuscular_hemoglobin | |
| | Mean_corpuscular_hemoglobin_concentration | |
| | Mean_corpuscular_volume | |
| | Mean_platelet_volume | |
| | Menopause_age_at_onset | |
| | Metabolic_syndrome | |
| | Metabolite_levels | |
| | Migraine | |
| | Multiple_sclerosis | |
| | Pancreatic_cancer | |
| | Phospholipid_levels_plasma | |
| | Platelet_counts | |
| | QRS_duration | |
| | Red_blood_cell_traits | |
| | Renal_cell_carcinoma | |
| | Renal_function-related_traits_BUN | |
| | Resting_heart_rate | |
| | Rheumatoid_arthritis | |
| | Schizophrenia | |
| | Systemic_lupus_erythematosus | |
| | Testicular_germ_cell_tumor | |
| | Triglycerides | |
| | Ulcerative_colitis | |
| | Vitiligo | |

TABLE 21

| Column A | | |
|---|---|---|
| Transcription | Column B | Column C |
| Factor | Disease/Condition | Treatment Agent |
| TF: BRD4 | Acute_lymphoblastic_leukemia_B- | CPI-203 |
| | cell_precursor | GW841819X |
| | Ankylosing_spondylitis | I-BET151 |
| | Bipolar_disorder | MS417 |
| | Body_mass_index | MS436 |
| | Celiac_disease | PFI-1 |
| | Cholesterol_total | XD14 |
| | Chronic_lymphocytic_leukemia | |
| | Crohns_disease | |
| | End-stage_coagulation | |
| | Esophageal_cancer_squamous_cell | |
| | Fasting_glucose- | |
| | related_traits_interaction_with_BMI | |
| | Fibrinogen | |
| | Glycated_hemoglobin_levels | |
| | HDL_cholesterol | |
| | Height | |
| | IgG_glycosylation | |
| | Inflammatory_bowel_disease | |
| | Interstitial_lung_disease | |
| | Juvenile_idiopathic_arthritis | |
| | Kawasaki_disease | |
| | Mean_corpuscular_hemoglobin | |
| | Mean_corpuscular_volume | |
| | Mean_platelet_volume | |
| | Menopause_age_at_onset | |
| | Metabolic_syndrome | |
| | Multiple_sclerosis | |
| | Parkinson_disease | |
| | Phospholipid_levels_plasma | |
| | Platelet_counts | |
| | Red_blood_cell_traits | |
| | Rheumatoid_arthritis | |
| | Systemic_lupus_erythematosus | |
| | Testicular_germ_cell_tumor | |
| | Triglycerides | |
| | Type_1_diabetes | |
| | Ulcerative_colitis | |

TABLE 22

| Column A | | |
|---|---|---|
| Tran- | | |
| scription | Column B | Column C |
| Factor | Disease/Condition | Treatment Agent |
| TF: | Asthma_and_hay_fever | PSEUDOEPHEDRINE |
| NFATC1 | Atopic_dermatitis | |
| | Basal_cell_carcinoma | |
| | Behcets_disease | |
| | Celiac_disease | |
| | Chronic_lymphocytic_leukemia | |
| | Crohns_disease | |
| | IgG_glycosylation | |
| | Inflammatory_bowel_disease | |
| | Juvenile_idiopathic_arthritis | |
| | Mean_corpuscular_hemoglobin | |
| | Mean_platelet_volume | |
| | Multiple_sclerosis | |
| | Platelet_counts | |
| | Primary_biliary_cirrhosis | |
| | Prostate_cancer | |
| | Red_blood_cell_traits | |
| | Rheumatoid_arthritis | |
| | Systemic_lupus_erythematosus | |
| | Systemic_sclerosis | |
| | Type_1_diabetes | |
| | Ulcerative_colitis | |

TABLE 23

| Column A | | |
|---|---|---|
| Transcription | Column B | Column C |
| Factor | Disease/Condition | Treatment Agent |
| TF: RUNX1 | Acute_lymphoblastic_leukemia_B- | METHACHOLINE CHLORIDE |
| | cell_precursor | |
| | Alzheimer_disease | |
| | Alzheimer_disease_late_onset | |
| | Ankylosing_spondylitis | |
| | Central_corneal_thickness | |
| | Coronary_heart_disease | |
| | Crohns_disease | |
| | Fibrinogen | |
| | HDL_cholesterol | |
| | Height | |
| | Inflammatory_bowel_disease | |
| | Juvenile_idiopathic_arthritis | |
| | Mean_corpuscular_hemoglobin | |
| | Mean_platelet_volume | |
| | Multiple_sclerosis | |
| | Platelet_counts | |
| | Red_blood_cell_traits | |
| | Rheumatoid_arthritis | |
| | Systemic_lupus_erythematosus | |
| | Takayasu_arteritis | |
| | Ulcerative_colitis | |

TABLE 24

| Column A | | |
|---|---|---|
| Transcription | Column B | Column C |
| Factor | Disease/Condition | Treatment Agent |
| TF: TCF7L2 | Basal_cell_carcinoma | REPAGLINIDE |
| | Breast_cancer | |
| | Chronic_lymphocytic_leukemia | |
| | Fasting_glucose- | |
| | related_traits_interaction_with_BMI | |
| | Fibrinogen | |
| | Height | |
| | Hodgkin_lymphoma | |
| | Inflammatory_bowel_disease | |
| | Lipoprotein- | |
| | associated_phospholipase_A2_activity_and_mass | |
| | Mean_corpuscular_hemoglobin | |
| | Mean_corpuscular_volume | |
| | Ovarian_cancer | |
| | Primary_biliary_cirrhosis | |
| | Primary_tooth_development_time_to_first_tooth_eruption | |
| | Red_blood_cell_traits | |
| | Schizophrenia | |
| | Serum_albumin_level | |
| | Systemic_lupus_erythematosus | |

TABLE 25

| Column A | | |
|---|---|---|
| Transcription | Column B | Column C |
| Factor | Disease/Condition | Treatment Agent |
| TF: PHF8 | Blood_metabolite_levels | DAMINOZIDE |
| | Esophageal_cancer_squamous_cell | |
| | Fasting_glucose- | |
| | related_traits_interaction_with_BMI | |
| | Glycated_hemoglobin_levels | |
| | HDL_cholesterol | |
| | Heart_rate | |
| | Height | |
| | Inflammatory_bowel_disease | |
| | Interstitial_lung_disease | |
| | Mean_corpuscular_hemoglobin | |
| | Mean_platelet_volume | |
| | Menopause_age_at_onset | |
| | Multiple_sclerosis | |
| | Phospholipid_levels_plasma | |
| | Red_blood_cell_traits | |
| | Schizophrenia | |
| | Systemic_lupus_erythematosus | |
| | Telomere_length | |

TABLE 26

| Column A | | |
|---|---|---|
| Transcription | Column B | Column C |
| Factor | Disease/Condition | Treatment Agent |
| TF: HNF4A | Blood_metabolite_levels | LINOLEIC ACID |
| | Blood_metabolite_ratios | |
| | Breast_cancer | |
| | C-reactive_protein | |
| | Cholesterol_total | |
| | Colorectal_cancer | |
| | HDL_cholesterol | |
| | Inflammatory_bowel_disease | |
| | LDL_cholesterol | |
| | Lipid_metabolism_phenotypes | |
| | Metabolic_syndrome | |
| | Metabolite_levels | |
| | Multiple_sclerosis | |
| | Primary_biliary_cirrhosis | |
| | Sphingolipid_levels | |
| | Triglycerides | |
| | Urate_levels | |

TABLE 27

| Column A | | |
|---|---|---|
| Transcription | Column B | Column C |
| Factor | Disease/Condition | Treatment Agent |
| TF: MED1 | ANCA-associated_vasculitis | 5-{2-[1-(1-METHYL-PROPYL)-7A- |
| | Blood_metabolite_levels | METHYL-OCTAHYDRO-I . . . |
| | Blood_metabolite_ratios | 5-{2-[1-(1-METHYL-PROPYL)-7A- |
| | Celiac_disease | METHYL-OCTAHYDRO-INDEN-4- |
| | Crohns_disease | YLIDENE]-ETHYLIDENE}-2- |
| | Educational_attainment | METHYLENE-CYCLOHEXANE-1,3- |
| | Graves_disease | DIOL |
| | Height | |
| | IgG_glycosylation | |
| | Inflammatory_bowel_disease | |
| | Kawasaki_disease | |
| | Prostate_cancer | |
| | Rheumatoid_arthritis | |
| | Systemic_lupus_erythematosus | |
| | Takayasu_arteritis | |
| | Type_1_diabetes | |
| | Vitiligo | |

TABLE 28

| Column A | | Column C |
|---|---|---|
| Transcription | Column B | Treatment |
| Factor | Disease/Condition | Agent |
| TF: NFKB2 | Celiac_disease | TRIPTOSAR |
| | Height | |
| | Inflammatory_bowel_disease | |
| | Mean_corpuscular_hemoglobin | |
| | Multiple_sclerosis | |
| | Primary_biliary_cirrhosis | |
| | Rheumatoid_arthritis | |
| | Systemic_lupus_erythematosus | |
| | Type_1_diabetes | |
| | Ulcerative_colitis | |
| | Urinary_metabolites_H-NMR_features | |
| | Vitiligo | |

TABLE 29

| Column A | | |
|---|---|---|
| Transcription | Column B | Column C |
| Factor | Disease/Condition | Treatment Agent |
| TF: | Acute_lymphoblastic_leukemia_B- | 9-ACETYL-2,3,4,9-TETRAHYDRO- |
| CREBBP | cell_precursor | 1H-CARBAZOL-1-ONE |
| | Celiac_disease | ISCHEMIN |
| | Crohns_disease | |
| | Inflammatory_bowel_disease | |
| | Mean_corpuscular_hemoglobin | |
| | Mean_corpuscular_volume | |
| | Multiple_sclerosis | |
| | Red_blood_cell_traits | |
| | Rheumatoid_arthritis | |
| | Systemic_lupus_erythematosus | |
| | Type_1_diabetes | |

TABLE 30

| Column A | | |
|---|---|---|
| Transcription | Column B | Column C |
| Factor | Disease/Condition | Treatment Agent |
| TF: STAT3 | Chronic_lymphocytic_leukemia | ATIPRIMOD |
| | Crohns_disease | DCL000217 |
| | Graves_disease | |
| | Inflammatory_bowel_disease | |
| | Juvenile_idiopathic_arthritis | |
| | Lipoprotein- | |
| | associated_phospholipase_A2_activity_and_mass | |
| | Multiple_sclerosis | |
| | Pancreatic_cancer | |
| | Systemic_lupus_erythematosus | |
| | Vitiligo | |

TABLE 31

| Column A | | |
|---|---|---|
| Transcription | Column B | Column C |
| Factor | Disease/Condition | Treatment Agent |
| TF: | Alzheimer_disease | CISPLATINUM |
| SMARCA4 | Blood_pressure | VINORELBINE |
| | Crohns_disease | |
| | Glycated_hemoglobin_levels | |
| | Inflammatory_bowel_disease | |
| | Mean_corpuscular_hemoglobin | |
| | Mean_corpuscular_volume | |
| | Red_blood_cell_traits | |
| | Systemic_lupus_erythematosus | |

TABLE 32

| Column A | | |
|---|---|---|
| Transcription | Column B | Column C |
| Factor | Disease/Condition | Treatment Agent |
| TF: BRD2 | Body_mass_index | ET BROMODOMAIN INHIBITOR |
| | Breast_cancer | GW841819X |
| | Cholesterol_total | I-BET151 |
| | Crohns_disease | ME BROMODOMAIN INHIBITOR |
| | Height | XD14 |
| | Inflammatory_bowel_disease | |
| | Mean_corpuscular_hemoglobin | |
| | Red_blood_cell_traits | |
| | Serum_albumin_level | |

TABLE 33

| Column A | | |
|---|---|---|
| Transcription | Column B | Column C |
| Factor | Disease/Condition | Treatment Agent |
| TF: STAT4 | Celiac_disease | LISOFYLLINE |
| | Crohns_disease | |
| | HDL_cholesterol | |
| | Height | |
| | Inflammatory_bowel_disease | |
| | Juvenile_idiopathic_arthritis | |
| | Multiple_sclerosis | |
| | Ulcerative_colitis | |

TABLE 34

| Column A | | |
|---|---|---|
| Transcription | Column B | Column C |
| Factor | Disease/Condition | Treatment Agent |
| TF: KDM5B | Glycated_hemoglobin_levels | PBIT |
| | Height | |
| | Inflammatory_bowel_disease | |
| | Lipid_metabolism_phenotypes | |
| | Lipoprotein- | |
| | associated_phospholipase_A2_activity_and_mass | |
| | Mean_corpuscular_hemoglobin | |
| | Multiple_sclerosis | |
| | Systemic_lupus_erythematosus | |

TABLE 35

| Column A | | |
|---|---|---|
| Transcription | Column B | Column C |
| Factor | Disease/Condition | Treatment Agent |
| TF: BRD3 | Esophageal_cancer_squamous_cell | GW841819X |
| | Height | I-BET151 |
| | Juvenile_idiopathic_arthritis | XD14 |
| | Mean_platelet_volume | |
| | Multiple_sclerosis | |
| | Rheumatoid_arthritis | |
| | Schizophrenia | |

TABLE 36

| Column A | | |
|---|---|---|
| Transcription | Column B | Column C |
| Factor | Disease/Condition | Treatment Agent |
| TF: EZH2 | Bone_mineral_density | EI1 |
| | C-reactive_protein | EPZ-6438 |
| | Fasting_glucose- | GSK126 |
| | related_traits_interaction_with_BMI | |
| | Inflammatory_bowel_disease | |
| | Ovarian_cancer | |
| | Prostate_cancer | |

TABLE 37

| Column A | | |
|---|---|---|
| Transcription | Column B | Column C |
| Factor | Disease/Condition | Treatment Agent |
| TF: ATF1 | Ankylosing_spondylitis | PSEUDOEPHEDRINE |
| | Colorectal_cancer | |
| | Mean_corpuscular_hemoglobin | |
| | Mean_corpuscular_volume | |
| | Red_blood_cell_traits | |
| | Systemic_lupus_erythematosus | |

TABLE 38

| Column A | | |
|---|---|---|
| Transcription | Column B | Column C |
| Factor | Disease/Condition | Treatment Agent |
| TF: CREB1 | Chronic_lymphocytic_leukemia | NALOXONE |
| | Glycated_hemoglobin_levels | |
| | Height | |
| | Mean_platelet_volume | |
| | Prostate_cancer | |
| | Schizophrenia | |

TABLE 39

| Column A | | |
|---|---|---|
| Transcription | Column B | Column C |
| Factor | Disease/Condition | Treatment Agent |
| TF: TP53 | Glycated_hemoglobin_levels | 1-(9-ETHYL-9H- |
| | Mean_platelet_volume | CARBAZOL-3-YL)- |
| | Multiple_sclerosis | N-METHYL- |
| | Rheumatoid_arthritis | METHANAMINE |
| | Testicular_germ_cell_tumor | DOXORUBICIN |

TABLE 40

| Column A | | |
|---|---|---|
| Transcription | Column B | Column C |
| Factor | Disease/Condition | Treatment Agent |
| TF: HNF4G | Blood_metabolite_levels | PALMITIC |
| | Blood_metabolite_ratios | ACID |
| | Metabolic_syndrome | |
| | Urate_levels | |

TABLE 41

| Column A | | Column C |
|---|---|---|
| Transcription | Column B | Treatment |
| Factor | Disease/Condition | Agent |
| TF: NR2C2 | Cholesterol_total | RETINOL |
| | Glycated_hemoglobin_levels | |
| | Mean_platelet_volume | |

TABLE 42

| Column A | | |
|---|---|---|
| Transcription | Column B | Column C |
| Factor | Disease/Condition | Treatment Agent |
| TF: SIRT6 | Mean_corpuscular_hemoglobin | PANOBINOSTAT |
| | Mean_corpuscular_volume | |
| | Red_blood_cell_traits | |

TABLE 43

| Column A | | |
|---|---|---|
| Transcription | Column B | Column C |
| Factor | Disease/Condition | Treatment Agent |
| TF: BRCA1 | Chronic_lymphocytic_leukemia | BMN673 |
| | Systemic_lupus_erythematosus | CARBOPLATIN |
| | | OLAPARIB |
| | | PLATINUM |
| | | RUCAPARIB |
| | | TAXANE |
| | | VELIPARIB |
| | | VINORELBINE |

TABLE 44

| Column A | | |
|---|---|---|
| Transcription | Column B | Column C |
| Factor | Disease/Condition | Treatment Agent |
| TF: NR1H2 | Alzheimer_disease | 1,1,1,3,3,3-HEXAFLUORO-2-{4- |
| | Glycated_hemoglobin_levels | [(2,2,2-TRIFLUOROET . . . |
| | | 1,1,1,3,3,3-HEXAFLUORO-2-{4- |
| | | [(2,2,2- |
| | | TRIFLUOROETHYL)AMINO]PHENYL}PROPAN- |
| | | 2-OL |
| | | 22R-HYDROXYCHOLESTEROL |
| | | 27-HYDROXYCHOLESTEROL |
| | | GW3965 |
| | | T0901317 |

TABLE 45

| Column A | | |
|---|---|---|
| Transcription | Column B | Column C |
| Factor | Disease/Condition | Treatment Agent |
| TF: KAT5 | Height | COENZYME A |
| | Schizophrenia | S-ACETYL-CYSTEINE |

TABLE 46

| Column A | | |
|---|---|---|
| Transcription | Column B | Column C |
| Factor | Disease/Condition | Treatment Agent |
| TF: | Schizophrenia | UREA |
| CTNNB1 | | |

TABLE 47

| Column A | | |
|---|---|---|
| Transcription | Column B | Column C |
| Factor | Disease/Condition | Treatment Agent |
| TF: KDM5A | Type_1_diabetes | PBIT |

TABLE 48

| Column A | | |
|---|---|---|
| Transcription | Column B | Column C |
| Factor | Disease/Condition | Treatment Agent |
| TF: PPARG | C-reactive_protein | (2S)-2-(4-CHLOROPHENOXY)- |
| | Fibrinogen | 3-PHENYLPROPANOIC ACID |
| | Rheumatoid_arthritis | (2S)-3-(1-{’-(2- |
| | | CHLOROPHENYL)-5- |
| | | METHYL-1,3-OXAZOL-4- |
| | | YL]METHYL}-1H-INDOL-5- |
| | | YL)-2-ETHOXYPROPANOIC |
| | | ACID |
| | | 3-FLUORO-N-[1-(4- |
| | | FLUOROPHENYL)-3-(2- |
| | | THIENYL)-1H-PYRAZOL-5- |
| | | YL]BENZENESULFONAMIDE |
| | | (4S,5E,7Z,10Z,13Z,16Z,19Z)-4- |
| | | HYDROXYDOCOSA- |
| | | 5,7,10,13,16,19- |
| | | HEXAENOIC ACID |
| | | (5R,6E,8Z,11Z,14Z,17Z)-5- |
| | | HYDROXYIOSA-6,8,11,14,17- |
| | | PENTAENOIC ACID |
| | | (8E,10S,12Z)-10-HYDROXY-6- |
| | | OXOOCTADECA-8,12-DIENOIC |
| | | ACID |
| | | (8R,9Z,12Z)-8-HYDROXY-6- |
| | | OXOOCTADECA-9,12-DIENOIC |
| | | ACID |
| | | AD-5061 |
| | | ALEGLITAZAR |
| | | BALSALAZIDE |
| | | BALSALAZIDE DISODIUM |
| | | BARDOXOLONE |
| | | BEZAFIBRATE |
| | | CIGLITAZONE |
| | | DB07509 |
| | | DICLOFENAC |
| | | FARGLITAZAR |
| | | FMOC-L-LEUCINE |
| | | GENISTEIN |
| | | GLIPIZIDE |
| | | GW0072 |
| | | GW1929 |
| | | GW7845 |
| | | GW9662 |
| | | IBUPROFEN |
| | | INDOMETHACIN |
| | | L-764406 |
| | | L-796449 |
| | | LINOLEIC ACID |
| | | LY-465608 |
| | | LY-510929 |
| | | METAGLIDASEN |
| | | MITIGLINIDE |
| | | MURAGLITAZAR |
| | | NATEGLINIDE |
| | | NAVEGLITAZAR |
| | | NETOGLITAZONE |
| | | NTZDPA |
| | | OLANZAPINE |
| | | OLSALAZINE SODIUM |
| | | PAT5A |
| | | PIOGLITAZONE |
| | | PIOGLITAZONE |
| | | HYDROCHLORIDE |
| | | RAGAGLITAZAR |
| | | REGLITAZAR |
| | | REPAGLINIDE |
| | | ROSIGLITAZONE |
| | | ROSIGLITAZONE MALEATE |
| | | ROSIGLITAZONE & |
| | | SIMVASTATIN |
| | | RS5444 |
| | | SB-219993 |
| | | SB-219994 |
| | | SULFASALAZINE |
| | | T131 |
| | | TELMISARTAN |
| | | TREPROSTINIL |
| | | TROGLITAZONE |
| | | ZOLEDRONIC ACID |

TABLE 49

| Column A | | |
|---|---|---|
| Transcription | Column B | Column C |
| Factor | Disease/Condition | Treatment Agent |
| TF: ZEB1 | Mean_corpuscular_hemoglobin | CYTARABINE |
| | Mean_corpuscular_volume | DOXORUBICIN |
| | Systemic_lupus_erythematosus | GEMCITABINE |
| | | SALINOMYCIN |

Dosage

As will be apparent to those skilled in the art, dosages outside of these disclosed ranges may be administered in some cases. Further, it is noted that the ordinary skilled clinician or treating physician will know how and when to interrupt, adjust, or terminate therapy in consideration of individual patient response.

In one aspect, the dosage of an agent disclosed herein, based on weight of the active compound, administered to an individual in need thereof may be about 0.25 mg/kg, 0.5 mg/kg, 0.1 mg/kg, 1 mg/kg, 2 mg/kg, 3 mg/kg, 4 mg/kg, 5 mg/kg, 6 mg/kg, or more of a subject's body weight. In another embodiment, the dosage may be a unit dose of about 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In one aspect, an agent disclosed herein may be present in an amount of from about 0.5% to about 95%, or from about 1% to about 90%, or from about 2% to about 85%, or from about 3% to about 80%, or from about 4%, about 75%, or from about 5% to about 70%, or from about 6%, about 65%, or from about 7% to about 60%, or from about 8% to about 55%, or from about 9% to about 50%, or from about 10% to about 40%, by weight of the composition.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms all utilizing dosage forms well known to those of ordinary skill in the pharmaceutical arts. The compositions may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A dosage regimen will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a symptom or effect of a disease can be readily determined by an ordinarily skilled physician

Compositions may include suitable dosage forms for oral, parenteral (including subcutaneous, intramuscular, intradermal and intravenous), transdermal, sublingual, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. Oral preparations include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. For topical or nasal administration, penetrants or permeation agents that are appropriate to the particular barrier to be permeated are used in the formulation. Such penetrants are generally known in the art. The pharmaceutical compositions are prepared by conventional techniques appropriate to the desired preparation containing appropriate amounts of the active ingredient, that is, one or more of the disclosed active agents or a pharmaceutically acceptable salt thereof according to the invention.

The dosage of an agent disclosed herein used to achieve a therapeutic effect will depend not only on such factors as the age, weight and sex of the patient and mode of administration, but also on the degree of inhibition desired and the potency of an agent disclosed herein for the particular disorder or disease concerned. It is also contemplated that the treatment and dosage of an agent disclosed herein may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

In one aspect, a method of treating a disease is disclosed, in which the method may comprise the step of identifying one or more, or two or more, or three or more, or four or more, or five or more, or six or more, or seven or more, or eight or more, or nine or more, or ten or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or more than 40 loci associated with a disease state as listed herein. The individual may have, or be suspected of having the disease. The method may further comprise the step of treating the individual with a compound that modulates the TF associated with the one or more loci.

### Examples

Application to a matrix of 213 phenotypes and 1,544 TF binding datasets identifies 2,264 significant associations for hundreds of TFs in 94 phenotypes, including prostate and breast cancers. Strikingly, nearly half of the systemic lupus erythematosus risk loci are occupied by the Epstein-Barr virus EBNA2 protein and 24 human TFs, revealing an important gene-environment interaction. Similar EBNA2-anchored associations also exist in multiple sclerosis, rheumatoid arthritis, inflammatory bowel disease, type 1 diabetes, juvenile idiopathic arthritis, and celiac disease. Instances of allele-dependent DNA binding and downstream effects on gene expression at plausibly causal autoimmune variants support a genetic mechanism of pathogenesis centered on EBNA2. Applicant's results nominate mechanisms operating across disease risk loci, suggesting new paradigms of disease origins.

The mechanisms generating genetic associations have proven difficult to elucidate for most diseases. Gene-environment interactions may explain the etiology of many autoimmune diseases¹⁻³. In particular, Epstein-Barr virus (EBV) infection has been implicated in the autoimmune mechanisms and epidemiology of systemic lupus erythematosus (SLE)⁴⁻⁷, increasing SLE risk by as much as 50-fold in children⁴. SLE patients also have elevated EBV loads in blood and early lytic viral gene expression⁶. Despite connections between EBV and multiple autoimmune diseases, the underlying molecular mechanisms remain unknown^{8,9}.

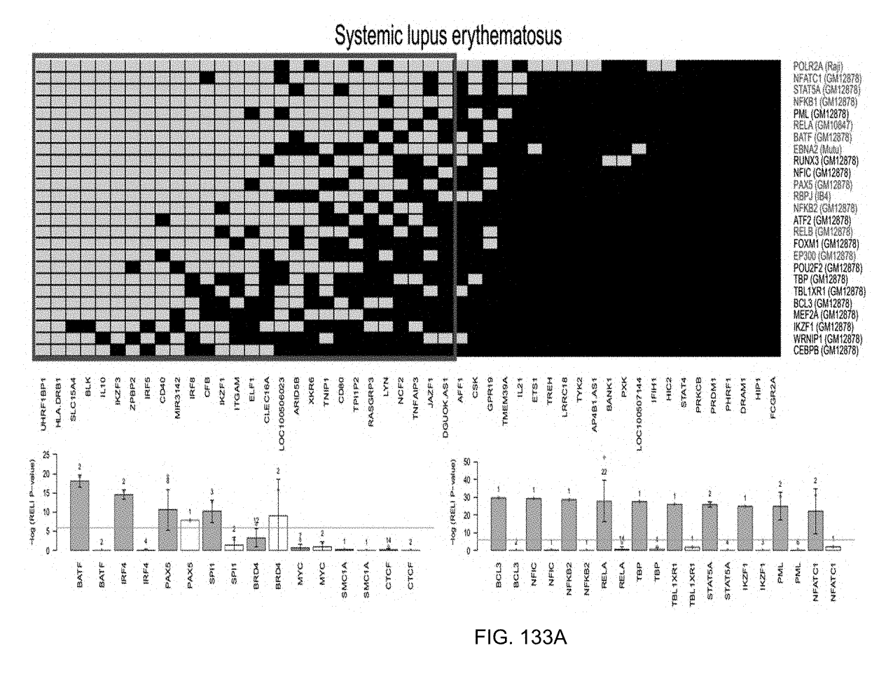

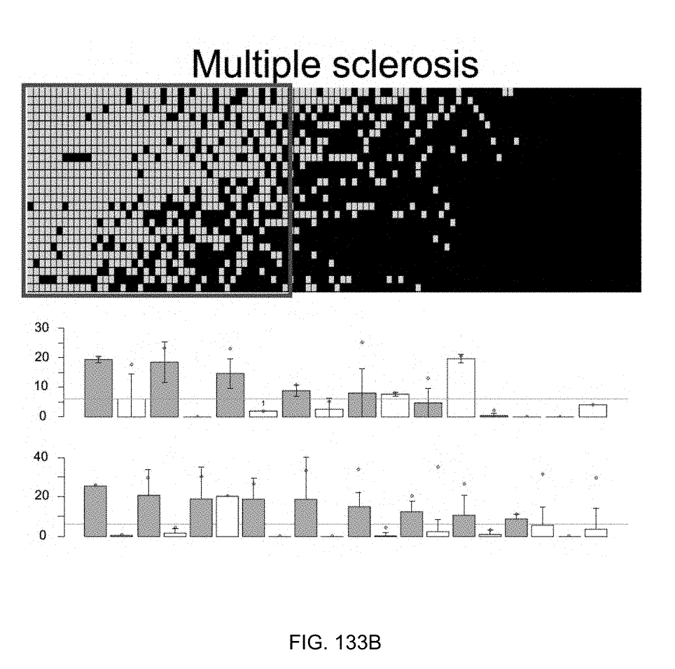

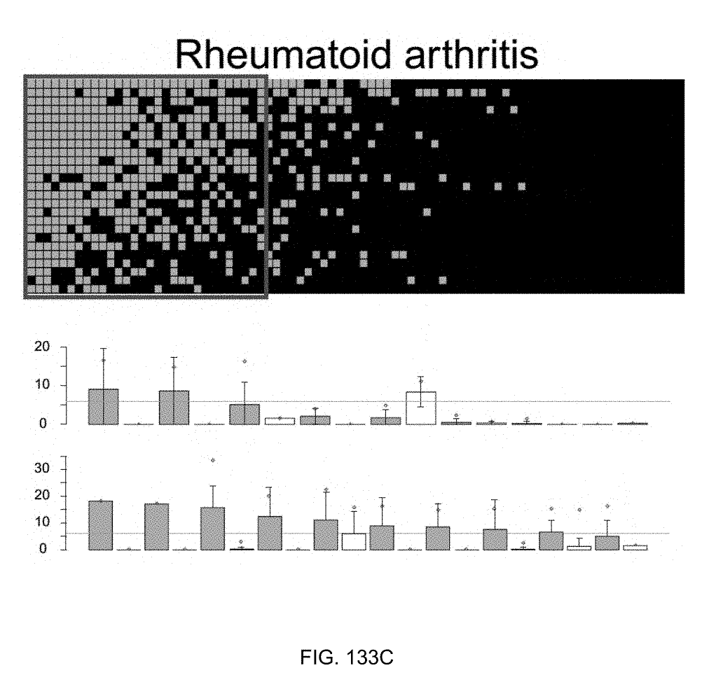

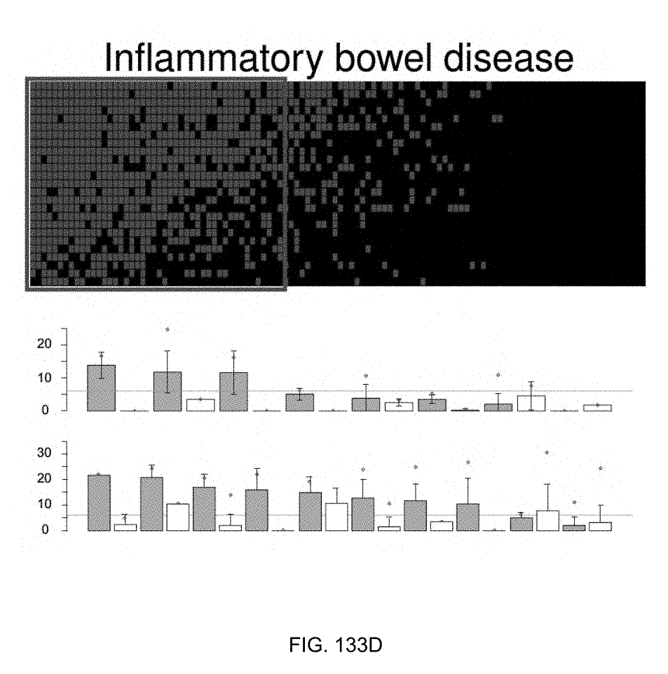

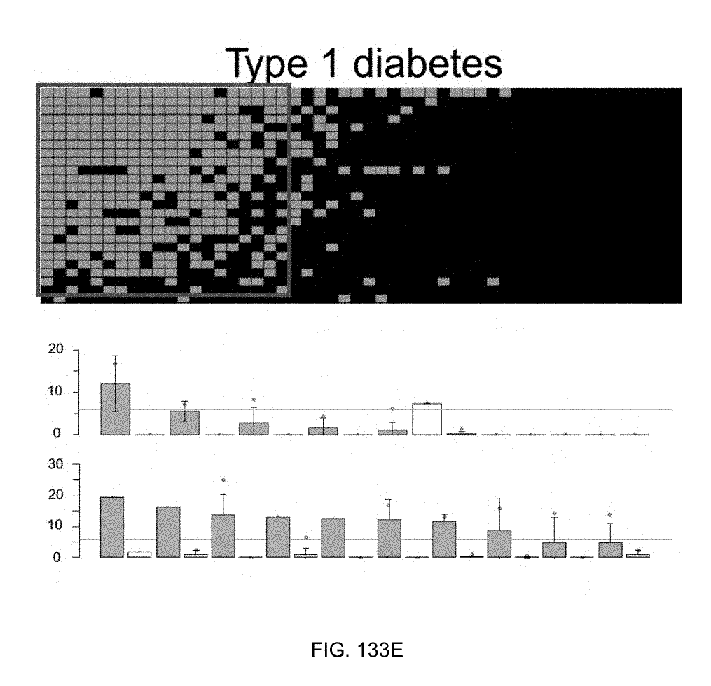

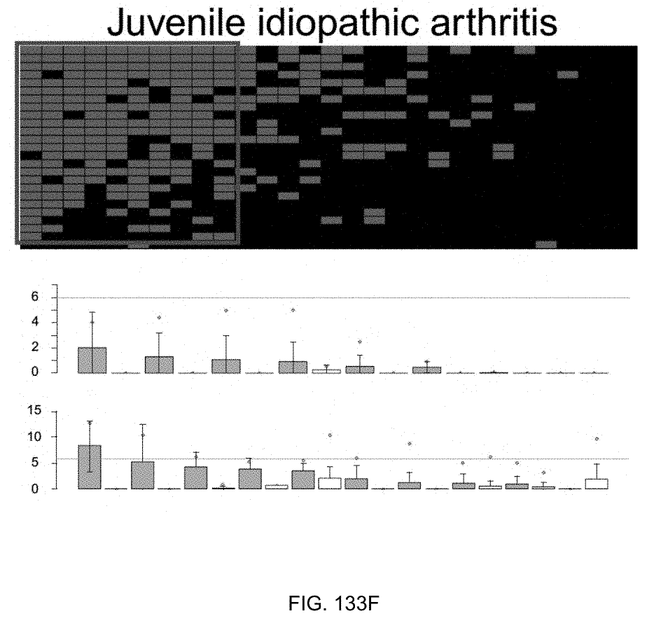

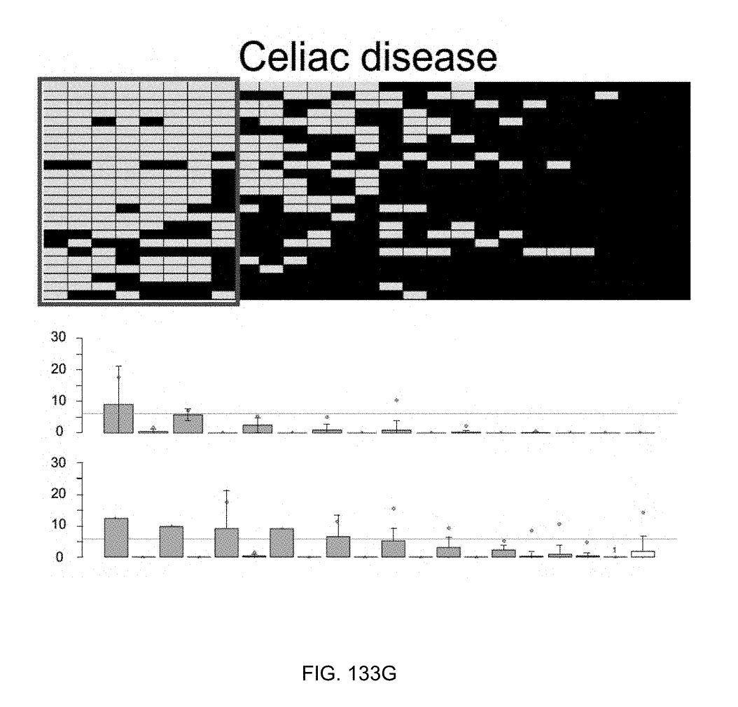

Genome wide association studies (GWASs) have identified >50 convincing European ancestry SLE loci (FIG. 133a), providing compelling evidence for germline DNA polymorphisms altering SLE risk¹⁰⁻¹³. Like most complex diseases, the great majority occur in likely gene regulatory regions^{14,15}. Applicant therefore asked if any of the DNA-interacting proteins encoded by EBV preferentially bind SLE risk loci. Applicant's analyses reveal powerful associations with an EBV gene product (EBNA2), providing a potential origin of gene-environment interaction, along with a set of human transcription factors and co-factors (TFs) in SLE and six other autoimmune diseases. Applicant present allele and EBV-dependent TF binding interactions and gene expression patterns that nominate cell types, molecular participants, and environmental contributions to disease mechanisms.

Intersection of Disease Risk Loci with TF-DNA Binding Interactions

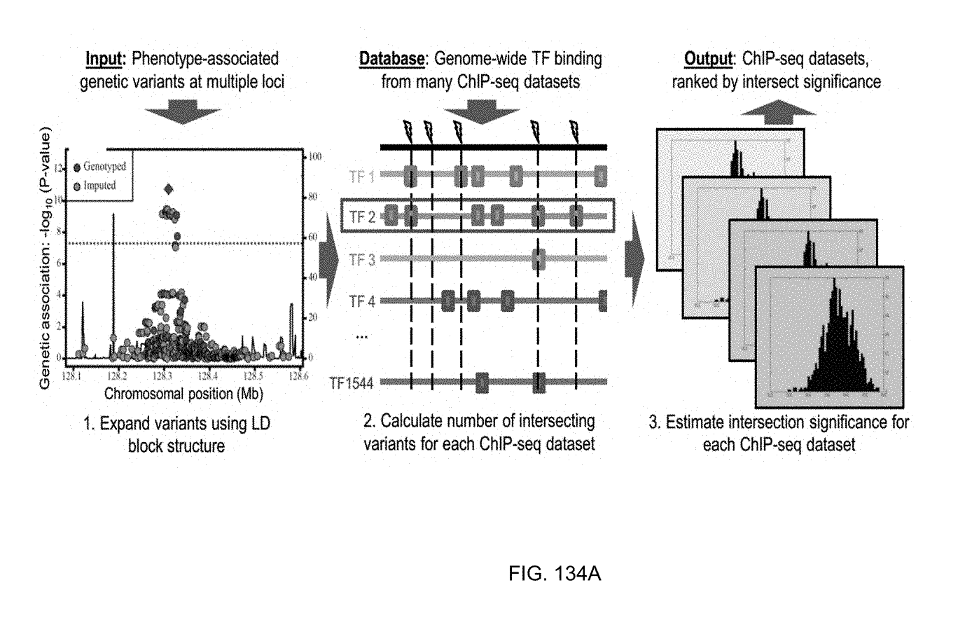

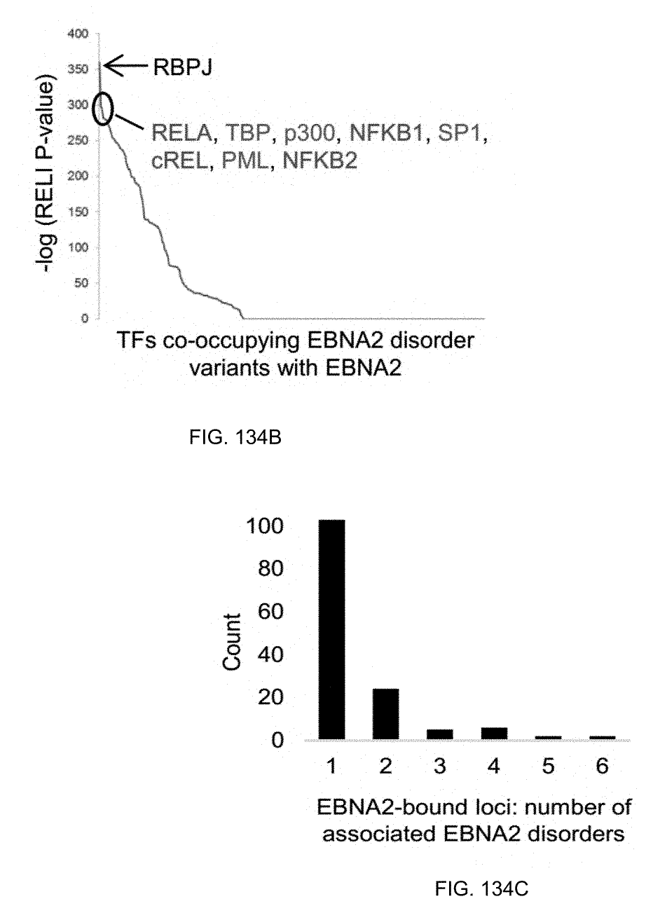

To identify TFs that bind a significant number of risk loci for a given disease, Applicant developed the RELI (Regulatory Element Locus Intersection) algorithm. RELI systematically estimates the significance of the intersection of the genomic coordinates of plausibly causal genetic variants and DNA sequences immunoprecipitated (through ChIP-seq) by a particular TF. Observed intersection counts are compared to a null distribution composed of variant sets chosen to match the disease loci in terms of allele frequency and linkage disequilibrium (LD) block structure (FIG. 134A). RELI is an extension of previous methods such as XGR¹⁶, which estimates the overlap between an input set of regions and genome-wide annotations, but does not explicitly replicate LD block structure in the null model.

Applicant first gauged the ability of RELI to capture known or suspected connections between TFs and diseases. The androgen receptor (AR) plays a well-established role in prostate cancer¹⁷, and RELI analysis revealed that AR binding sites in VCaP cells significantly intersect prostate cancer-associated loci (17 of 52 loci, Relative Risk (RR)=3.7, corrected P-value (Pc)<10⁻⁶, Table 1). Similarly, binding sites for GATA3 in MCF7 cells significantly intersect breast cancer variants18 (Pc<10⁻¹⁰, Table 1). Consistent with EBV contributing to multiple sclerosis (MS)¹⁹⁻²¹ and results from a recent study²², RELI reveals that the EBV-encoded EBNA2 protein occupies 44 of the 109 MS loci in Mutu B cells (Pc<10⁻²⁹, Table 1). Prostate and breast cancer loci do not significantly intersect EBNA2 peaks, nor do the loci of certain inflammatory diseases such as systemic sclerosis (Table 1). Collectively, these observations illustrate that predictions made by RELI are specific and consistent with previously established disease mechanisms.

TABLE
Intersection of TF ChIP-seq datasets with multiple genetic loci of diseases and phenotypes.
Detailed results are presented in Supplementary Data 3.

| Phenotype | Cell line | TF | Number | Fraction | RR | P_{c} & P* |
|---|---|---|---|---|---|---|
| Prostate Ca | VCaP + Dht_18 hr | AR | 17 | 0.33 | 3.70 | 2.60E−07 |
| Breast Ca | MCF7 + Estradiol | GATA3 | 22 | 0.36 | 3.87 | 7.45E−11 |
| MS | Mutu | EBNA2 | 44 | 0.40 | 4.66 | 6.34E−30 |
| SSc | Mutu | EBNA2 | 2 | 0.10 | — | NS |
| SSc | IB4 | EBNA2 | 1 | 0.05 | — | NS |
| SSc | GM12878 | EBNA2 | 0 | 0.00 | — | NS |
| SLE | Mutu | EBNA2 | 26 | 0.49 | 5.96 | 1.09E−25 |
| SLE | IB4 | EBNA2 | 10 | 0.19 | 7.46 | 1.09E−11 |
| SLE | GM12878 | EBNA2 | 10 | 0.19 | 8.57 | 1.94E−13 |
| SLE | IB4 | EBNA-LP | 4 | 0.08 | — | NS |
| SLE | Mutu | EBNA3C | 5 | 0.09 | — | NS |
| SLE | Raji | EBNA1 | 0 | 0.00 | — | NS |
| SLE | Akata | Zta | 0 | 0.00 | — | NS |
| SLE* | Mutu* | EBNA2* | 25* | 0.63* | 2.85* | 1.81E−11* |
| SLE* | IB4* | EBNA2* | 10* | 0.25* | 3.61* | 2.44E−06* |
| SLE* | GM12878* | EBNA2* | 10* | 0.25* | 4.97* | 1.22E−09* |

*RELI null model limited to EBV-infected B cell line open chromatin regions (see text). RR = relative risk. Pc = RELI Bonferroni corrected P-value. NS = Pc > 10E⁻⁶. All disease ancestries are European. Ca = cancer. MS = multiple sclerosis. SSc = systemic sclerosis. SLE = systemic lupus erythematosus.

Applicant assembled 53 European ancestry SLE loci (P<5×10⁻⁸) with risk allele frequencies >1%, constituting 1,359 plausibly causal SLE variants. To explore the possible environmental contribution from EBV, Applicant evaluated the ChIP-seq data from EBV-infected B cells for the EBV gene products EBNA1, EBNA2 (three datasets), EBNA3C, EBNA-LP, and Zta (Supplementary Data 2). EBNA2 occupies loci that significantly intersect SLE risk loci in all three available ChIP-seq datasets (Table 1). For example, 26 of 53 European SLE GWAS loci contain DNA immunoprecipitated by EBNA2 in the Mutu B cell line, an almost 6-fold enrichment (Pc<10⁻²⁴). No association was detected for the other EBV-encoded proteins. To examine the possibility that these results might simply be explained by enrichment of SLE loci in B cell open chromatin regions, Applicant restricted the RELI null model to variants located in DNase hypersensitive regions in EBV-infected B cells. With this higher stringency null model, all of the EBNA2 associations remained significant. Thus, the associations Applicant detect between SLE risk loci and EBNA2 cannot simply be explained by the previously established strong co-localization between SLE risk loci and B cell regulatory regions in the genome²³.

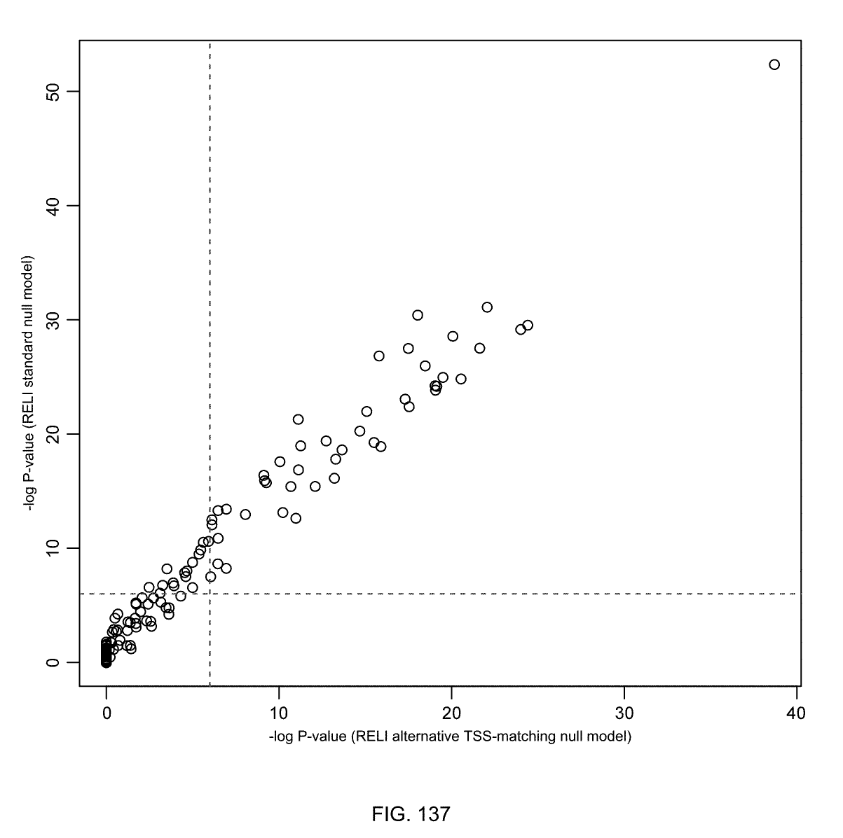

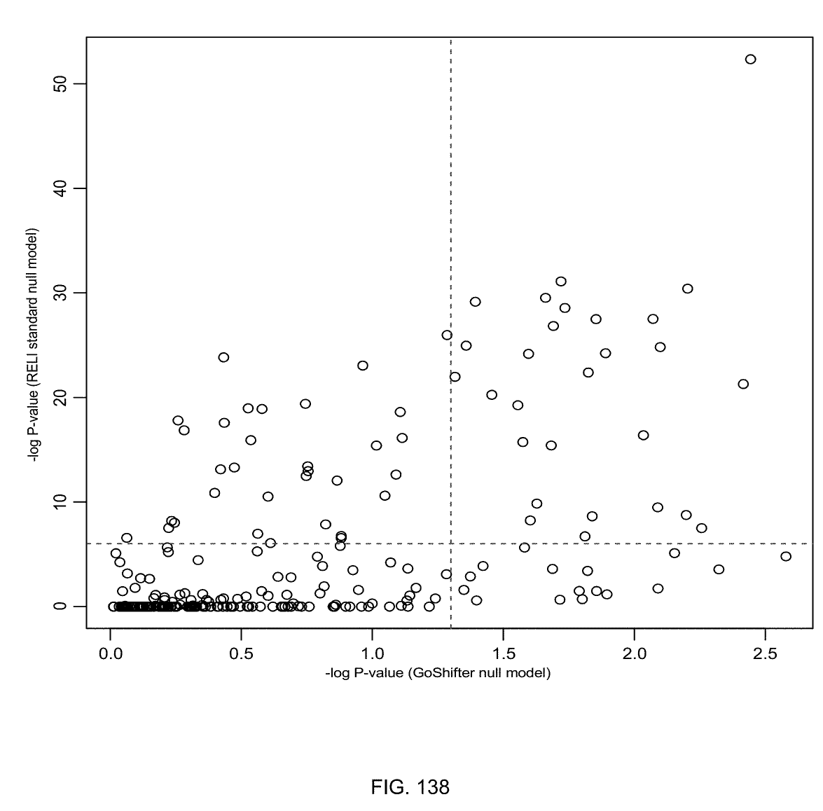

Applicant next applied RELI to a large collection of human TF ChIP-seq datasets (1,544 experiments evaluating 344 TFs and 221 cell lines). In total, 132 ChIP-seq datasets involving 60 unique TFs strongly intersect SLE loci (10-53<Pc<10-6). 109 (83%) of the experiments were performed in EBV-infected B cell lines, with impressive fidelity between datasets. Nearly identical results were obtained using a null model that also takes the distance to the nearest gene transcription start site into account (FIG. 137). Likewise, similar results were obtained using the null model employed by the GoShifter²⁴ method (FIG. 138). Similar results were also obtained with an expanded set of all 83 SLE risk loci published to date (regardless of ancestry)¹⁰⁻¹³ or when separately examining SLE risk loci by ancestry. Strikingly, 20 of these 60 TFs participate in “EBV super-enhancers”, which enable proliferation and survival of EBV-infected B cells²⁵. The human TFs in question largely bind the same loci occupied by EBNA2, comprising an optimal cluster of 25 TFs and 28 SLE risk loci (FIG. 133A).

If EBV is involved in SLE pathogenesis, then the absence of EBV, and hence EBNA2, should diminish the observed associations with SLE risk loci. For eight TFs, ChIP-seq datasets are available in both EBNA2-expressing (EBV-infected) and EBV negative B cell lines.

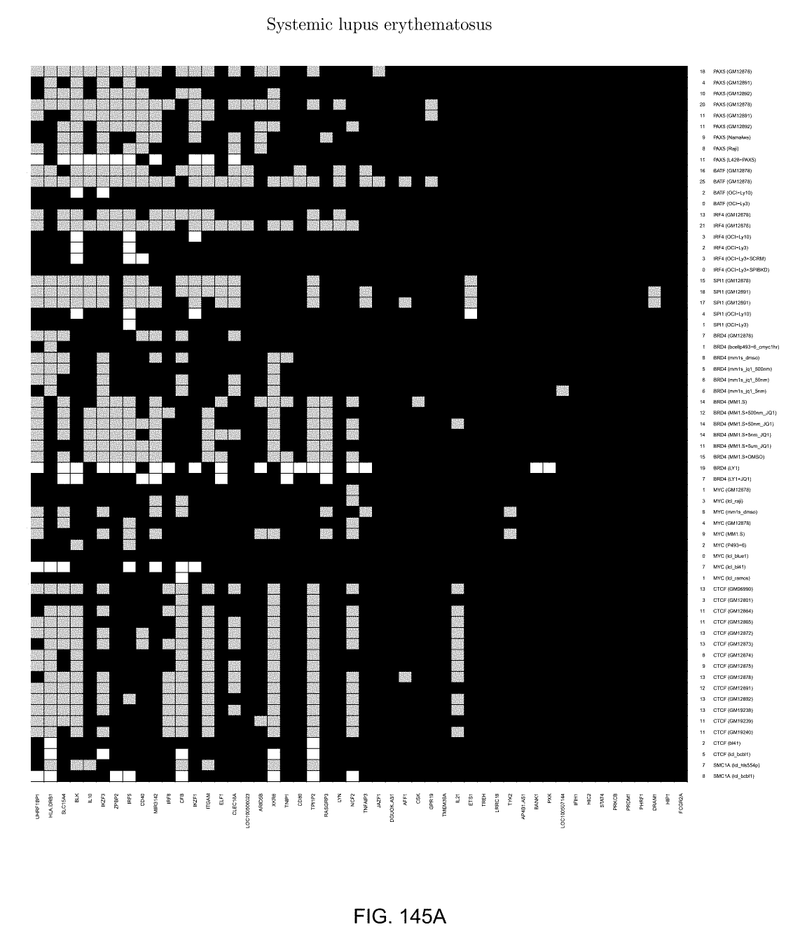

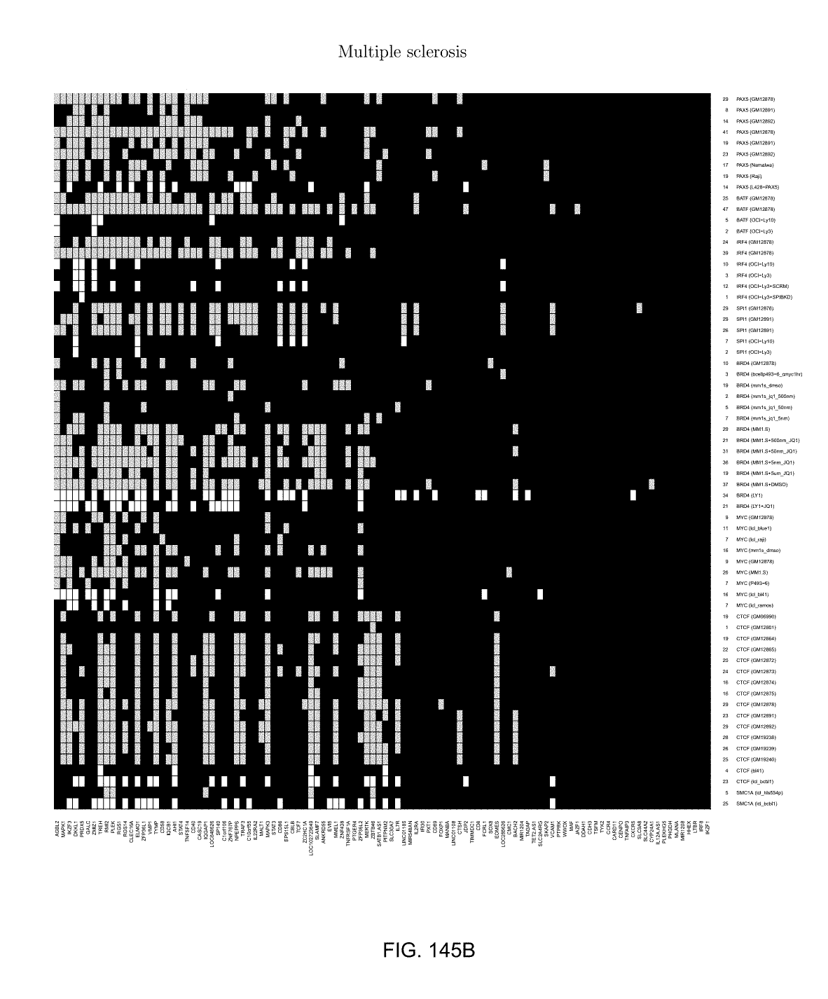

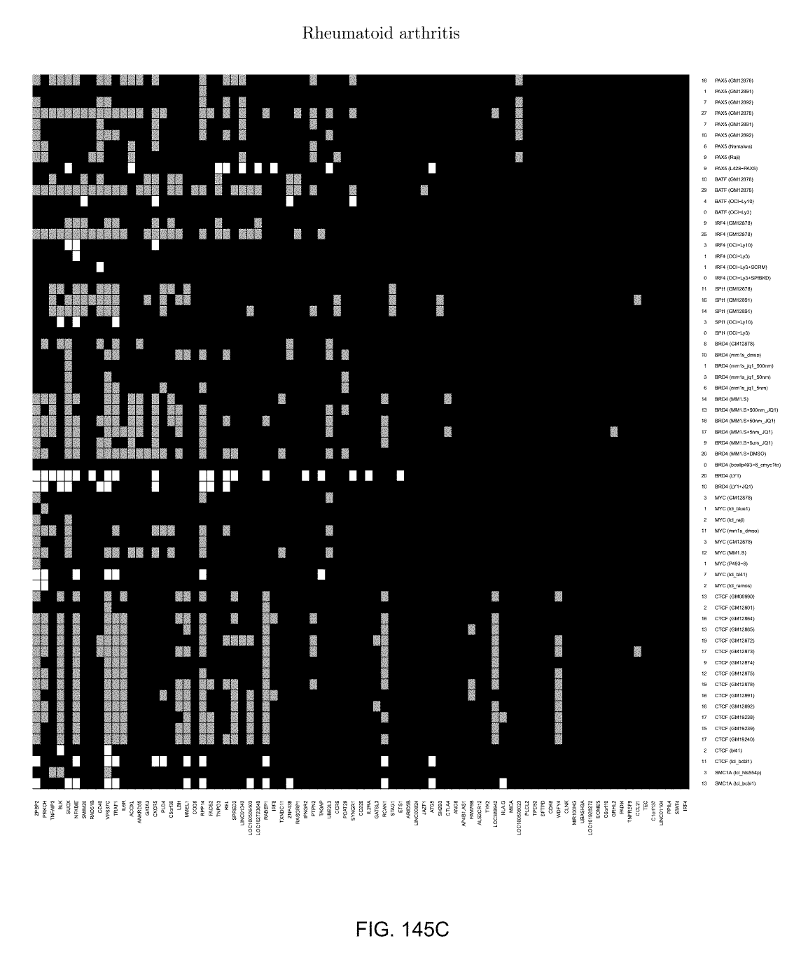

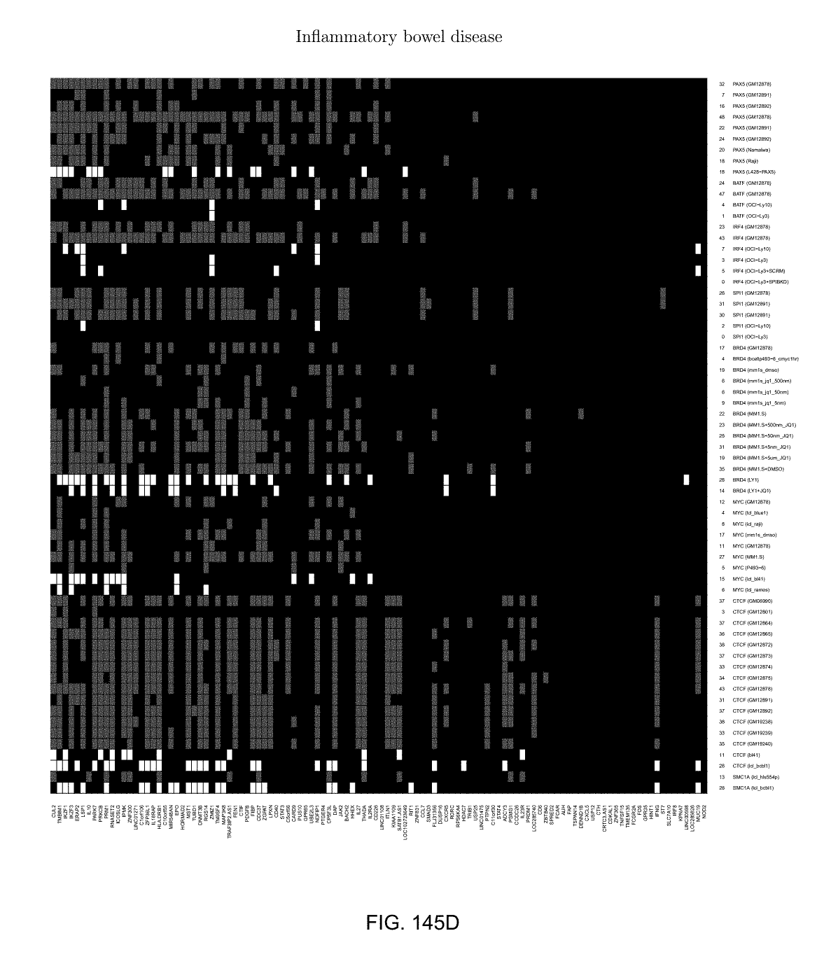

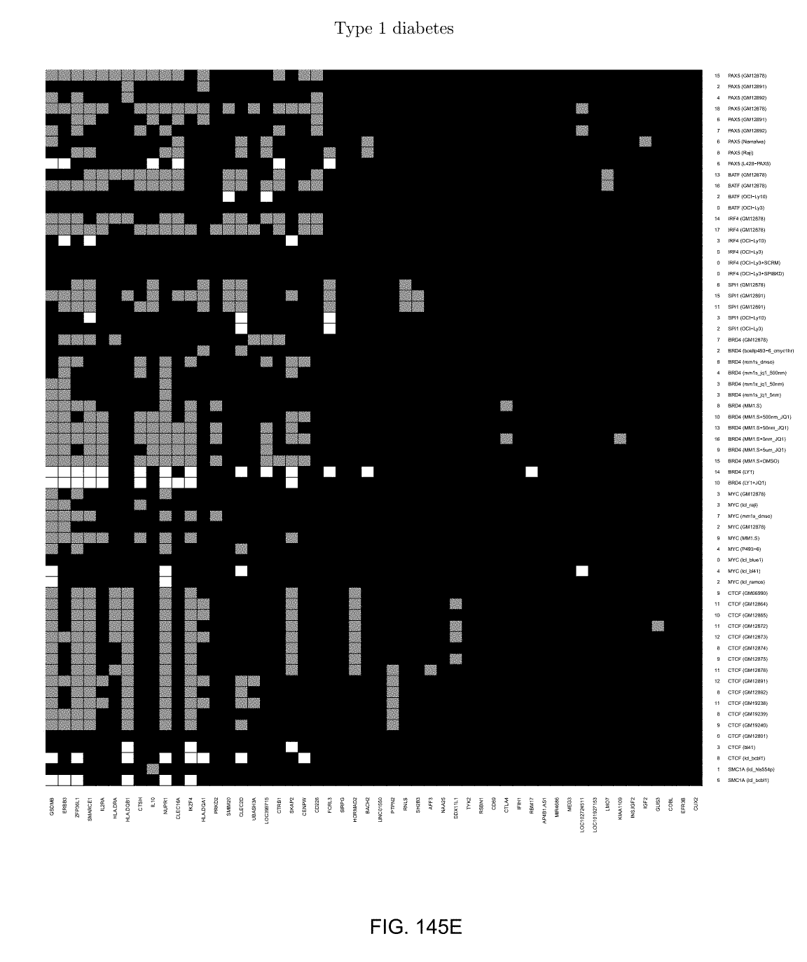

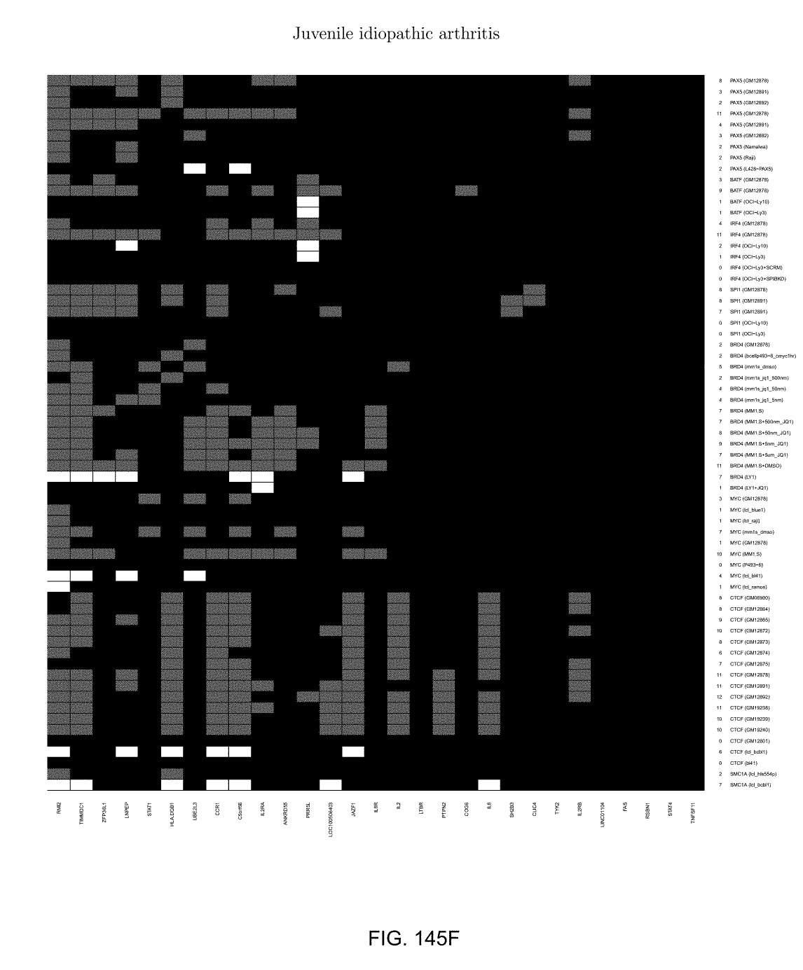

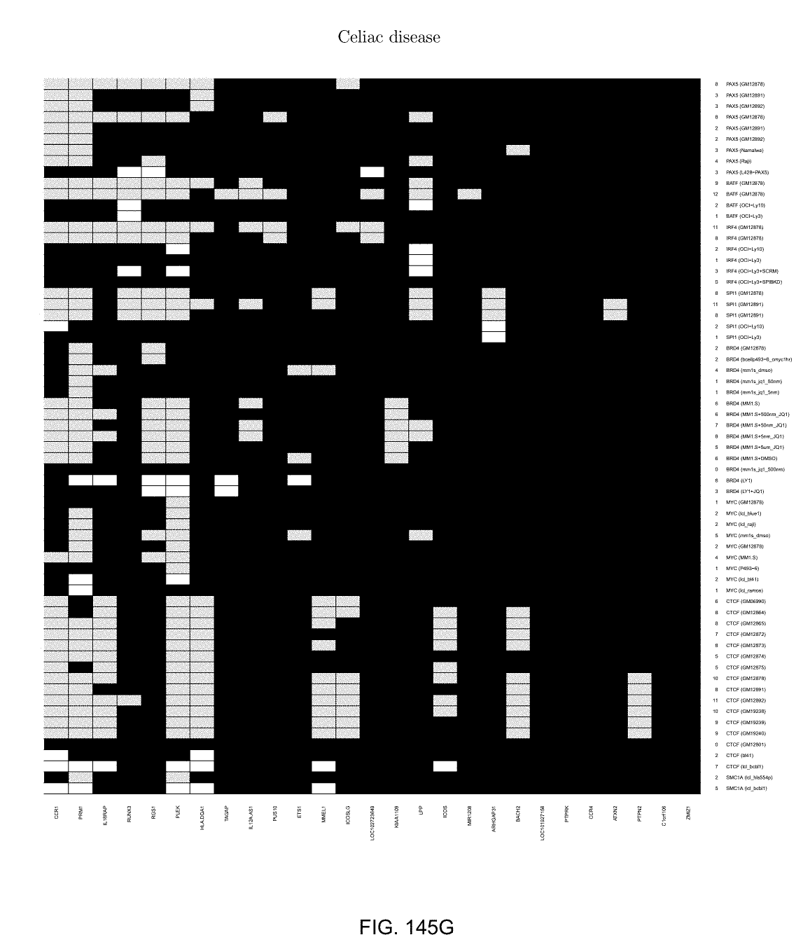

Notably, the four TFs with the strongest RELI P-values in EBV-infected B cells (BATF, IRF4, PAXS, and SPI1) have weaker P-values in EBV negative B cells (FIG. 133A, bottom left panel, FIG. 145), consistent with these TFs occupying many SLE risk loci only in the presence of EBV. Further, all of the datasets for the ten TFs with the strongest RELI P-values were performed in EBV-infected B cells, and none of the other cell types available for these TFs show significant association (FIG. 133A, bottom right panel). For example, 22 ChIP-seq datasets are available in EBV-infected B cells for the NFκB subunit RELA. Of these, 20 significantly intersect with SLE risk loci (10⁻⁵³<Pc<10⁻¹⁷), while none of the remaining 14 available RELA datasets in any other cell type have significant intersection. Previous studies have demonstrated that EBV activates the NFκB pathway, thereby supporting the validity of this result²⁶⁻²⁸. Combined with the striking intersection between EBNA2 binding and SLE loci, these data strongly suggest an important role for EBV and EBV-infected B cells in SLE.

EBNA2-Occupied Genomic Sites Intersect Autoimmune-Associated Loci

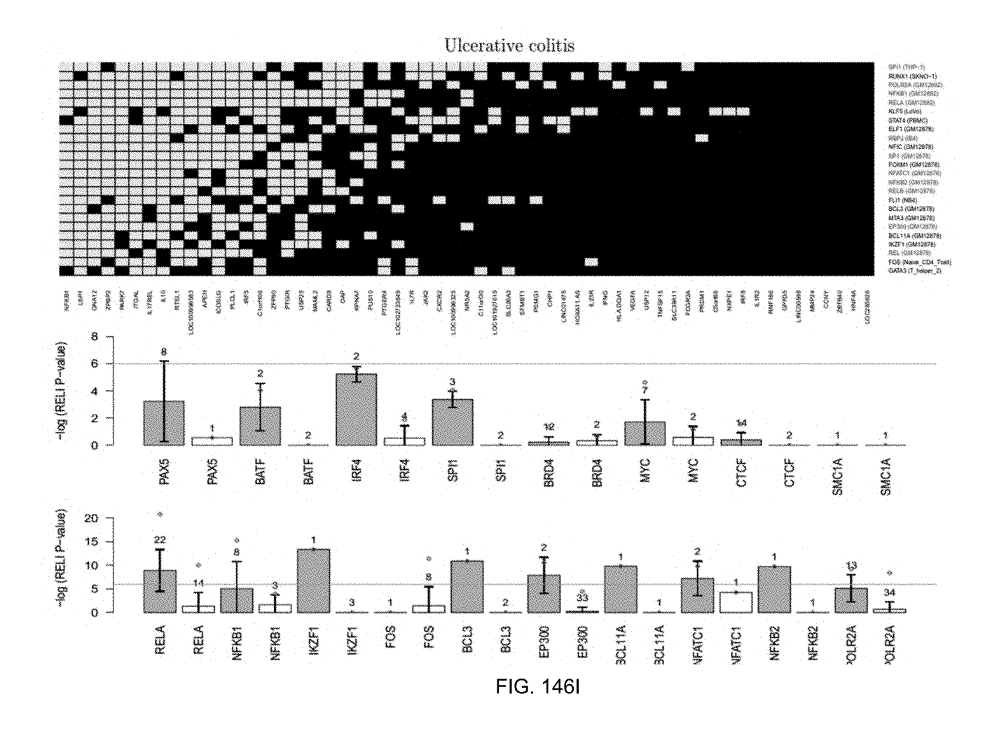

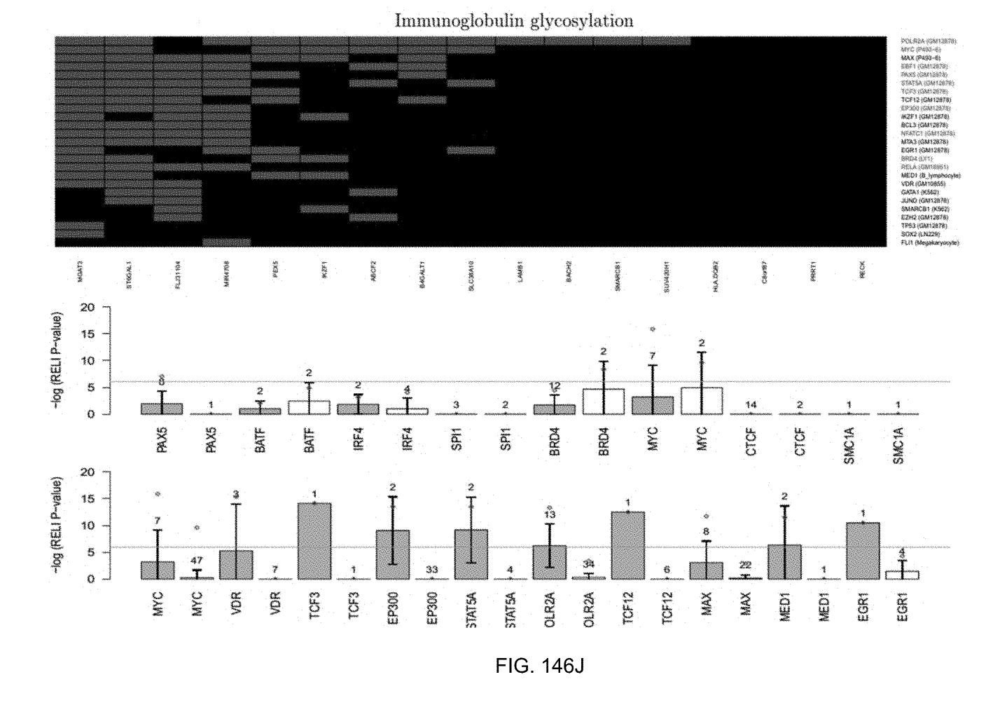

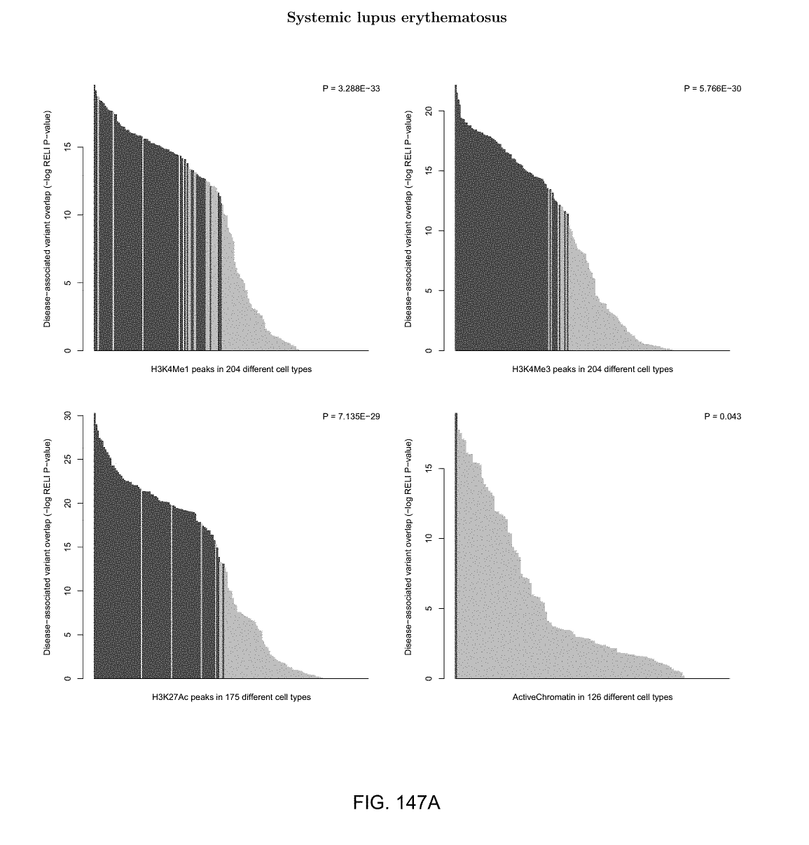

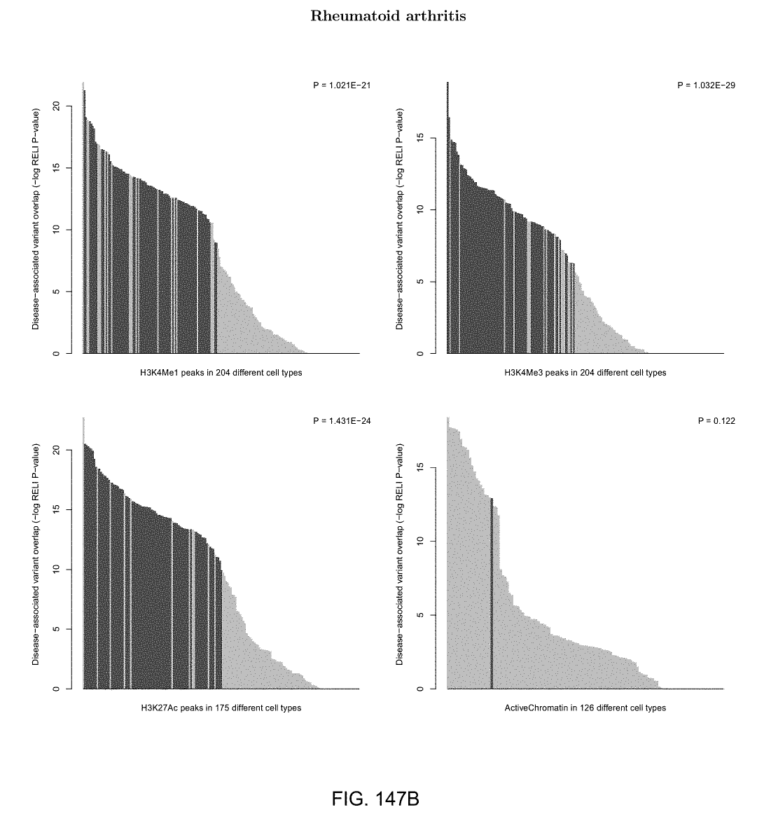

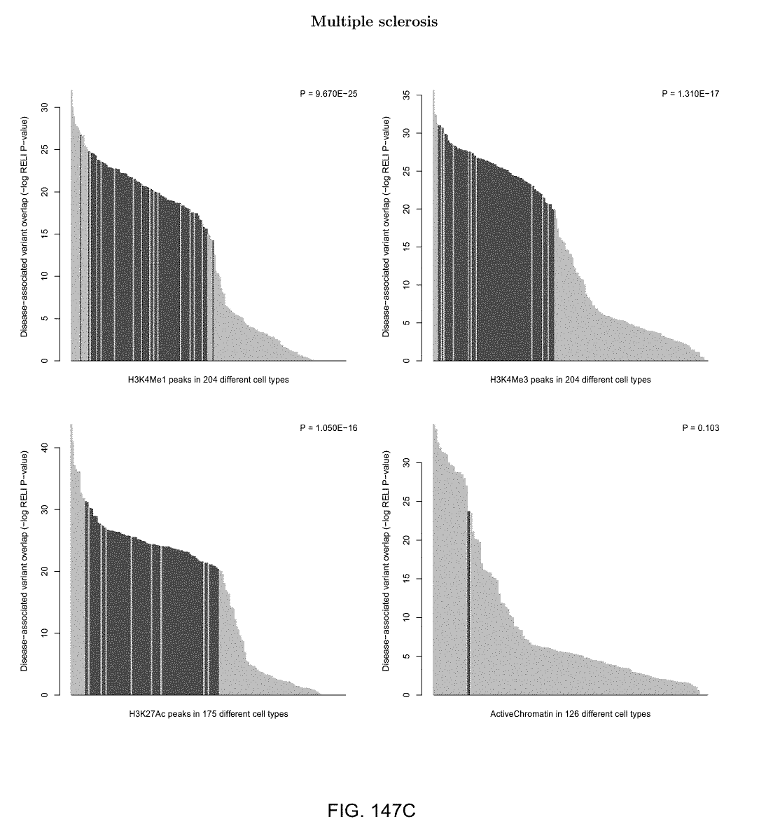

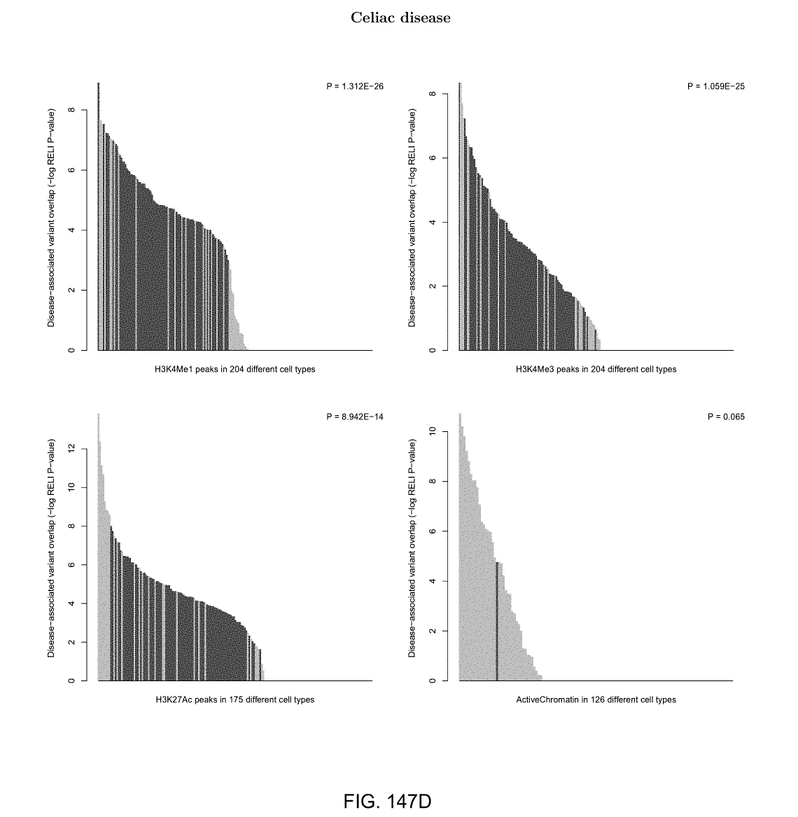

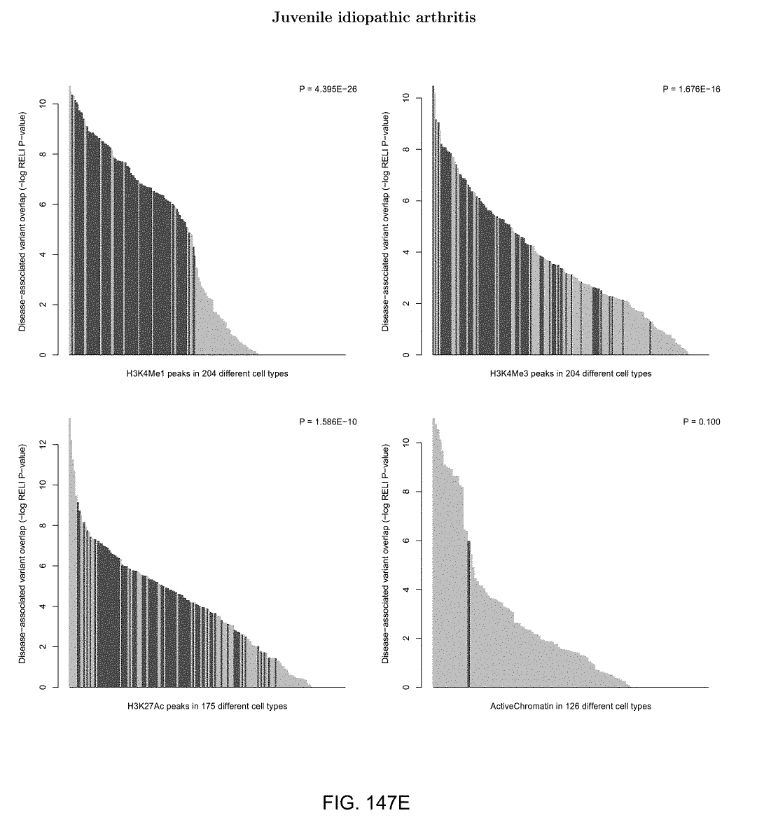

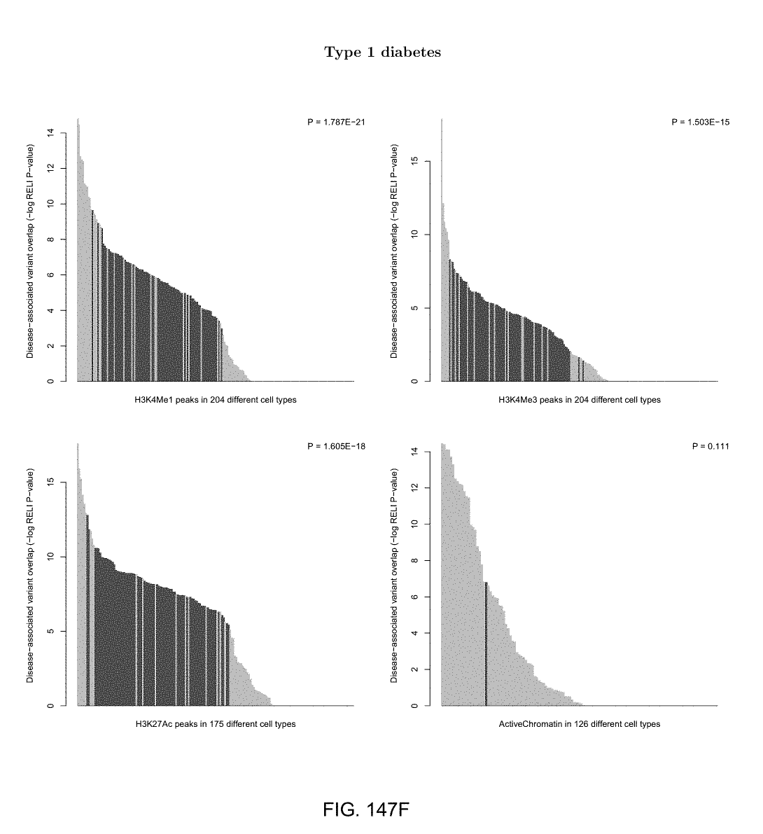

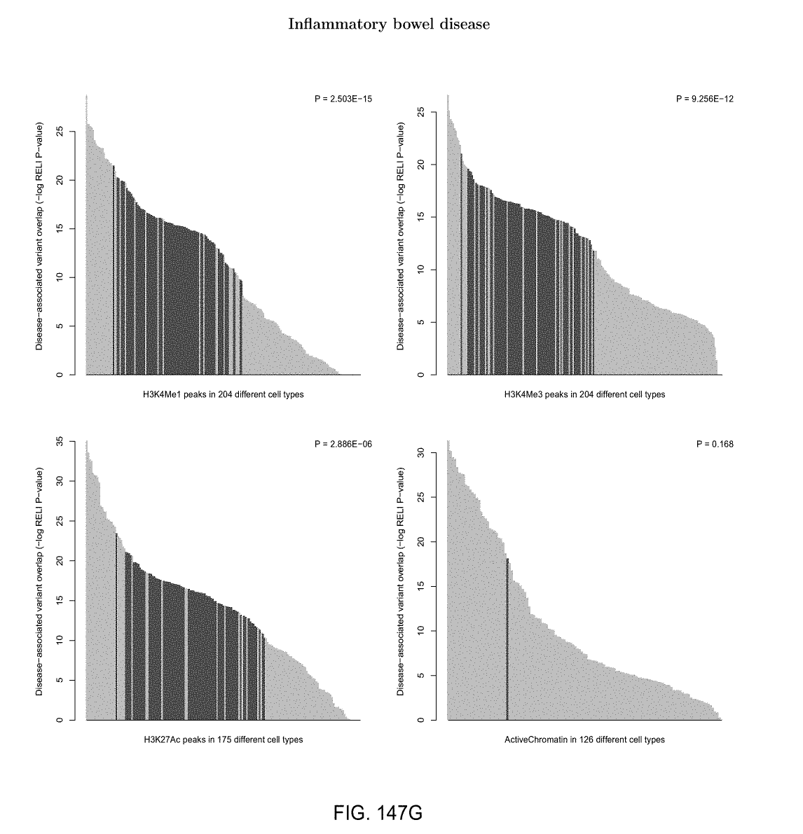

Applicant applied RELI to 213 diseases and phenotypes obtained from the NHGRI GWAS catalog′ and other sources, revealing nine phenotypes displaying strong EBNA2 association in addition to SLE and MS: rheumatoid arthritis (RA), inflammatory bowel disease (IBD), type 1 diabetes (T1D), juvenile idiopathic arthritis (JIA), celiac disease (CelD), chronic lymphocytic leukemia (CLL), Kawasaki disease (KD), ulcerative colitis (UC), and immunoglobulin glycosylation (IgG) (FIG. 147A-G). Applicant designate the seven disorders among these with particularly strong EBNA2 associations (Pc<10⁻⁸) the “EBNA2 disorders.” A recent study performed statistical fine-mapping of the variants for six of the seven EBNA2 disorders (IBD was not included)³⁰. Of the resulting 1,953 candidate causal variants, 130 overlap with EBNA2 ChIP-seq peaks in Mutu B cells (RR=8.7, Pc<10⁻¹³²). Notably, this represents the second-ranked ChIP-seq dataset out of the 1,544 considered, trailing only POLR2A ChIP-seq performed in CD4+T cells (FIG. 147A-G). Thus, the overlap between EBNA2 ChIP-seq peaks and loci associated with the EBNA2 disorders is even stronger when only considering statistically likely causal variants.

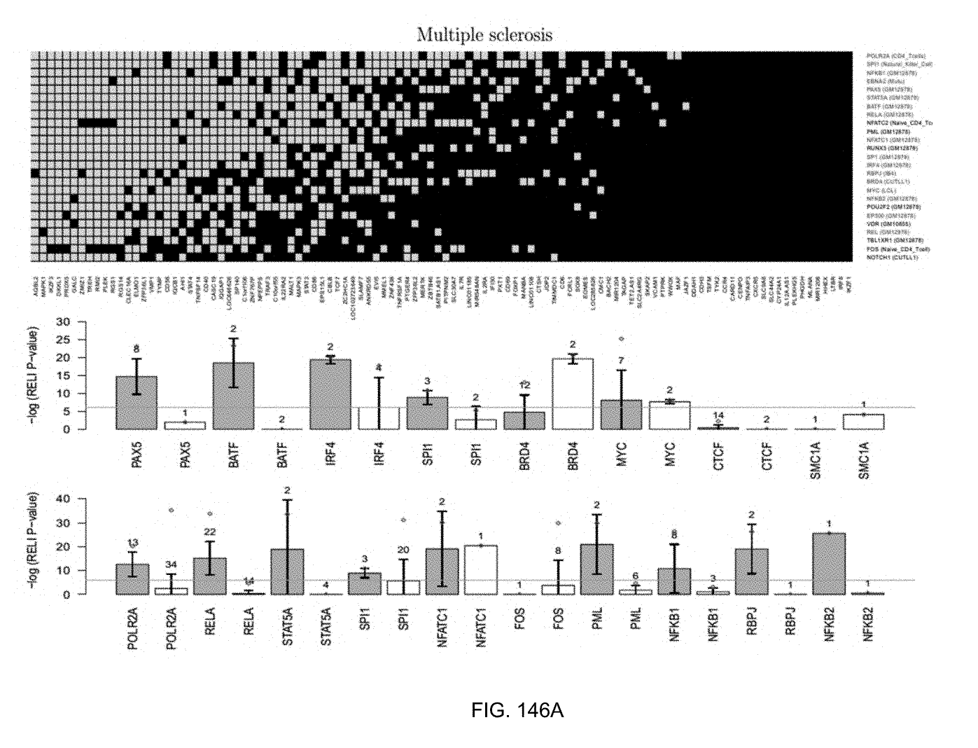

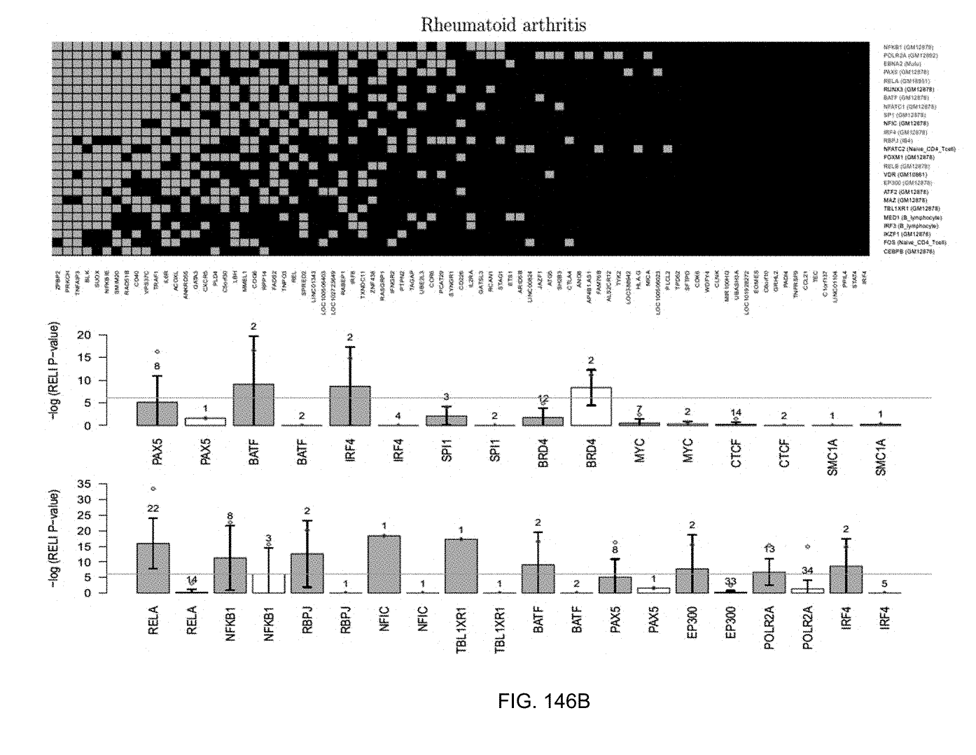

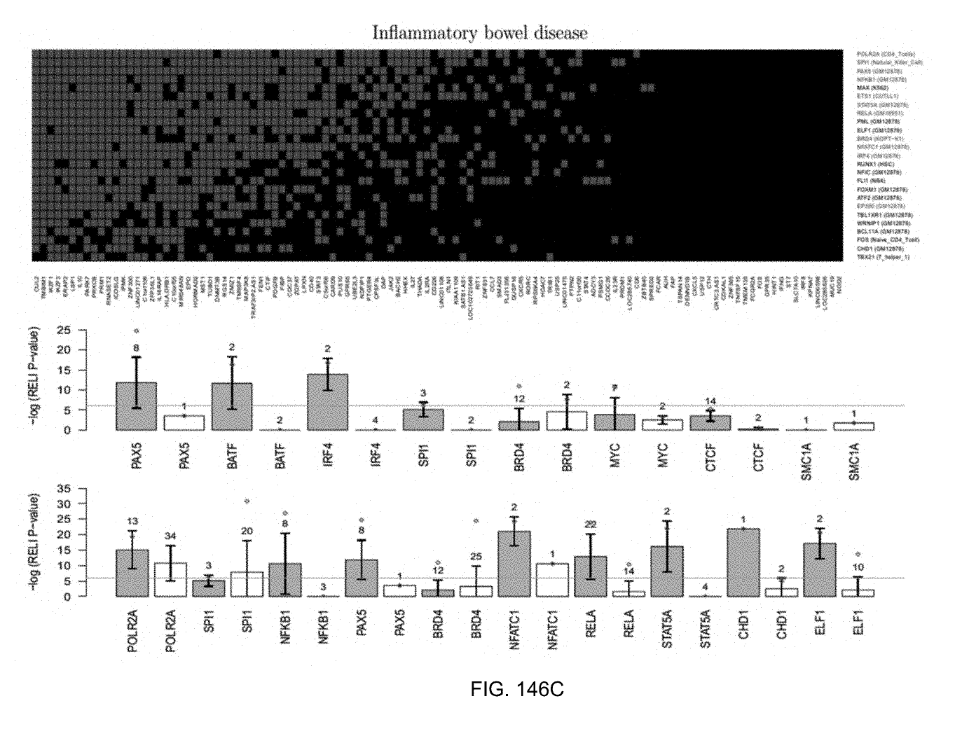

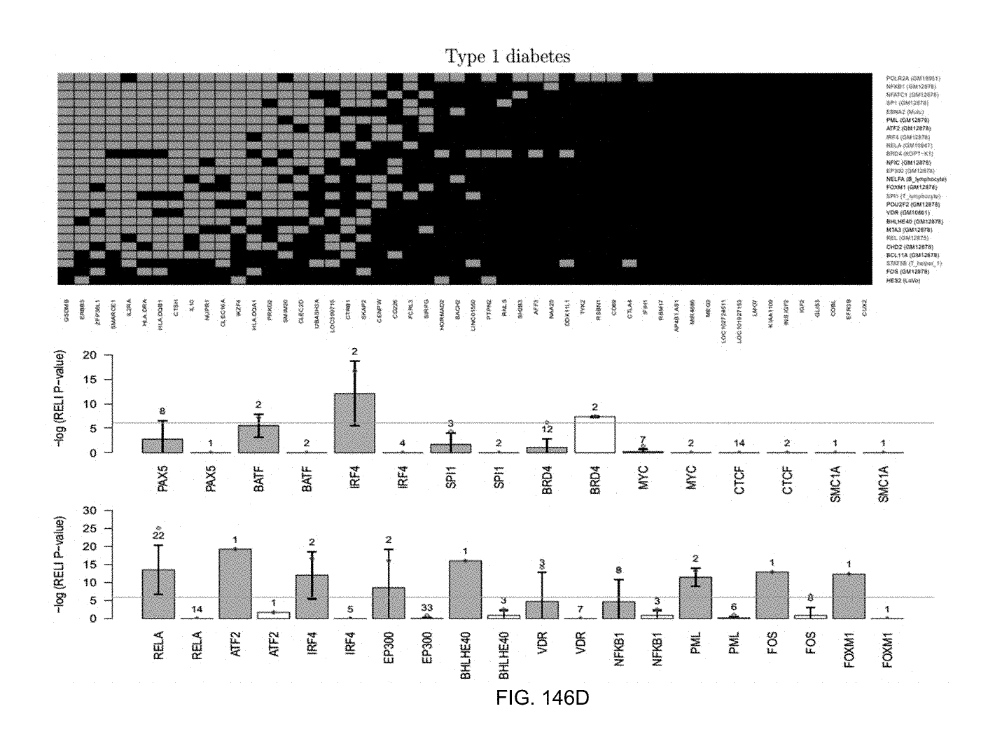

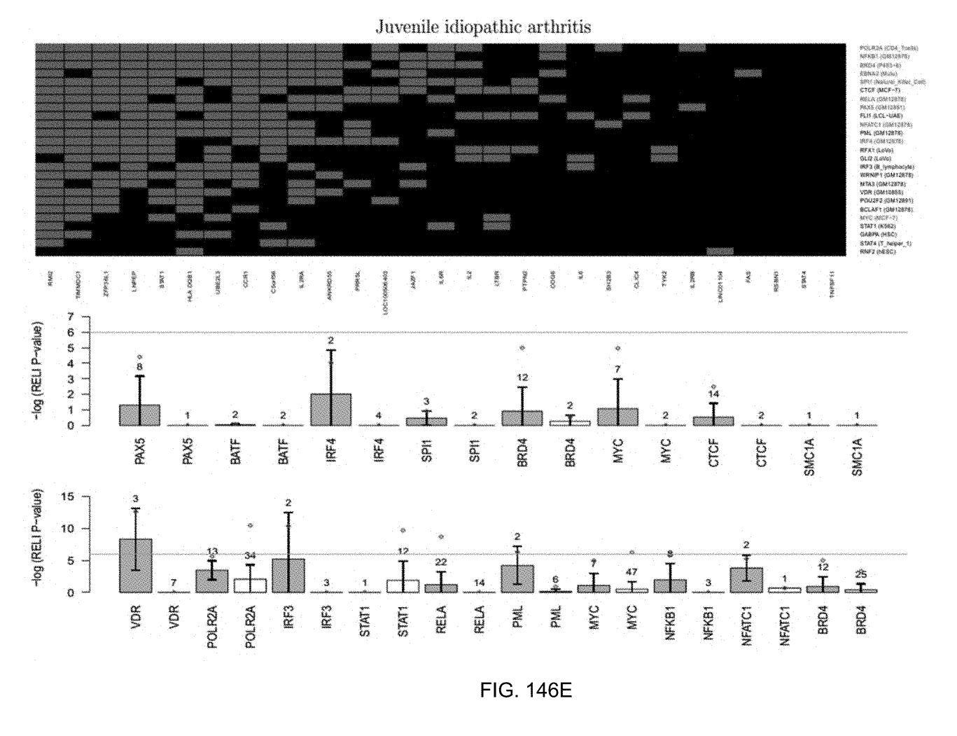

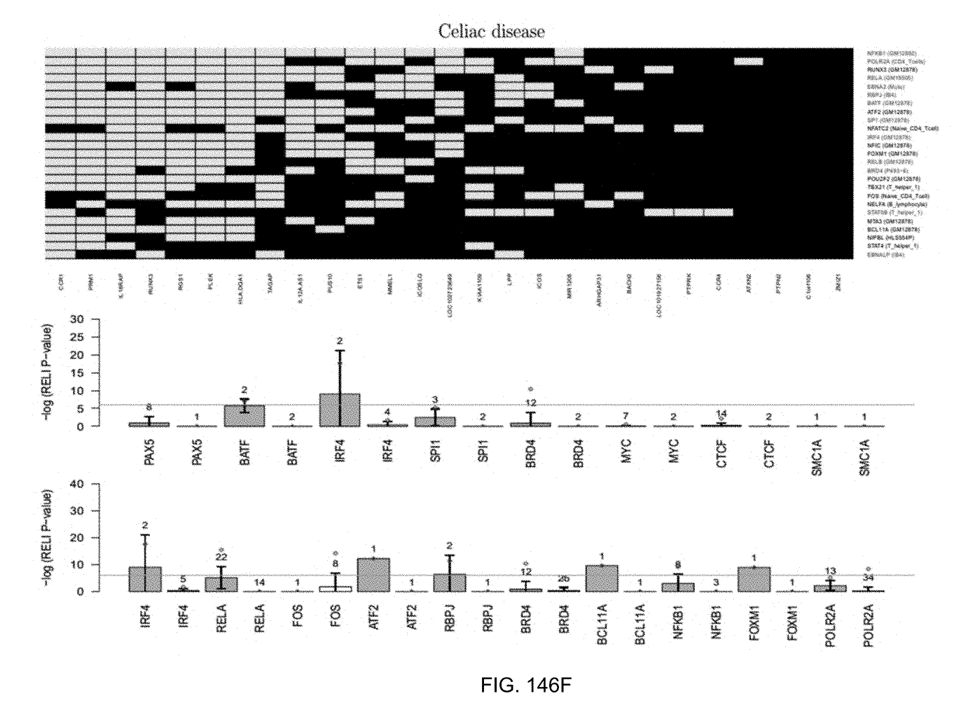

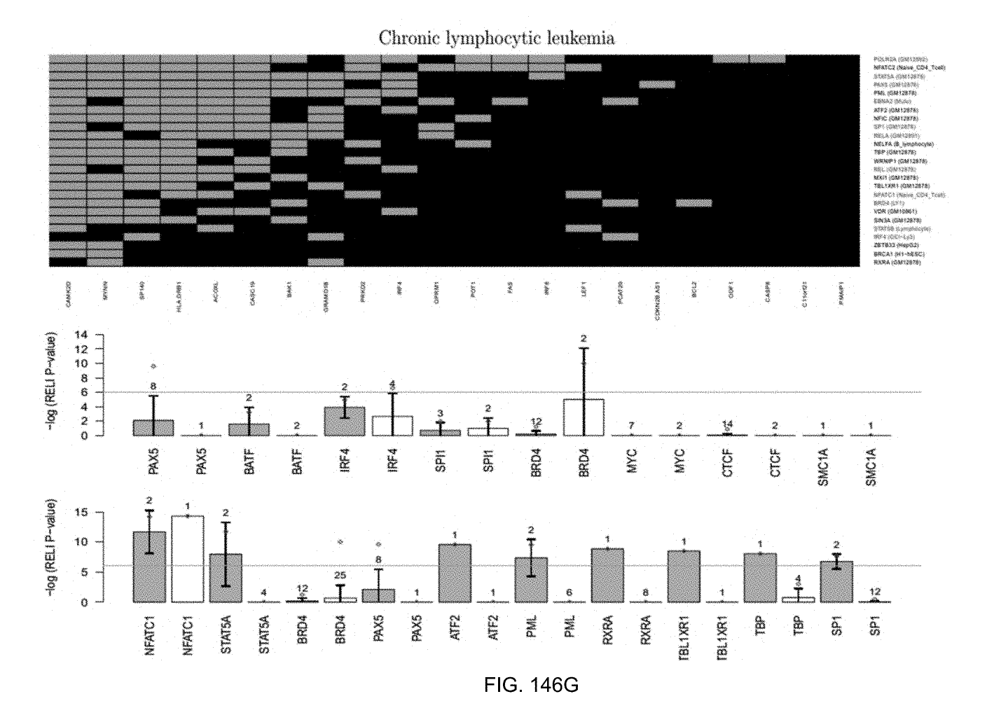

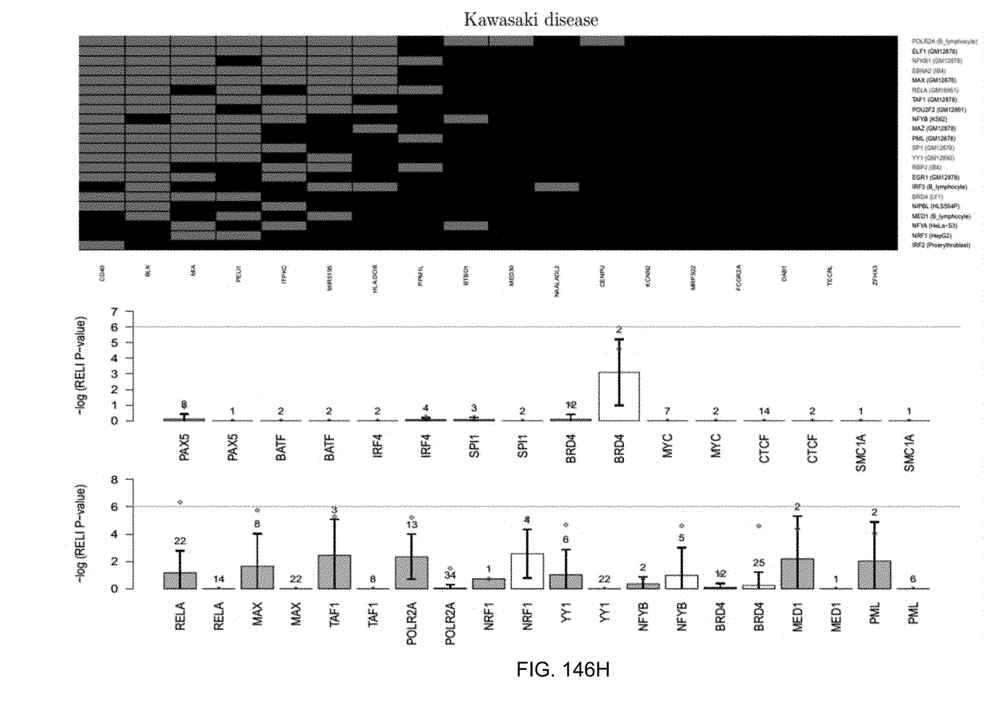

Consistent with the SLE results (FIG. 133A), the same TFs cluster with distinguishing loci for each disorder (FIG. 133B-G). Further, there is also a stronger association in EBV-infected than in EBV negative cells for most TFs, and the 10 most associated TFs consistently intersect more strongly in EBV-infected B cells than in other cell types (FIG. 133B-G, FIG. 146A-J). Hierarchical clustering identifies a core set of 47 TFs binding to 142 loci risk loci across the seven EBNA2 disorders. RBPJ, an established EBNA2 co-factor³¹⁻³³, has the most similar binding profile to EBNA2 across loci, as expected.

In order to identify additional EBNA2 co-factor candidates, Applicant isolated EBNA2 disorder-associated variants located within EBNA2 ChIP-seq peaks and evaluated them using RELI. This analysis confirms the importance of RBPJ, followed by members of the basal transcriptional machinery (TBP and p300), and NFκB subunits (which are involved in EBNA2-mediated gene activation′) (FIG. 134B). Interestingly, predicted EBNA2 co-factors vary with disease phenotype; for example, EBNA2 and EBNA3C are highly synergistic at the disease loci of three of the EBNA2 disorders (IBD, MS, and CelD), but rarely coincide at loci for the other four diseases.

The particular TFs tend to be shared across the EBNA2 disorders, but the loci they occupy are less frequently shared. No EBNA2-bound locus is associated with all seven EBNA2 disorders; most loci are unique to only one disorder (FIG. 133C). Thus, the loci occupied by EBNA2 in each disorder are largely distinct from one another. One counterexample involves the IKZF3 locus encoding the Aiolos TF, a key regulator in B lymphocyte activation³⁵, with genetic variants from five different EBNA2 disorders intersecting EBNA2 ChIP-seq peaks.

If changes in gene regulation explain these results, then expression trait quantitative loci (eQTLs), ChIP-seq peaks for Pol-II, and histone marks associated with active gene regulatory regions should be relatively concentrated at the risk loci occupied by EBNA2. These predictions are indeed true for each of the seven EBNA2 disorders (FIG. 134D). For example, <1% of all common variants in the human genome are eQTLs in EBV-infected B cell lines (FIG. 134D). This value rises to 2.3% for common variants located within open chromatin in EBV-infected B cell lines, and rises further to 2.7% for common variants within EBNA2 ChIP-seq peaks (FIG. 134D, upper left panel, bars labeled “Common variants”). Thus, there is a slight trend for a common variant located within an EBNA2 ChIP-seq peak to influence gene expression in EBV-infected B cell lines. Strikingly, this relationship is >10-fold increased for EBNA2 disorder-associated variants—27.8% of EBNA2 disorder variants that are located within EBNA2 ChIP-seq peaks are also eQTLs, a value significantly greater than EBNA2 disorder variants located within DNase-seq peaks (20.5%, P<10-5, Welch's one-sided t-test) or EBNA2 disorder variants in general (10.4%, P<10′) (FIG. 134D, upper left panel, bars labeled “EBNA2 disorder variants”). Similar trends hold for the other data types examined (FIG. 134D). These results identify the EBV-infected B cell as a potential etiologic source for the operation of genetic risk in these disorders. Further, they indicate that EBNA2 disorder variants located within EBNA2 ChIP-seq peaks likely influence downstream gene expression levels. In aggregate, these results hint at the potential complexity and magnitude of the environmental influence of EBNA2 upon host gene expression in the EBV infected B cell.

EBNA2 Participates in Allele-Dependent Formation of Transcription Complexes at Disease Risk Loci

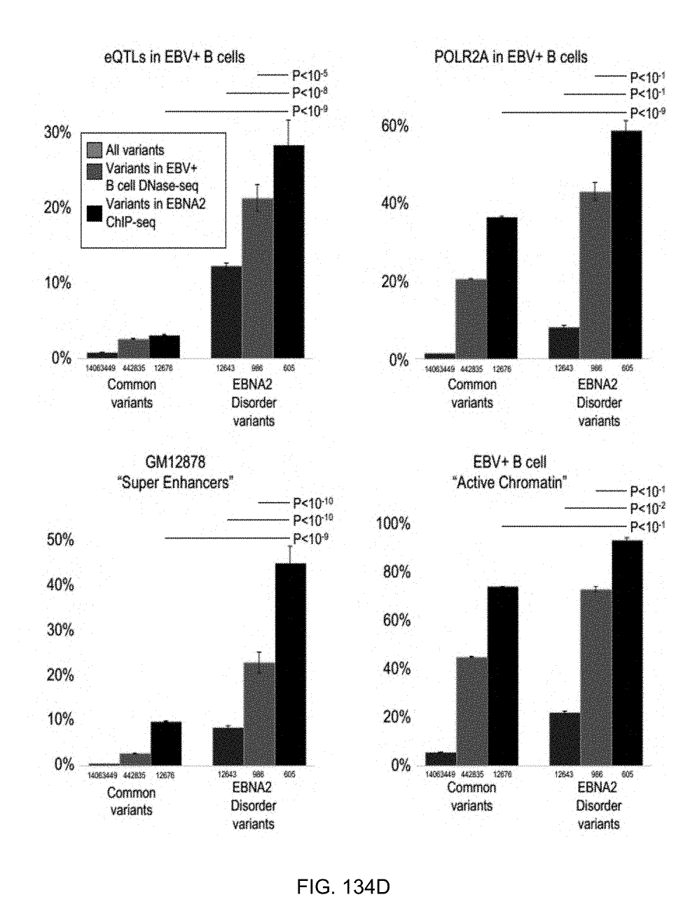

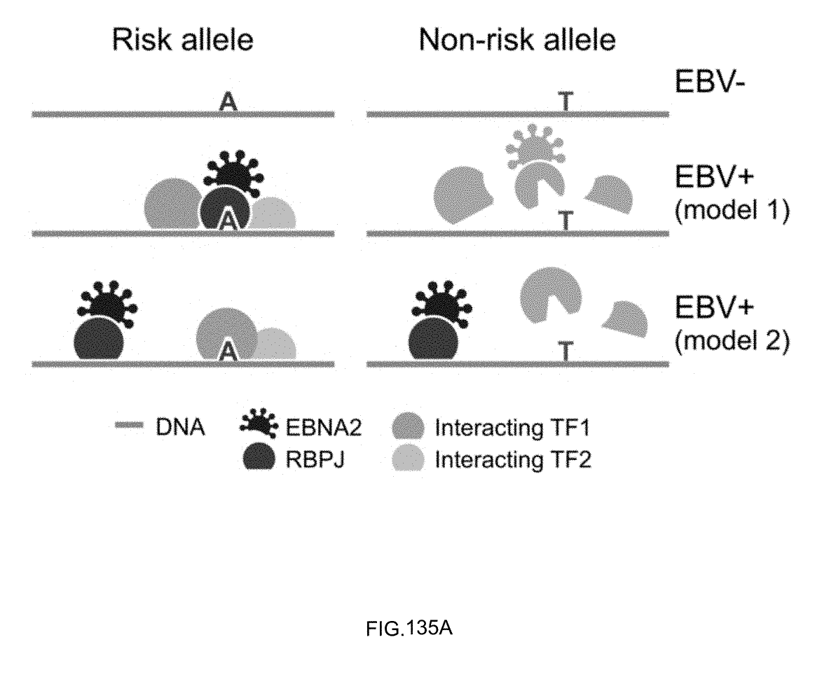

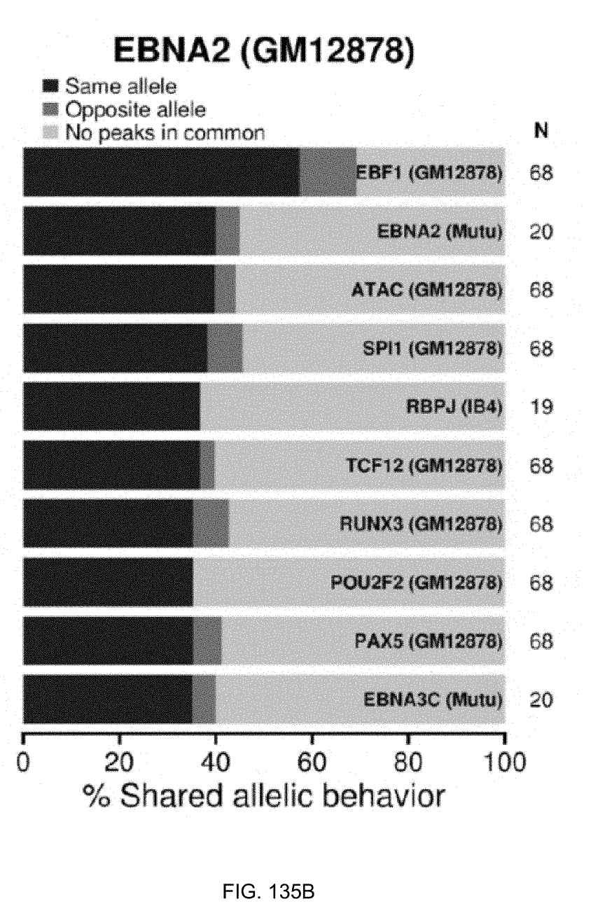

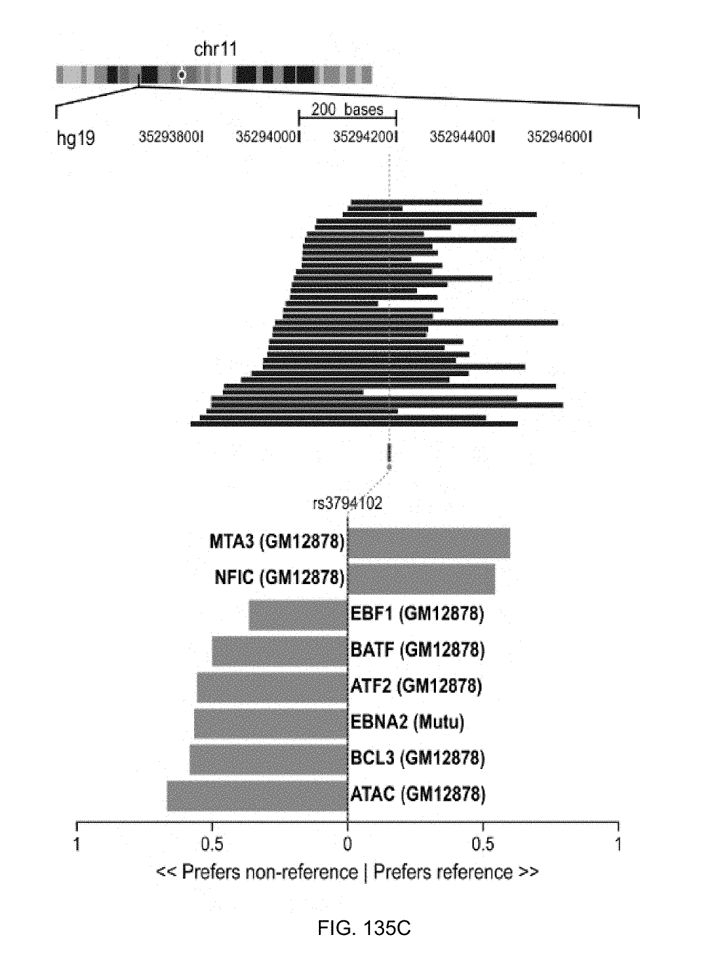

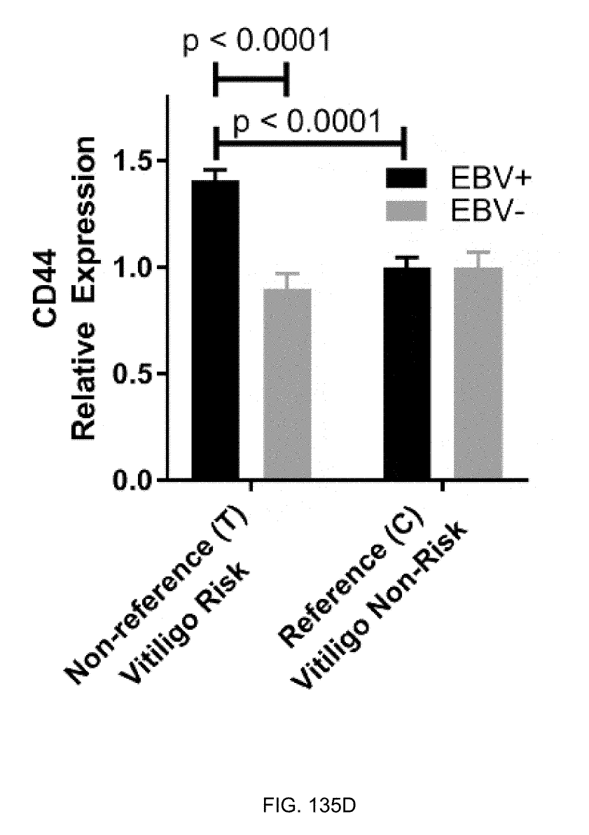

The observed associations (FIG. 133A-G) are genetic if and only if they are driven by causal allelic differences. Since EBNA2 imitates the binding of NOTCH to RBPJ, converting RBPJ from suppression to activation³⁶, genetic variants at these loci could alter the binding of RBPJ (or another TF to which EBNA2 binds) or enable allele-dependent binding of a TF that requires the presence of EBNA2 (FIG. 135A). Re-analysis of ChIP-seq data provides a means to identify allele-dependent protein binding events on a genome-wide scale—in cases where a given variant is heterozygous in the cell assayed, both alleles are available for the TF to bind, offering a natural control for one another since the only variable that has changed is the allele. Applicant therefore developed the MARIO (Measurement of Allelic Ratio Informatics Operator) pipeline to estimate allele-dependent protein binding by weighing imbalance between the number of reads for each allele, the total number of reads available at the variant, and the number and consistency of available experimental replicates (see Methods). MARIO is an easy-to-use, modular tool that extends existing methods³⁷⁻⁴⁰ by (1) calculating a score that explicitly reflects reproducibility across experimental replicates; (2) reducing run-time via utilization of multiple computational cores; and (3) allowing the user to directly provide genotyping data as input. To identify heterozygotes, Applicant genotyped five EBV-infected B cell lines with available ChIP-seq data and performed genome-wide imputation (see Supplementary Methods). Applicant applied MARIO and a related method, ABC³⁷, to a deeply sequenced (˜190 million reads) GM12878 ATAC-seq dataset (GEO accession GSM1155957) and observed strong agreement between the 2,214 resulting scores (Spearman correlation of 0.98 (P<10⁻¹⁵)). Thus, the scores produced by MARIO are largely consistent with scores produced by a related method.

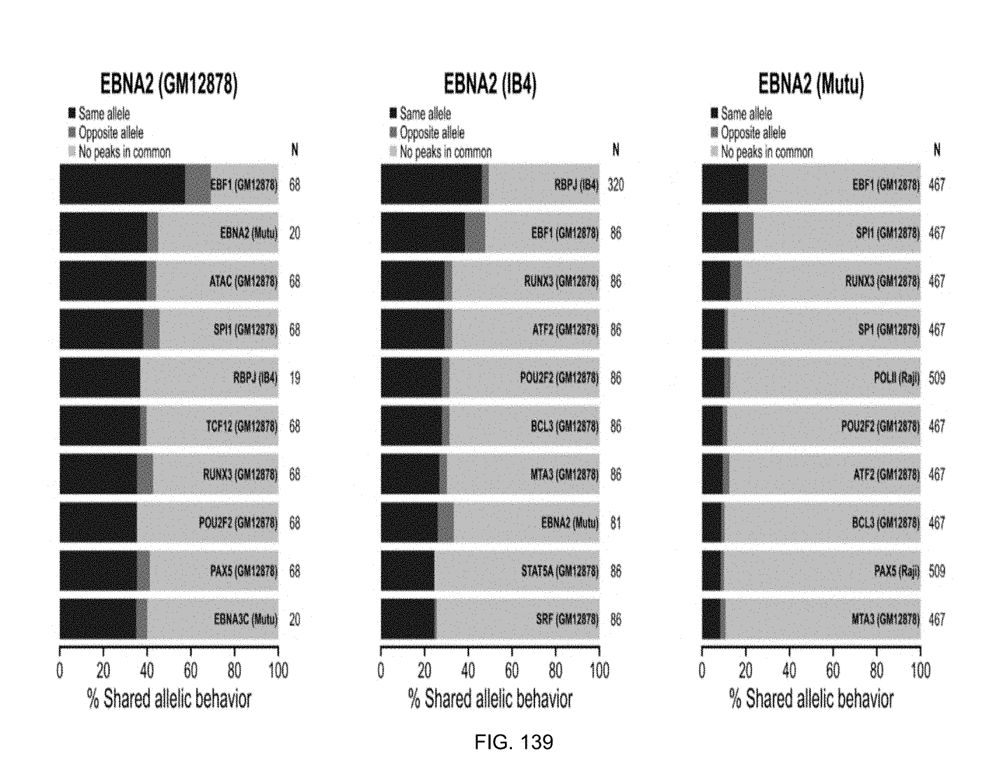

Applicant applied MARIO to 271 ChIP-seq datasets performed in the five genotyped cell lines, altogether assessing 98 different molecules. Since EBNA2 binds DNA through co-factors, Applicant first asked if the variants displaying EBNA2 allele-dependent binding might also coincide with similarly altered binding of other TFs. This analysis revealed strong concordance of allele-dependent binding events both within and across cell types. For example, Applicant identified 68 heterozygous common variants located within allele-dependent EBNA2 GM12878 ChIP-seq peaks. EBF1, whose binding is globally influenced by EBNA2³⁶, has a coincident ChIP-seq peak favoring the same allele at 39 (57%) of these loci, as opposed to only 8 (11%) on the opposite allele (P<10′, binomial test, FIG. 135B). Similar results were obtained when pairing EBNA2 binding in GM12878 with EBNA2 binding in Mutu cells, with established partners SPI1 and RBPJ, or with ATAC-seq chromatin occupancy data (FIG. 135B). Analogous results are obtained with EBNA2 ChIP-seq data in Mutu and IB4 cell lines (FIG. 139). In total, MARIO confidently identified 23 variants associated with 12 different autoimmune diseases displaying allele-dependent EBNA2 binding in at least one cell type (Table 2). Most of these variants also involve allele-dependent host protein binding, chromatin accessibility, or presence of histone marks such as H3K27ac. Together, these results suggest that many autoimmune-associated variants may act by modifying host gene regulatory programs via altered binding of EBNA2 and additional proteins.

TABLE 2
Allele-dependent binding of EBNA2 to autoimmune-associated genetic variants.
Examples of EBNA2 ChIP-seq-derived allele-dependent binding to
heterozygous autoimmune-associated variants.

| | | | Reads | Reads | Str. | | Tag SNP and r² |
|---|---|---|---|---|---|---|---|
| Gene(s) | rs ID | ARS | (Str.) | (Weak) | Base | Disease(s) | with allelic SNP |
| CD37* | rs5828386 | 0.69 | 55 | 18 | G | MS | MS: rs8107548, r² = 0.940 |
| CD37* | rs1465697^{#} | 0.57 | 57 | 29 | C | MS | MS: rs8107548, r² = 0.959 |
| HLA-DQA1 | rs9271693^{#} | 0.66 | 27 | 3 | C | IBD, UC | IBD: rs477515, r² = 0.824 |
| | | | | | | | UC: rs9268853, r² = 0.885 |
| HLA-DQA1 | rs9271588^{#} | 0.50 | 22 | 11 | C | SjS⁷² | SjS: same |
| HLA-DQB1\hat{ }\hat{ } | rs3129763 | 0.52 | 11 | 0 | A | CLL, SSc | CLL: rs674313, r² = 0.854 |
| | | | | | | | SSc: same |
| IKZF2* | rs996032^{#} | 0.65 | 27 | 6 | A | SLE (AS) | SLE: rs3768792, r² = 0.888 |
| CCR1 | rs68181568 | 0.64 | 21 | 0 | C | CelD | CelD: rs13098911, |
| | | | | | | | r² = 0.919 |
| RERE\hat{ } | rs2401138 | 0.63 | 48 | 20 | C | V | V: rs4908760, r² = 0.827 |
| TMIBIM1* | rs2382818^{#} | 0.61 | 31 | 12 | A | IBD | IBD: rs2382817, r² = 1.0 |
| CLEC16A\hat{ }\hat{ } | rs7198004 | 0.59 | 16 | 0 | G | SLE | SLE: rs12599402, |
| | | | | | | | r² = 0.963 |
| CLEC16A | rs998592 | 0.50 | 10 | 0 | C | SLE | SLE: rs12599402, |
| | | | | | | | r² = 0.927 |
| CD44\hat{ }\hat{ } | rs3794102^{#} | 0.58 | 30 | 13 | G | V | V: rs10768122, r² = 1.0 |
| BLK\hat{ } | rs2736335 | 0.53 | 19 | 8 | A | KD, KD | KD: rs2254546, r² = 1.0 |
| | | | | | | (AS), SLE, | SLE: rs7812879, r² = 0.929 |
| | | | | | | SLE (AS), | |
| | | | | | | SLE (multi) | |
| PRKCQ | rs947474 | 0.52 | 11 | 0 | A | T1D, RA⁷³ | TID: same |
| | | | | | | | RA: same |
| TNIP1* | rs2233287 | 0.52 | 17 | 7 | G | Ssc | Ssc: same |
| RHOH\hat{ }\hat{ } | rs13136820 | 0.52 | 141 | 86 | T | GD | GD: rs6832151. r² = 0.939 |
| DQ658414 | rs73318382 | 0.50 | 10 | 0 | A | SLE, SLE | SLE: rss7095329; r² = 1.0 |
| MIR3142, | | | | | | (AS), SLE | |
| MIR164A)* | | | | | | (multi) | |
| RMI/2\hat{ } | rs34437200 | 0.49 | 10 | 2 | A | CelD, IBD, | CelD: rs12928822; |
| | | | | | | JIA, MS | r² = 0.841 |
| | | | | | | | IBD: rs529866; r² = 0.948 |
| | | | | | | | JIA: rs66718203; r² = 0.841 |
| | | | | | | | MS: rs6498184; r² = 0.965 |
| ZFP36L1 | rs194749^{#} | 0.47 | 11 | 4 | C | IBD, T1D | IBD: same |
| | | | | | | | TID: rs1465788; r² = 0.814 |
| HLA- | rs532098^{#} | 0.41 | 24 | 15 | G | SLE | SLE = same |
| DQB1\hat{ }\hat{ } | | | | | | | |
| HLA-DRB1, | rs674313 | 0.41 | 24 | 15 | G | CLL, SSc | CLL: same |
| HLA-DRB5 | | | | | | | SSc: rs3129763; r² = 0.863 |
| PPIF\hat{ }\hat{ } | rs1250567 | 0.41 | 8 | 3 | T | MS | MS: rs1782645; r² = 0.8475 |
| TAGAP* | rs1738074 | 0.40 | 47 | 32 | T | CelD, MS⁷⁴ | CelD: same |
| | | | | | | | MS: same |

All allelic results are from Mutu cells, except for the RMI2 locus, which uses EBNA2 GM12878 ChIP-seq data. Each variant was assigned to a gene using the following procedure. If the variant is located within the promoter (+/− 5 kb) of a gene expressed in EBV infected B cells (median RPKM of 2 or more based on GTEx55 data, assign to that gene (indicated with *). Otherwise, if the variant is located within a Hi-C chromatin looping region in GM12878 EBV infected B cells⁷⁵, assign it to the closest interacting gene that is
expressed in EBV infected B cells (indicated with \hat{ }\hat{ }). Otherwise, if the variant is located within a Hi-C chromatin looping region in primary B cells⁷⁶, assign it to the closest interacting gene that is expressed in EBV infected B cells (indicated with \hat{ }). Otherwise, assign the variant to the nearest gene that is expressed in EBV infected B cells. Variants marked with a ^{#} are eQTLs for the indicated gene in at least one EBV infected B cell dataset^{55,77-84}. ARS: Allelic Reproducibility Score” (see Supplementary Methods). Reads (Strong (Str.)) and Reads (Weak) indicate the number of ChIP-seq reads mapping to the strong and weak allele, respectively. Str Base is the base with more reads. r² values derived from European ancestry frequencies are provided. All r² values are greater than 0.80 when matching for ancestry. All disease associations are taken from the original disease lists, with the exception of three additional associations-citations are provided for these. Disease abbreviations: MS, multiple sclerosis; IBD, inflammatory bowel disease; UC, ulcerative colitis; SLE, systemic lupus erythematosus; CLL, chronic lymphocytic lymphoma; SSc, systemic sclerosis; SjS, Sjögren's syndrome; CelD, celiac disease; V, vitiligo; KD, Kawasaki's disease; T1D, Type 1 Diabetes; GD, Graves disease; JIA, juvenile idiopathic arthritis. GWAS results for diseases are in the European ancestry (EU), except as indicated (East Asian (AS)).

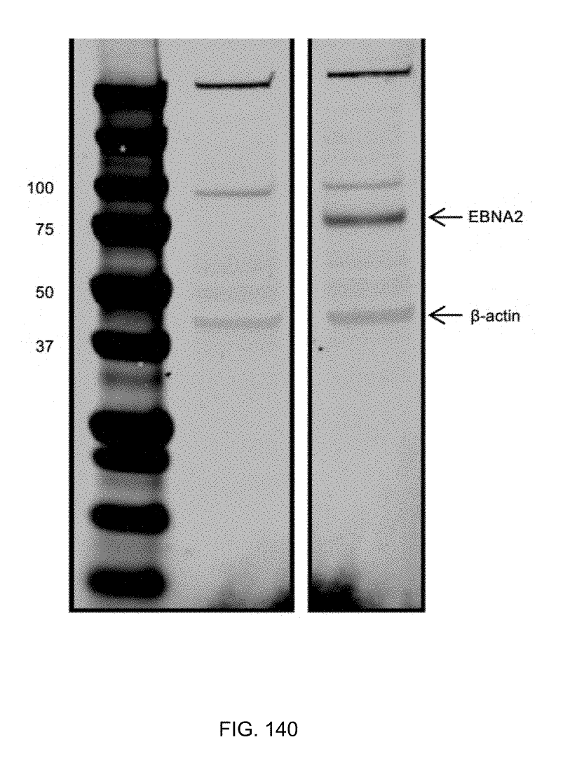

To detect potential downstream effects of allelic EBNA2 binding, Applicant measured genome-wide gene expression levels by RNA-seq in Ramos, an EBV negative B cell line that can support an EBV infection. Applicant confirmed the expected presence or absence of EBNA2 by sequencing and western blot (FIG. 140). Applicant identified a total of 89 genes with significant EBV-dependent alterations in gene expression, confirming that EBV modulates the expression of human genes. These results are highly consistent with a previous gene expression study and the literature (see Supplementary Methods).

Applicant next searched for autoimmune-associated variants that might impact EBNA2 binding, resulting in allelic expression of a nearby gene. This analysis was dependent on the small subset of genetic variants satisfying four necessary criteria: the variant must be (1) plausibly causal for an autoimmune disorder; (2) immunoprecipitated by EBNA2; (3) heterozygous in the cell line assayed; and (4) proximal to a plausible target mRNA that contains a heterozygous variant in Ramos cells (to detect allelic expression). For example, the 23 EBNA2 variants listed satisfy the first three criteria, but only five satisfy the fourth criterion of being within 50kb of a potential target gene containing a heterozygous variant in the Ramos cell line.

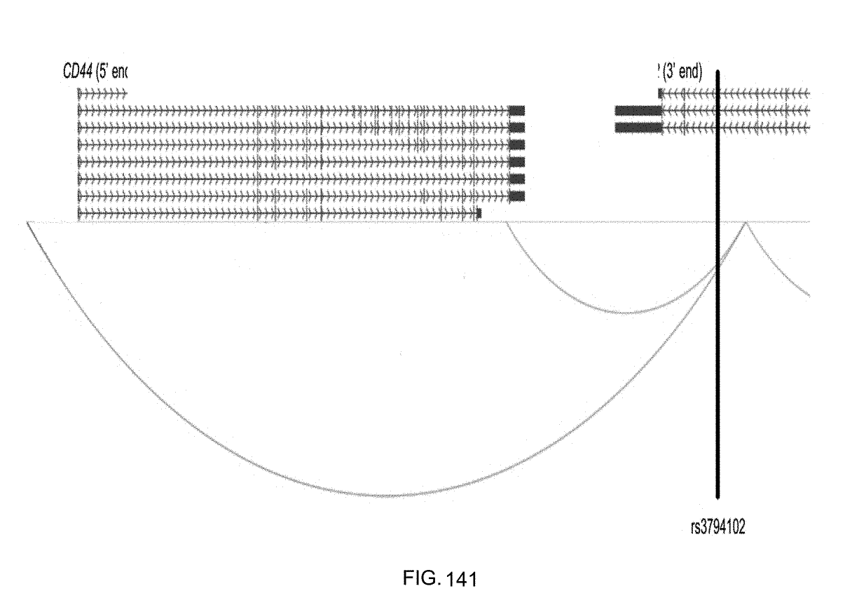

Despite these limitations, Applicant's approach identified autoimmune-associated variants displaying allelic EBNA2 binding and allelic expression of a nearby gene. For example, rs3794102, a variant strongly associated with vitiligo (P<10⁻⁹), has significantly skewed allelic binding of eight proteins—EBNA2, its suspected co-factor EBF136, and chromatin accessibility all favor the non-reference ‘G’ vitiligo risk allele (FIG. 135C, FIG. 140). Intriguingly, the proteins favoring the ‘G’ allele are considered activators, whereas the two ‘A’ allele proteins are repressors, suggesting that the variant and virus might act synergistically as an allelic switch. rs3794102, which is located within an intron of SLC1A2 (a gene for which Applicant detect no RNA-seq reads), loops to the promoter of the neighboring CD44 gene based on Hi-C experiments performed in GM12878 (FIG. 141). rs3794102 is also an established eQTL for CD44 in EBV-infected B cell lines (P<10⁻¹¹, ‘MRCE’ dataset, RTeQTL database′), and particular isoforms of CD44 are dependent on the presence of EBNA242. In Applicant's data, CD44 expression is 6.8-fold higher in EBV-infected Ramos cells compared to uninfected Ramos cells (P=0.00015). Further, Applicant identified a heterozygous genetic variant (rs8193) in strong LD with rs3794102 (r2=0.87) located within the CD44 gene body with 12 ‘T’ allele RNA-seq reads and only 5 ‘C’ allele reads in EBV-infected Ramos cells, and no detectable reads in Ramos cells lacking EBV. Applicant independently confirmed this result with allelic qPCR, observing a significant increase in expression for the T relative to the C allele in EBV infected Ramos cells, with significantly lower levels of expression in the absence of EBV (FIG. 3d). CD44 is a transmembrane glycoprotein involved in B cell migration and activation. Taken together, these results suggest that the ‘G’ vitiligo risk allele enhances formation of an EBNA2-dependent gene activation complex, resulting in elevated expression of CD44, and consequent increased B cell migration and/or activation. Applicant also identified a variant (rs947474) associated with T1D (Table 2) located near PRKCQ, another gene with allele- and EBV-dependent expression in Applicant's data (not shown). Intriguingly, PRKCQ plays an established role in activation of the EBV lytic cycle⁴³. Together, these examples establish that multiple autoimmune variants may alter binding events of protein complexes containing EBNA2 and host proteins, resulting in EBV-controlled allele-dependent host gene expression.

Autoimmune-Associated Genetic Mechanisms in EBV-Infected B Cells

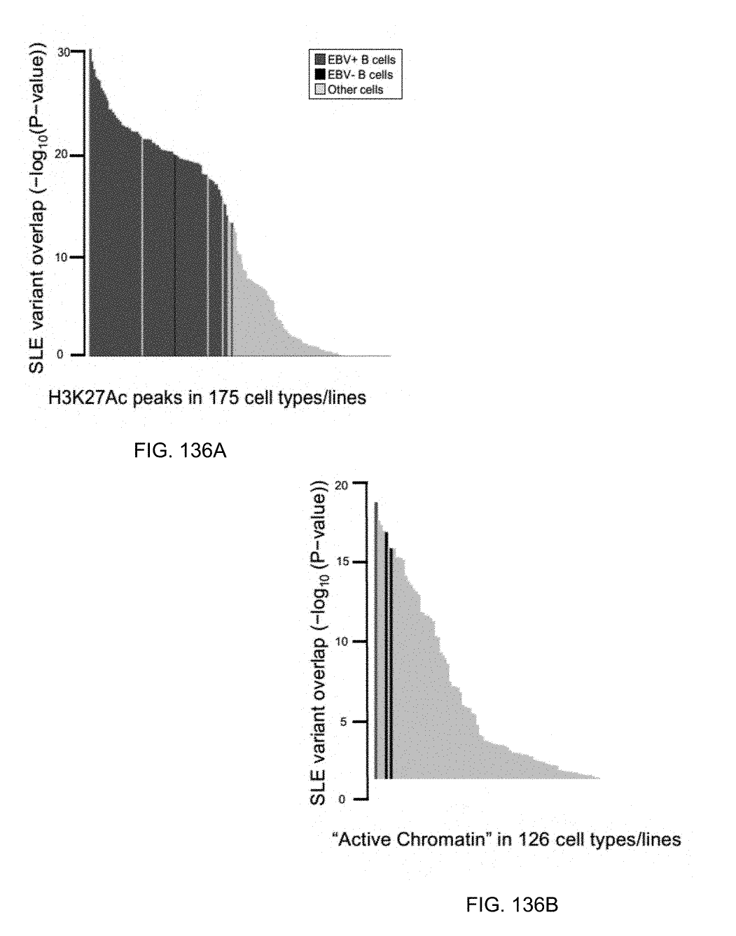

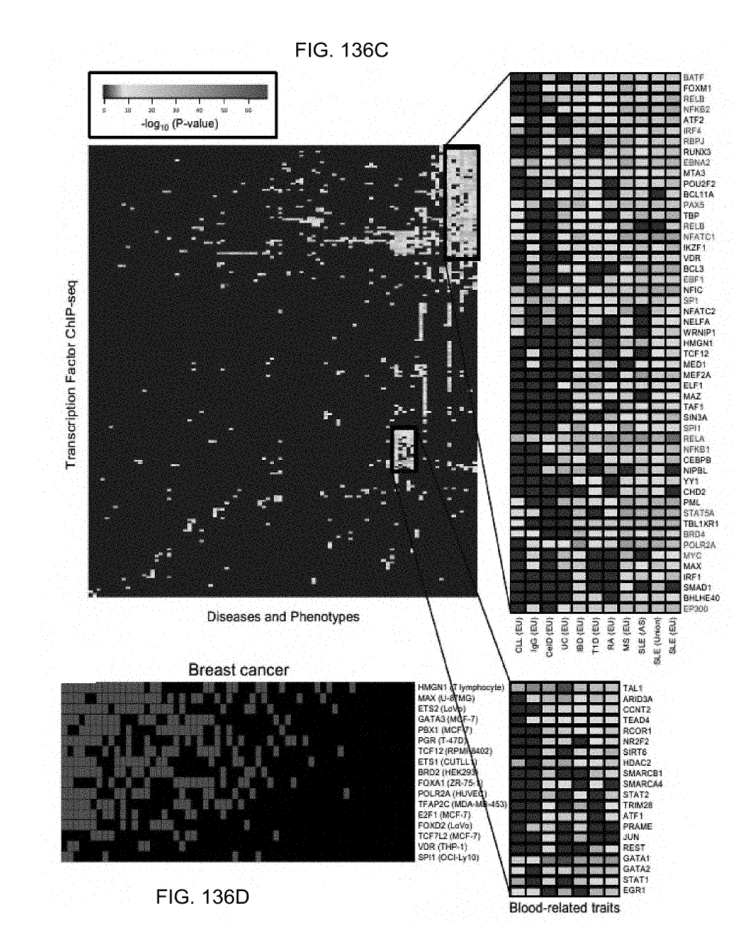

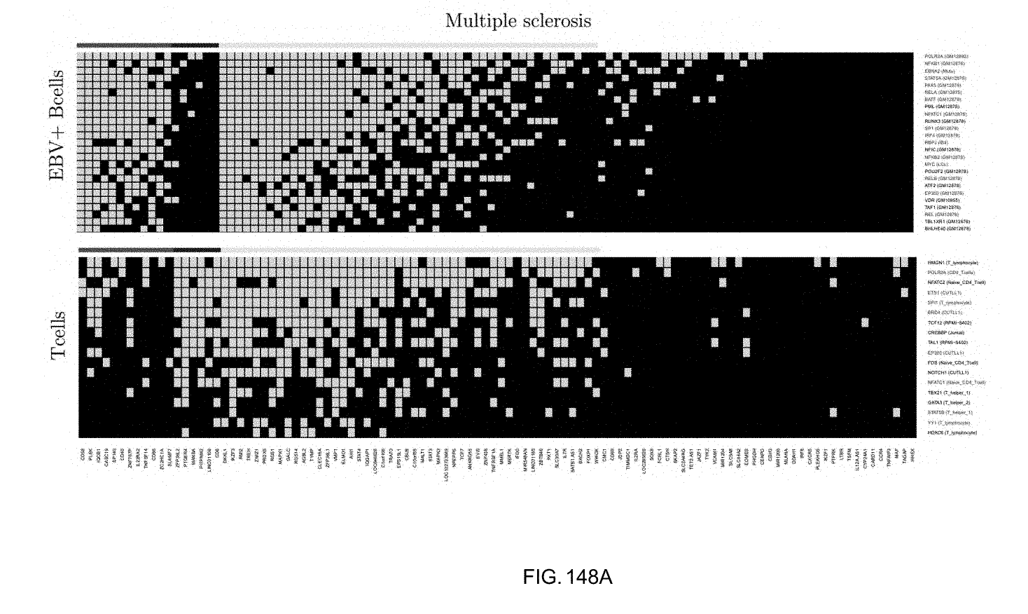

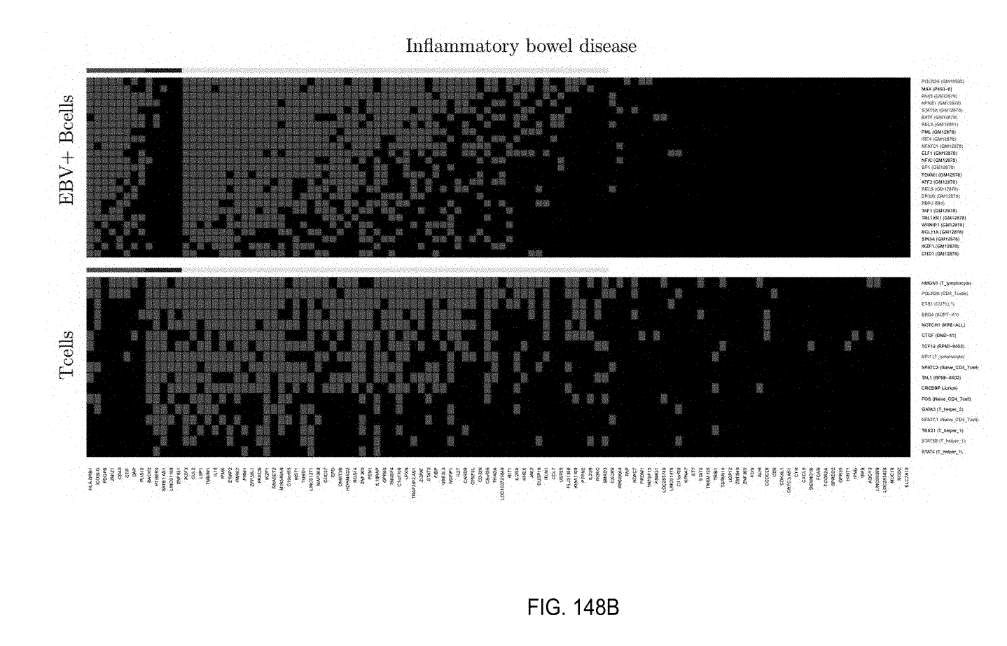

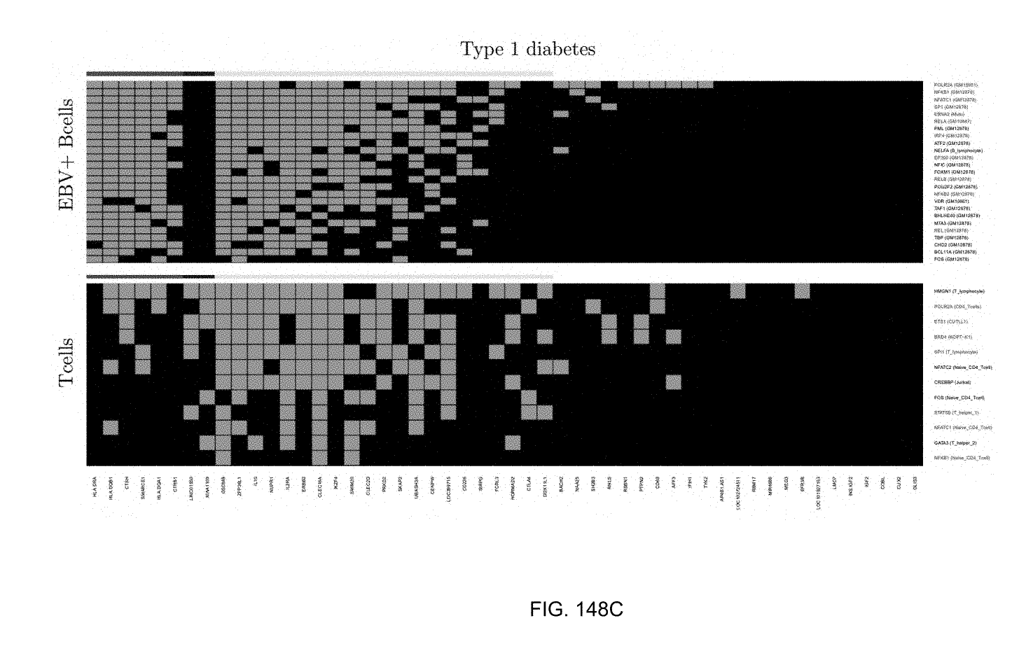

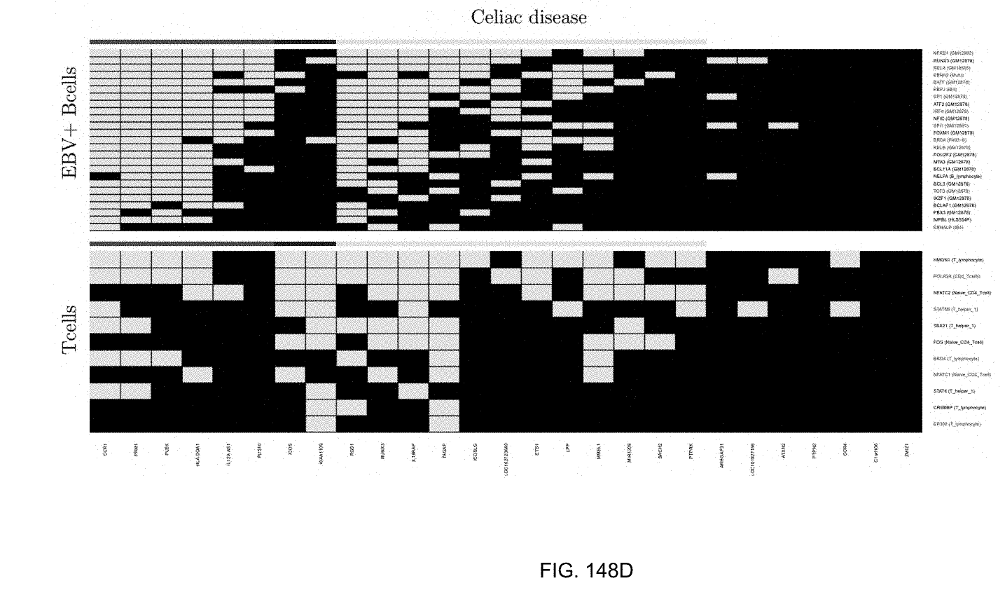

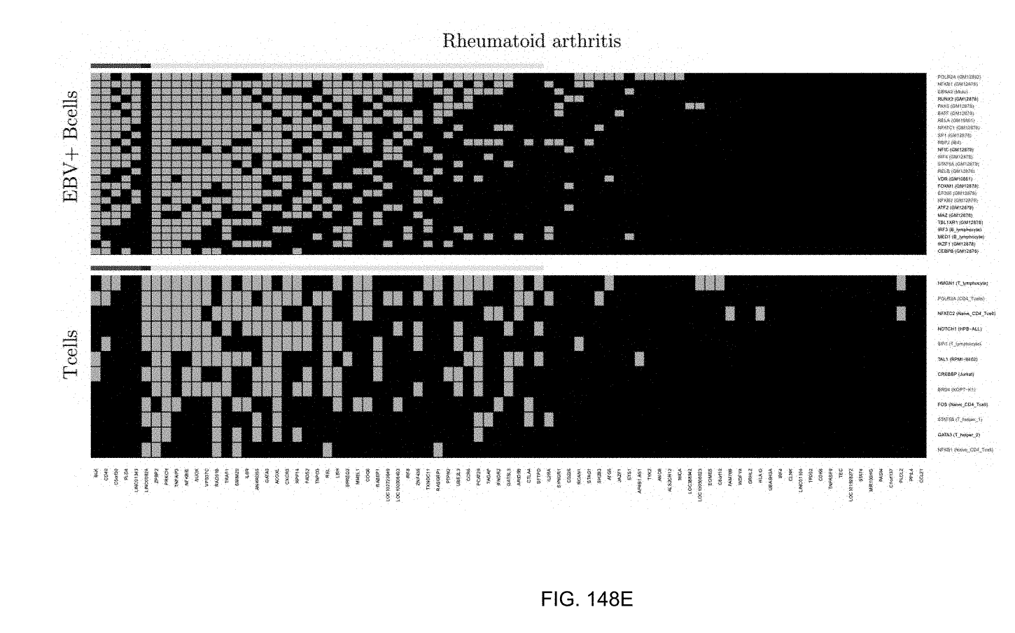

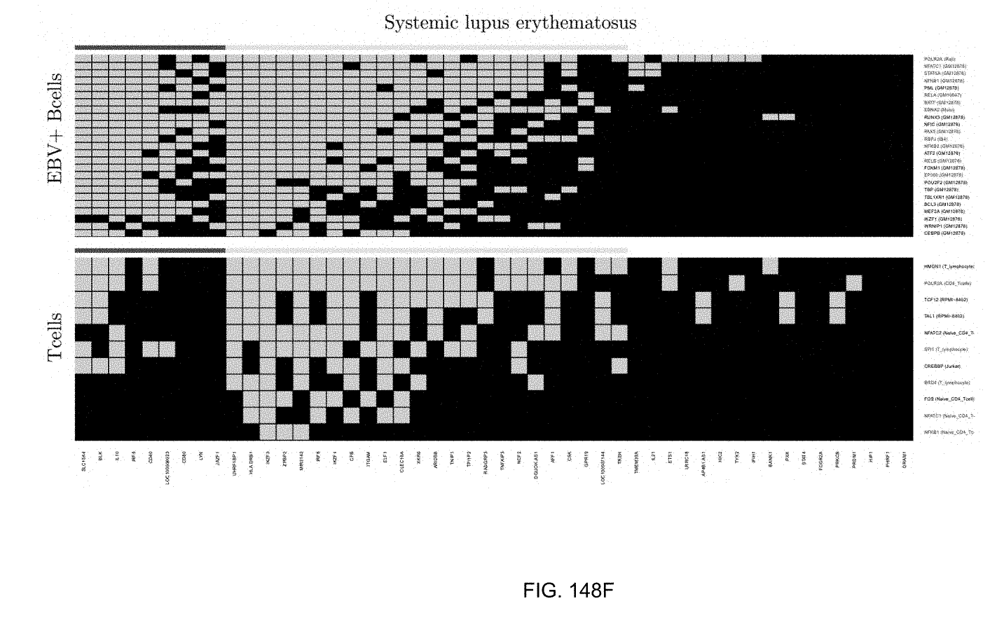

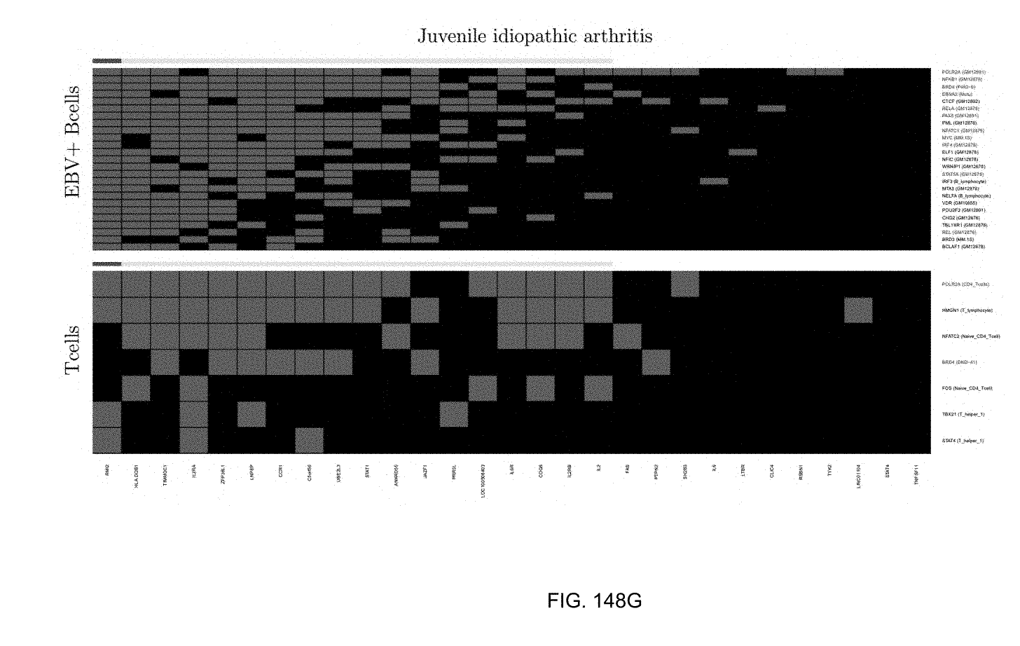

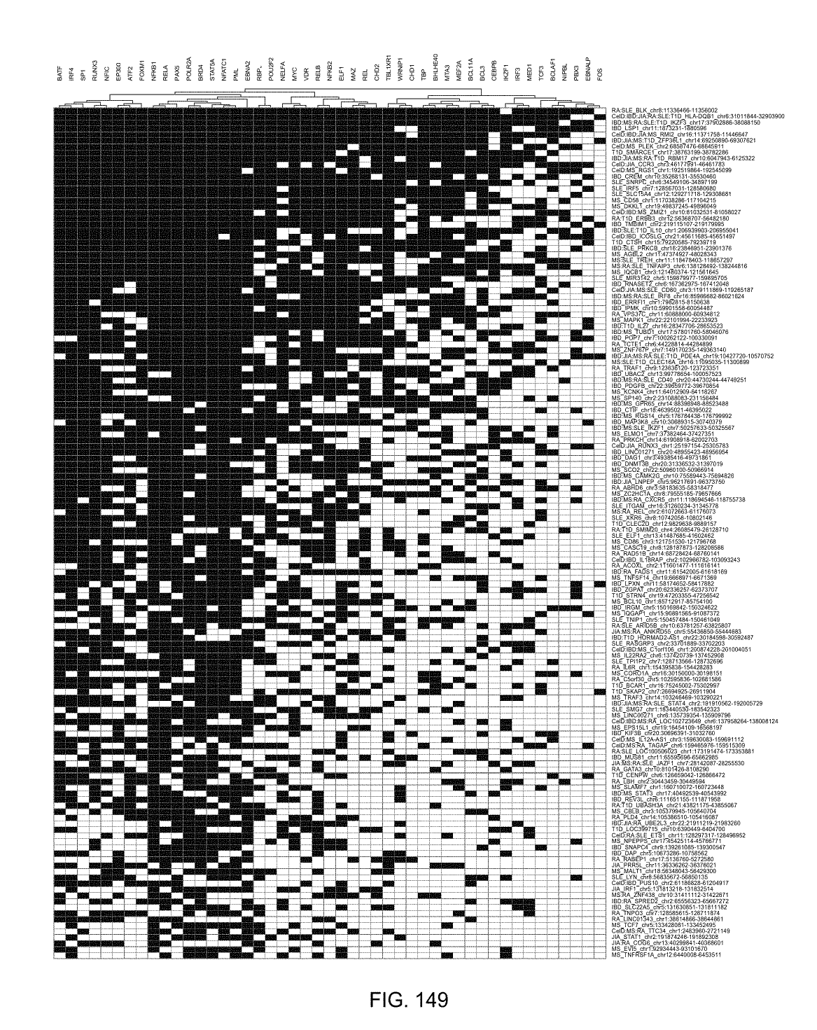

Applicant next used RELI to rank cell types by their relative importance to each of the EBNA2 disorders, based on the intersection between disease-associated variants and likely regulatory regions in that cell type. This procedure revealed a clear enrichment for EBV-infected B cells in SLE. For example, of the 175 H3K27ac ChIP-seq datasets available, the highest ranked 30 datasets are all from EBV-infected B-cell lines (FIG. 136A). Analogous results are obtained for “active chromatin marks” (a model based on combinations of various histone marks44) (FIG. 136B), H3K4me3, and H3K4me1, for SLE and virtually all of the seven EBNA2 disorders (FIG. 147). Collectively, these results support the EBV-infected B cell being an origin for genetic risk for each of the seven EBNA2 disorders. This analysis also reveals a likely involvement of other immune cell types in these disorders, including T cells, natural killer cells, and monocytes. Although there are limited TF ChIP-seq data available for these cell types, one or more of the EBNA2 disorders are associated with 17 of the available T cell TF ChIP-seq datasets. Further, several EBNA2 disorder loci appear to be specific to T cells. For example, six MS-associated loci are largely T cell-specific, collectively intersecting 67 T cell ChIP-seq datasets, compared to only 12 EBV-infected B cell datasets (FIG. 148A-G). Together, these results are consistent with multiple shared regulatory mechanisms acting across autoimmune risk loci, some common between cell types and others being exclusive to a certain cell type.

RELI Identifies Relationships Between Particular TFs and Many Diseases

Extension of RELI analysis to GWAS data for 213 phenotypes produced 2,264 significant (Pc<10⁻⁶) TF-disease connections. In addition to the EBNA2-related associations, clustering of these results reveals a large grouping of hematopoietic phenotypes and well-established blood cell regulators such as GATA1 and TAL1 (FIG. 136C). Other associations suggest additional mechanisms, many of which are supported by independent lines of evidence from other studies, such as GATA3, FOXA1, and TCF7L2 in breast cancer (FIG. 136D), and AR, NR3C1, and EZH2 in prostate cancer. In total, application of these methods produces results nominating global disease mechanisms for 94 different diseases or phenotypes, providing new directions for understanding their origins.

### Discussion

Efforts to understand the gene-environment interaction of SLE loci with EBV have revealed that EBNA2 and its associated human TFs occupy a significant fraction of autoimmune risk loci. Further analyses suggest that multiple causal autoimmune variants may act through allele-dependent binding of these proteins, resulting in downstream alterations in gene expression. In this scenario, the relevant TFs and gene expression changes must occur in the cell type that alters disease risk. Collectively, Applicant's data identify the EBV-infected B cell as a possible site for gene action in multiple autoimmune diseases, with the caveat that existing data are biased, having been predominantly collected in this cell type. Notably, four of the top 20 TFs that co-occupy EBNA2 disorder loci with EBNA2 are targeted by at least one available drug (MED1, EP300, NFKB1, and NFKB2)⁴⁵, and a recent study shows that the C-terminal domain of the BS69/ZMYND11 protein can bind to and inhibit EBNA2⁴⁶. These results offer promise for the development of future therapies for manipulating the action of these proteins in individuals harboring risk alleles at EBNA2-bound loci.

The disclosed results nominate particular TFs and cell types for 94 phenotypes, providing mechanisms possibly explaining the molecular and cellular origins of disease risk for experimental verification and exploration.

Methods Summary

Applicant compiled and curated a set of 99,733 variants associated with or in strong linkage disequilibrium with 213 phenotypes (based upon direct genotyping and/or standard variant imputation). Applicant collected a set of 2,511 functional genomics datasets (ChIP-seq for specific proteins, ChIP-seq for histone marks, DNase-seq, and eQTLs) from a variety of sources. Applicant developed a novel algorithm, RELI (Regulatory Element Locus Intersection), to estimate the significance of the intersection between the variants associated with a given phenotype and a given functional genomics dataset. To identify allelic binding of proteins within ChIP-seq datasets, Applicant genotyped five EBV-infected B cell lines, and developed a novel pipeline called MARIO (Measurement of Allelic Ratios Informatics Operator) to detect allelic read count imbalance at heterozygotes in the assayed cell line. To identify gene expression patterns dependent upon both genotype and EBV, Applicant performed RNA-seq in Ramos B cell lines with or without EBV infection. Details are provided in the Supplementary Methods.

Collection and Processing of Datasets

Applicant compiled a large collection of genetic and functional genomic datasets from a variety of sources. Phenotype-associated genetic variants were largely obtained from the NHGRI GWAS catalog²⁹. This catalog does not contain candidate gene studies, including those from the widely-used ImmunoChip platform⁴⁷. For SLE, MS, SSc, RA, and JIA, peer-reviewed literature was thus curated to maximize the number and accuracy of loci. Only associations exceeding genome-wide significance (P<5×10⁻⁸) were considered. Datasets were separated and annotated by ancestry, except where noted. Phenotypes were filtered to only include those with five or more associated loci separated by at least 500 kb, following Farh et al.³⁰. Loci containing multiple variants were restricted to the single most strongly associated variant, and subsequently expanded to incorporate variants in strong linkage disequilibrium (LD) (r2>0.8) with this variant using Plink⁴⁸. The resulting variants in each locus are referred to as plausibly causal.

Functional genomics data, including ChIP-seq and DNase-seq, were obtained from a variety of sources, including ENCODE⁴⁹ (downloaded on 4/14), Roadmap epigenomics⁵⁰ (6/15), Cistrome⁵¹ (12/15), PAZAR⁵² (4/14), ReMap-ChIP⁵³ (8/15), and Gene Expression Omnibus⁵⁴. ChIP-seq datasets containing less than 500 peaks were removed. The genomic coordinates of the peaks for each dataset were stored as .bed files. eQTLs were obtained from GTExPortal⁵⁵ (1/16), the Pritchard lab eQTL database (http://eqthuchicago.edu/) (4/14), and the Harvard eQTL database (https://www.hsph.harvard.edu/liming-liang/software/eqtl/) (4/14). TF binding motif models in the form of position frequency matrices were obtained from Cis-BP (build 1.02)⁵⁶.

Regulatory Element Locus Intersection (RELI) Algorithm

Applicant created the RELI algorithm to search for potential shared regulatory mechanisms acting across phenotype-associated loci. In brief, RELI takes a set of variants as input, expands the set using LD blocks, and calculates the statistical intersection of the resulting loci with every dataset in a compendium (e.g., ChIP-seq datasets) (FIG. 134A). In Step 1, RELI accepts a set of variants associated with a given phenotype. The sequencing data available from 1,000 Genomes⁵⁷ is then used to identify all variants with r2>0.8 with any input variant. At each locus, each variant is assigned to a single LD block based on its highest r2 value. LD blocks are chosen to match the ancestry of the input variant set (European, Asian, African, etc.). In Step 2, the observed intersection is recorded between each LD block and each dataset, based on their genomic coordinates. If any variant in a given LD block intersects a given dataset, that LD block/dataset pair is marked as an “intersection”. In Step 3, the expected intersection is estimated between each LD block and each dataset. The most strongly associated variant is chosen as the reference variant for the LD block. A distance vector is then generated providing the distance (in bases) of each variant in the LD block from this reference variant. A random genomic variant with approximately matched allele frequencies to the reference variant is then selected from dbSNP⁵⁸, and genomic coordinates of artificial variants are created that are located at the same relative distances from this random variant using the distance vector. Members of this artificial LD block are then intersected with each dataset, as for the observed intersections. This strategy takes into account the distance between variants in the input LD blocks, while eliminating any ‘double counting’ that might occur due to multiple variants in the block intersecting the same dataset. Applicant repeated this simulation procedure 2,000 times, generating a null distribution. 2,000 repetitions are sufficient for the P-values to stabilize (data not shown). The intersection significance between the input variant set and each dataset is then estimated by comparing the observed counts to the distribution of expected counts. The expected intersection distributions are Gaussian, and can hence be used to calculate Z-scores and P-values. The final reported P-values are Bonferroni corrected (Pc) for the 1,544 TF datasets tested. Applicant also calculated the relative risk by dividing the observed intersection by the expected intersection.

RELI was designed to be flexible in terms of the null models it employs. The default null model, as described above, uses all common variants in the genome. Applicant also considered a higher-stringency null model by only considering common variants located within DNase-seq peaks in any of the 22 available EBV-infected B cell line datasets. This null model thus controls for the known association of SLE-associated variants with regulatory regions in B cells²³.

Applicant identified the optimal clusters depicted as red boxes in FIG. 133A-G using the following procedure, which compares the observed number of TF/locus intersections to results from simulations. First, loci (X-axis) and TFs (Y-axis) were sorted in decreasing order of the number of intersections (colored boxes in the heatmap). Applicant then iteratively considered every possible sub-matrix boundary, starting at the upper left corner. In each simulation trial, the total number of intersections is kept fixed, but the locations of the intersecting positions are randomly permuted across loci. A Gaussian null distribution is obtained from 10,000 random trials. P-values are calculated for each sub-matrix by comparing the observed number of intersections falling within the sub-matrix to the null distribution, using a standard Z-score transformation. The optimal cluster is defined as the sub-matrix with the best P-value.

Cell Line Genotyping and Imputation

Without genotyping data, it is not possible to distinguish between perfect allelic imbalance at a heterozygous variant (e.g., 10 reads on one allele and 0 on the other) and homozygosity. Applicant therefore genotyped five EBV-infected B cell lines that had previously been used for ChIP-seq experiments. Genotyping was performed as previously described⁵⁹ on Illumina OMNI-5 genotyping arrays using Infinium2 chemistry. Genotypes were called using the Gentrain2 algorithm within Illumina Genome Studio. Quality control on the variants from autosomal chromosomes was performed as previously described⁵⁹. Quality control data cleaning was performed in the context of a larger batch of non-disease controls to allow for the assessment of data quality. Briefly, all cell lines had call rates >99%, only common variants (minor allele frequency >0.01) were included, and all variants were previously shown to be in Hardy-Weinberg equilibrium in control populations at P>0.000159. To detect associated variants that were not directly genotyped on the OMNI-5, Applicant performed genome-wide imputation using overlapping 150 kb sections of the genome with IMPUTE2⁶⁰ and used a composite imputation reference panel of pre-phased integrated haplotypes from the 1,000 Genomes Project sequence data freeze from June 2014. Imputed genotypes were required to meet or exceed a probability threshold of 0.9, an information measure of >0.5, and the same quality-control criteria threshold described above for the genotyped markers.

Detection of Allele-Dependent Sequencing Reads Using MARIO

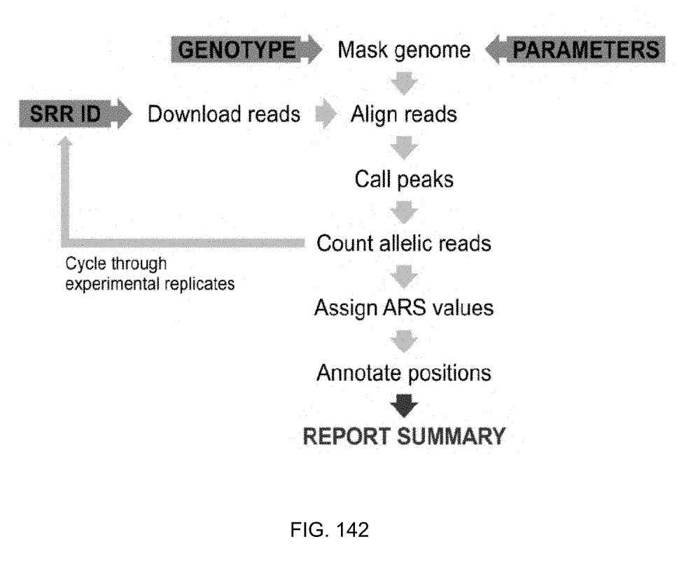

Applicant developed the MARIO (Measurement of Allelic Ratio Informatics Operator) pipeline to identify allele-dependent behavior at heterozygous variants in functional genomics datasets such as ChIP-seq. In brief, the pipeline downloads a set of reads, aligns them to the genome, calls peaks using MACS2⁴⁴ (parameters: --nomodel--extsize 147-g hs-q 0.01), identifies allele-dependent behavior at heterozygotes within peaks (described below), and annotates the results (FIG. 142).

To estimate the significance of the degree of allelic imbalance of a given ChIP-seq, ATAC-seq, or DNase-seq dataset at a given heterozygote, Applicant developed a value called the ARS (Allelic Reproducibility Score). The ARS is based on a combination of two predictive variables for a given heterozygous variant of a given dataset—the total number of reads available at the variant and the imbalance between the number of reads for each allele. Other variables were tested and deemed uninformative (see below). The ARS value also accounts for the number of available experimental replicates, and the degree to which they agree. ARS values were calibrated using seven TFs with ChIP-seq datasets available in four replicate experiments in GM12878 or K562 cell lines: SPI1 (set 1), SPI1 (set 2), NRSF, REST, RNF2, YY1 and ZBTB33. The presence of multiple replicates monitoring binding of the same TF in the same cell type enables the estimation of the degree to which allelic behavior is reproducible, given the values of the predictive variables.

ARS Values were Defined and Calculated Using the Following Procedure:

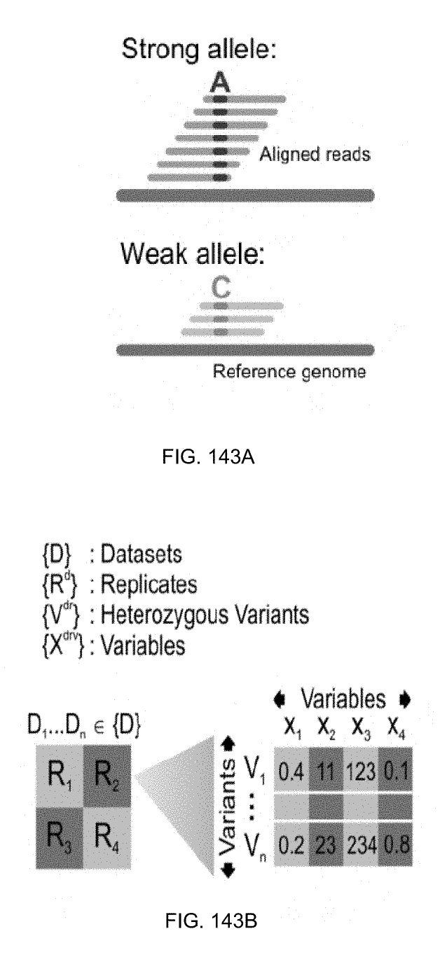

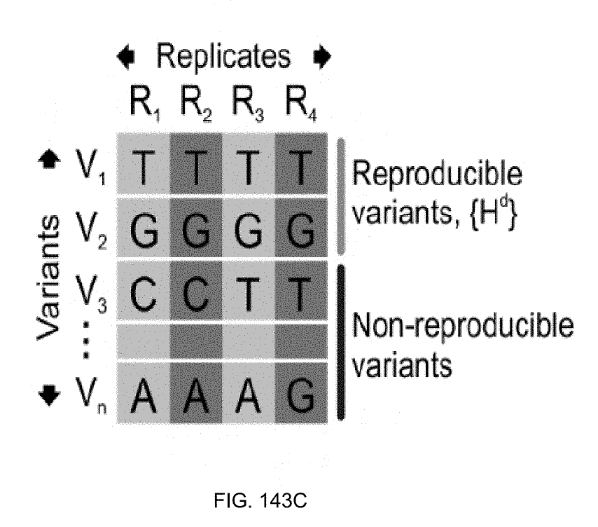

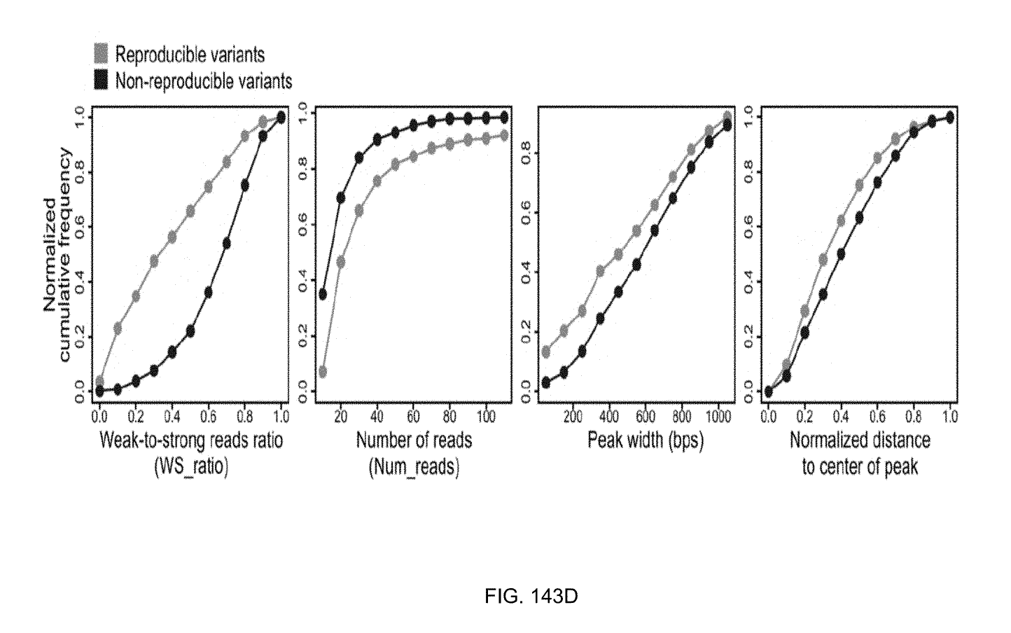

1) Determine the number of reads mapping to each allele of each heterozygous variant in each replicate. The pipeline was applied to each experimental replicate and counted the number of reads that overlap each heterozygous variant, corresponding to the two alternative alleles. All duplicate reads were removed using the “MarkDuplicates” tool from the PICARD software package (https://broadinstitute.github.io/picard/). Before mapping reads using Bowtie2⁶¹ (parameters-N 1--np 0--n-ceil 10--no-unal), Applicant masked all common variants in the GrCh37 (hg19) reference genome to N. This step removed bias generated by reads carrying non-reference alleles. Applicant designated the allele with the greater number of reads the strong allele, and the other the weak allele (FIG. 143A).

2) Identify predictive variables of reproducible allele-dependent behavior across replicates. Applicant identified variables that are predictive of reproducible allelic behavior across multiple ChIP-seq replicates within a dataset. Applicant collected a set of seven datasets, {D}, with each dataset comprised of four experimental replicates, {R} (FIG. 143). Each replicate contains a set of variants {V} that are heterozygous in the given cell type. For each of these variants, Applicant calculated the value of four variables {X}: the ratio between the number of weak and strong allele reads, the total number of reads available at the variant, distance to peak center, and peak width.

Applicant evaluated the performance of each of these variables using a true-positive set of reproducible variants. This set was created by identifying all variants that share the same strong allele across all four replicates (FIG. 143C). Each variable was assessed based upon its ability to effectively separate reproducible variants from all other variants, which Applicant designate non-reproducible variants. The reproducible variants are enriched for allelic behavior, whereas the non-reproducible variants are depleted (FIG. 143D, left-most panel). This analysis produced two variables predictive of reproducible allelic binding: the ratio between the number of weak and strong allele reads (WS_ratio), and the total number of reads available at the variant (num_reads), which Applicant designate the predictive variables.

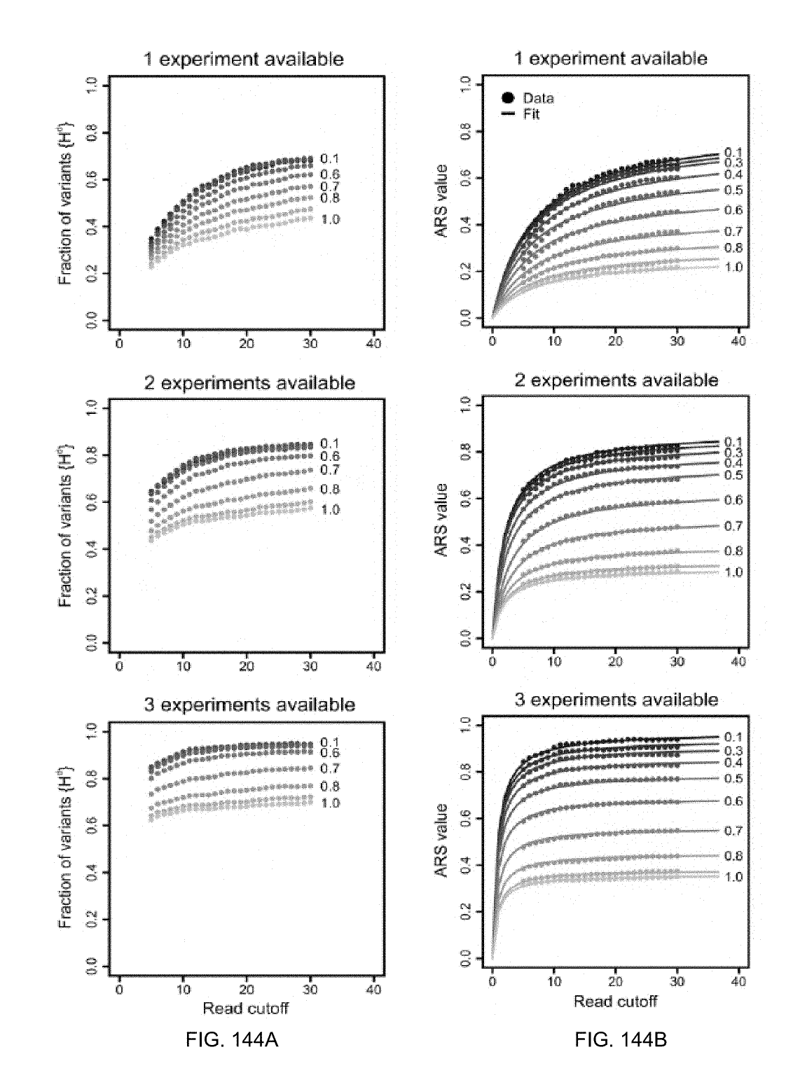

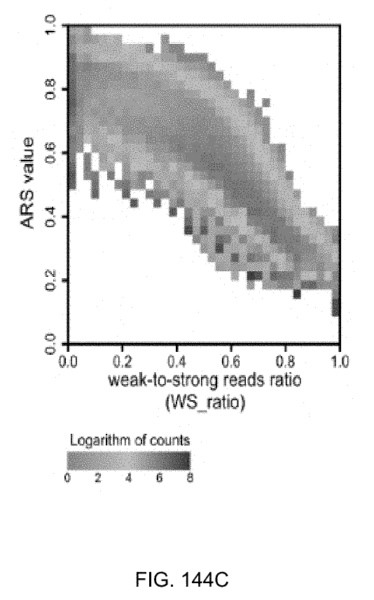

3) Determine a function mapping the values of the predictive variables to a single ARS value. Applicant next created a function for mapping the values of predictive variables for any heterozygous variant to a single ARS value estimating the degree of reproducible allelic behavior. Applicant developed a scheme that accounts for the fact that any given dataset might contain any number of experimental replicates, with agreement between a larger number of replicates being a desirable trait. Within each of the seven datasets in the set {D}, all possible combinations of one, two, or three replicates is considered. Without loss of generality, the procedure for the case of two replicates is described, which considers the subsets {R1,R2}, {R1,R3}, {R1,R4}, etc. The set {H} of reproducible variants is first identified (as described above) for each subset. The WS_ratio is transformed into ranges, {(0-0.1), (0-0.2), (0-0.3), . . . (0-1)}, and for each range, the fraction of variants that are contained in the reproducible variant set as a function of num_reads is calculated (FIG. 144A). It is noted that, at this stage, this fraction still accounts for all variants, both allelic and non-allelic. Each curve is adjusted by the normalized cumulative frequency of non-allelic variants within the given range. For example, consider the WS_ratio=0.3 curve (FIG. 144A). Each point on this curve is divided by a single value representing the normalized cumulative frequency of the non-reproducible variants. This is obtained from the Y-axis at the X=0.3 position in the WS_ratio plot depicted in FIG. 143D. Before dividing, 1 is added to this value to avoid divide-by-zero errors. Collectively, this approach selectively penalizes non-allelic behavior by accounting for the proportion of non-allelic variants within each curve. These values were averaged across the seven datasets, yielding the final ARS values. This entire procedure is repeated for the cases of one, two, or three available replicates, generating the points shown in FIG. 144B. Curves were fit to these points using a saturating function:

$${ARS}_{w} = \frac{{A}_{w}}{1 + {B}_{w} \times r} - {A}_{w} ,$$

where w is the WS_ratio, r is num_reads, and Aw and Bw are the fitting parameters. The resulting functions yield ARS values for any given heterozygous variant in any dataset, as a function of the number of experimental replicates, the WS_ratio, and num_reads. As a final step, when multiple replicates are available, an ARS value is only reported for a variant if the strong allele is consistent in the majority of cases, to account for the possibility of a failed experiment. A direct interpretation of the ARS values can be seen in the relationship between ARS values and the WS_ratio (FIG. 144C).

The corresponding NCBI experiment run identifiers for the seven ChIP-seq datasets with four available replicates are: NRSF (SRR1176035, SRR1176037, SRR1176039, SRR1176050), REST (SRR400395, SRR400396, SRR400397, SRR400398), RNF2 (SRR400400, SRR400401, SRR400402, SRR400403), SPI1 (set 1) (SRR1176055, SRR1176056, SRR1176057, SRR1176058), SPI1 (set 2) (SRR351880, SRR351881, SRR578180, SRR578181), YY1 (SRR351719, SRR351720, SRR578174, SRR578175), ZBTB33 (SRR1176059, SRR1176060, SRR1176061, SRR1176062).

EBV Infection of Ramos Cells.

All cells were confirmed to be free of mycoplasma infection using PlasmaTest (InvivoGen, San Diego, Calif.) prior to use in experiments. Wild-type EBV was prepared from supernatants of B95-8 cells cultured in RPMI medium 1640 supplemented with 10% FBS for two weeks. Briefly, the cells were pelleted and the virus suspension was filtered through 0.45 μM Millipore filters. The concentrated virus stocks were aliquoted and stored at −80° C.

Applicant infected ˜2×10⁶ Ramos Cells (ATCC CRL-1596) in the presence of growth medium containing 2 μg/ml of phytohemagglutinin (PHA) for 4 hours. The infected cells were washed, cultured in growth media, and observed daily for multinuclear giant cell formation and morphological changes characteristic of EBV-infected B cells. After 10 passages, the infection was confirmed by measuring the expression of viral EBNA2 protein levels (FIG. 140). EBV-infected Ramos cells were enriched by flow cytometry (LMP-1 (Abcam 78113)).

RNA-Seq

RNA was isolated from Ramos cell lines with and without EBV infection using the mirVANA Isolation Kit (Ambion). RNA sequencing targeting 150 million mappable 125 basepair reads from paired-end, poly-A enriched libraries was performed at the CCHMC DNA Sequencing and Genotyping Core Facility at CCHMC. Sequencing reads were aligned to the GrCh37 (hg19) build of the human genome using TopHat⁶² and Bowtie2⁶¹ with Ensembl⁶³ RNA transcript annotations as a guide. In parallel, these data were aligned to the EBV genome (NCBI). As expected, 0 reads mapped in the EBV negative dataset, whereas 7,349 reads mapped in the EBV-infected dataset. 82.8% of the sequence reads aligned specifically to the human transcriptome, with a 2.6% increase in the aligned reads in the EBV negative samples. No abnormal quality control (QC) flags were identified following QC analysis with the software FastQC (http://www.bioinformatics.babraham.ac.uk/projects/fastqc/). For allelic analysis, sequencing reads were aligned to the GrCh37 (hg19) build of the human genome using Hisat2⁶⁴. Differential expression analysis was performed using Cufflinks⁶⁵.

As additional QC, Applicant further compared the results to a study examining host gene expression changes to EBV infection in primary B cells²⁸. Of the 80 genes whose expression is significantly altered by the presence of EBV in Applicant's study, 18 of them are also significantly differentially expressed in this dataset. Further, among the 80 differentially expressed genes detected, many of them represent classic host genes whose expression is modulated by EBV. Some gene expression is increased by the virus, while the expression of other genes is decreased. In all of these cases, the data agree with the established paradigm. Genes whose expression is activated by EBV include CD44⁶⁶, TNFAIP2⁶⁷, MX1⁶⁸, and IFI44⁶⁹; genes with lower expression include VAV3⁷⁰ and CD99⁷¹.

Allelic qPCR

gDNA and RNA were extracted from Ramos cells with and without B95.8 EBV infection using the DNeasy Blood & Tissue Kit (Qiagen) and mirVana miRNA Isolation Kit (Invitrogen), respectively. RNA was treated with DNase using the TURBO DNA-free Kit (Ambion) and converted to cDNA using the High-Capacity RNA-to-cDNA Kit (Applied Biosystems). qPCR was performed with a single set of Taqman genotyping primers (Applied Biosystems) to rs8193 using the ABI 7500 PCR system. Fold change of expression was calculated with 2-ΔΔCT values, where cDNA was normalized to gDNA.

Data Availability

RNA-seq data are available in the Gene Expression Omnibus (GEO) database under accession number GSE93709. Full datasets and results, including disease variants (with alleles) and all RELI and MARIO output, are provided in the Supplementary Material.

Code Availability

The final RELI and MARIO source code, with documentation, will be made freely available under the GNU General Public License on the Weirauch Lab Bitbucket page: https://bitbucket.org/account/user/weirauchlab/projects/ci

### REFERENCES

1 Fujinami, R. S., von Herrath, M. G., Christen, U. & Whitton, J. L. Molecular mimicry, bystander activation, or viral persistence: infections and autoimmune disease. Clin Microbiol Rev 19, 80-94, doi:10.1128/CMR.19.1.80-94.2006 (2006).
2 Ercolini, A. M. & Miller, S. D. The role of infections in autoimmune disease. Clinical and experimental immunology 155, 1-15, doi:10.1111/j.1365-2249.2008.03834.x (2009).
3 Sener, A. G. & Afsar, I. Infection and autoimmune disease. Rheumatol Int 32, 3331-3338, doi:10.1007/s00296-012-2451-z (2012).
4 James, J. A. et al. An increased prevalence of Epstein-Barr virus infection in young patients suggests a possible etiology for systemic lupus erythematosus. J Clin Invest 100, 3019-3026, doi:10.1172/JC1119856 (1997).
5 Hanlon, P., Avenell, A., Aucott, L. & Vickers, M. A. Systematic review and meta-analysis of the sero-epidemiological association between Epstein-Barr virus and systemic lupus erythematosus. Arthritis research & therapy 16, R3, doi:10.1186/ar4429 (2014).
6 McClain, M. T. et al. Early events in lupus humoral autoimmunity suggest initiation through molecular mimicry. Nat Med 11, 85-89, doi:10.1038/nm1167 (2005).
7 Harley, J. B. & James, J. A. Epstein-Barr virus infection induces lupus autoimmunity. Bulletin of the NYU hospital for joint diseases 64, 45-50 (2006).
8 Ascherio, A. & Munger, K. L. EBV and Autoimmunity. Curr Top Microbiol Immunol 390, 365-385, doi:10.1007/978-3-319-22822-8_15 (2015).
9 Draborg, A. H., Duus, K. & Houen, G. Epstein-Barr virus in systemic autoimmune diseases. Clinical & developmental immunology 2013, 535738, doi:10.1155/2013/535738 (2013).
10 Vaughn, S. E., Kottyan, L. C., Munroe, M. E. & Harley, J. B. Genetic susceptibility to lupus: the biological basis of genetic risk found in B cell signaling pathways. Journal of leukocyte biology 92, 577-591, doi:10.1189/jlb.0212095 (2012).
11 Alarcon-Riquelme, M. E. et al. Genome-Wide Association Study in an Amerindian Ancestry Population Reveals Novel Systemic Lupus Erythematosus Risk Loci and the Role of European Admixture. Arthritis Rheumatol 68, 932-943, doi:10.1002/art.39504 (2016).
12 Bentham, J. et al. Genetic association analyses implicate aberrant regulation of innate and adaptive immunity genes in the pathogenesis of systemic lupus erythematosus. Nat Genet 47, 1457-1464, doi:10.1038/ng.3434 (2015).
13 Sun, C. et al. High-density genotyping of immune-related loci identifies new SLE risk variants in individuals with Asian ancestry. Nat Genet 48, 323-330, doi:10.1038/ng.3496 (2016).
14 Maurano, M. T. et al. Systematic localization of common disease-associated variation in regulatory DNA. Science 337, 1190-1195, doi:10.1126/science.1222794 (2012).
15 Hindorff, L. A. et al. Potential etiologic and functional implications of genome-wide association loci for human diseases and traits. Proc Natl Acad Sci USA 106, 9362-9367, doi:10.1073/pnas.0903103106 (2009).
16 Fang, H., Knezevic, B., Burnham, K. L. & Knight, J. C. XGR software for enhanced interpretation of genomic summary data, illustrated by application to immunological traits. Genome Med 8, 129, doi:10.1186/s13073-016-0384-y (2016).
17 Schweizer, M. T. & Yu, E. Y. Persistent androgen receptor addiction in castration-resistant prostate cancer. J Hematol Oncol 8, 128, doi:10.1186/s13045-015-0225-2 (2015).
18 Asch-Kendrick, R. & Cimino-Mathews, A. The role of GATA3 in breast carcinomas: a review. Hum Pathol 48, 37-47, doi:10.1016/j.humpath.2015.09.035 (2016).
19 Almohmeed, Y. H., Avenell, A., Aucott, L. & Vickers, M. A. Systematic review and meta-analysis of the sero-epidemiological association between Epstein Barr virus and multiple sclerosis. PLoS One 8, e61110, doi:10.1371/journal.pone.0061110 (2013).
20 Pender, M. P. & Burrows, S. R. Epstein-Barr virus and multiple sclerosis: potential opportunities for immunotherapy. Clinical & translational immunology 3, e27, doi:10.1038/cti.2014.25 (2014).
21 Marquez, A. C. & Horwitz, M. S. The Role of Latently Infected B Cells in CNS Autoimmunity. Front Immunol 6, 544, doi:10.3389/fimmu.2015.00544 (2015).
22 Ricigliano, V. A. et al. EBNA2 binds to genomic intervals associated with multiple sclerosis and overlaps with vitamin D receptor occupancy. PloS one 10, e0119605, doi:10.1371/journal.pone.0119605 (2015).
23 Hu, X. et al. Integrating autoimmune risk loci with gene-expression data identifies specific pathogenic immune cell subsets. American journal of human genetics 89, 496-506, doi:10.1016/j.ajhg.2011.09.002 (2011).
24 Trynka, G. et al. Disentangling the Effects of Colocalizing Genomic Annotations to Functionally Prioritize Non-coding Variants within Complex-Trait Loci. American journal of human genetics 97, 139-152, doi:10.1016/j.ajhg.2015.05.016 (2015).
25 Zhou, H. et al. Epstein-Barr virus oncoprotein super-enhancers control B cell growth. Cell host & microbe 17, 205-216, doi:10.1016/j.chom.2014.12.013 (2015).
26 Gewurz, B. E. et al. Canonical NF-kappaB activation is essential for Epstein-Barr virus latent membrane protein 1 TES2/CTAR2 gene regulation. J Virol 85, 6764-6773, doi:10.1128/JVI.00422-11 (2011).
27 Ersing, I., Bernhardt, K. & Gewurz, B. E. NF-kappaB and IRF7 pathway activation by Epstein-Barr virus Latent Membrane Protein 1. Viruses 5, 1587-1606, doi:10.3390/v5061587 (2013).
28 Price, A. M. et al. Analysis of Epstein-Barr virus-regulated host gene expression changes through primary B-cell outgrowth reveals delayed kinetics of latent membrane protein 1-mediated NF-kappaB activation. J Virol 86, 11096-11106, doi:10.1128/JVI.01069-12 (2012).
29 Welter, D. et al. The NHGRI GWAS Catalog, a curated resource of SNP-trait associations. Nucleic Acids Res 42, D1001-1006, doi:10.1093/nar/gkt1229 (2014).
30 Farh, K. K. et al. Genetic and epigenetic fine mapping of causal autoimmune disease variants. Nature 518, 337-343, doi:10.1038/nature13835 (2015).
31 Zimber-Strobl, U. et al. Epstein-Barr virus nuclear antigen 2 exerts its transactivating function through interaction with recombination signal binding protein RBP-J kappa, the homologue of Drosophila Suppressor of Hairless. EMBO J 13, 4973-4982 (1994).
32 Grossman, S. R., Johannsen, E., Tong, X., Yalamanchili, R. & Kieff, E. The Epstein-Barr virus nuclear antigen 2 transactivator is directed to response elements by the J kappa recombination signal binding protein. Proc Natl Acad Sci USA 91, 7568-7572 (1994).
33 Henkel, T., Ling, P. D., Hayward, S. D. & Peterson, M. G. Mediation of Epstein-Barr virus EBNA2 transactivation by recombination signal-binding protein J kappa. Science 265, 92-95 (1994).
34 Scala, G. et al. Epstein-Barr virus nuclear antigen 2 transactivates the long terminal repeat of human immunodeficiency virus type 1. J Virol 67, 2853-2861 (1993).
35 Wang, J. H. et al. Aiolos regulates B cell activation and maturation to effector state. Immunity 9, 543-553 (1998).
36 Lu, F. et al. EBNA2 Drives Formation of New Chromosome Binding Sites and Target Genes for B-Cell Master Regulatory Transcription Factors RBP-jkappa and EBF1. PLoS Pathog 12, e1005339, doi:10.1371/journal.ppat.1005339 (2016).
37 Bailey, S. D., Virtanen, C., Haibe-Kains, B. & Lupien, M. ABC: a tool to identify SNVs causing allele-specific transcription factor binding from ChIP-Seq experiments. Bioinformatics 31, 3057-3059, doi:10.1093/bioinformatics/btv321 (2015).
38 Buchkovich, M. L. et al. Removing reference mapping biases using limited or no genotype data identifies allelic differences in protein binding at disease-associated loci. BMC medical genomics 8, 43, doi:10.1186/s12920-015-0117-x (2015).
39 Kumasaka, N., Knights, A. J. & Gaffney, D. J. Fine-mapping cellular QTLs with RASQUAL and ATAC-seq. Nat Genet 48, 206-213, doi:10.1038/ng.3467 (2016).
40 Shi, W., Fornes, O., Mathelier, A. & Wasserman, W. W. Evaluating the impact of single nucleotide variants on transcription factor binding. Nucleic Acids Res 44, 10106-10116, doi:10.1093/nar/gkw691 (2016).
41 Ma, B., Huang, J. & Liang, L. RTeQTL: Real-Time Online Engine for Expression Quantitative Trait Loci Analyses. Database: the journal of biological databases and curation 2014, doi:10.1093/database/bau066 (2014).
42 Kryworuckho, M., Diaz-Mitoma, F. & Kumar, A. CD44 isoforms containing exons V6 and V7 are differentially expressed on mitogenically stimulated normal and Epstein-Barr virus-transformed human B cells. Immunology 86, 41-48 (1995).
43 Gonnella, R. et al. PKC theta and p38 MAPK activate the EBV lytic cycle through autophagy induction. Biochim Biophys Acta 1853, 1586-1595, doi:10.1016/j.bbamcr.2015.03.011 (2015).
44 Ernst, J. et al. Mapping and analysis of chromatin state dynamics in nine human cell types. Nature 473, 43-49, doi:10.1038/nature09906 (2011).
45 Griffith, M. et al. DGIdb: mining the druggable genome. Nature methods 10, 1209-1210, doi:10.1038/nmeth.2689 (2013).
46 Harter, M. R. et al. BS69/ZMYND11 C-Terminal Domains Bind and Inhibit EBNA2. PLoS Pathog 12, e1005414, doi:10.1371/journal.ppat.1005414 (2016).
47 Trynka, G. et al. Dense genotyping identifies and localizes multiple common and rare variant association signals in celiac disease. Nat Genet 43, 1193-1201, doi:10.1038/ng.998 (2011).
48 Purcell, S. et al. PLINK: a tool set for whole-genome association and population-based linkage analyses. American journal of human genetics 81, 559-575, doi:10.1086/519795 (2007).
49 Consortium, E. P. An integrated encyclopedia of DNA elements in the human genome. Nature 489, 57-74, doi:10.1038/nature11247 (2012).
50 Roadmap Epigenomics, C. et al. Integrative analysis of 111 reference human epigenomes. Nature 518, 317-330, doi:10.1038/nature14248 (2015).
51 Liu, T. et al. Cistrome: an integrative platform for transcriptional regulation studies. Genome Biol 12, R83, doi:10.1186/gb-2011-12-8-r83 (2011).
52 Portales-Casamar, E. et al. The PAZAR database of gene regulatory information coupled to the ORCA toolkit for the study of regulatory sequences. Nucleic Acids Res 37, D54-60, doi:10.1093/nar/gkn783 (2009).
53 Griffon, A. et al. Integrative analysis of public ChIP-seq experiments reveals a complex multi-cell regulatory landscape. Nucleic Acids Res 43, e27, doi:10.1093/nar/gku1280 (2015).
54 Barrett, T. et al. NCBI GEO: archive for functional genomics data sets--update. Nucleic Acids Res 41, D991-995, doi:10.1093/nar/gks1193 (2013).
55 Consortium, G. T. Human genomics. The Genotype-Tissue Expression (GTEx) pilot analysis: multitissue gene regulation in humans. Science 348, 648-660, doi:10.1126/science.1262110 (2015).
56 Weirauch, M. T. et al. Determination and inference of eukaryotic transcription factor sequence specificity. Cell 158, 1431-1443, doi:10.1016/j.cell.2014.08.009 (2014).
57 Genomes Project, C. et al. A global reference for human genetic variation. Nature 526, 68-74, doi:10.1038/nature15393 (2015).
58 Smigielski, E. M., Sirotkin, K., Ward, M. & Sherry, S. T. dbSNP: a database of single nucleotide polymorphisms. Nucleic Acids Res 28, 352-355 (2000).
59 Kottyan, L. C. et al. Genome-wide association analysis of eosinophilic esophagitis provides insight into the tissue specificity of this allergic disease. Nat Genet 46, 895-900, doi:10.1038/ng.3033 (2014).
60 Verma, S. S. et al. Imputation and quality control steps for combining multiple genome-wide datasets. Frontiers in genetics 5, 370, doi:10.3389/fgene.2014.00370 (2014).
61 Langmead, B. & Salzberg, S. L. Fast gapped-read alignment with Bowtie 2. Nature methods 9, 357-359, doi:10.1038/nmeth.1923 (2012).
62 Trapnell, C., Pachter, L. & Salzberg, S. L. TopHat: discovering splice junctions with RNA-Seq. Bioinformatics 25, 1105-1111, doi:10.1093/bioinformatics/btp120 (2009).
63 Flicek, P. et al. Ensembl 2013. Nucleic Acids Res 41, D48-55, doi:10.1093/nar/gks1236 (2013).
64 Kim, D., Langmead, B. & Salzberg, S. L. HISAT: a fast spliced aligner with low memory requirements. Nature methods 12, 357-360, doi:10.1038/nmeth.3317 (2015).
65 Trapnell, C. et al. Transcript assembly and quantification by RNA-Seq reveals unannotated transcripts and isoform switching during cell differentiation. Nat Biotechnol 28, 511-515, doi:10.1038/nbt.1621 (2010).
66 Birkenbach, M., Josefsen, K., Yalamanchili, R., Lenoir, G. & Kieff, E. Epstein-Barr virus-induced genes: first lymphocyte-specific G protein-coupled peptide receptors. J Virol 67, 2209-2220 (1993).
67 Chen, C. C. et al. NF-kappaB-mediated transcriptional upregulation of TNFAIP2 by the Epstein-Barr virus oncoprotein, LMP1, promotes cell motility in nasopharyngeal carcinoma. Oncogene 33, 3648-3659, doi:10.1038/onc.2013.345 (2014).
68 Craig, F. E. et al. Gene expression profiling of Epstein-Barr virus-positive and -negative monomorphic B-cell posttransplant lymphoproliferative disorders. Diagn Mol Pathol 16, 158-168, doi:10.1097/PDM.0b013e31804f54a9 (2007).
69 Smith, N. et al. Induction of interferon-stimulated genes on the IL-4 response axis by Epstein-Barr virus infected human b cells; relevance to cellular transformation. PLoS One 8, e64868, doi: 10.1371/journal.pone.0064868 (2013).
70 Portis, T., Dyck, P. & Longnecker, R. Epstein-Barr Virus (EBV) LMP2A induces alterations in gene transcription similar to those observed in Reed-Sternberg cells of Hodgkin lymphoma. Blood 102, 4166-4178, doi:10.1182/blood-2003-04-1018 (2003).
71 Lee, I. S., Shin, Y. K., Chung, D. H. & Park, S. H. LMP1-induced downregulation of CD99 molecules in Hodgkin and Reed-Sternberg cells. Leuk Lymphoma 42, 587-594, doi:10.3109/10428190109099318 (2001).
72 Li, Y. et al. A genome-wide association study in Han Chinese identifies a susceptibility locus for primary Sjogren's syndrome at 7q11.23. Nat Genet 45, 1361-1365, doi:10.1038/ng.2779 (2013).
73 Okada, Y. et al. Genetics of rheumatoid arthritis contributes to biology and drug discovery. Nature 506, 376-381, doi:10.1038/nature12873 (2014).
74 International Multiple Sclerosis Genetics, C. et al. Genetic risk and a primary role for cell-mediated immune mechanisms in multiple sclerosis. Nature 476, 214-219, doi:10.1038/nature10251 (2011).
75 Mifsud, B. et al. Mapping long-range promoter contacts in human cells with high-resolution capture Hi-C. Nat Genet 47, 598-606, doi:10.1038/ng.3286 (2015).
76 Javierre, B. M. et al. Lineage-Specific Genome Architecture Links Enhancers and Non-coding Disease Variants to Target Gene Promoters. Cell 167, 1369-1384 e1319, doi:10.1016/j.cell.2016.09.037 (2016).
77 Liang, L. et al. A cross-platform analysis of 14,177 expression quantitative trait loci derived from lymphoblastoid cell lines. Genome Res 23, 716-726, doi:10.1101/gr.142521.112 (2013).
78 Stranger, B. E. et al. Population genomics of human gene expression. Nat Genet 39, 1217-1224, doi:10.1038/ng2142 (2007).
79 Veyrieras, J. B. et al. High-resolution mapping of expression-QTLs yields insight into human gene regulation. PLoS Genet 4, e1000214, doi:10.1371/journal.pgen.1000214 (2008).
80 Pickrell, J. K. et al. Understanding mechanisms underlying human gene expression variation with RNA sequencing. Nature 464, 768-772, doi:10.1038/nature08872 (2010).
81 Montgomery, S. B. et al. Transcriptome genetics using second generation sequencing in a Caucasian population. Nature 464, 773-777, doi:10.1038/nature08903 (2010).
82 Mangravite, L. M. et al. A statin-dependent QTL for GATM expression is associated with statin-induced myopathy. Nature 502, 377-380, doi:10.1038/nature12508 (2013).
83 Dimas, A. S. et al. Common regulatory variation impacts gene expression in a cell type-dependent manner. Science 325, 1246-1250, doi:10.1126/science.1174148 (2009).
84 Gaffney, D. J. et al. Dissecting the regulatory architecture of gene expression QTLs. Genome Biol 13, R7, doi:10.1186/gb-2012-13-1-r7 (2012).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a disease with a therapeutic agent, in an individual in need thereof.

2. The method of claim 1, wherein said disease is selected from one or more diseases or conditions listed in column B of Table 1, wherein said therapeutic agent is selected from an agent listed in column C of Table 1, and wherein said therapeutic agent is administered in an amount effective to modulate the transcription factor ESR1.

3. The method of claim 1, wherein said disease is selected from one or more diseases or conditions listed in column B of Table 2, wherein said therapeutic agent is selected from an agent listed in column C of Table 2, and wherein said therapeutic agent is administered in an amount effective to modulate the transcription factor ESR2.

4. The method of claim 1, wherein said disease is selected from one or more diseases or conditions listed in column B of Table 3, wherein said therapeutic agent is selected from an agent listed in column C of Table 3, and wherein said therapeutic agent is administered in an amount effective to modulate the transcription factor AR.

5. The method of claim 1, wherein said disease is selected from one or more diseases or conditions listed in column B of Table 4, wherein said therapeutic agent is selected from an agent listed in column C of Table 4, and wherein said therapeutic agent is administered in an amount effective to modulate the transcription factor PGR.

6. The method of claim 1, wherein said disease is selected from one or more diseases or conditions listed in column B of Table 5, wherein said therapeutic agent is selected from an agent listed in column C of Table 5, and wherein said therapeutic agent is administered in an amount effective to modulate the transcription factor HDAC2.

7. The method of claim 1, wherein said disease is selected from one or more diseases or conditions listed in column B of Table 6, wherein said therapeutic agent is selected from an agent listed in column C of Table 6, and wherein said therapeutic agent is administered in an amount effective to modulate the transcription factor NR3C1.

8. The method of claim 1, wherein said disease is selected from one or more diseases or conditions listed in column B of Table 7, wherein said therapeutic agent is selected from an agent listed in column C of Table 7, and wherein said therapeutic agent is administered in an amount effective to modulate the transcription factor VDR.

9. The method of claim 1, wherein said disease is selected from one or more diseases or conditions listed in column B of Table 8, wherein said therapeutic agent is selected from an agent listed in column C of Table 8, and wherein said therapeutic agent is administered in an amount effective to modulate the transcription factor RXRA.

10. The method of claim 1, wherein said disease is selected from one or more diseases or conditions listed in column B of Table 9, wherein said therapeutic agent is selected from an agent listed in column C of Table 9, and wherein said therapeutic agent is administered in an amount effective to modulate the transcription factor RARG.

11. The method of claim 1, wherein said disease is selected from one or more diseases or conditions listed in column B of Table 10, wherein said therapeutic agent is selected from an agent listed in column C of Table 10, and wherein said therapeutic agent is administered in an amount effective to modulate the transcription factor NFKB1.

12. The method of claim 1, wherein said disease is selected from one or more diseases or conditions listed in column B of Table 11, wherein said therapeutic agent is selected from an agent listed in column C of Table 11, and wherein said therapeutic agent is administered in an amount effective to modulate the transcription factor CHD1.

13. The method of claim 1, wherein said disease is selected from one or more diseases or conditions listed in column B of Table 12, wherein said therapeutic agent is selected from an agent listed in column C of Table 12, and wherein said therapeutic agent is administered in an amount effective to modulate the transcription factor NOTCH1.

14. The method of claim 1, wherein said disease is selected from one or more diseases or conditions listed in column B of Table 13, wherein said therapeutic agent is selected from an agent listed in column C of Table 13, and wherein said therapeutic agent is administered in an amount effective to modulate the transcription factor STAT5B.

15. The method of claim 1, wherein said disease is selected from one or more diseases or conditions listed in column B of Table 14, wherein said therapeutic agent is selected from an agent listed in column C of Table 14, and wherein said therapeutic agent is administered in an amount effective to modulate the transcription factor HDAC1.

16. The method of claim 1, wherein said disease is selected from one or more diseases or conditions listed in column B of Table 15, wherein said therapeutic agent is selected from an agent listed in column C of Table 15, and wherein said therapeutic agent is administered in an amount effective to modulate the transcription factor CDK9.

17. The method of claim 1, wherein said disease is selected from one or more diseases or conditions listed in column B of Table 16, wherein said therapeutic agent is selected from an agent listed in column C of Table 16, and wherein said therapeutic agent is administered in an amount effective to modulate the transcription factor HDAC6.

18. The method of claim 1, wherein said disease is selected from one or more diseases or conditions listed in column B of Table 17, wherein said therapeutic agent is selected from an agent listed in column C of Table 17, and wherein said therapeutic agent is administered in an amount effective to modulate the transcription factor JUN.

19. The method of claim 1, wherein said disease is selected from one or more diseases or conditions listed in column B of Table 18, wherein said therapeutic agent is selected from an agent listed in column C of Table 18, and wherein said therapeutic agent is administered in an amount effective to modulate the transcription factor HDAC8.

20. The method of claim 1, wherein said disease is selected from one or more diseases or conditions listed in column B of Table 19, wherein said therapeutic agent is selected from an agent listed in column C of Table 19, and wherein said therapeutic agent is administered in an amount effective to modulate the transcription factor EP300.

21. The method of claim 1, wherein said disease is selected from one or more diseases or conditions listed in column B of Table 20, wherein said therapeutic agent is selected from an agent listed in column C of Table 20, and wherein said therapeutic agent is administered in an amount effective to modulate the transcription factor MYC.

22. The method of claim 1, wherein said disease is selected from one or more diseases or conditions listed in column B of Table 21, wherein said therapeutic agent is selected from an agent listed in column C of Table 21, and wherein said therapeutic agent is administered in an amount effective to modulate the transcription factor BRD4.

23. The method of claim 1, wherein said disease is selected from one or more diseases or conditions listed in column B of Table 22, wherein said therapeutic agent is selected from an agent listed in column C of Table 22, and wherein said therapeutic agent is administered in an amount effective to modulate the transcription factor NFATC1.

24. The method of claim 1, wherein said disease is selected from one or more diseases or conditions listed in column B of Table 23, wherein said therapeutic agent is selected from an agent listed in column C of Table 23, and wherein said therapeutic agent is administered in an amount effective to modulate the transcription factor RUNX1.

25. The method of claim 1, wherein said disease is selected from one or more diseases or conditions listed in column B of Table 24, wherein said therapeutic agent is selected from an agent listed in column C of Table 24, and wherein said therapeutic agent is administered in an amount effective to modulate the transcription factor TCF7L2.

26. The method of claim 1, wherein said disease is selected from one or more diseases or conditions listed in column B of Table 25, wherein said therapeutic agent is selected from an agent listed in column C of Table 25, and wherein said therapeutic agent is administered in an amount effective to modulate the transcription factor PHF8.

27. The method of claim 1, wherein said disease is selected from one or more diseases or conditions listed in column B of Table 26, wherein said therapeutic agent is selected from an agent listed in column C of Table 26, and wherein said therapeutic agent is administered in an amount effective to modulate the transcription factor HNF4A.

28. The method of claim 1, wherein said disease is selected from one or more diseases or conditions listed in column B of Table 27, wherein said therapeutic agent is selected from an agent listed in column C of Table 27, and wherein said therapeutic agent is administered in an amount effective to modulate the transcription factor MED1.

29. The method of claim 1, wherein said disease is selected from one or more diseases or conditions listed in column B of Table 28, wherein said therapeutic agent is selected from an agent listed in column C of Table 28, and wherein said therapeutic agent is administered in an amount effective to modulate the transcription factor NFKB2.

30. The method of claim 1, wherein said disease is selected from one or more diseases or conditions listed in column B of Table 29, wherein said therapeutic agent is selected from an agent listed in column C of Table 29, and wherein said therapeutic agent is administered in an amount effective to modulate the transcription factor CREBBP.

31. The method of claim 1, wherein said disease is selected from one or more diseases or conditions listed in column B of Table 30, wherein said therapeutic agent is selected from an agent listed in column C of Table 30, and wherein said therapeutic agent is administered in an amount effective to modulate the transcription factor STAT3.

32. The method of claim 1, wherein said disease is selected from one or more diseases or conditions listed in column B of Table 31, wherein said therapeutic agent is selected from an agent listed in column C of Table 31, and wherein said therapeutic agent is administered in an amount effective to modulate the transcription factor SMARCA4.

33. The method of claim 1, wherein said disease is selected from one or more diseases or conditions listed in column B of Table 32, wherein said therapeutic agent is selected from an agent listed in column C of Table 32, and wherein said therapeutic agent is administered in an amount effective to modulate the transcription factor BRD2.

34. The method of claim 1, wherein said disease is selected from one or more diseases or conditions listed in column B of Table 33, wherein said therapeutic agent is selected from an agent listed in column C of Table 33, and wherein said therapeutic agent is administered in an amount effective to modulate the transcription factor STAT4.

35. The method of claim 1, wherein said disease is selected from one or more diseases or conditions listed in column B of Table 34, wherein said therapeutic agent is selected from an agent listed in column C of Table 34 and wherein said therapeutic agent is administered in an amount effective to modulate the transcription factor KDM5B.

36. The method of claim 1, wherein said disease is selected from one or more diseases or conditions listed in column B of Table 35, wherein said therapeutic agent is selected from an agent listed in column C of Table 35, and wherein said therapeutic agent is administered in an amount effective to modulate the transcription factor BRD3.

37. The method of claim 1, wherein said disease is selected from one or more diseases or conditions listed in column B of Table 36, wherein said therapeutic agent is selected from an agent listed in column C of Table 36, and wherein said therapeutic agent is administered in an amount effective to modulate the transcription factor EZH2.

38. The method of claim 1, wherein said disease is selected from one or more diseases or conditions listed in column B of Table 37, wherein said therapeutic agent is selected from an agent listed in column C of Table 37, and wherein said therapeutic agent is administered in an amount effective to modulate the transcription factor ATF1.

39. The method of claim 1, wherein said disease is selected from one or more diseases or conditions listed in column B of Table 38, wherein said therapeutic agent is selected from an agent listed in column C of Table 38, and wherein said therapeutic agent is administered in an amount effective to modulate the transcription factor CREB1.

40. The method of claim 1, wherein said disease is selected from one or more diseases or conditions listed in column B of Table 39, wherein said therapeutic agent is selected from an agent listed in column C of Table 39, and wherein said therapeutic agent is administered in an amount effective to modulate the transcription factor TP53.

41. The method of claim 1, wherein said disease is selected from one or more diseases or conditions listed in column B of Table 40, wherein said therapeutic agent is selected from an agent listed in column C of Table 40, and wherein said therapeutic agent is administered in an amount effective to modulate the transcription factor HNF4G.

42. The method of claim 1, wherein said disease is selected from one or more diseases or conditions listed in column B of Table 41, wherein said therapeutic agent is selected from an agent listed in column C of Table 41, and wherein said therapeutic agent is administered in an amount effective to modulate the transcription factor NR2C2.

43. The method of claim 1, wherein said disease is selected from one or more diseases or conditions listed in column B of Table 42, wherein said therapeutic agent is selected from an agent listed in column C of Table 42, and wherein said therapeutic agent is administered in an amount effective to modulate the transcription factor SIRT6.

44. The method of claim 1, wherein said disease is selected from one or more diseases or conditions listed in column B of Table 43, wherein said therapeutic agent is selected from an agent listed in column C of Table 43 and wherein said therapeutic agent is administered in an amount effective to modulate the transcription factor BRCA1.

45. The method of claim 1, wherein said disease is selected from one or more diseases or conditions listed in column B of Table 44, wherein said therapeutic agent is selected from an agent listed in column C of Table 44, and wherein said therapeutic agent is administered in an amount effective to modulate the transcription factor NR1H2.

46. The method of claim 1, wherein said disease is selected from one or more diseases or conditions listed in column B of Table 45, wherein said therapeutic agent is selected from an agent listed in column C of Table 45, and wherein said therapeutic agent is administered in an amount effective to modulate the transcription factor KAT5.

47. The method of claim 1, wherein said disease is selected from one or more diseases or conditions listed in column B of Table 46, wherein said therapeutic agent is selected from an agent listed in column C of Table 46 and wherein said therapeutic agent is administered in an amount effective to modulate the transcription factor CTNNB1.

48. The method of claim 1, wherein said disease is selected from one or more diseases or conditions listed in column B of Table 47, wherein said therapeutic agent is selected from an agent listed in column C of Table 47, and wherein said therapeutic agent is administered in an amount effective to modulate the transcription factor KDM5A.

49. The method of claim 1, wherein said disease is selected from one or more diseases or conditions listed in column B of Table 48, wherein said therapeutic agent is selected from an agent listed in column C of Table 48, and wherein said therapeutic agent is administered in an amount effective to modulate the transcription factor PPARG.

50. The method of claim 1, wherein said disease is selected from one or more diseases or conditions listed in column B of Table 49, wherein said therapeutic agent is selected from an agent listed in column C of Table 49, and wherein said therapeutic agent is administered in an amount effective to modulate the transcription factor ZEB1.

51. A method of treating a disease comprising the step of identifying one or more, or two or more, or three or more, or four or more, or five or more, or six or more, or seven or more, or eight or more, or nine or more, or ten or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or more than 40 loci associated with said disease in an individual suspected of having or having said disease, and treating said individual with a compound that modulates a TF associated with said one or more loci.
